(19) United States
(12) Patent Application Publication
(10) Pub. No.: US 2023/0083639 A1
(43) Pub. Date: Mar. 16, 2023

(54) SYSTEMS, DEVICES AND METHODS FOR PERFORMING MEDICAL PROCEDURES IN THE INTESTINE

(71) Applicant: Fractyl Health, Inc., Lexington, MA (US)

(72) Inventors: Jay D. Caplan, Boston, MA (US); Thomas C. Kochem, Watertown, MA (US); Sara Morneau, Lincoln, MA (US); Michael Biasella, Medford, MA (US); R. Maxwell Flaherty, Topsfield, MA (US); J. Christopher Flaherty, Nottingham, NH (US)

(73) Assignee: Fractyl Health, Inc., Lexington, MA (US)

(21) Appl. No.: 17/721,937

(22) Filed: Apr. 15, 2022

Related U.S. Application Data
(63) Continuation of application No. PCT/US20/56627, filed on Oct. 21, 2020.
(60) Provisional application No. 62/923,710, filed on Oct. 21, 2019.
(60) Provisional application No. 63/067,159, filed on Aug. 18, 2020.

(30) Foreign Application Priority Data
Oct. 21, 2019 (US) 62923710
Aug. 18, 2020 (US) 63067159

(51) Int. Cl. A61B 18/14

(52) U.S. Cl. CPC A61B 18/1492; A61B 2018/00577

(57) ABSTRACT

A system for performing a medical procedure on mucosal tissue in an intestine of a patient is provided. The system including a catheter a catheter for insertion into the intestine, and a console. The catheter comprises: a shaft including a distal portion, and a functional assembly on the distal portion of the shaft. The functional assembly is configured to receive fluid. The console comprises: at least one pumping assembly configured to deliver the fluid to the functional assembly; and a connector configured to operably attach the catheter to the console. The functional assembly is configured to treat target tissue of the intestine of the patient. Methods of treating intestinal mucosal tissue are also described.

### RELATED APPLICATIONS

This application is a continuation of PCT Application No. PCT/US2020/056627 (Attorney Docket No. 41714-720.601), filed Oct. 21, 2020; which claims the benefit of: U.S. Provisional Patent Application Ser. No. 62/923,710 (Attorney Docket No. 41714-720.101; Client Docket No. MCT-050-PR1), entitled “Systems, Devices, and Methods for Performing Medical Procedures in the Intestine”, filed Oct. 21, 2019; and U.S. Provisional Patent Application Ser. No. 63/067,159 (Attorney Docket No. 41714-720.102; Client Docket No. MCT-050-PR2), entitled “Systems, Devices, and Methods for Performing Medical Procedures in the Intestine”, filed Aug. 18, 2020; the contents of which are each incorporated herein by reference in their entirety.

This application is related to: U.S. patent application Ser. No. 13/945,138 (Attorney Docket No. 41714-703.301; Client Docket No. MCT-001-US), entitled “Devices and Methods for the Treatment of Tissue”, filed Jul. 18, 2013; U.S. patent application Ser. No. 15/917,480 (Attorney Docket No. 41714-703.302; Client Docket No. MCT-001-US-CON1), entitled “Devices and Methods for the Treatment of Tissue”, filed Mar. 9, 2018; U.S. patent application Ser. No. 16/438,362 (Attorney Docket No. 41714-704.302; Client Docket No. MCT-002-US-CON1), entitled “Heat Ablation Systems, Devices and Methods for the Treatment of Tissue”, filed Jun. 11, 2019; U.S. patent application Ser. No. 14/515,324 (Attorney Docket No. 41714-705.301; Client Docket No. MCT-003-US), entitled “Tissue Expansion Devices, Systems and Methods”, filed Oct. 15, 2014; U.S. patent application Ser. No. 16/711,236 (Attorney Docket No. 41714-706.302; Client Docket No. MCT-004-US-CON1), entitled “Electrical Energy Ablation Systems, Devices and Methods for the Treatment of Tissue”, filed Dec. 11, 2019; U.S. patent application Ser. No. 14/609,334 (Attorney Docket No. 41714-707.301; Client Docket No. MCT-005-US), entitled “Ablation Systems, Devices, and Methods for the Treatment of Tissue”, filed Jan. 29, 2015; U.S. patent application Ser. No. 14/673,565 (Attorney Docket No. 41714-708.301; Client Docket No. MCT-009-US), entitled “Methods, Systems and Devices for Performing Multiple Treatments on a Patient”, filed Mar. 30, 2015; U.S. patent application Ser. No. 16/379,554 (Attorney Docket No. 41714-709.302; Client Docket No. MCT-013-US-CON1), entitled “Methods, Systems and Devices for Reducing the Luminal Surface Area of the Gastrointestinal Tract”, filed Apr. 9, 2019; U.S. patent application Ser. No. 14/917,243 (Attorney Docket No. 41714-710.301; Client Docket No. MCT-023-US), entitled “Systems, Methods and Devices for Treatment of Target Tissue”, filed Mar. 7, 2016; U.S. patent application Ser. No. 16/267,771 (Attorney Docket No. 41714-711.302; Client Docket No. MCT-024-US-CON1), entitled “Systems, Devices and Methods for the Creation of a Therapeutic Restriction in the Gastrointestinal Tract”, filed Feb. 5, 2019; U.S. patent application Ser. No. 16/742,645 (Attorney Docket No. 41714-715.301; Client Docket No. MCT-025-US), entitled “Intestinal Catheter Device and System”, filed Jan. 14, 2020; U.S. patent application Ser. No. 16/900,563 (Attorney Docket No. 41714-712.501; Client Docket No. MCT-027-US-CIP1), entitled “Injectate Delivery Devices, Systems and Methods”, filed Jun. 12, 2020; U.S. patent application Ser. No. 16/900,563 (Attorney Docket No. 41714-712.501; Client Docket No. MCT-027-US-CIP1), entitled “Injectate Delivery Devices, Systems and Methods”, filed Jun. 12, 2020; U.S. patent application Ser. No. 16/798,117 (Attorney Docket No. 41714-714.303; Client Docket No. MCT-028-US-CIP1-CON2), entitled “Systems, Devices and Methods for Performing Medical Procedures in the Intestine”, filed Feb. 21, 2020; U.S. patent application Ser. No. 15/812,969 (Attorney Docket No. 41714-714.302; Client Docket No. MCT-028-US-CIP2-CON1), entitled “Systems, Devices and Methods for Performing Medical Procedures in the Intestine”, filed Nov. 14, 2017; U.S. patent application Ser. No. 15/406,572 (Attorney Docket No. 41714-713.301; Client Docket No. MCT-029-US), entitled “Methods and Systems for Treating Diabetes and Related Diseases and Disorders”, filed Jan. 13, 2017; U.S. patent application Ser. No. 16/400,491 (Attorney Docket No. 41714-716.301; Client Docket No. MCT-035-US), entitled “Systems, Devices and Methods for Performing Medical Procedures in the Intestine”, filed May 1, 2019; U.S. patent application Ser. No. 16/905,274 (Attorney Docket No. 41714-717.301; Client Docket No. MCT-036-US), entitled “Material Depositing System for Treating a Patient”, filed Jun. 18, 2020; International PCT Patent Application Serial Number PCT/US2019/54088 (Attorney Docket No. 41714-718.301; Client Docket No. MCT-037-PCT), entitled “Systems and Methods for Deposition Material in a Patient”, filed Oct. 1, 2019; International PCT Patent Application Serial Number PCT/US2020/025925 (Attorney Docket No. 41714-719.601; Client Docket No. MCT-040-PCT), entitled “Systems, Devices and Methods for Treating Metabolic Medical Conditions”, filed Mar. 31, 2020; and U.S. Provisional Patent Application Ser. No. 62/960,340 (Attorney Docket No. 41714-721.101; Client Jan. 13, 2020; U.S. Provisional Patent Application Ser. No. 62/961,340 (Attorney Docket No. 41714-722.101; Client Docket No. MCT-051-PR1), entitled “Automated Tissue Treatment Devices, Systems, and Methods”, filed Jan. 15, 2020; United States Client Docket No. MCT-041-PR1), entitled “Systems, Devices and Methods for Treating Diabetes”, filed Mar. 18, 2020; U.S. Provisional Patent Application Ser. No. 63/042,356 (Attorney Docket No. 41714-724.101; Client Docket No. MCT-034-PR1), entitled “Tissue Treatment System with Fluid Delivery Console”, filed Jun. 22, 2020; U.S. Provisional Patent Application Ser. No. 63/076,737 (Attorney Docket No. 41714-723.102; Client Docket No. MCT-041-PR2), entitled “Systems, Devices and Methods for Treating Diabetes”, filed Sep. 10, 2020; U.S. Provisional Patent Application Ser. No. 63/085,375 (Attorney Docket No. 41714-723.103; Client Docket No. MCT-041-PR3), entitled “Systems, Devices and Methods for Treating Diabetes”, filed Sep. 30, 2020; the contents of each of which is incorporated herein by reference in its entirety for all purposes.

### TECHNICAL FIELD

The embodiments disclosed herein relate generally to systems, devices and methods for performing medical procedures in the intestine of a patient.

### BACKGROUND OF THE INVENTION

Numerous diagnostic and therapeutic procedures are performed in the small and large intestine, as well as other locations of the gastrointestinal tract. Devices used in these procedures can be difficult to maneuver and otherwise operate, and often have limited functionality. There is a need for improved systems and devices for treating and diagnosing tissue of the intestine, as well as a need for methods of treating intestinal tissue as a new or improved therapy for various diseases and disorders.

### SUMMARY

According to an aspect of the present inventive concepts, a system for performing a medical procedure in the intestine of a patient, the system comprising: a catheter for insertion into the intestine, the catheter comprising: a shaft including a distal portion, and a functional assembly on the distal portion of the shaft, and the functional assembly is configured to receive fluid; and a console comprising: at least one pumping assembly configured to deliver the fluid to the functional assembly; and a connector configured to operably attach the catheter to the console. The functional assembly is configured to treat target tissue of the intestine of the patient.

In some embodiments, the target tissue treated comprises at least a relatively continuous portion of intestinal surface tissue.

In some embodiments, the target tissue treated comprises multiple discrete portions of intestinal surface tissue.

In some embodiments, the functional assembly is configured to radially expand and/or contract. The functional assembly can comprise one or more tissue contacting portions configured to contact tissue of the intestine when expanded. The functional assembly can be configured to treat tissue. The functional assembly can be configured to expand tissue.

In some embodiments, the functional assembly comprises one or more fluid delivery elements configured to deliver fluid into and/or onto tissue. The fluid delivery elements can deliver an expansion fluid to expand the tissue. The fluid delivery elements can deliver an ablative fluid to ablate the tissue. The fluid delivery elements can deliver a neutralizing fluid to reduce trauma to the tissue. The neutralizing fluid can be configured to limit the volume of tissue that is treated. The fluid delivery elements can be equally distributed about the functional assembly to provide a circumferential delivery of fluid into tissue. The functional assembly can further include one or more ports within which the fluid delivery elements are advanced. The functional assembly can be configured to expand tissue by delivering injectate into the tissue via the fluid delivery elements. The functional assembly can deliver injectate at a varied flow rate in an open loop or closed loop configuration. The system can monitor one or more pressures achieved during the delivery of injectate into tissue via a sensor. The system can regulate the delivery of the injectate based on the pressure measured by the sensor. The at least one pumping assembly can comprise one, two, or more syringe pumps each operably attached to a syringe comprising the fluid, and the syringes can be operably attached the fluid delivery elements. Each syringe pump can comprise a distal and/or proximal sensor configured to ensure a full injection stroke during the delivery of fluid into tissue via the fluid delivery elements. Each syringe pump can comprise a homing sensor configured to recognize a starting position of the syringe pump and a finish sensor configured to recognize a completion position of the syringe pump following a full injection stroke. Each syringe can automatically refill with fluid following the delivery of fluid into tissue via the fluid delivery elements. Each syringe pump can be configured to operate at a maximum speed if the pressure of the fluid is maintained below a threshold. The pressure threshold can be no more than 110 psi. Each syringe pump can be configured to operate at a speed less than the maximum speed if the pressure is above the threshold. The system can monitor one or more parameters of each of the syringe pumps during the delivery of fluid into tissue via the fluid delivery elements. The system can alert when a syringe pump generates a pressure that is at least 10% greater than a threshold. The system can alert when a component of the syringe pump does not translate for at least 3 seconds. The system can alert when a syringe pump generates a pressure that is lower than an expected pressure. The system can reduce performance when a syringe pump parameter is not within a normal range. Each fluid delivery element can be operably attached to a unique syringe pump. Two or more fluid delivery elements can be operably attached to the same syringe pump. At least two syringes can comprise dissimilar fluids. At least two syringes can comprise similar fluids.

In some embodiments, the system further comprises one or more sensors configured to produce a signal. The sensors can produce a signal related to a parameter of the system. The sensors can produce a signal related to a parameter of the patient. The patient parameter can comprise a parameter selected from the group consisting of: a parameter of the intestine; a parameter related to the anatomical geometry of a portion of the intestine; a parameter related to force and/or pressure applied to tissue of the intestine; a parameter related to a pressure within tissue of the intestine; a parameter related to temperature of tissue of the intestine; and combinations thereof. The one or more sensors can comprise a camera configured to provide an image, and the signal provided by the sensors can comprise the image or an analysis of the image. The camera can provide an image of the target tissue being treated, and the system can be configured to modify the target tissue treatment based on the image and/or analysis provided. The camera can provide an image prior to, during and/or after a tissue expansion procedure and/or the target tissue treatment. The system can be configured to modify the treatment of the target tissue based on signals produced by the one or more sensors. The system can further comprise a user interface configured to allow an operator to modify one or more operating parameters of the console and/or the catheter based on information provided via signals produced by the sensors. The console can further comprise one or more algorithms configured to adjust one or more operating parameters of the system based on information provided via signals produced by the sensors. The algorithm can be configured to adjust the temperature of fluid within one or more reservoirs of the console. The system can comprise a neutralizing fluid, and the algorithm can be configured to adjust an operating parameter of the system based on the temperature of the neutralizing fluid. The algorithm can be configured to determine an expanded size of the functional element and bias the expanded size toward a diameter that is smaller than the diameter of the intestine at the location of the target tissue. The sensors can produce a signal related to a tissue expansion parameter, and the system can be configured to assess the tissue expansion quality and/or quantity based on the signal. The sensors can produce a signal related to a tissue expansion parameter selected from the group consisting of: color, density and/or saturation related to injected dye or particles which alter tissue appearance; temperature of tissue; texture, length and/or diameter of villi or mucosal features, such as spacing between villi or other intestinal tissue features that can change due to tissue expansion; electrical resistance, impedance and/or capacitance of tissue; pressure and/or force of peristaltic contractions; compliance of tissue and/or the entire duodenum in radial and/or axial directions; chemical composition of film adhered to mucosa; types, quantities and/or locations of bacterial colonies present; and combinations thereof. The system can be configured to determine the quality of the target tissue treatment based on signals produced by the sensors. The target tissue treatment can comprise the treating of at least two segments of intestinal tissue, and the system can be configured to modify the parameters of a subsequent treatment of a second segment of intestinal tissue based on signals produced by the sensors during a prior treatment of a first segment of intestinal tissue.

In some embodiments, the system further comprises at least one reservoir, and each pumping assembly is attached to a reservoir via one or more conduits. Each reservoir can store and supply fluid to the catheter via a pumping assembly. Each reservoir can extract and store fluid from the catheter via a pumping assembly. Each reservoir can store an ablative fluid, a neutralizing fluid, an agent, and/or an injectate to be supplied and/or extracted from the catheter via a pumping assembly. A first reservoir can comprise an ablative fluid at a temperature above 44° C. and a second reservoir can comprise a neutralizing fluid at a temperature below 37° C. At least two pumping assemblies can be configured to operate simultaneously during a drawdown procedure of the functional assembly, and the drawdown procedure can be configured to perform a radial contraction of the functional assembly that can be initiated when a leak within the system is detected. During the drawdown procedure a first pumping assembly can be configured to extract ablative fluid from the functional assembly via a first conduit and a second pumping assembly can be configured to simultaneously deliver a neutralizing fluid to the functional assembly via a second conduit. The console can further include a sensor configured to monitor one or more parameters of a pumping assembly and/or a reservoir. The console can be configured to disable the at least one pumping assembly when an undesired condition of a pumping assembly and/or a reservoir is detected by the sensor. The at least one reservoir can comprise a first reservoir including a neutralizing fluid and a second reservoir comprising an ablative fluid. The at least one pumping assembly can provide fluid to radially expand the functional assembly and/or extracts fluid to radially compact the functional assembly. The at least one pumping assembly can radially compact the functional assembly prior to the removal of the catheter from within the patient.

In some embodiments, the console further comprises one or more settings that can be changed manually via an operator and/or automatically via the system. The console settings can comprise one or more parameters of the catheter, console, and/or other component of the system. The console settings can comprise one or more parameters selected from the group consisting of: delivery rate of fluid into the functional assembly; withdrawal rate of fluid from the functional assembly; delivery rate of fluid into tissue; rate of energy delivered into tissue; peak energy level delivered into tissue; average energy delivery rate delivered into tissue; amount of energy delivered into tissue during a time period; temperature of an ablative fluid; temperature of a neutralizing fluid; temperature of the functional assembly; pressure of the functional assembly; pressure of fluid delivered into the functional assembly; pressure of fluid delivered into tissue; duration of energy delivery; time of energy delivery; translation rate of the functional assembly; rotation rate of the functional assembly; a flow rate; a recirculation rate; a heating rate or temperature; a cooling rate or temperature; a sampling rate of a sensor; and combinations thereof. The console can comprise an algorithm and/or a controller, and the console can modify a target tissue treatment parameter via the algorithm and/or the controller.

In some embodiments, the connector comprises a tubing set comprising multiple lumens. The console can further comprise a heating element and a cooling element, and the heating element can be configured to heat fluid within the connecting assembly, and the cooling element can be configured to cool fluid within the connector. The tubing set can be reusable. The tubing set can be disposable.

In some embodiments, the system is configured to modify the target tissue treatment by modifying the volume of fluid within the functional assembly and/or by modifying the mixing of fluid within the functional assembly.

In some embodiments, the functional assembly further comprises a baffle configured to improve fluid mixing and/or occupy a volume within the functional assembly.

In some embodiments, the catheter further comprises multiple conduits in fluid communication with the functional assembly. A first conduit can comprise an inflow tube configured to deliver fluid to the functional assembly and a second conduit can comprise an outflow tube configured to extract fluid from the functional assembly.

In some embodiments, the system is configured to treat the target tissue by delivering fluid to the functional assembly at a flow rate of at least 500 ml/min, at least 1000 ml/min, at least 2000 ml/min, or at least 2500 ml/min.

In some embodiments, the system is configured to treat the target tissue based on one or more parameters determined during a luminal sizing measurement of the intestine. The luminal sizing measurement can be performed after an expansion of tissue in the intestine. The console can comprise an algorithm, and the luminal sizing measurement can be determined by the algorithm.

In some embodiments, the functional assembly further comprises one or more ports configured to extract fluid from a segment of the intestine, and extraction of the fluid collapses the inner wall of the intestine onto the functional assembly. The collapsing of the intestine onto the functional assembly can be configured to modify the treatment of the target tissue.

In some embodiments, the system further comprises a desufflation and/or aspiration tool that extracts fluid from the intestine, and extraction of the fluid collapses the inner wall of the intestine onto the functional assembly. The collapsing of the intestine onto the functional assembly can be configured to modify the treatment of the target tissue.

In some embodiments, one, two, or more components of the system are insulated to protect an operator from contacting a surface that increases in temperature during use.

In some embodiments, the system further comprises an endoscope including a working channel, and the catheter is configured to be slidingly inserted through the working channel such that the functional assembly can be positioned proximate the target tissue to be treated.

In some embodiments, the catheter shaft comprises a variable stiffness along its length. A proximal portion of the catheter shaft can comprise a stiffness greater than a distal portion of the catheter shaft.

In some embodiments, the catheter shaft comprises a lumen through which a stiffening wire can be advanced to increase the stiffness of the catheter shaft.

In some embodiments, the system further comprises a guidewire, and the catheter is configured to be advanced over the guidewire such that the functional assembly can be positioned proximate the target tissue to be treated. The catheter can be configured to be advanced over the guidewire without the use of an endoscope.

In some embodiments, the functional assembly is configured to receive ablative fluid to ablate target tissue and neutralizing fluid to cool tissue, and the duration of ablation and/or the duration of cooling is based on the temperature of the ablative fluid and/or the neutralizing fluid.

In some embodiments, the system is configured to treat the target tissue with an ablative fluid, and to deliver a neutralizing fluid prior to, during, and/or after the treatment with the ablative fluid, and the delivery of the neutralizing fluid is configured to limit ablation of non-target tissue.

In some embodiments, the target tissue treatment comprises: positioning the functional assembly proximate target tissue; delivering a first cooling fluid to the functional assembly for a first period of time; and subsequently delivering an ablative fluid to the functional assembly for a second period of time. The target tissue treatment can further comprise delivering a second cooling fluid to the functional assembly for a third period of time after the delivery of ablative fluid. The second cooling fluid can be configured to neutralize the effects of the ablative fluid. The second cooling fluid can be configured to prevent ablation of non-target tissue. The target tissue treatment can comprise treating multiple target tissue locations, and the second cooling fluid can be delivered after the delivery of the ablative fluid at each target tissue portion. The target tissue treatment can comprise treating multiple target tissue locations, and the second cooling fluid can be delivered after the delivery of ablative fluid at all target tissue portions.

In some embodiments, the system comprises a single catheter with a functional assembly of a pre-determined expanded diameter. The pre-determined expanded diameter can comprise a diameter between 19 mm and 32 mm. The system can be configured to perform a tissue expansion procedure prior to the functional assembly treating the target tissue. The system can be configured to aspirate fluid from the intestine proximate the target tissue prior to the functional assembly treating the target tissue.

In some embodiments, the catheter is configured to perform a tissue expansion procedure and a tissue ablation procedure in a co-registered arrangement. The system can be configured to aspirate fluid from the intestinal lumen of the patient at a location proximate the target tissue.

In some embodiments, the catheter comprises a shaft with a length sufficient to take a long path through the stomach and reach the distal duodenum and/or proximal jejunum, and the shaft length is insufficient to reach the distal jejunum.

In some embodiments, the catheter comprises a distal portion with sufficient flexibility to perform a function selected from the group consisting of: prevent undesired forces on the luminal wall of the small intestine; prevent undesired forces on outside bends of the luminal wall of the small intestine; enhance tracking within the small intestine; improve circumferential apposition of the functional assembly with the luminal wall of the small intestine; improve apposition of the functional assembly with an inside bend of the small intestine; and combinations thereof.

In some embodiments, the catheter comprises three fluid delivery elements positioned 120° apart along a circumference of the functional assembly, and the three fluid delivery elements are configured to deliver fluid to submucosal tissue to cause circumferential expansion of the submucosal tissue.

In some embodiments, the functional assembly comprises at least one tissue capture port configured to capture tissue into which tissue-expanding fluid is to be delivered, and the depth of the layer of tissue to be expanded is based on the geometry of the at least one tissue capture port.

In some embodiments, the functional assembly comprises at least one tissue capture port configured to capture tissue into which tissue-expanding fluid is to be delivered when a vacuum is applied to the at least one tissue capture port, and the depth of the layer of tissue to be expanded is based on the level of the vacuum applied to the at least one tissue capture port.

In some embodiments, the system is configured to provide enough mixing of fluid within the functional assembly to achieve precise uniformity of depth of tissue ablation.

In some embodiments, the system is configured to provide ablative fluid comprising steam to the functional assembly, and the system is further configured to provide enough mixing of the steam within the functional assembly to achieve precise uniformity of depth of tissue ablation.

In some embodiments, the system is configured to perform the treatment of the target tissue without the use of aspiration. The system can be configured to control contact of the functional assembly with luminal wall tissue by controlling the volume of fluid within the functional assembly and/or the pressure of the fluid within the functional assembly.

In some embodiments, the system comprises an algorithm configured to control the duration of ablative fluids within the functional assembly. The algorithm can control the duration with a minimum duration of 500 msec and/or a maximum duration of 12 seconds.

In some embodiments, the system is configured to initiate the treatment of a second axial segment of tissue after a recovery period has elapsed since the completion of treatment of a previous treatment of a first axial segment. The recovery period can comprise a time period of no more than 30 minutes, no more than 20 minutes, and/or no more than 15 minutes.

In some embodiments, the system comprises a user interface configured to require successful performance of a first step prior to providing access to a second step.

In some embodiments, the system is configured to monitor performance and detect faults.

In some embodiments, the system comprises a system clock configured to record a temporal variable of the system. The temporal variable can comprise the duration since the performance of a tissue expansion procedure. The system can be configured to prevent the delivery of energy to tissue if the duration exceeds a threshold. The threshold can comprise a maximum time of 45 minutes, 30 minutes, 15 minutes, 10 minutes, and/or 5 minutes.

In some embodiments, the system comprises an energy delivery counter. The system can be configured to disable energy delivery after a threshold of energy deliveries is reached. The threshold can comprise a maximum of 30 energy deliveries, 20 energy deliveries, and/or 15 energy deliveries. The system can be configured to provide an alert if a threshold of energy deliveries has not been performed and the procedure is being completed. The threshold can comprise a minimum of 2, 3, 4, and/or 5 energy deliveries.

In some embodiments, the system further comprises a positioning assembly configured to monitor the position of at least the functional assembly. The system can be configured to prevent the delivery of energy to tissue if the functional assembly has moved above a threshold distance since the performance of a tissue expansion procedure. The threshold distance can comprise a distance of at least 2 mm, 5 mm, and/or 10 mm.

In some embodiments, the system comprises an algorithm configured to reduce ablation of non-target tissue. The system can comprise one or more sensors configured to produce signals, and the algorithm can be configured to analyze the sensor signals, and if an undesired ablation state can be identified via the analysis, modify the delivery of energy. The undesired ablation state can comprise an undesired state of the system and/or an undesired state at a treatment location.

In some embodiments, the system is configured to perform multiple sequential ablations without the need to reverse any adverse condition of the functional assembly. The system can be configured to perform the multiple sequential ablations without the need to remove the catheter from the patient.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of a system for performing a medical procedure in the intestine of a patient, consistent with the present inventive concepts.

FIG. 2 is a schematic view of another system for performing a medical procedure on the small intestine of a patient, consistent with the present inventive concepts.

FIGS. 3A-3D are camera views of a series of steps for expanding tissue and treating target tissue at a single axial segment of intestine, consistent with the present inventive concepts.

FIG. 4 is a method of performing a medical procedure including performing a tissue expansion with a functional assembly, and subsequently treating target tissue with the same or a different functional assembly, consistent with the present inventive concepts.

FIG. 5 is a method of performing a tissue treatment that includes activating a functional assembly based on an image, consistent with the present inventive concepts.

FIG. 6 is a method of marking tissue and performing a tissue treatment based on the tissue marking, consistent with the present inventive concepts.

FIG. 7 is a sectional view of the distal portion of a system including an endoscope and a treatment device inserted into a duodenum of a patient, consistent with the present inventive concepts.

FIG. 8 is a flow chart of a method of treating a patient, consistent with the present inventive concepts.

FIG. 9 is a flow chart of a method of preparing a treatment device, consistent with the present inventive concepts.

FIG. 10 is a flow chart of a method of expanding tissue with a treatment device, consistent with the present inventive concepts.

FIG. 11 is a flow chart of a method of ablating or otherwise treating tissue with a treatment device, consistent with the present inventive concepts.

FIG. 12 is a schematic view of a console operably attached to a connecting assembly and a treatment device, consistent with the present inventive concepts.

FIGS. 12A-12E are schematic views of various operational states of the console, consistent with the present inventive concepts.

FIG. 12F is a flow chart of a method of performing a disinfecting procedure for the console and the connecting assembly, consistent with the present inventive concepts.

FIGS. 13A-13C are schematic views of various operational states of the console, consistent with the present inventive concepts.

FIG. 13D is a flow chart of a method for filling conduits and components of the console with fluid, consistent with the present inventive concepts.

FIGS. 14A-14G are schematic views of various procedural operational states of the console, consistent with the present inventive concepts.

FIG. 14H is a flow chart of a method of treating a patient, consistent with the present inventive concepts.

FIG. 15 is a chart of tissue temperature during a treatment procedure, consistent with the present inventive concepts.

FIGS. 16 and 16A-16D are perspective, top, side, side sectional, and sectional views of an embodiment of a port of a treatment device, respectively, consistent with the present inventive concepts.

FIGS. 17A-17C are side sectional views of an embodiment of a port of a treatment device, consistent with the present inventive concepts.

FIGS. 18 and 18A-18D are a side sectional view and cross sectional views, respectively, of an embodiment of a distal portion of a treatment device, consistent with the present inventive concepts.

FIG. 19 is a schematic view of another embodiment of a system for performing a medical procedure in the intestine of a patient, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein.

As used herein, the singular forms “a,” “an” and “the” are intended to include the plural forms as well, unless the context clearly indicates otherwise.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms “first”, “second”, “third”, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terms “attachment”, “attached”, “attaching”, “connection”, “connected”, “connecting” and the like, where used herein, are to be taken to include any type of connection between two or more components. The connection can include an “operable connection” or “operable attachment” which allows multiple connected components to operate together such as to transfer information, power, and/or material (e.g. an agent to be delivered) between the components. An operable connection can include a physical connection, such as a physical connection including a connection between two or more: wires or other conductors (e.g. an “electrical connection”), optical fibers, wave guides, tubes such as fluid transport tubes, and/or linkages such as translatable rods or other mechanical linkages. Alternatively or additionally, an operable connection can include a non-physical or “wireless” connection, such as a wireless connection in which information and/or power is transmitted between components using electromagnetic energy. A connection can include a connection selected from the group consisting of: a wired connection; a wireless connection; an electrical connection; a mechanical connection; an optical connection; a sound propagating connection; a fluid connection; and combinations of one or more of these.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath”, “below”, “lower”, “above”, “upper”, and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described herein. As used herein, the term “vacuum level” refers to a measure of a vacuum wherein the lower the pressure, the greater the vacuum level.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “threshold” refers to a maximum level, a minimum level and/or range of values. In some embodiments, a system parameter is maintained above a threshold, below a threshold and/or within a threshold, to cause a desired effect (e.g. efficacious therapy) and/or to prevent or otherwise reduce or limit (hereinafter “prevent”) an undesired event (e.g. a device or clinical adverse event). In some embodiments, a system parameter is maintained above a first threshold (e.g. above a first temperature threshold) and below a second threshold (e.g. below a second temperature threshold). In some embodiments, a threshold value is determined to include a safety margin, such as to cause a desired effect and/or prevent an undesired event as the system parameter slightly crosses the threshold (e.g. to account for patient variability, system variability, tolerances, and the like).

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

As used herein, the term “functional element” is to be taken to include one or more elements constructed and arranged to perform a function. A functional element can comprise one or more transducers and/or one or more sensors. In some embodiments, a functional element is configured to deliver energy and/or otherwise treat tissue (e.g. a functional element configured as a treatment element). Alternatively or additionally, a functional element can be configured to record one or more parameters, such as a patient physiologic parameter; a patient anatomical parameter (e.g. a tissue geometry parameter); a patient environment parameter; and/or a system parameter. In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described herein) comprising a liquid or gas (e.g. a vapor) configured to ablate or otherwise treat tissue. A functional element can comprise a reservoir, such as an expandable balloon configured to receive an ablative fluid. A “functional assembly” can comprise an assembly constructed and arranged to perform a function, such as is described herein. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy, mechanical energy (e.g. a transducer comprising a motor or a solenoid), magnetic energy, and/or a different electrical signal. Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these. Alternatively or additionally, a transducer can comprise a mechanism, such as a valve; a grasping element; an anchoring mechanism; an electrically-activated mechanism; a mechanically-activated mechanism and/or a thermally activated mechanism.

As used herein, the term “ablative temperature” refers to a temperature at which tissue necrosis or other desired tissue treatment occurs (e.g. a temperature sufficiently hot or sufficiently cold to cause tissue necrosis). As used herein, the term “ablative fluid” refers to one or more liquids, gases (e.g. vapors), gels, and/or other fluids whose thermal properties cause tissue necrosis and/or another desired tissue treatment (e.g. one or more fluids at an ablative temperature). Alternatively or additionally, “ablative fluid” can refer to one or more fluids whose chemical properties (at room temperature, body temperature or otherwise) cause tissue necrosis or another desired tissue treatment. A tissue treatment element (e.g. a functional element) of the present inventive concepts can comprise one or more ablative fluids.

As used herein, the term “tissue contacting surface” refers to a surface of a system or device component that makes physical contact with tissue, such as a portion of an external surface of an expandable component (e.g. a portion of a balloon's surface) which contacts tissue once expanded. In some embodiments, tissue contacting a tissue contacting surface directly receives energy from the tissue contacting surface of the expandable components, however tissue in proximity (e.g. below or alongside) also receives energy (e.g. via conduction of the delivered energy and/or a resultant heat energy).

As used herein, the term “conduit” can refer to: a tube, such as a tube with a lumen therethrough; or a lumen, such as a lumen of a single or multi-lumen shaft. In some embodiments, conduit refers to both a lumen and the wall surrounding the lumen (e.g. a lumen of a multi-lumen shaft). In some embodiments, a conduit comprises one or more of: a wire; a translatable rod; a filament; a cable; and/or an optical fiber.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is an object of the present inventive concepts to provide systems, methods and devices for safely and effectively treating and/or diagnosing a volume of tissue (the “target tissue”), such as to treat and/or diagnose a patient disease or disorder. Target tissue can comprise one or more target tissue segments or other target tissue portions, such as target tissue located in the intestine of a patient. Clinical procedures in the duodenum and other locations of the small intestine are challenging for a number of reasons, such as those caused by the long distance between the mouth and the intestine and the complexities of the gastrointestinal passageway encountered (including passage through the stomach) during device (e.g. catheter) insertion and operation. Intestinal diameter varies along its length, and effective devices must accommodate this variation. The intestine is quite distensible in the longitudinal and radial directions, further complicating device (e.g. catheter) manipulation and operation (e.g. delivery of energy to tissue). Mobility of intestinal mucosa relative to muscularis is present, as well as mobility of the full wall, but can result in undesired stretching, compression and intussusception. The duodenum is normally closed and may require insufflation to sufficiently open (e.g. for visualization). The insufflation medium (e.g. gas) moves through the intestine, so more may need to be delivered, while excess gas causes discomfort or other adverse effect for the patient. Duodenal and other intestinal tissue tends to stretch or compress as a device is advanced or retracted, respectively, such as to cause retrograde expulsion of devices if a stabilization force is not maintained. It is difficult to manipulate and control devices that include treatment and other elements positioned in the small intestine. The small intestine wraps around the pancreas, and the curvature is quite variable from patient to patient. The length of the intestine along an outer curve is longer than that along an inner curve. In many procedures, there is a desire to avoid damage to the ampulla of Vater (e.g. to avoid restricting bile and/or pancreatic fluid), and this tissue can be difficult to visualize or otherwise identify. There are relatively few endoscopically visualizable landmarks in the intestine, making it difficult to know where in the intestine a portion (e.g. a distal portion) of a device is positioned. Access to the intestine through the stomach via an over-the wire catheter loses one-to-one motion between a proximal handle and a distal portion of the device, as slack can accumulate in the stomach during advancement and slack can be relieved from the stomach during withdrawal. Accessing the intestine can include entering the intestine through the pylorus, a small sphincter, from the stomach, and in obese patients, large stretchable stomachs make it difficult to direct a device to the pylorus. The intestinal mucosa has a very irregular surface due to plicae circulares and mucosal villi and performing a treatment (e.g. an ablation treatment) of the intestinal mucosa is quite different from a treatment procedure performed in the stomach or esophagus, because of this irregularity. Peristalsis present in the small intestine is dynamic and unpredictable and can alter functional element, functional assembly and/or other device component position and/or contact level with tissue. The intestine is not only thin-walled, but the thickness of the wall is highly variable, even within small axial segments of the small intestine, thus complicating preferential ablation of inner layers versus outer layers of the small intestine. The muscularis is innervated and scars and/or stenoses easily, and as such, even minimal trauma to the muscularis should be avoided.

Target tissue can comprise one or more layers of a portion of tubular or non-tubular tissue, such as tissue of an organ or tissue of the gastrointestinal (GI) tract of a patient, such as tissue of the small intestine or large intestine. The systems and devices of the present inventive concepts can include one or more functional assemblies and/or functional elements configured to treat target tissue, such as a treatment element comprising fluid at an ablative temperature delivered to a balloon (ablative temperature fluid and/or balloon filled with ablative fluid each referred to singly or collectively as a “functional element” or a “treatment element” of the present inventive concepts). One or more functional elements can be provided in, on and/or within an expandable functional assembly or other radially deployable mechanism. Functional assemblies and/or functional elements can be configured to treat target tissue (e.g. deliver energy to target tissue), such as to modify target tissue (e.g. to modify the secretions from the target tissue and/or absorption of the target tissue), ablate target tissue (e.g. to cause the replacement of the target tissue with “new tissue”) and/or to cause a reduction in the surface area of target tissue (e.g. the luminal surface area of an inner wall of tubular tissue) at and/or proximate to one or more locations where the treatment was performed (e.g. at and/or proximate the location where energy was delivered). The luminal or other tissue treatment can occur acutely and/or it can take place over time, such as days, weeks or months. A tissue surface area reduction can correspond to a reduction in mucosal surface area available to function in an absorptive, neuronal signaling, and/or a hormonal secretory capacity. A target tissue treatment can result in the replacement of target tissue with new tissue with different absorptive and/or secretory capacity and/or other desirable effect related to replacement and/or modification of target tissue. In some embodiments, the target tissue treatment is performed to reduce the density of the crypts in the small intestine (e.g. reduce the density of the crypts in the duodenum and/or the jejunum). This reduction in crypt density can be performed to reduce the likelihood of undesired mucosal thickening after the target tissue treatment. The treatment of target tissue with the systems, devices and methods of the present inventive concepts can provide a therapeutic benefit to the patient, such as to treat one or more diseases or disorders of the patient, as described in detail herein.

Each functional assembly (e.g. treatment assembly) can comprise at least one functional element (e.g. tissue treatment element) such as a tissue treatment element selected from the group consisting of: ablative fluid delivered to a balloon or other expandable fluid reservoir; energy delivery element mounted to an expandable functional assembly such as an electrode or other energy delivery element configured to deliver radiofrequency (RF) energy and/or microwave energy; light delivery element configured to deliver laser or other light energy; fluid delivery element (e.g. needle or nozzle) configured to deliver ablative fluid directly onto and/or into tissue; sound delivery element such as an ultrasonic and/or subsonic sound delivery element; and combinations of one or more of these. Numerous forms of functional assemblies and/or functional elements can be included. In some embodiments, the functional assemblies and/or the one or more functional elements contained therein are configured as described in: applicant's co-pending U.S. patent application Ser. No. 13/945,138, entitled “Devices and Methods for the Treatment of Tissue”, filed Jul. 18, 2013; applicant's co-pending U.S. patent application Ser. No. 16/438,362, entitled “Heat Ablation Systems, Devices and Methods for the Treatment of Tissue”, filed Jun. 11, 2019; applicant's co-pending U.S. patent application Ser. No. 16/711,236, entitled “Electrical Energy Ablation Systems, Devices and Methods for the Treatment of Tissue”, filed Dec. 11, 2019; and/or applicant's co-pending U.S. patent application Ser. No. 14/609,334, entitled “Ablation Systems, Devices and Methods for the Treatment of Tissue”, filed Jan. 29, 2015.

The treatment assemblies and/or treatment elements of the present inventive concepts can be constructed and arranged to deliver one or more treatments (e.g. deliver energy, deliver a chemically ablative fluid, mechanically abrade and/or otherwise treat tissue) directly to a particular area of tissue, the “energy delivery zone” or simply “delivery zone”. During a single delivery of treatment, a treatment element can be constructed and arranged to deliver treatment to a relatively continuous surface of tissue (e.g. a continuous surface of intestinal tissue in contact with a balloon filled with ablative fluid or a surface of tissue onto which a chemically ablative fluid is sprayed, coated or otherwise delivered). In these continuous-surface treatment delivery embodiments, the delivery zone comprises the continuous surface of tissue receiving the treatment directly. Alternatively, a treatment element can be constructed and arranged to deliver treatment to multiple discrete portions of a tissue surface (e.g. an intestinal tissue surface), with one or more tissue surface portions in-between other surface portions that do not directly receive energy or other treatment from the treatment element. In these segmented-surface treatment delivery embodiments, the delivery zone is defined by a periphery of the multiple tissue surface area portions receiving treatment, similar to a “convex hull” or “convex envelope” used in mathematics to define an area including a number of discrete locations that define a periphery. A delivery zone can comprise two or more contiguous or non-contiguous delivery zones, and multiple delivery zones can be treated sequentially and/or simultaneously.

For example, in embodiments where the treatment element is hot fluid (e.g. ablative fluid at a sufficiently high temperature to cause tissue necrosis) positioned within a balloon, the delivery zone comprises all tissue surfaces contacted by the balloon that directly receive ablative thermal energy from the ablative fluid through the balloon. In embodiments where the treatment element is a balloon filled with cold fluid (e.g. ablative fluid at a sufficiently low temperature to cause tissue necrosis), the delivery zone can comprise all tissue surfaces contacted by the balloon that have heat directly extracted from them by the cold fluid (e.g. at a sufficiently cold temperature to treat the tissue). In embodiments where the treatment element is an array of electrodes configured to deliver electrical energy (e.g. RF energy) to tissue, the delivery zone can comprise an area defined by the electrodes on the periphery of the array (e.g. a convex hull as described above), such as when the electrodes are positioned and energy is delivered to treat relatively the entire surface of tissue within the periphery. In embodiments where the treatment element comprises one or more fluid delivery elements delivering ablative fluid directly onto tissue (e.g. an ablative fluid whose chemical nature modifies tissue, at body temperature or otherwise), the delivery zone can comprise a surface defined by the periphery of tissue locations receiving the ablative fluid, such as when the ablative fluid is delivered (e.g. sprayed or otherwise applied, such as via a nozzle and/or a sponge) to relatively the entire surface within the periphery. In embodiments where the treatment element comprises one or more light delivery elements such as those that deliver laser energy to tissue, the delivery zone can comprise a surface area defined by the periphery of tissue locations receiving the light energy, such as when light is delivered at a set of locations and with a magnitude of energy configured to treat relatively the entire surface of tissue within the periphery. In these embodiments, light can be delivered to relatively the entire energy delivery zone, or to a large number (e.g. greater than 100) of tissue locations within the periphery of the delivery zone (e.g. making up less than 50%, less than 20% or less than 10% of the total surface area of the delivery zone). In embodiments where the treatment element comprises one or more sound delivery elements such as those that deliver sub-sonic and/or ultrasonic sound energy to tissue, the delivery zone can comprise a surface area defined by the periphery of tissue locations receiving the sound energy, such as when ablative sound energy is delivered at a set of locations and with a magnitude of energy configured to treat relatively the entire surface of tissue within the periphery. In embodiments in which the treatment element comprises a mechanical cutter or other abrasion element, the delivery zone can comprise a surface defined by all tissue dissected, cut, mechanically disrupted and/or otherwise modified during a single abrading step of the mechanical abrader.

A delivery zone can comprise a cumulative set of delivery zones that receive treatment simultaneously and/or sequentially, by one or more tissue treatment elements, such as those described herein. A delivery zone can comprise a first delivery zone defined when a treatment element treats target tissue in a first treatment delivery, plus a second delivery zone defined when the treatment element treats target tissue in a second treatment delivery, and so on. In these embodiments, the treatment element can be translated, rotated and/or otherwise repositioned between treatments (e.g. energy delivery), where each delivery zone is associated with the position of the treatment element during each treatment. Multiple delivery zones can receive treatment in a single procedure, such as within a period of less than twenty-four hours. A delivery zone can comprise a set of multiple delivery zones treated by two or more treatment elements, or multiple delivery zones treated by a single treatment element. In some embodiments, treatment of target tissue comprises a treatment in which energy is delivered to a relatively continuous portion of intestinal surface tissue, such that the target tissue treated includes at least this relatively continuous portion of surface tissue. Alternatively or additionally, treatment of target tissue can comprise a treatment in which energy is delivered to multiple discrete (non-continuous) portions of intestinal surface tissue, such that one or more surface portions not receiving energy are dispersed within surface portions that do receive energy (e.g. such that the target tissue treated comprises multiple discrete portions of intestinal surface tissue).

Target tissue treated by each energy delivery and/or other treatment delivery comprises the tissue directly receiving treatment (i.e. the tissue defined by the delivery zone) plus “neighboring tissue” which is also modified by the associated treatment delivery. The neighboring tissue can comprise tissue alongside, below (e.g. in a deeper tissue layer) and/or otherwise proximate the delivery zone tissue. The neighboring tissue treatment can be due to one or more of: conduction and/or convection of heat or cold from the delivery zone; flow of ablative fluid from the delivery zone; flow of toxins or other agents that occur during cell degradation and/or cell death; radiation; luminescence, light dissipation; and other energy and/or chemical propagation mechanisms. In some embodiments, an area (i.e. the delivery zone) comprising an inner surface of mucosal tissue directly receives treatment from one or more treatment elements (e.g. an ablative fluid contained within a balloon), and the total volume of target tissue treated by that single treatment delivery includes: the delivery zone tissue (i.e. surface mucosal tissue directly receiving energy and/or other treatment from the treatment element); surface mucosal tissue in close proximity (e.g. adjacent) to the delivery zone tissue; and mucosal and potentially submucosal tissue layers beneath (deeper than) the delivery zone tissue and the treated adjacent surface mucosal tissue.

In some embodiments, a “treatment neutralizing” procedure is performed after one or more treatments (e.g. energy deliveries), such as a treatment neutralizing cooling procedure performed after one or more treatment elements deliver heat to treat target tissue, or a treatment neutralizing warming procedure performed after one or more treatment elements deliver cryogenic energy to treat target tissue. In these embodiments, the treatment neutralizing cooling or warming fluid can be delivered to the same functional assembly (e.g. an expandable functional assembly comprising a balloon) delivering the heat or cryogenic treatment, respectively, and/or the neutralizing fluid can be delivered directly to tissue by the same or different functional assembly or functional element. In some embodiments, a functional element delivers an ablating agent to target tissue (e.g. a chemical or other agent configured to cause target tissue necrosis or otherwise treat target tissue), and a treatment neutralizing procedure comprises delivery of a neutralizing agent (by the same or different functional element) to target and/or non-target tissue to reduce continued ablation due to the delivered caustic ablative fluid (e.g. a base to neutralize a delivered acid or an acid to neutralize a delivered base).

Each functional assembly and/or functional element of the present inventive concepts can be configured to be positioned proximate one or more intestinal and/or other locations of the patient, such as to perform a function (e.g. perform a treatment, deliver fluid and/or record data) at one or more contiguous or discontiguous tissue locations. Target tissue to be treated (e.g. ablated) comprises a three dimensional volume of tissue, and can include a first portion, a treatment portion, whose treatment has a therapeutic benefit to a patient; as well as a second portion, a “safety-margin” portion, whose treatment has minimal or no adverse effects to the patient. “Non-target tissue” can be identified (e.g. prior to and/or during the medical procedure), wherein the non-target tissue comprises tissue whose treatment by the treatment assembly and/or treatment element should be reduced or avoided such as to reduce or prevent an undesired effect to the patient.

The target tissue treatment can cause one or more modifications of the target tissue such as a modification selected from the group consisting of: modification of cellular function; cell death; apoptosis; instant cell death; cell necrosis; denaturing of cells; removal of cells; and combinations of one or more of these. In some embodiments, the target tissue treatment is configured to create scar tissue. Target tissue can be selected such that after treatment the treated target tissue and/or the tissue that replaces the target tissue functions differently than the pre-treated target tissue, such as to have a therapeutic benefit for the patient. The modified and/or replacement tissue (singly or collectively “treated tissue”) can exhibit different properties than the pre-treated target tissue (“target tissue” herein), such as different properties that are used to treat a patient disease or disorder. The treated tissue can have different secretions and/or quantities of secretions than the target tissue, such as to treat diabetes, hypercholesterolemia and/or another patient disease or disorder. The treated tissue can have different absorptive properties than the target tissue, such as to treat insulin resistance, diabetes, a metabolic condition, and/or another patient disease or disorder. The treated tissue can have a different surface topography than the target tissue, such as a modification of the topography of the inner wall of the GI tract that includes a smoothing or flattening of its inner surface, such as a modification in which the luminal surface area of one or more segments of the GI tract is reduced after treatment. The treated tissue can have a lower density of crypts than the target tissue. The effect of the treatment (e.g. the effect on the target tissue) can occur acutely, such as within twenty-four hours, or after longer periods of time, such as greater than twenty-four hours or greater than one week.

Target tissue to be treated can comprise two or more discrete tissue segments, such as two or more axial segments of the GI tract. Each tissue segment can comprise a full (e.g. approximately 360°) or partial (e.g. <300°) circumferential segment of the tissue segment. Multiple tissue segments can be treated with the same or different functional elements (e.g. treatment elements), and they can be treated simultaneously or in sequential steps (e.g. sequential energy delivery steps that deliver energy to multiple delivery zones). Multiple tissue segments can be treated in the same or different clinical procedures (e.g. procedures performed on different days). In some embodiments, a series of tissue segments comprising a series of axial segments of the GI tract are treated in a single clinical procedure. The first and second tissue segments can be directly adjacent, they can contain overlapping portions of tissue, and/or there can be gaps between the segments. Dissimilarities in treatment elements can include type and/or amount of energy to be delivered by an energy delivery-based treatment element. Dissimilarities in target tissue treatments can include: target tissue area treated; target tissue volume treated; target tissue length treated; target tissue depth treated; target tissue circumferential portion treated; ablative fluid type, volume and/or temperature delivered to a reservoir such as a balloon; ablative fluid type, volume and/or temperature delivered directly to tissue; energy delivery type; energy delivery rate and/or amount; peak energy delivered; average temperature of target tissue achieved during target tissue treatment; maximum temperature of target tissue achieved during target tissue treatment; temperature profile of target tissue treatment; duration of target tissue treatment; surface area reduction achieved by target tissue treatment; and combinations of one or more of these.

Target tissue can include tissue of the duodenum, such as tissue including substantially all or a portion of the mucosal layer of one or more axial segments of the duodenum (e.g. including all or a portion of the plicae circulares and/or the crypts), such as to treat diabetes, hypercholesterolemia and/or another patient disease or disorder, such as while leaving the duodenum anatomically connected after treatment. Target tissue can include one or more portions of a tissue layer selected from the group consisting of: mucosa; mucosa through superficial submucosa; mucosa through mid-submucosa; mucosa through deep-submucosa; and combinations of one or more of these. Replacement tissue can comprise cells that have migrated from one or more of: gastric mucosa; jejunal mucosa; an untreated portion of the duodenum whose mucosal tissue functions differently than the treated mucosal tissue functions prior to treatment; and combinations of one or more of these. Replacement tissue can include one or more tissue types selected from the group consisting of: scar tissue; normal intestinal mucosa; gastric mucosa; and combinations of one or more of these. In some embodiments, replacement tissue comprises tissue that has been delivered onto and/or into tissue by a catheter of the present inventive concepts. In some embodiments, target tissue includes a treatment portion comprising the mucosal layer of the duodenum, and a safety-margin portion comprising a near-full or partial layer of the submucosal layer of the duodenum. In some embodiments, the target tissue comprises nearly the entire mucosal layer of the duodenum, and it can include a portion of the pylorus contiguous with the duodenal mucosa and/or a portion of the jejunum contiguous with the duodenal mucosa. In some embodiments, the target tissue comprises all or a portion of the duodenal mucosa distal to the ampulla of Vater (e.g. avoiding tissue within at least 0.5 cm, 1.0 cm or 1.5 cm from the ampulla of Vater, while including tissue within 3 cm, 5 cm, 10 cm or 15 cm distal to the ampulla of Vater). In these embodiments, the target tissue can comprise at least 10%, at least 15%, at least 25%, at least 30% or at least 50% of the duodenal mucosa distal to the ampulla of Vater. Alternatively or additionally, the target tissue can comprise no more than 70% or no more than 90% of the duodenal mucosa distal to the ampulla of Vater. In these embodiments, tissue proximal to and/or proximate the ampulla of Vater can comprise non-target tissue (i.e. tissue whose treatment is avoided or at least reduced).

In some embodiments, the target tissue comprises at least a portion of duodenal mucosal tissue, and the systems, methods and devices of the present inventive concepts are configured to counteract duodenal mucosal changes that cause an intestinal hormonal impairment leading to insulin resistance in patients. In these embodiments, the therapy provided can improve the body's ability to process sugar and dramatically improve glycemic control for patients with insulin resistance and/or Type 2 diabetes. In some embodiments, target tissue is treated to prevent and/or reduce cognitive decline (e.g. Alzheimer's Disease), such as by improving sugar metabolism in the brain, overcoming insulin resistance in the brain, reducing toxicity of beta amyloid, reducing oxidative stress, and/or reducing inflammation in the brain associated with neuronal death. In some embodiments, target tissue is treated to: prevent liver fibrosis and/or cirrhosis (e.g. non-alcoholic fatty liver disease NAFLD or non-alcoholic steatohepatitis NASH); reduce liver fat; reduce oxidative stress; and/or reduce inflammation in the liver associated with liver fibrosis and toxicity.

Hormones released from the intestinal mucosa play an important role in modulating glucose homeostasis, and different axial segments of the intestinal mucosa release different hormones in the fasting and post-prandial state, in order to modulate blood glucose in the fasting and post-prandial states, respectively. After a meal, the proximal intestinal mucosa senses the intestine for ingested glucose and releases a collection of hormones in response to this signal. These hormones initiate the process of insulin release into the bloodstream after a meal, but they also induce some insulin resistance to prevent the released insulin from causing hypoglycemia before the body has a chance to absorb the ingested glucose. One such hormone that plays a role in this is GIP. Distal gut hormones (produced in the jejunum or a more distal location), on the contrary, allow the release of more insulin but also play a role in helping the body now become sensitive to its circulating insulin. Teleologically, the explanation for this difference in the type of gut hormones produced by different segments of the intestine is that enough glucose will have been absorbed by the time nutrients reach the distal intestine to allow the insulin to begin to function to reduce blood glucose levels. Releasing different hormones at different times (e.g. from different segments of the intestine) enables the body to absorb and process glucose in such a way as to avoid hypoglycemia (blood sugars that are too low) and hyperglycemia (blood sugars that are too high). In this way, intestinal hormonal signaling is important for whole body glucose homeostasis in the fasting and post-prandial states. The treatment can also lead to weight loss through decreased absorption of nutrients, increased sensation of satiety, altered food preferences, increased energy expenditure, and combinations of one or more of these.

In patients with Type 2 Diabetes, a lifetime of exposure to fat and sugar can lead to intestinal changes that occur in regions with the highest exposure to these nutrients, predominantly in the proximal intestine. These changes are characterized by an excess proximal intestinal mucosa's hormonal contribution to the fasting and post-prandial glucose homeostasis. The net result of these intestinal changes is to create a condition of insulin resistance and impaired glucose tolerance. Treatment of duodenal mucosal tissue with the systems, devices and methods of the present inventive concepts can be performed to alter the intestinal mucosal hormone production from the region of treated tissue. The treated tissue can then have an altered hormonal secretion pattern that affects blood glucose levels in the fasting and post-prandial states. The tissue treatment of the present inventive concepts can be performed to effect duodenal mucosal tissue secretion of GIP and/or GLP-1. The tissue treatment can lead to changes in the blood levels of GIP and/or GLP-1 (and other gut hormones) that can lead to changes in glucose homeostasis in the fasting and/or post-prandial states. The treatment can lead to changes in insulin and/or glucagon secretion from the pancreas and/or insulin and/or glucagon levels in the bloodstream. The treatment can lead to changes in pancreatic beta cell function and/or health through direct hormonal consequences of the treated duodenal tissue and/or indirectly through improved blood glucose levels. In some embodiments, the treatment of the present inventive concepts is configured to at least one of reduce a blood glucose level and/or reduce a lipoprotein level.

Treatment of intestinal tissue (e.g. duodenal mucosal tissue) can be performed to treat a disease and/or disorder selected from the group consisting of: diabetes; pre-diabetes; impaired glucose tolerance; insulin resistance; obesity or otherwise being overweight; a metabolic disorder and/or disease; and combinations of one or more of these. In some embodiments, treatment of intestinal tissue (e.g. at least duodenal mucosal tissue) using the systems, devices and/or methods of the present inventive concepts can be performed to treat one or more disease and/or disorder selected from the group consisting of: Type 2 diabetes; Type 1 diabetes; “Double diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's; neuropathy; diabetic nephropathy; retinopathy; heart disease; diabetic heart disease; heart failure; diabetic heart failure; and combinations of one or more of these. A near full circumferential portion (e.g. approximately) 360° of the mucosal layer of one or more axial segments of GI tissue can be treated. In some embodiments, less than 360° of one or more axial segments of tubular tissue is treated, such as one or more circumferential portions less than 350°, or between 300° and 350°, such as to prevent a full circumferential scar from being created at the one or more axial segment locations.

Target tissue can be selected to treat two or more patient diseases or disorders, such as two or more patient diseases or disorders as described herein.

Target tissue can comprise tissue of the terminal ileum, such as to treat hypercholesterolemia and/or diabetes. In these embodiments, the target tissue can extend into the proximal ileum and/or the colon.

Target tissue can comprise gastric mucosal tissue, such as tissue regions that produce ghrelin and/or other appetite regulating hormones, such as to treat obesity and/or an appetite disorder.

Target tissue can comprise tissue selected from the group consisting of: large and/or flat colonic polyps; margin tissue remaining after a polypectomy; and combinations of one or more of these. These tissue locations can be treated to treat residual cancer cells.

Target tissue can comprise at least a portion of the intestinal tract afflicted with inflammatory bowel disease, such that Crohn's disease and/or ulcerative colitis can be treated.

Target tissue can comprise GI tissue selected to treat Celiac disease and/or to improve intestinal barrier function.

The functional assemblies, functional elements, systems, devices and methods of the present inventive concepts can be configured to avoid ablating or otherwise adversely affecting certain tissue, termed “non-target tissue” herein. Depending on the location of tissue intended for treatment (i.e. target tissue), different non-target tissue can be applicable. In certain embodiments, non-target tissue can comprise tissue selected from the group consisting of: gastrointestinal adventitia; duodenal adventitia; the tunica serosa; the tunica muscularis; the outermost partial layer of the submucosa; papilla and/or other portions of the ampulla of Vater; pancreas; bile duct; pylorus; and combinations of one or more of these.

In some embodiments, two or more clinical procedures are performed in which one or more volumes of target tissue are treated in each clinical procedure, such as is described in applicant's co-pending U.S. patent application Ser. No. 14/673,565, entitled “Methods, Systems and Devices for Performing Multiple Treatments on a Patient”, filed Mar. 30, 2015. For example, a second clinical procedure can be performed at least twenty-four hours after the first clinical procedure, such as a second clinical procedure performed within 6 months of a first clinical procedure or a clinical procedure performed after at least 6 months after the first clinical procedure. The first and second clinical procedures can be performed using similar or dissimilar methods, and they can be performed using similar or dissimilar systems and/or devices (e.g. performed with similar or dissimilar treatment and/or other functional elements). The first and second clinical procedures can treat similar or dissimilar volumes of target tissue (e.g. similar or dissimilar amounts of tissue treated and/or locations of tissue treated), and they can deliver energy to similar or dissimilar sets of multiple delivery zones. In some embodiments, the first and second clinical procedures can include treating and/or delivering energy to contiguous and/or overlapping regions of the GI tract either in the circumferential and/or axial dimensions. In other embodiments, the first and second clinical procedures can include the treatment of disparate regions of the GI tract (such as disparate regions of the duodenum, ileum, and/or stomach). The first and second clinical procedures can be performed using similar or dissimilar devices (e.g. catheters). The first and second clinical procedures can comprise similar or dissimilar deliveries of energy to treat the target tissue. The first and second clinical procedures can be performed at similar or dissimilar temperatures. The second clinical procedure can be performed based on diagnostic results collected after the first clinical procedure has been performed, such as when the diagnostic results are based on a biopsy of mucosal tissue.

The functional assemblies, treatment assemblies, treatment elements and other functional elements of the present inventive concepts can comprise an expandable element or otherwise be configured to automatically and/or manually expand or traverse in at least one radial direction. Typical expandable elements include but are not limited to: an inflatable balloon; a radially expandable cage or stent; one or more radially deployable arms; an expandable helix; an unfurlable compacted coiled structure; an unfurlable sheet; an unfoldable compacted structure; and combinations of one or more of these. In some embodiments, an expandable element can comprise a radially expandable tube, such as a sheet of material resiliently biased in a radially expanded condition that can be compacted through a furling operation, or a sheet of material resiliently biased in a radially compact condition that can be expanded through an unfurling operation. An expandable element can comprise a foldable sheet, such as a sheet configured to be folded to be radially compacted and/or to be unfolded to radially expand. In some embodiments, an expandable element expands to contact tissue, such as to expand to a diameter similar to the diameter of the luminal wall tissue into which the expandable element has been placed. In some embodiments, an expandable element expands to be closer to wall tissue, but remain at a distance (e.g. a fixed or pre-determined distance) from the tissue surface, such as when the tissue is subsequently brought into contact with all or a portion of an expanded functional assembly or functional element (e.g. using insufflation fluid withdrawal techniques). In some embodiments, an expandable element expands to be larger than the diameter of the luminal wall tissue into which the expandable element has been placed, such as to improve the quality of the apposition of the expandable element against the uneven surface of the tissue. In these embodiments, the fully expanded diameter of an expandable element would be configured to avoid a diameter large enough to cause lasting mechanical damage to the apposed tissue and/or to tissue proximate the apposed tissue. In some embodiments, the expansion of an expandable element (e.g. the expansion of an expandable functional assembly) is monitored and/or varied (e.g. decreased and/or increased), such as to accommodate or otherwise compensate for peristalsis or other muscle contractions that occur in the GI tract (e.g. contractions that occur when a foreign body is present in the GI tract) and/or varied to accommodate changes in GI lumen diameter imposed by aspects of the procedure itself.

Any device (e.g. catheter) of the present inventive concepts can include one or more functional elements comprising one or more treatment elements configured to deliver energy to one or more delivery zones, to treat at least a portion of target tissue. Any device can include one or more functional elements comprising one or more fluid delivery elements, such as one or more nozzles or needles configured to deliver fluid toward and/or into tissue. The fluid delivery elements can be constructed and arranged to deliver fluid to perform a function selected from the group consisting of: expanding one or more tissue layers; warming or cooling tissue; removing debris or other substance from a tissue surface; delivering energy to a delivery zone comprising a continuous or segmented surface; treating target tissue; and combinations of one or more of these. Any of the expandable functional assemblies of the present inventive concepts can include one or more other functional elements, such as are described herein. The treatment elements and/or other functional elements (e.g. fluid delivery elements) can be mounted on, within (e.g. within the wall) and/or inside of an expandable element such as a balloon or expandable cage. In some embodiments, one or more functional elements is not mounted to an expandable element, such as those attached to a shaft or other non-expandable catheter component.

In some embodiments, a catheter comprises at least one functional element configured to deliver energy to a delivery zone such as to ablate target tissue. Examples of ablation-based functional elements include but are not limited to: ablative fluids, such as hot or cold ablative fluids delivered to a balloon and/or directly to target tissue; one or more fluid delivery elements configured to deliver ablative fluid directly to target tissue; an RF and/or microwave energy delivery element such as one or more electrodes; an ultrasonic and/or subsonic transducer such as one or more piezo crystals configured to ablate tissue with ultrasonic or subsonic energy, respectively, sound waves; a laser energy delivery element such as one or more optical fibers, laser diodes, prisms and/or lenses; a rotating ablation element; a circumferential array of ablation elements; and combinations of one or more of these.

The expandable elements of the present inventive concepts comprising balloons can be divided into two general categories: those that are composed of a substantially elastic material, such as silicone, latex, low-durometer polyurethane, and the like; and those that are composed of a substantially inelastic material, such as polyethylene terephthalate (PET), nylon, high-durometer polyurethane and the like. A third category includes balloons which include both elastic and inelastic portions. Within the category of elastic balloons, two subcategories exist: a first sub-category wherein a combination of material properties and/or wall thickness can be combined to produce a balloon that exhibits a measurable pressure-threshold for inflation (i.e. the balloon becomes inflated only after a minimum fluidic pressure is applied to the interior of the balloon); and a second sub-category, wherein the balloon expands elastically until an elastic limit is reached which effectively restricts the balloon diameter to a maximum value. The individual properties of the balloons in each of these categories can be applied to one or more advantages in the specific embodiments disclosed herein, these properties integrated singly or in combination. By way of example only, one or more of the following configurations can be employed: a highly elastic balloon can be used to achieve a wide range of operating diameters during treatment (e.g. during operation a desired balloon diameter can be achieved by adjustment of a combination of fluid temperature and pressure); a substantially inelastic balloon or a balloon that reaches its elastic limit within a diameter approximating a target tissue diameter (e.g. a duodenal mucosal diameter) can be used to achieve a relatively constant operating diameter that will be substantially independent of operating pressure and temperature; a balloon with a pressure-threshold for inflation can be used to maintain an uninflated diameter during relatively low pressure conditions of fluid flow and then achieve a larger operating diameter at higher pressure conditions of flow. Pressure-thresholded balloons can be configured in numerous ways. In one embodiment, a balloon is configured to have a relatively thick wall in its uninflated state, such as to maximize an electrically and/or thermally insulating effect while the balloon is maintained in this uninflated state. The balloon can be further configured such that its wall thickness decreases during radial expansion (e.g. to decrease an electrically and/or thermally insulating effect). In another embodiment, a balloon is configured to have a relatively small diameter in its uninflated state (e.g. a diameter that is small relative to the inner diameter of tubular target tissue such as the diameter of the mucosal layer of duodenal wall tissue), such as to minimize or completely eliminate apposition between the balloon and the surrounding tissue to minimize heat, RF and/or other energy transfer into the surrounding tissue until the balloon is fully inflated. In another embodiment, a balloon and an ablation system or catheter are configured to circulate a flow of fluid through the balloon (e.g. an elastic balloon or an inelastic balloon) at a sufficiently low enough pressure to prevent apposition of the balloon or other catheter component with target tissue, such as to pre-heat one or more surfaces of the ablation system or ablation device that are in fluid communication with the balloon. In this configuration, when the balloon or other ablation element is positioned to deliver energy to target tissue, the temperature of the balloon or other ablation element will be at a desired level or it will rapidly and efficiently reach the desired level for treatment (i.e. minimal heat loss to the fluid path components due to the pre-heating or pre-cooling). These configurations provide a method of delivering energy to tissue with an ablative fluid filled balloon. A “thermal priming” procedure can be performed prior to one or more target tissue treatments, such as to improve thermal response time of one or more portions of the catheter. Ablative fluid filled balloon catheters as well as thermal priming devices and methods can be configured as is described in applicant's co-pending U.S. patent application Ser. No. 16/438,362, entitled “Heat Ablation Systems, Devices and Methods for the Treatment of Tissue”, filed Jun. 11, 2019.

A fluid evacuation procedure can be performed on one or more internal locations of the catheters, functional assemblies and/or functional elements of the present inventive concepts, such as when a negative pressure is applied (e.g. to a device lumen) to purge or otherwise evacuate fluid from one or more locations. A fluid evacuation procedure can be performed prior to a thermal priming procedure and/or prior to delivering ablative fluid to a treatment element.

At times during target tissue treatment when it is desirable to initiate, increase and/or otherwise modify the treatment of tissue by one or more treatment elements (e.g. a fluid delivery element delivering ablative fluid, a mechanically abrasive element, a hot or cold fluid balloon delivering a thermal energy to tissue and/or an electrode delivering RF energy), the diameter of the treatment assembly and/or treatment element (e.g. the diameter of a balloon, deployable cage, expandable tube or other expandable assembly) can be increased in situ to move a treatment element closer to target tissue and/or to change the contact force between the treatment element and the target tissue. At times during treatment when it is desirable to stop or otherwise decrease the amount of tissue treatment, the diameter of the treatment assembly and/or treatment element can be reduced in situ, such as to prevent or otherwise reduce delivery of energy or other treatment to the target tissue by eliminating or reducing tissue contact of one or more treatment elements (e.g. electrodes, abrasive surfaces or ablative fluid-filled balloons). For those cases where the native diameter of the target tissue varies substantially within a delivery zone, then a highly elastic or compliant balloon or other expandable element can be employed, such as a balloon or deployable cage which can be adjusted to achieve a wide range of operating diameters.

Alternatively or additionally, to initiate, increase and/or otherwise modify the treatment of tissue by one or more functional elements (e.g. a fluid delivery element delivering ablative fluid, a mechanically abrasive element, a hot or cold fluid balloon delivering thermal energy to or from tissue and/or an electrode delivering RF energy), the diameter of the target tissue can be decreased in situ to move target tissue closer to a treatment element and/or to change the contact force between the target tissue and the treatment element. To stop or otherwise decrease ablation of tissue, the diameter of tissue neighboring a treatment element can be increased in situ, such as to prevent or otherwise reduce delivery of energy or other treatment to the target tissue by eliminating or reducing tissue contact of one or more treatment elements (e.g. electrodes, abrasive surfaces or ablative fluid filled balloons). The diameter of the tissue proximate a functional assembly can be increased or decreased, independent of the functional assembly diameter, by means of delivering and/or withdrawing a fluid, to and/or from a body lumen (e.g. a lumen of a segment of the intestine) surrounded by target tissue, such as by using standard GI insufflation techniques. Typical insufflation fluids include but are not limited to: gases such as carbon dioxide or air; liquids such as water or saline solution; and combinations of one or more of these. The insufflation fluids can be introduced through a catheter, through an endoscope such as an endoscope through which the catheter is inserted, and/or via another device placed proximate the target tissue. Delivery of insufflation fluids can be performed to move target tissue away from one or more functional elements, such as to stop transfer of energy to target tissue at the end of a treatment of target tissue as described herein. Alternatively or additionally, delivery of insufflation fluids can be performed to manipulate tissue, such as to distend and/or elongate tissue. Extraction of these insufflation fluids and/or the application of a vacuum or other negative pressure can be used to decrease the diameter of the target tissue, such as to bring the target tissue in closer proximity to one or more functional elements and/or to increase the contact force between target tissue and one or more functional elements, also as described herein. In this tissue diameter-controlled approach, a functional assembly including a balloon that can be maintained at a substantially constant diameter can be desirable, such as a substantially inelastic balloon such as a balloon with an elastic-limit.

The systems of the present inventive concepts can include one or more tissue expansion catheters that comprise one or more functional elements configured as fluid delivery elements. In these embodiments, the one or more functional elements can comprise one or more needles, nozzles, and/or fluid jets configured to deliver one or more fluids or other injectates to tissue, such as to expand target tissue and/or tissue proximate the target tissue (e.g. safety margin tissue) prior to treatment of target tissue by a tissue treatment element. The expanded tissue layer acts as a safety volume of tissue, reducing the specificity of the treatment (e.g. ablation) required and/or the need to protect the underlying non-target tissue from damage. In some embodiments, a vacuum pressure can be used to manipulate tissue and/or to maintain proximity between a portion of a tissue expansion device and tissue. The vacuum can be provided by one or more vacuum sources, such as via one or more operator adjustable vacuum sources.

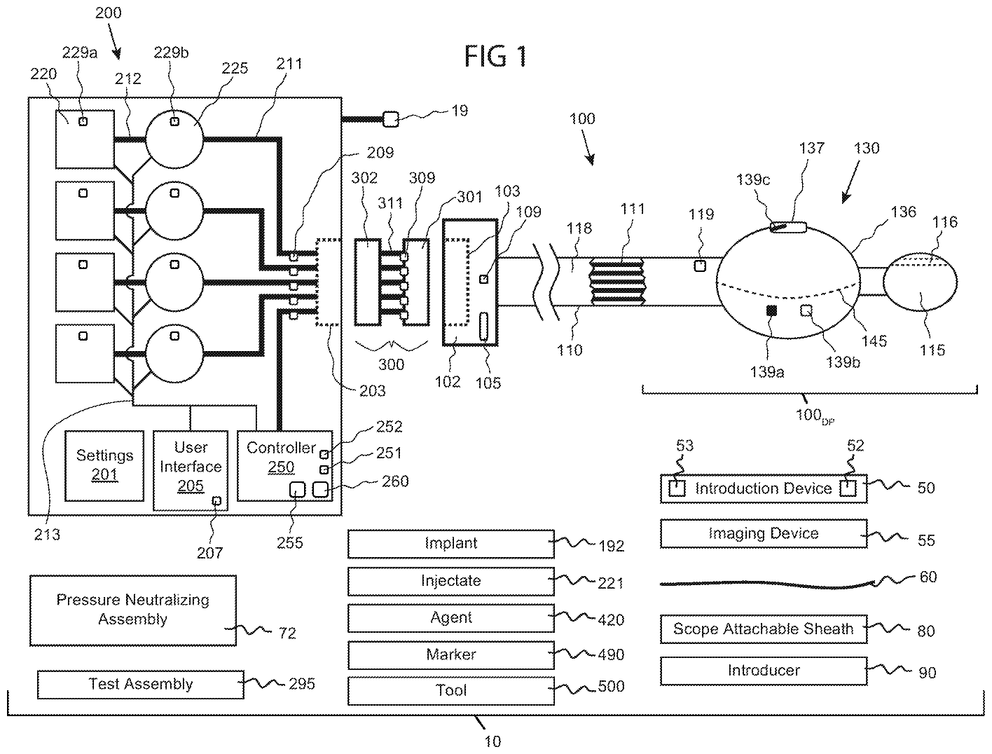

Referring now to FIG. 1, a schematic view of a system for performing a medical procedure on a patient is illustrated, consistent with the present inventive concepts. The medical procedure can comprise a diagnostic procedure (e.g. a diagnostic and/or prognostic procedure), a therapeutic procedure, or a combined diagnostic and therapeutic procedure. System 10 comprises one or more treatment devices, catheter 100 (e.g. a catheter, flexible instrument, and/or other elongate device for insertion into a patient), and a console, console 200, which operably attaches to the one or more catheters 100 (e.g. attaches to two, three or more catheters 100). Catheter 100 comprises an elongate shaft, shaft 110, which can comprise one or more shafts (e.g. shafts with flexible and/or rigid segments). In some embodiments, shaft 110 comprises multiple shafts in a spiraled configuration (e.g. helical configuration) such as is described in applicant's co-pending U.S. patent application Ser. No. 16/742,645, entitled “Intestinal Catheter Device and System”, filed Jan. 14, 2020. In some embodiments, shaft 110 comprises a non-circular cross section (e.g. to “hug” a second device such as an endoscope simultaneously inserted into the patient). In some embodiments, shaft 110 comprises one or more of: a braided portion; a tapered portion; an insertable stiffening mandrel; a variable stiffness portion; and combinations of one or more of these. Shaft 110 can comprise one, two, or more materials selected from the group consisting of: polyether block amide (PEBA); Pebax 5533; Pebax 6333; Pebax 4033; a lubricious additive; a visualizable material such as a radiopaque material and/or an ultrasonically reflective material; and combinations of these.

Catheter 100 comprises one or more functional assemblies, functional assembly 130 shown, which can be configured to radially expand and/or contract. Functional assembly 130 can be positioned on a distal portion of catheter 100 (e.g. on the distal end or a distal portion of shaft 110), distal portion 100_{DP}. In some embodiments, functional assembly 130 comprises a non-circular cross section (e.g. a cross section with a geometry configured to “hug” a second device such as an endoscope simultaneously inserted into the patient). Functional assembly 130 can comprise one or more tissue-contacting portions, as described herein (e.g. side walls of functional assembly 130 that contact inner wall tissue of the intestine or other GI lumen). Functional assembly 130 can comprise a tissue-contacting surface area (e.g. when expanded) of between 500 mm² to 3500 mm², such as a tissue contacting surface area of approximately between 1000 mm² and 2000 mm², or approximately between 1250 mm² and 1750 mm², or approximately 1500 mm². In some embodiments, functional assembly 130 comprises an expanded diameter of approximately 18 mm, 19 mm, 22 mm, 25 mm, 28 mm, or 32 mm. In some embodiments, system 10 includes a single catheter 100 with a functional assembly 130 configured to expand to a single, pre-determined diameter (e.g. when comprising a balloon made from a non-compliant material), such as a diameter of between 19 mm and 32 mm, or between 23 mm and 28 mm, such as a diameter of approximately 24 mm, 26 mm or 28 mm. In these embodiments, functional assembly 130 can deliver energy (e.g. thermal energy delivered by filling functional assembly 130 with ablative fluid) to successfully ablate target tissue in various segments of the small intestine (e.g. duodenum), such as multiple segments of different diameters and/or one or more single segments with a varying diameter along its length. For example, energy can be delivered by an expanded functional assembly 130 to various diameter segments after a tissue expansion procedure is performed. For example, the energy can be delivered after a tissue expansion (e.g. as described herein) that normalizes the variability in intestinal segment diameter. In some embodiments, a tissue expansion procedure creates a desired diameter to be present in the intestinal segment, such as to create a diameter that is compatible with a functional assembly 130 of a pre-determined diameter subsequently delivering energy to perform a safe and effective ablation at the segment. Alternatively or additionally, energy can be delivered by an expanded functional assembly 130 to various diameter segments after an aspiration procedure is performed proximate the segment receiving the energy (e.g. an aspiration procedure performed using an aspiration port of catheter 100 and/or an aspiration port of an endoscope). In some embodiments, energy is delivered by an expanded functional assembly 130 to tissue (e.g. to target tissue) during the performance of a tissue expansion procedure by functional assembly 130. In some embodiments, functional assembly 130 comprises a tissue-contacting length (e.g. when expanded) of between 10 mm and 40 mm, such as a length of approximately 15 mm, 20 mm, 25 mm or 30 mm. This tissue-contacting length represents the “treatment length” of the functional assembly 130. In some embodiments, system 10 includes a first catheter 100a comprising a functional assembly 130a with a first geometry, and a second catheter 100b comprising a functional assembly 130b with a second geometry that is similar or different than the first geometry (e.g. a different length, expanded diameter; and/or tissue contacting surface area). In some embodiments, functional assembly 130b is configured to deliver a different form of ablative energy than functional assembly 130a.

In some embodiments, shaft 110 passes through all or a portion of functional assembly 130. In other embodiments, functional assembly 130 is positioned on a distal end of shaft 110.

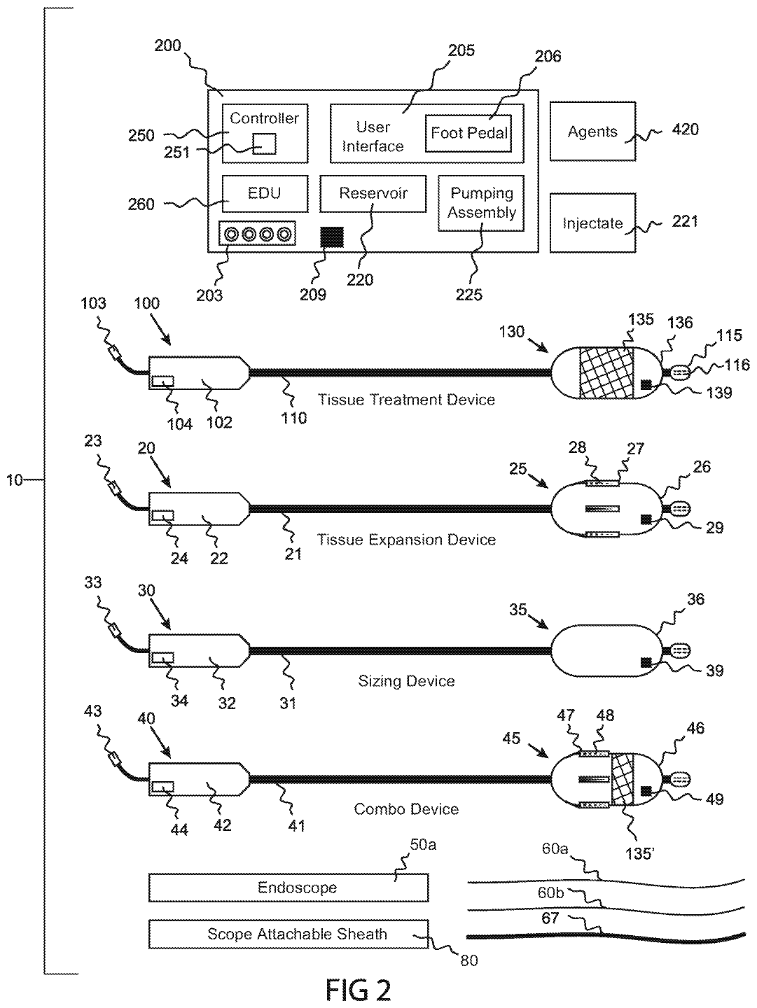

Catheter 100 can comprise one or more catheters of similar construction and arrangement (e.g. and include similar components) as one or more of catheters 100, 20, 30 and/or 40 of FIG. 2, each described in detail herein. Catheter 100 can be constructed and arranged to perform a medical procedure in an intestine of the patient, such as a procedure in the small intestine (e.g. in the duodenum) and/or in the large intestine. In some embodiments, system 10 further comprises a connecting assembly, connecting assembly 300 which can be constructed and arranged to operably attach (e.g. fluidly, mechanically, electrically and/or optically connect) catheter 100 to console 200. In alternate embodiments, catheter 100 can operably attach directly to console 200, without connecting assembly 300. Console 200 can be of similar construction and arrangement as console 200 of FIG. 2, also described in detail herein.

System 10 can further comprise body introduction device 50, guidewire 60, sheath 80 (e.g. an endoscope-attachable sheath), introducer 90 (e.g. an introducer sheath), injectate 221 and/or agent 420, each of which can be of similar construction and arrangement to the similar components described in detail herein in reference to FIG. 2. Body introduction device 50 can comprise an endoscope, a laparoscopic port and/or a vascular introducer. Body introduction device 50 can comprise a camera, such as camera 52 shown, and a display, not shown but such as a display of console 200 and/or another display used to display an image (i.e. camera view) provided by camera 52. In some embodiments, device 50 comprises an endoscope and includes a cap, scope cap 53 shown, which is attached (or attachable) to a distal end of the endoscope, such as to limit tissue collapse that would limit visualization provided by camera 52. Scope cap 53 can extend between 2-6 mm in front of camera 52. In some embodiments, scope cap 53 is of similar construction and arrangement to the Reveal® distal attachment cap manufactured by US Endoscopy.

In some embodiments, system 10 further comprises imaging device 55, which can comprise an imaging device constructed and arranged to provide an image of the patient's anatomy (e.g. inner wall or any part of the intestine of the patient) and/or an image of all or part of catheter 100 and/or other portion of system 10. Imaging device 55 can comprise an imaging device selected from the group consisting of: endoscope camera; visible light camera; infrared camera; X-ray imager; fluoroscope; Ct Scanner; MRI; PET Scanner; ultrasound imaging device; and combinations of one or more of these. In some embodiments, a patient image is used to set, confirm and/or adjust one or more system 10 parameters, such as is described herein in reference to FIG. 5, such as when imaging device 55 comprises one or more sensors of the present inventive concepts configured to produce a signal.

In some embodiments, system 10 further comprises functional element 19 comprising a sensor, transducer, and/or other functional element. Functional element 19 can be operably attached to console 200 or another component of system 10. Functional element 19 can comprise a sensor configured to produce a signal which can be used to modify a parameter of system 10. In some embodiments, functional element 19 comprises a sensor configured to measure a patient parameter, such as a patient parameter selected from the group consisting of: a patient physiologic parameter; blood pressure; heart rate; pulse distention; glucose level; blood glucose level; blood C-peptide level; blood glucagon level; blood insulin level; blood gas level; hormone level; GLP-1 level; GIP level; EEG; LFP; respiration rate; breath distention; perspiration rate; temperature; gastric emptying rate; peristaltic frequency; peristaltic amplitude; a patient anatomical parameter such as tissue geometry information; a patient environment parameter such as room pressure or room temperature; and combinations of one or more of these.

In some embodiments, system 10 further comprises one or more tools, tool 500 shown, such as a tool 500 described herein.

In some embodiments, system 10 comprises one or more sensors, such as when one or more functional elements of system 10 are configured as a sensor, such as functional elements 19, 109, 119, 139, 229 and/or 309 shown, such as is described in detail herein. Each of the system 10 sensors can be configured to produce a signal related to a patient parameter and/or a system 10 parameter. For purposes herein, a signal “related” to a parameter shall include signals that directly represent the parameter, as well as signals that provide information that can be correlated to or in any way relate to the parameter. For example, a sensor (e.g. a temperature or pressure sensor) placed proximate tissue or a component of system 10 can directly represent a parameter (e.g. the temperature or pressure, respectively) of or within locations proximate that tissue or component, respectively. Alternatively, a sensor placed at one location (e.g. one location within system 10), can provide a signal that can be analyzed to produce information representing a parameter at a different location (e.g. a different location within system 10 or a location within the patient). For example, a temperature or pressure measured at one location (e.g. within console 200, connecting assembly 300 and/or a proximal portion of catheter 100) can correlate to a temperature or pressure at a different location (e.g. proximate and/or within functional assembly 130). Correlation of signals provided by a sensor of system 10 to a parameter at a location distant from the sensor can be accomplished by one or more algorithms of system 10, such as algorithm 251 described herein.

In some embodiments, a system 10 sensor is configured to produce a signal related to an anatomic and/or physiologic parameter of the patient, such as a parameter selected from the group consisting of: a parameter of the intestine; a parameter related to the anatomical geometry of a portion of the intestine; a parameter related to force and/or pressure applied to tissue (e.g. tissue of the intestine); a parameter related to a pressure within tissue (e.g. tissue within the luminal surface of the intestine); a parameter related to temperature of tissue (e.g. tissue of the intestine); and combinations of one or more of these. In some embodiments, one or more sensors of system 10 comprise a camera configured to provide an image, and the signal provided by the sensor comprises the image or an analysis of the image. The signal provided by the sensor can relate to a patient parameter (e.g. a patient physiologic or anatomical parameter) or a system 10 parameter (e.g. a functional assembly 130 parameter).

In some embodiments, a system 10 sensor is configured to produce a signal related to a parameter of one or more components of system 10, such as a component of console 200, connecting assembly 300 and/or catheter 100. For example, the signal produced by one or more sensors of system 10 can be related to a functional assembly 130 parameter, such as a parameter selected from the group consisting of: pressure within functional assembly 130; force applied to and/or by a portion of functional assembly 130; temperature of at least a portion of functional assembly 130; temperature of fluid within functional assembly 130; state of expansion of functional assembly 130; position of functional assembly 130 (e.g. position of functional assembly 130 relative to the patient's anatomy): and combinations of one or more of these.

In some embodiments, system 10 is configured to perform a therapeutic procedure selected from the group consisting of: a tissue removal procedure such as a tissue removal procedure in which mucosal intestinal tissue is removed; a tissue ablation procedure such as a tissue ablation procedure in which at least intestinal mucosal tissue is ablated; a tissue expansion procedure such as a tissue expansion procedure configured to create a safety margin of tissue (e.g. safety margin of expanded submucosal tissue for a subsequent ablation of mucosal tissue) and/or a tissue expansion procedure configured to create a therapeutic restriction; and combinations of one or more of these.

In some embodiments, system 10 is configured to treat one or more medical conditions (e.g. patient diseases and/or disorders), such as are described herein. For example, system 10 can be configured to treat diabetes, such as Type 2 diabetes, Type 1 diabetes, “Double diabetes” and/or gestational diabetes. In some embodiments, system 10 is configured to treat hypercholesterolemia, such as when target tissue treated by functional assembly 130 includes tissue of the terminal ileum. In some embodiments, system 10 is configured to treat both diabetes and hypercholesterolemia. In some embodiments, system 10 is configured such that functional assembly 130 treats a part of the intestine exhibiting inflammatory bowel disease, ulcerative colitis and/or chronic ulcers.

System 10 can be constructed and arranged to cause functional assembly 130 to expand one or more layers of tissue (e.g. submucosal tissue), and/or to treat target tissue (e.g. target tissue comprising mucosal tissue of the duodenum or other intestinal mucosa). System 10 can be further constructed and arranged to avoid adversely affecting non-target tissue, as described in detail herein and in applicant's co-pending U.S. patent application Ser. No. 13/945,138, entitled “Devices and Methods for the Treatment of Tissue”, filed Jul. 18, 2013.

In some embodiments, system 10 includes a catheter 100 which is configured to both expand tissue (e.g. submucosal tissue of the small intestine) as well as ablate or otherwise treat tissue (e.g. ablate mucosal tissue of the small intestine), such as when functional assembly 130 is configured to deliver energy (e.g. thermal energy) to target tissue (e.g. mucosal tissue) as well as deliver tissue-expanding fluid to submucosal tissue (e.g. submucosal tissue proximate the target tissue). In some embodiments, catheter 100 performs a tissue expansion step and an energy delivery step in a “co-registered arrangement”. For example, functional assembly 130 can be positioned at an intestinal location, after which a tissue expansion procedure is performed by functional assembly 130 (e.g. via one or more fluid delivery elements 139c). Subsequently, and without translation or rotation, energy (e.g. thermal energy) can be delivered by functional assembly 130 to target tissue located proximate functional assembly 130, such as to ablate the target tissue. In these embodiments, catheter 100 or another component of system 10 can aspirate fluid from the lumen proximate the target tissue, as described herein.

In some embodiments, system 10 is configured to treat NAFLD and/or NASH (“NAFLD/NASH” herein), such as is described in applicant's issued U.S. Pat. No. 9,757,535, entitled “Systems, Devices and Methods for Performing Medical Procedures in the Intestine”, filed Sep. 23, 2016. In these embodiments, system 10 can be configured to treat patients inflicted with NAFLD/NASH as well as diabetes (e.g. Type 2 diabetes).

In some embodiments, system 10 is configured to treat a patient that is taking insulin, such as when catheter 100 is used to treat duodenal mucosa and agent 420 comprises a GLP-1 receptor agonist, and the patient stops taking insulin. In these embodiments, the metabolic conditions of these patients can be improved or at least maintained (e.g. HbA1c level or other metabolic condition marker is not made significantly worse by the removal of insulin therapy).

In some embodiments, the target tissue treatment is performed to reduce the density of the crypts in the small intestine (e.g. reduce the density of the crypts in the duodenum and/or the jejunum). This reduction in crypt density can be performed to reduce the likelihood of undesired mucosal thickening after the treatment of the present inventive concepts is performed (e.g. to treat insulin resistance, Type 2 diabetes, and/or other medical conditions as described herein). In some embodiments, the target tissue treatment of the present inventive concepts is configured to reduce crypt density at least 20%, at least 40%, or at least 60% in the area of the treatments (e.g. in the area of ablative energy delivery). In some embodiments, the target tissue treatment of the present inventive concepts is configured to reduce crypt density of the duodenum (i.e. the entire duodenum) at least 5%, or at least 10%.

In some embodiments, system 10 is constructed and arranged to alter intestinal microbiota, such as to perform a treatment that affects a patient's gut flora in a way that leads to an improvement in weight and/or metabolic status (e.g. to treat Type 2 diabetes). Catheter 100 and functional assembly 130 can be configured to treat target tissue including intestinal mucosa such as to destroy local bacteria and/or modify the microbiome in the treated tissue area. Target tissue can include tissue regions where the microbiota contributes to the incidence or maintenance of metabolic disease.

In some embodiments, system 10 is constructed and arranged to reduce or otherwise alter the surface area of intestinal mucosa, such as is described in applicant's co-pending U.S. patent application Ser. No. 16/379,554, entitled “Methods, Systems and Devices for Reducing the Luminal Surface Area of the Gastrointestinal Tract”, filed Apr. 9, 2019. In some embodiments, system 10 is configured to reduce or otherwise alter the surface area of intestinal mucosa as a treatment for diabetes, a metabolic disease, obesity and/or hypercholesterolemia. In these embodiments, treatment of target tissue comprising mucosal folds and/or other mucosal tissue results in intestinal mucosa with reduced plicae circulares and delayed recovery or regrowth of intestinal villi. The treatment provided by system 10 can comprise a durable treatment effect that reduces the total absorptive surface area of the treated region. Alternatively or additionally, the treatment provided by system 10 can reduce enteroendocrine cell and/or absorptive cell quantities in the intestine by reducing the geometric complexity of the intestinal surface, such as by a target tissue treatment comprising ablation of intestinal tissue to a certain depth (mucosa alone; mucosa and superficial submucosa; mucosa through mid-submucosa; or mucosa through deep submucosa) that induces the healing response that leads to elimination of plicae circulares and blunting of villi for a prolonged period of time (at least 2 weeks, at least 6 weeks, at least 6 months or at least one year).

In some embodiments, system 10 is configured to treat a sufficient amount (e.g. a sufficient volume) of duodenal mucosa to provide an improvement in a patient's diabetes, such as is described in applicant's co-pending U.S. patent application Ser. No. 15/406,572, entitled “Methods and Systems for Treating Diabetes and Related Diseases and Disorders”, filed Jan. 13, 2017.

In some embodiments, system 10 is configured to create a therapeutic restriction in a patient, such as is described in applicant's co-pending U.S. patent application Ser. No. 16/267,771, entitled “Systems, Devices and Methods for the Creation of a Therapeutic Restriction in the Gastrointestinal Tract”, filed Feb. 5, 2019. In some embodiments, the therapeutic restriction is created at a location selected from the group consisting of: within mucosal tissue; within submucosal tissue; between mucosal and submucosal tissue; and combinations thereof. In some embodiments, the therapeutic restriction is created at a location selected from the group consisting of: lower stomach; pylorus; proximal small intestine; duodenum; proximal jejunum; distal small intestine; distal jejunum; ileum; and combinations thereof. In some embodiments, the therapeutic restriction is created in a location selected from the group consisting of: colon; rectum; anal sphincter and combinations thereof. The therapeutic restriction can be created by injecting (e.g. via one or more fluid delivery elements 139c) a volume of injectate 221 configured to create a 1 mm or greater separation between the mucosa and muscularis. In some embodiments, the therapeutic restriction is created by injecting (e.g. via one or more fluid delivery elements 139c) a volume of injectate 221 of at least 1.0 mL. The therapeutic restriction can be created by injecting a volume of injectate 221 of at least 3.0 mL, or at least 4.0 mL. In some embodiments, the therapeutic restriction is created by injecting a volume of injectate 221 of no more than 20.0 mL. The therapeutic restriction can be created by injecting a volume of injectate 221 of no more than 10.0 mL, or no more than 8.0 mL. In some embodiments, the therapeutic restriction comprises an axial length between 1 mm and 100 mm. The therapeutic restriction can comprise an axial length between 1 mm and 20 mm. The therapeutic restriction created using system 10 can comprise an axial length of at least 5 mm or at least 10 mm, or an axial length of no more than 100 mm, no more than 50 mm, or no more than 20 mm. In some embodiments, the therapeutic restriction comprises an inner diameter (e.g. diameter of its open portion) that is less than or equal to 10 mm. The therapeutic restriction can comprise an inner diameter less than or equal to 5 mm, 4 mm, 3 mm, 2 mm or 1 mm. In some embodiments, the therapeutic restriction comprises an inner diameter that is between 1% and 50% (e.g. 99% to 50% narrowing, respectively) of the inner diameter of the luminal segment prior to creation of the therapeutic restriction. The therapeutic restriction can comprise an inner diameter that is between 1% and 20% of the inner diameter of the luminal segment prior to creation of the therapeutic restriction. The inner diameter of the therapeutic restriction can increase over time, such as via the therapeutic restriction volume decreasing over time such as via absorption, migration or other reduction of the delivered injectate 221. The inner diameter of the therapeutic restriction can increase to an inner diameter that is between 11% and 20% of the inner diameter of the luminal segment prior to creation of the therapeutic restriction. The therapeutic restriction can comprise an inner diameter that is between 1% and 10% of the inner diameter of the luminal segment prior to creation of the therapeutic restriction. The therapeutic restriction can comprise an inner diameter that is between 1% and 5% of the inner diameter of the luminal segment prior to creation of the therapeutic restriction.

System 10 can be constructed and arranged to perform one or more diagnostic procedures (e.g. one or more diagnostic and/or prognostic procedures). In some embodiments, system 10 is constructed and arranged to perform a lumen sizing procedure, such as a procedure in which one or more diameters of one or more lumen locations in the intestine are determined (e.g. estimated). In these embodiments, the relative location at which the diameter is determined can be maintained at a pressure at or near room pressure (e.g. via one or more lumens of catheter 100 and/or body introduction device 50). System 10 can be constructed and arranged to perform a patient imaging procedure, such as a procedure in which a patient image is collected, such as a patient image that includes functional assembly 130 positioned in a segment of the intestine. System 10 can be constructed and arranged to perform a tissue sampling procedure, such as in a biopsy procedure. In some embodiments, system 10 is constructed and arranged to perform a diagnostic and/or other procedure selected from the group consisting of: assessment of mucosal thickness and/or hypertrophy, such as while using OCT or similar imaging technologies; assessment of wall thickness, such as via endoscopic ultrasound or similar imaging technologies; visualization of enteroendocrine cell populations, such as via molecular imaging techniques or antibody labeling; assessment of the location of the ampulla of Vater, such as via bile acid labeling; and combinations of one or more of these. In some embodiments, system 10 is constructed and arranged to perform a therapeutic and/or other procedure selected from the group consisting of: an obesity treatment procedure, such as an endoluminal implant of a balloon or other volume reducing and/or restricting device in the stomach or small intestine, a suturing or anastomosing procedure to reduce and/or restrict gastrointestinal volume, and/or an intestinal bypass; a procedure including the injection of sclerosing material configured to induce scar formation; a procedure including the injection of material to create a therapeutic restriction; a procedure including the injection of drugs or other agents into the submucosal space; a microbial transplantation procedure, such as to alter gut microbial populations; and combinations of one or more of these.

In some embodiments, system 10 is constructed and arranged to perform a patient assessment, such as a patient screening to determine if an intestinal tissue ablation (e.g. a duodenal mucosa ablation) would benefit the patient. In these embodiments, system 10 and/or the methods of the present inventive concepts can be configured to compare glucagon administered orally (PO) versus glucagon administered intravenously (IV). Data gathered can include the difference in the patient's ability to suppress glucagon after a meal. Patients whose ability to suppress glucagon falls below a threshold can be selected to receive a treatment of the present inventive concepts (e.g. an ablation or other treatment to at least the duodenal mucosa). Alternatively or additionally, analysis of fasting and/or postprandial glucagon can be compared to a threshold, and patients whose level is above the threshold can be selected to receive a treatment of the present inventive concepts (e.g. a treatment to at least the duodenal mucosa).

Catheter 100 of system 10 includes shaft 110, typically a flexible shaft comprising one or more lumens. In some embodiments, shaft 110 comprises a length that is long enough to treat distal segments of the duodenum and/or proximal segments of the jejunum, without being a length long enough to reach to the distal jejunum. For example, shaft 110 can be configured to be arranged in a “long path” through the stomach, and to comprise a length that is long enough to treat distal segments of the duodenum and/or proximal segments of the jejunum, without being a length long enough to reach to the distal jejunum. The long path position is achieved when a portion of shaft 401 is pressing against a wall of the stomach, following the curvature of the stomach from the end of the esophagus to the pylorus, such as is described in applicant's co-pending U.S. Provisional Patent Application Ser. No. 62/960,340, entitled “Tissue Treatment Devices, Systems, and Methods”, filed Jan. 13, 2020. Shaft 110 may be sufficiently flexible to cause it to take a long path through the stomach. In some embodiments, shaft 110 comprises varied flexibility along its length (e.g. a shaft with a variable wall thickness and/or variable stiffness of material). In some embodiments, distal portion 100_{DP} of catheter 100 (e.g. the distal portion of shaft 110) is sufficiently flexible to prevent placing undesired forces on the luminal wall of the small intestine. For example, distal portion 100_{DP} can be of sufficient flexibility to prevent forces on outside bends or otherwise enhance tracking within the lumen during translation of catheter 100 and/or to improve circumferential apposition (e.g. improve contact on inside bends) of functional assembly 130 during energy delivery to target tissue. In some embodiments, the distal portion of shaft 110, tip 115, comprises the bulbous geometry shown. Tip 115 can comprise a bulbous geometry with a diameter of at least 4 mm and/or a diameter less than or equal to 15 mm. In some embodiments, tip 115 comprises an inflatable bulbous tip. In some embodiments, tip 115 comprises a variable stiffness, such as to aid in directional translation of tip 115. An operator graspable handle, handle 102 is positioned on the proximal end of shaft 110. Handle 102 can comprise a user interface, such as user interface 105 shown. User interface 105 can comprise one or more user input components and/or user output components. User interface 105 can comprise one or more user input components configured to allow an operator to modify one or more console 200 operating parameters, settings 201, such as an operator-based modification based on information provided via a signal produced by a sensor of system 10. User interface 105 can comprise a control (e.g. control 104 described herein in reference to FIG. 2) and/or other user input component selected from the group consisting of: switch; keyboard; membrane keypad; knob; lever; touchscreen; and combinations of one or more of these. User interface 105 can comprise a user output component selected from the group consisting of: light such as an LED; display; touchscreen; audio transducer such as a buzzer or speaker; tactile transducer such as an eccentric rotational element; and combinations of one or more of these.

As described hereabove, catheter 100 further includes functional assembly 130, which can be positioned on a distal portion 100_{DP} of catheter 100 as shown. Functional assembly 130 can be constructed and arranged to perform a patient diagnosis and/or perform a patient treatment, such as a diagnosis or treatment performed on tissue of the intestine (e.g. mucosal and/or submucosal tissue of the intestine). In some embodiments, functional assembly 130 comprises an expandable assembly constructed and arranged to radially expand as determined by an operator of system 10. Functional assembly 130 can comprise an expandable element selected from the group consisting of: an inflatable balloon (e.g. balloon 136 as shown); a radially expandable cage or stent; one or more radially deployable arms; an expandable helix; an unfurlable compacted coiled structure; an unfurlable sheet; an unfoldable compacted structure; and combinations of one or more of these. Functional assembly 130 is shown in a radially expanded state in FIG. 1. Balloon 136 can comprise a compliant balloon, a non-compliant balloon and/or a balloon with compliant and non-compliant sections, as described herein. Balloon 136 can comprise one, two, or more materials that result in a compliance of balloon 136 of less than 10%, such as a compliance between 0.01% and 8%. In some embodiments, balloon 136 comprises PET. Balloon 136 can comprise one, two, or more materials that result in balloon 136 comprising a thermal conductivity with a “k value” of at least 0.20, such as at least 0.25, such as at least 0.30, such as at least 0.40, such as at least 0.45, such as approximately 0.50. Balloon 136 can comprise one, two, or more additives, such as metal additives configured to increase thermal conductivity of balloon 136 and/or one or more additives configured to enhance a visual property (e.g. radiopacity or ultrasonic reflectance) of balloon 136. Balloon 136 can comprise an outer diameter of no more than 32 mm, such as a diameter of approximately 24 mm. Balloon 136 can comprise a pressure-thresholded balloon, also as described herein. Balloon 136 can comprise a multi-layer construction, such as a construction with different materials positioned in different layers of balloon 136. In some embodiments, at least the distal portion of catheter 100, distal portion 100_{DP}, is constructed and arranged to be: inserted through an endoscope such as body introduction device 50; inserted alongside an endoscope; inserted over a guidewire such as guidewire 60; inserted through a sheath such as scope attachable sheath 80; and/or inserted through an introducer such as introducer 90 shown (e.g. an introducer sheath).

Positioned within shaft 110 are one or more conduits or lumens, conduits 111. Conduits 111 can comprise a conduit selected from the group consisting of: a fluid transport conduit (e.g. a tube or lumen configured to deliver fluids to functional assembly 130 and/or extract fluids from functional assembly 130); a tube comprising a lumen; a tube comprising a translatable rod; a hydraulic tube; a pneumatic tube; a tube configured to provide a vacuum (e.g. provide a vacuum to port 137 described herein); a lumen of shaft 110; an inflation lumen; a lumen configured to provide a vacuum (e.g. provide a vacuum to port 137); a fluid delivery lumen; a wire such as an electrically conductive wire; a linkage; a cable; a rod; a flexible filament; an optical fiber; and combinations of one or more of these. One or more conduits 111 can be configured to: transport fluid (e.g. deliver fluid and/or extract fluid); extract fluid; provide a positive pressure; provide a vacuum; and combinations of one or more of these. One or more conduits 111 can comprise a hollow tube, such as a tube comprising polyimide and/or a tube comprising a braid, such as a braided polyimide tube. One or more conduits 111 can be configured to allow the transport of: power, signals and/or materials such as fluids. A conduit 111 can be configured to slidingly receive a guidewire (e.g. guidewire 60), such as for over-the-wire delivery of catheter 100, such as when a conduit 111 is operably connected to a guidewire lumen of tip 115, lumen 116 shown. Alternatively, guidewire lumen 116 can both enter and exit distal portion 100_{DP} of catheter 100 (e.g. enter and exit tip 115, as shown in FIG. 1), such as for rapid-exchange manipulation of catheter 100 over a guidewire. In some embodiments, one or more conduits 111 can be translated within shaft 110 (e.g. advanced and/or retracted), such as to change the position of a distal end of a conduit 111 (e.g. to change the position of an outflow tube or inflow tube within functional assembly 130).

Shaft 110 can comprise one or more functional elements, such as functional element 119 shown. Functional element 119 can be positioned on (e.g. on the outer surface of), in (e.g. within the wall of) and/or within (e.g. within a lumen of) shaft 110. Functional element 119 can be positioned proximate (e.g. nearby, on, in and/or within) one or more conduits 111, such as when functional element 119 comprises a valve, heating element and/or cooling element configured to exert a force and/or alter the temperature of one or more fluids passing within a conduit 111.

Functional assembly 130 can comprise one or more functional elements, functional element 139, such as treatment element 139a, sensor 139b and/or fluid delivery element 139c, all shown in FIG. 1. Each functional element 139 can comprise a sensor, a transducer and/or other functional element, as described in detail herein.

In some embodiments, one or more functional elements 139 are constructed and arranged as a tissue treatment element of the present inventive concepts, as described herein, such as when treatment element 139a comprises an energy delivery element configured to treat target tissue of the intestine. Treatment element 139a can be of similar construction and arrangement as treatment element 135 described herein in reference to FIG. 2. Treatment element 139a can comprise a treatment element selected from the group consisting of: an ablative fluid (e.g. an ablative fluid to be maintained within balloon 136 and/or an ablative fluid to be delivered onto tissue such as via a fluid delivery element 139c); an electrode configured to deliver radiofrequency (RF) or other electrical energy to tissue; an optical element (e.g. a lens or a prism) configured to deliver laser and/or other light energy to tissue; a sound energy delivery element such as a piezo crystal configured to deliver ultrasound or subsonic sound energy to tissue; an agent delivery element such as a needle, nozzle or other fluid delivery element configured to deliver an ablative or other agent onto and/or into tissue; and combinations of one or more of these. In some embodiments, treatment element 139a comprises fluid at an ablative temperature (e.g. sufficiently hot or cold to ablate tissue). In these embodiments, treatment element 139a can comprise fluid whose temperature changes, such as when system 10 is configured to introduce a fluid both at an ablative temperature and fluid at a neutralizing temperature (e.g. a cooling fluid or a warming fluid, respectively), such as when fluid at a neutralizing temperature is delivered within functional assembly 130 before and/or after fluid at an ablative temperature is delivered within functional assembly 130, as described in detail herein.

In some embodiments, one or more functional elements 139 are constructed and arranged to perform a diagnosis and/or prognosis (“diagnosis” herein), such as when sensor 139b comprises a sensor configured to sense a physiologic parameter of intestinal tissue of the patient. Sensor 139b can comprise one or more sensors, such as are described in detail herein.

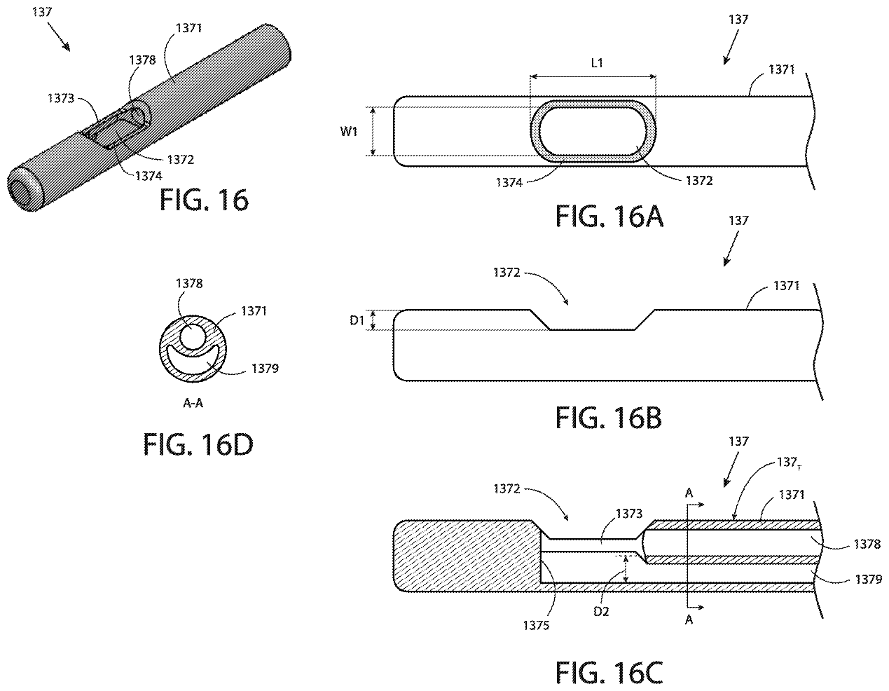

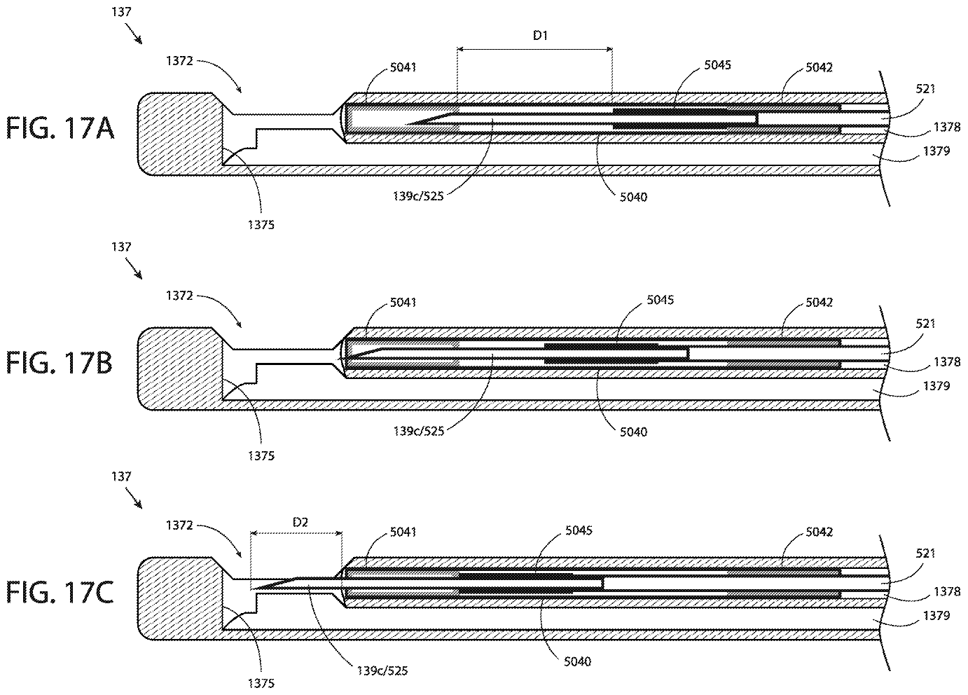

In some embodiments, one or more functional elements 139 are constructed and arranged to expand tissue, such as when fluid delivery element 139c comprises one or more of: needle; nozzle; fluid jet; iontophoretic fluid delivery element; an opening in functional assembly 130 (e.g. an opening in balloon 136); and/or any fluid delivery element configured to deliver fluid into and/or onto tissue (e.g. into submucosal tissue). Delivery of fluid into and/or onto tissue by catheter 100 can be configured to: protect at least a portion of tissue from an ablative treatment (e.g. limit damage to non-target tissue); perform an ablative treatment (e.g. treat target tissue); and/or to neutralize an ablative treatment (e.g. limit damage to non-target tissue). In some embodiments, fluid delivery element 139c comprises an element (e.g. a needle or fluid jet) configured to deliver fluid into tissue, such as submucosal tissue, to expand the tissue receiving the injected fluid. Alternatively or additionally, fluid delivery element 139c can comprise an element (e.g. a nozzle) configured to deliver fluid onto tissue, such as ablative fluid delivered onto tissue to ablate and/or remove tissue or neutralizing fluid configured to reduce tissue trauma (e.g. limit the volume of tissue ablated). Fluid delivery element 139c can comprise a needle selected from the group consisting of: a straight needle; a curved needle; a single lumen needle; a multiple lumen needle; and combinations of one or more of these. Fluid delivery element 139c can be positioned proximate and/or within a port, such as port 137 shown. Port 137 includes an opening (e.g. opening 1372 described herein in reference to FIGS. 16 and 17) sized and arranged to capture tissue, such as tissue into which a fluid delivery element 139c is configured to deliver fluid (e.g. penetrate and deliver fluid or simply deliver fluid). Port 137 can be placed on top of balloon 136 and/or recessed into balloon 136 (e.g. positioned within a recess of balloon 136 or other component of functional assembly 130). Port 137 can be engaged between layers of balloon 136, such as when balloon 136 comprises multiple layers including an outer layer (e.g. a layer of PET material) that surrounds at least a portion of port 137. In some embodiments, port 137 comprises an insulating element (e.g. port 137 comprises at least a portion that functions as a thermal insulator), such as an insulating element configured to prevent full circumferential ablation of an axial segment of intestine. Alternatively, port 137 can be constructed of materials that have a relatively high thermal conductivity, to efficiently transfer ablative heat or ablative cold to tissue. Port 137 can be positioned on a tissue-contacting portion of balloon 136 as shown. Port 137 can be attached to a source of vacuum, such as vacuum provided by a conduit 111, such that port 137 can engage with the tissue (e.g. capture the tissue within an opening of port 137). Port 137 can be constructed and arranged such that tissue can be drawn into port 137, such as when tissue is drawn into port 137 prior to delivery of fluid by fluid delivery element 139c into tissue, as described herein. In some embodiments, catheter 100 comprises multiple ports 137 and multiple corresponding fluid delivery elements 139c, such as two, three or more pairs of a port 137 with an associated fluid delivery element 139c (e.g. equally spaced about a circumference of balloon 136). For example, three ports 137 can be separated by approximately 120 degrees to provide an equal distribution of fluid delivery elements 139c about balloon 136, such as to provide a circumferential delivery of fluid into tissue. One or more functional elements 139 can be attached to one or more conduits 111 and each can be configured to be translated (e.g. translated within a port 137). In some embodiments, the geometry of port 137 (e.g. depth, width, and/or length of the opening of port 137 that captures the tissue) is based on the depth of the layer of intestinal tissue to be expanded (e.g. the depth to receive fluid from the associated fluid delivery element 139c). For example, a first port 137 that has an opening with a depth that is greater than the opening of a second port 137 will capture deeper layers of tissue to be subsequently accessed by and receive fluid from an advanced fluid delivery element 139c. In some embodiments, port 137 comprises a depth of at least 1.0 mm, and/or a depth of no more than 2.5 mm, such as a depth of approximately 1.3 mm. In some embodiments, system 10 can select a level of vacuum to cause shallow or deeper layers of intestinal tissue to be captured by port 137 (e.g. higher pressure will cause more tissue to be captured by port 137), such as to similarly adjust the depth of the layer receiving tissue-expanding fluid from the associated fluid delivery element 139c.

In some embodiments, catheter 100 (e.g. each port 137) comprises proximal and/or distal stops (e.g. a mechanical stop) configured to define (e.g. limit) a translation distance of each fluid delivery element 139c within port 137. A proximal stop can be included to limit retraction of a fluid delivery element 139c within its associated port 137. A distal stop can be included to limit advancement of a fluid delivery element 139c within its associated port 137.

In some embodiments, port 137 comprises a surface (e.g. a distal end or other tissue-contacting surface) with an atraumatic contour (e.g. a rounded contour), such as to minimize injury to tissue that makes contact with the distal end of port 137.

In some embodiments, prior to the delivery of injectate 221 (e.g. prior to advancement of fluid delivery elements 139c), system 10 is configured to confirm the vacuum level provided by console 200 to a lumen 111 fluidly attached to port 137 (e.g. lumen 1379 of FIG. 17A-C) exceeds a threshold (e.g. as measured by a sensor-based functional element 299 of console 200), such as a vacuum of at least −4.4 psi, such as a vacuum of at least −6.0 psi. System 10 can be configured to prevent the advancement of fluid delivery elements 139c if the vacuum provided by console 200 does not exceed the threshold. System 10 can be configured to prevent the advancement of fluid delivery elements 139c if a vacuum is not detected by system 10 (e.g. due to a sensor or other console 200 component failure).

Translation of a fluid delivery element 139c can be limited by one or more mechanical stops constructed and arranged to limit advancement and/or retraction of fluid delivery element 139c. One or more fluid delivery elements 139c and an associated fluidly attached conduit 111 can be biased by one or more springs, such as one or more springs positioned in handle 102. Fluid delivery element 139c and an associated functional assembly 130 can be of similar construction and arrangement as those described herein in reference to catheter 20 and/or catheter 40 of FIG. 2, or as described in applicant's co-pending U.S. patent application Ser. No. 16/900,563, entitled “Injectate Delivery Devices, Systems and Methods”, filed Jun. 12, 2020. One or more fluid delivery element 139c can comprise a straight or a curved needle. One or more fluid delivery elements 139c can be constructed and arranged to enter tissue at an angle between 0° and 90°, such as at an angle between 30° and 60°.

In some embodiments, port 137 can be configured to engage tissue (e.g. when a vacuum is applied to port 137 via one or more conduits 111), after which target tissue can be treated by treatment element 139a. Engagement of tissue by port 137 can be used to stretch or otherwise manipulate tissue such that a safe and effective treatment of target tissue can be performed by treatment element 139a, such as when treatment element 139a comprises fluid at an ablative temperature or an array of electrodes configured to deliver RF energy. In these embodiments, catheter 100 can be configured to treat target tissue without performing an associated tissue expansion procedure (e.g. without expanding tissue in proximity to the target tissue to be treated).

Functional assembly 130 can be configured to treat target tissue, such as when functional element 139 comprises ablative fluid introduced into balloon 136 or when functional element 139 comprises one or more other energy delivery elements as described herein. Functional assembly 130 can be constructed and arranged to treat a full or partial circumferential axial segment of intestinal tissue (e.g. intestinal mucosa). System 10 can be configured to treat multiple axial segments of tissue, such as multiple relatively contiguous or discontiguous segments of mucosal tissue treated simultaneously and/or sequentially. The multiple segments can comprise overlapping and/or non-overlapping borders.

Catheter 100 is configured to operably attach to console 200. In some embodiments, catheter 100 attaches directly to console 200. In other embodiments, attachment assembly 300 is positioned and operably attached between catheter 100 and console 200, such as to transfer materials (such as injectate 221, agent 420, hydraulic and/or pneumatic fluid, ablative fluids and/or other fluids), energy (such as ablative fluids and/or electromagnetic energy), and/or data between catheter 100 and console 200. Attachment assembly 300 comprises a first end, end 301, which attaches to catheter 100 via a connector of handle 102, connector 103 as shown. Attachment assembly 300 further comprises a second end, end 302, which attaches to console 200 via a connector, connector 203 of console 200 as shown. Attachment assembly 300 includes one or more conduits, conduits 311 shown. Conduits 311 of attachment assembly 300 operably attach conduits 111 of catheter 100 to associated conduits of console 200, conduits 211 shown. Attachment assembly 300 can comprise a cassette configuration configured to operably attach to console 200. Attachment assembly 300 can comprise one or more flexible portions (e.g. coiled tubes and/or filaments) that allow movement of catheter 100 relative to console 200, such as to extend catheter 100 away from console 200 and toward a table onto which a patient is positioned. Attachment assembly 300 can comprise one or more functional elements 309, such as an array of functional elements 309, each positioned proximate a conduit 311. Each functional element 309 can comprise one or more sensors, transducers and/or other functional elements as described in detail herein.

Console 200 is configured to operably control and/or otherwise interface with catheter 100. In some embodiments, console 200 comprises one or more pumps or other fluid propulsion devices, pumping assemblies 225 (four shown in FIG. 1), which can each be attached to a reservoir, reservoir 220, via one or more conduits, conduits 212 shown. Each reservoir 220 can be constructed and arranged to store and supply fluids to catheter 100 and/or to extract fluids from catheter 100, such as is described herein in reference to system 10 of FIG. 2. An ablative fluid, a neutralizing fluid, agent 420 and/or injectate 221 can be placed or otherwise positioned within one or more reservoirs 220, such as to be transported by one or more pumping assemblies 225 into one or more conduits 111 of catheter 100 (e.g. via conduits 211 of console 200 and optionally via conduits 311 of connecting assembly 300). In some embodiments, console 200 is constructed and arranged to deliver a neutralizing fluid (e.g. a cooling fluid or warming fluid contained within a reservoir 220), then an ablative fluid (e.g. a hot fluid and/or a cryogenic fluid, respectively, contained within one or more reservoirs 220). In these embodiments, console 200 can be further constructed and arranged to subsequently deliver (i.e. after the ablation step), the same or a different neutralizing fluid (e.g. a cooling or warming fluid contained within a reservoir 220). In some embodiments, a first reservoir 220 provides an ablative fluid comprising a hot fluid at a temperature above 44° C., such as above 65° C., above 75° C., above 85° C. or above 95° C., and a second reservoir 220 provides a neutralizing fluid comprising a cooling fluid below 37° C., such as below 25° C., such as below 20° C. or below 15° C. In some embodiments, the neutralizing fluid comprises a cooling fluid maintained at approximately 15° C., such as a temperature between 9° C. and 25° C. In some embodiments, a first reservoir 220 provides an ablative fluid comprising a cryogenic fluid, and a second reservoir 220 provides a neutralizing fluid comprising a warming fluid at or above 37° C.

Alternatively or additionally, console 200 can be configured to provide RF and/or light energy to functional assembly 130 to ablate or otherwise treat tissue, and a cooling step can be performed (e.g. via a neutralizing fluid provided by a reservoir 220 comprising fluid below 37° C.) prior to and/or after the delivery of the RF and/or light energy. In some embodiments, system 10 comprises two return paths, one for recovery of ablative fluid (e.g. hot fluid), and one for recovery of neutralizing fluid (e.g. cooling fluid), such as via separate conduits 111, 311 and/or 211. In these embodiments, two separate pumping assemblies 225 can be fluidly attached to the separate return paths.

Console 200 comprises one or more console settings 201 that can be varied, such as a change made manually (e.g. by a clinician or other operator of system 10), and/or automatically by system 10. Console 200 comprises an electronic, mechanical, optical, fluidic, and/or other controller, controller 250 shown (e.g. a central processing unit and/or microcontroller). Controller 250 can comprise one or more signal processors, such as signal processor 252 shown. Signal processor 252 can be configured to analyze one or more sensor signals, such as to modify one or more settings 201 of console 200. Controller 250 and/or signal processor 252 can comprise one or more algorithms, algorithm 251 shown, which can be configured to perform one or more mathematical or other functions, such as to compare one or more sensor signals (e.g. compare the signal itself or a mathematical derivation of the signal) to a threshold. Console settings 201 can comprise one or more parameters (e.g. system parameters as also referred to herein) of catheter 100, console 200 and/or any component of system 10. Console settings 201 can comprise one or more parameters selected from the group consisting of: delivery rate of fluid into functional assembly 130; withdrawal rate of fluid from functional assembly 130; delivery rate of fluid into tissue; rate of energy delivered into tissue; peak energy level delivered into tissue; average energy delivery rate delivered into tissue; amount of energy delivered into tissue during a time period; temperature of an ablative fluid (e.g. temperature of an ablative fluid in reservoir 220, console 200, functional assembly 130 and/or catheter 100); temperature of a neutralizing fluid (e.g. temperature of a neutralizing fluid in reservoir 220, console 200, functional assembly 130 and/or catheter 100); temperature of functional assembly 130; pressure of functional assembly 130; pressure of fluid delivered into functional assembly 130; pressure of fluid delivered into tissue; duration of energy delivery; time of energy delivery (e.g. time of day of or relative time compared to another step); translation rate such as translation rate of a functional assembly 130; rotation rate such as rotation rate of a functional assembly 130; a flow rate; a recirculation rate; a heating rate or temperature; a cooling rate or temperature; a sampling rate (e.g. a sampling rate of a sensor); and combinations of one or more of these. In some embodiments, one or more console settings 201 comprise a setting related to a system 10 parameter selected from the group consisting of: pressure and/or volume of a fluid delivered to shaft 110 to change the stiffness of shaft 110 (e.g. to modify pushability and/or trackability); pressure and/or volume of a fluid delivered to and/or extracted from functional assembly 130 for inflation and/or deflation (e.g. to obtain apposition of ports 137 and/or to anchor functional assembly 130 in the intestine); pressure and/or volume of a fluid delivered to one or more conduits 111, each configured as a fluid transport tube to provide a fluid, injectate 221, to one or more fluid delivery elements 139c (described herein) such as to advance and/or retract one or more fluid delivery elements 139c and/or to deliver injectate 221 into tissue (e.g. submucosal tissue); pressure and/or volume of a fluid within one or more conduits 111, each configured to provide a vacuum to one or more ports 137 to engage the one or more ports 137 with tissue and/or to cause a fluid delivery element to engage (e.g. penetrate) tissue; a force used to advance and/or retract one or more conduits 111 and/or one or more fluid delivery elements 139c; and combinations of one or more of these. In some embodiments, one or more console settings 201 comprise a setting related to a system 10 parameter selected from the group consisting of: temperature, flow rate, pressure and/or duration of fluid delivered to catheter 100 and/or functional assembly 130; temperature, flow rate, pressure and/or duration of fluid contained within functional assembly 130 and/or circulating loops (e.g. conduits 111, 211 and/or 311) of system 10: and combinations of one or more of these. System 10 can be configured to adjust one or more console settings 201 based on one or more signals produced by one or more sensors of system 10. Based on the one or more sensor signals, system 10 can be configured to modify a console setting 201 to cause: stopping delivery of fluid and/or energy to and/or by functional assembly 130; delivering additional fluid into functional assembly 130 and/or into tissue (e.g. adjust fluid delivery rate); delivering neutralizing and/or other additional fluid into functional assembly 130 and/or into and/or onto tissue; adjusting the pressure of functional assembly 130; adjusting the volume of functional assembly 130; withdrawing (e.g. rapidly withdrawing) fluid from functional assembly 130 and/or other fluid-carrying portion of catheter 100; and combinations of one or more of these. In some embodiments, algorithm 251 is configured to determine an injectate 221 delivery parameter, such as the amount (e.g. volume and/or mass) of injectate 221 to be delivered by catheter 100.

Controller 250 can further comprise a self-diagnostic routine (e.g. as defined and/or performed via algorithm 251), continuous system self-monitoring (CSSM) routine, CSSM 255, as described herein.

In some embodiments, system 10 adjusts, via algorithm 251, a functional assembly 130 parameter based on a signal of a sensor of system 10. In these embodiments, a functional assembly 130 parameter can be adjusted during performance of a procedural step, such as an ablation step or a tissue expansion step. The functional assembly 130 parameter adjusted can comprise a parameter selected from the group consisting of: volume of functional assembly 130; diameter of functional assembly 130; pressure of functional assembly 130; force applied to tissue by functional assembly 130; and combinations of one or more of these. The functional assembly 130 parameter can be adjusted to prevent excessive force being applied to the intestinal wall or to maintain a minimum apposition level of functional assembly 130 with tissue of the intestine.

In some embodiments, console 200 comprises a first reservoir 220 containing hot fluid for ablation, a second reservoir 220 comprising cooling fluid at a first temperature (e.g. a temperature less than 37° C. but more than 10° C.), and a third reservoir 220 comprising fluid at a second temperature cooler than the first temperature (e.g. a temperature less than 6° C., such as a temperature between 2° C. and 4° C.). Fluid from the third reservoir 220 can be delivered into the second reservoir 220 (e.g. after one or more steps including cooling and ablation of tissue have been performed).

In some embodiments, console 200 comprises a first reservoir 220 containing hot fluid for ablation at a first temperature (e.g. approximately 55° C.), and a second reservoir 220 comprising hot fluid for ablation at a second temperature (e.g. approximately 95° C.). Fluid from the first reservoir 220 and the second reservoir 220 can be delivered to functional assembly 130 for equal time periods. In these embodiments, console 200 can further comprise a third reservoir 220 comprising cooling fluid, such as when console 200 is configured to deliver hot fluid from the first reservoir 220, followed by hot fluid from the second reservoir 220, followed by cooling fluid from the third reservoir 220. Console 200 can be further configured to deliver the cooling fluid prior to the delivery of the hot fluid from the first reservoir 220. In some embodiments, fluid from a reservoir 220 is delivered for a time period determined based on the temperature of fluid in that reservoir and/or based on the temperature of fluid in a separate reservoir 220, as described herein. For example, the amount of ablative fluid delivered by a reservoir 220 containing hot fluid can be adjusted based on the temperature of cooling fluid in a different reservoir 220.

In some embodiments, console 200 comprises two functional elements 209, a first functional element 209a comprising a heating element and a second functional element 209b comprising a cooling element. In these embodiments, connecting assembly 300 can comprise a tubing set configured to be engaged with console 200 to allow the first functional element 209a to transfer heat into fluid within connecting assembly 300 and the second functional element 209b to extract heat from (i.e. cool) fluid within connecting assembly 300. In these embodiments, system 10 can avoid the need for heated and/or cooled reservoirs 220, such as when console 200 further comprises a disposable fluid supply (e.g. used in a single clinical procedure) that is fluidly attached to connecting assembly 300. Connecting assembly 300 can comprise a reusable tubing set (e.g. used in multiple clinical procedures performed on the same or different patients). Connecting assembly 300 can comprise a tubing set comprising multiple lumens (e.g. multiple tubes each with one or more lumens, or a single tube with multiple lumens), such as at least a first lumen configured to deliver inflation fluid (e.g. deliver inflation fluid to functional assembly 130 to perform a tissue expansion procedure and/or a tissue sizing procedure), and at least two lumens configured to deliver a recirculating fluid (e.g. to recirculate ablative fluid and/or neutralizing fluid within functional assembly 130 during a tissue ablation procedure).

Console 200 can comprise a user interface, user interface 205 shown, which can deliver commands to controller 250 (e.g. commands from a clinician or other operator of system 10), and/or receive information (e.g. to be displayed) from controller 250. In some embodiments, console 200 comprises an energy delivery unit, EDU 260 shown, such as an energy delivery unit configured to provide one or more of: thermal energy such as heat energy or cryogenic energy; electromagnetic energy such as radiofrequency (RF) energy; light energy such as light energy provided by a laser; sound energy such as subsonic energy or ultrasonic energy; chemical energy (e.g. a chemically ablative substance); and combinations of one or more of these. EDU 260 can be of similar construction and arrangement as EDU 260 described herein in reference to FIG. 2. Console 200 can further comprise conduits 211 which can be operably connected to catheter 100 (e.g. operably connected to one or more conduits 111 or other components of catheter 100). Conduits 211 can comprise one or more fluid transport tubes fluidly attached to pumping assemblies 225 and/or any filament bundle operably attached to controller 250 and comprising one or more filaments selected from the group consisting of: a tube comprising a lumen; a tube comprising a translatable rod; a hydraulic tube; a pneumatic tube; a tube configured to provide a vacuum (e.g. provide a vacuum to port 137); a lumen of shaft 110; an inflation lumen; a fluid delivery lumen; a wire such as an electrically conductive wire; a linkage; a rod; a flexible filament; an optical fiber; and combinations of one or more of these. Controller 250 can be operably connected to one or more of reservoirs 220, pumping assemblies 225 and/or user interface 205 via a bus (e.g. an electronic communication and/or power transfer bus), bus 213 shown. Bus 213 can comprise one or more wires, optical fibers or other conduits configured to provide power, transmit data and/or receive data.

In some embodiments, console 200 is configured to operably expand functional assembly 130, such as with a liquid or gas provided by a reservoir 220 and propelled by an associated pumping assembly 225. In some embodiments, console 200 is configured to deliver fluid to tissue via one or more fluid delivery elements 139c, such as with a fluid (e.g. injectate 221) provided by a reservoir 220 and propelled by an associated pumping assembly 225. In some embodiments, console 200 is configured to deliver ablative fluid to functional assembly 130, such as ablative fluid provided by a reservoir 220 and propelled by an associated pumping assembly 225. In these embodiments, ablative fluid can be recirculated to and from functional assembly 130 by console 200. In some embodiments, console 200 is configured to deliver energy, such as electromagnetic or other energy, to functional assembly 130, such as via controller 250. Each of these embodiments is described in detail herein in reference to system 10 of FIG. 2.

One or more reservoirs 220 can each comprise one more functional elements 229a and/or one or more pumping assemblies 225 can each comprise one or functional elements 229b. Each functional element 229a and/or 229b (singly or collectively functional element 229) can comprise one or more sensors, transducers, and/or other functional elements. In some embodiments, one or more functional elements 229 comprise a heating element or a chilling element configured to heat or chill fluid within a reservoir 220 and/or a pumping assembly 225. Alternatively or additionally, one or more functional elements 229 comprise a sensor, such as a temperature sensor, pressure sensor and/or a flow rate sensor configured to measure the temperature, pressure and/or flow rate, respectively, of fluid within, flowing into, and/or flowing out of a reservoir 220 and/or pumping assembly 225.

In some embodiments, controller 250 comprises one or more algorithms, such as algorithm 251 configured to operatively adjust one or more operating parameters of console 200 and/or catheter 100 (generally console settings 201), such as an algorithm configured to analyze data provided by one or more sensors of system 10. Algorithm 251 can be configured to adjust (e.g. increase, decrease) the temperature of fluids within console 200 (e.g. in one or more reservoirs 220), such as an adjustment performed based on a temperature, pressure, and/or flow measurement performed by a sensor of system 10, and/or a patient parameter (e.g. a patient physiologic parameter) measurement performed by a sensor of system 10. In some embodiments, algorithm 251 causes an adjustment of a console setting 201 based on the measured temperature of a neutralizing fluid (e.g. cold fluid) of system 10. Algorithm 251 can be configured to correlate a signal received by one or more sensors of system 10 positioned at a first location, to a parameter of system 10 or the patient at a second location distant from the first location (e.g. a second location proximal or distal to the first location). For example, a measured temperature or pressure within console 200 (e.g. via functional element 229a or 229b), connecting assembly 300 (e.g. via functional element 309), and/or catheter 100 (e.g. via functional element 119 and/or 139b), can provide a signal related to a parameter at a remote location, such as a parameter of functional assembly 130 or the patient. Algorithm 251 can be configured to analyze a signal received from a first location, and to produce parameter information correlating to a second location.

In some embodiments, console 200 is constructed and arranged to operably attach and control multiple catheters 100, such as two or more catheters 100 of similar construction and arrangement to catheters 100, 20, 30 and/or 40 described herein in reference to FIG. 2.

In some embodiments, injectate 221 comprises a material selected from the group consisting of: water; saline; a gel; a hydrogel; a protein hydrogel; a cross-linked hydrogel; a cross-linked polyalkyleneimine hydrogel; autologous fat; collagen; bovine collagen; human cadaveric dermis; hyaluronic acid; calcium hydroxylapatite; polylactic acid; semi-permanent PMMA; dermal filler; gelatin; mesna (sodium 2-sulfanylethanesulfonate); and combinations of one or more of these. In some embodiments, injectate 221 comprises beads (e.g. pyrolytic carbon-coated beads) suspended in a carrier (e.g. a water-based carrier gel). In some embodiments, injectate 221 comprises a solid silicone elastomer (e.g. heat-vulcanized polydimethylsiloxane) suspended in a carrier, such as a bio-excretable polyvinylpyrrolidone (PVP) carrier gel. In some embodiments, injectate 221 has an adjustable degradation rate, such as an injectate 221 comprising one or more cross linkers in combination with polyalkylene imines at specific concentrations that result in hydrogels with adjustable degradation properties. In some embodiments, injectate 221 and/or agent 420 comprises living cells, such as living cells injected into the mucosa or submucosa of the intestine to provide a therapeutic benefit.

In some embodiments, injectate 221 comprises a visualizable and/or otherwise detectable (e.g. magnetic) material (e.g. in addition to one or more materials of above) selected from the group consisting of: a dye; a visible dye; indigo carmine; methylene blue; India ink; SPOT′ dye; a visualizable media; radiopaque material; radiopaque powder; tantalum; tantalum powder; ultrasonically reflective material; magnetic material; ferrous material; and combinations of one or more of these.

In some embodiments, injectate 221 comprises a material selected from the group consisting of: a peptide polymer (e.g. a peptide polymer configured to stimulate fibroblasts to produce collagen); polylactic acid; polymethylmethacrylate (PMMA); a hydrogel; ethylene vinyl alcohol (EVOH); a material configured to polymerize EVOH; dimethyl sulfoxide (DMSO); saline; material harvested from a mammalian body; autologous material; fat cells; collagen; autologous collagen; bovine collagen; porcine collagen; bioengineered human collagen; dermis; a dermal filler; hyaluronic acid; conjugated hyaluronic acid; calcium hydroxylapatite; fibroblasts; a sclerosant; an adhesive; cyanoacrylate; a pharmaceutical agent; a visualizable material; a radiopaque material; a visible dye; ultrasonically reflective material; and combinations of one or more of these. As described herein, in some embodiments, a volume of injectate 221 is delivered into tissue to create a therapeutic restriction (e.g. a therapeutic restriction with an axial length between 1 mm and 20 mm), as described herein, or as is described in applicant's co-pending U.S. patent application Ser. No. 16/267,771, entitled “Systems, Devices and Methods for the Creation of a Therapeutic Restriction in the Gastrointestinal Tract”, filed Feb. 5, 2019. In some embodiments, a volume of injectate 221 is delivered into tissue to create a safety margin of tissue that is created (e.g. an expanded tissue layer is created) prior to an ablation procedure, as is described herein.

In some embodiments, injectate 221 comprises a fluorescent-labeled material or other biomarker configured to identify the presence of a biological substance, such as to identify diseased tissue and/or other tissue for treatment by functional assembly 130 (e.g. to identify target tissue). For example, injectate 221 can comprise a material configured to be identified by imaging device 55 (e.g. identify a visualizable change to injectate 221 that occurs after contacting one or more biological substances). In these embodiments, imaging device 55 can comprise a molecular imaging device, such as when imaging device 55 comprises a molecular imaging probe and injectate 221 comprises an associated molecular imaging contrast agent. In these embodiments, injectate 221 can be configured to identify diseased tissue and/or to identify a particular level of one or more of pH, tissue oxygenation, blood flow, and the like. Injectate 221 can be configured to be delivered onto the inner surface of intestinal or other tissue, and/or to be delivered into tissue (i.e. beneath the surface).

In some embodiments, injectate 221 comprises an isotonic solution configured to prevent or otherwise reduce cell damage upon delivery into tissue.

In some embodiments, injectate 221 comprises a thermally-insulating fluid.

In some embodiments, injectate 221 comprises a fluid at a temperature less than the body temperature of the patient (e.g. less than 37° C.).

As described hereabove, system 10 can include agent 420, which can include one or more drugs or other agents delivered to the patient (e.g. orally, transdermally, via injection, or otherwise). In some embodiments, agent 420 comprises a material selected from the group consisting of: anti-peristaltic agent, such as L-menthol (i.e. oil of peppermint); glucagon; buscopan; hycosine; somatostatin; a diabetic medication; an analgesic agent; an opioid agent; a chemotherapeutic agent; a hormone; and combinations of one or more of these.

In some embodiments, agent 420 comprises cells delivered into the intestine, such as living cells delivered into intestinal mucosa or submucosa via a fluid delivery element 139c or otherwise.

As described hereabove, system 10 can comprise one or more sensors, transducers and/or other functional elements, such as functional element 109, functional element 119 and/or functional element 139 (e.g. 139a, 139b and/or 139c) of catheter 100 and/or functional element 209 and/or functional element 229 (e.g. 229a and/or 229b) of console 200. In some embodiments, system 10 comprises connecting assembly 300 which can include one or more functional elements 309.

In some embodiments, one or more functional elements 109, 119, 139, 209, 229 and/or 309 comprise a transducer selected from the group consisting of: an energy converting transducer; a heating element; a cooling element such as a Peltier cooling element; a drug delivery element such as an iontophoretic drug delivery element; a magnetic transducer; a magnetic field generator; a sound generator; an ultrasound wave generator such as a piezo crystal; a light producing element such as a visible and/or infrared light emitting diode; a motor; a pressure transducer; a vibrational transducer; a solenoid; a fluid agitating element; and combinations of one or more of these.

In some embodiments, one or more functional elements 109, 119, 139, 209, 229 and/or 309 comprise a visualizable element, such as an element selected from the group consisting of: a radiopaque marker; an ultrasonically visible marker; an infrared marker; a marker visualizable by a camera such as an endoscopic camera; a marker visualizable by an MRI, a chemical marker; and combinations of one or more of these.

In some embodiments, one or more of functional elements 109, 119, 139, 209, 229 and/or 309 comprise a sensor configured to produce a signal, the sensor selected from the group consisting of: physiologic sensor; blood glucose sensor; blood gas sensor; blood sensor; respiration sensor; EKG sensor; EEG sensor; neuronal activity sensor; blood pressure sensor; flow sensor such as a flow rate sensor; volume sensor (e.g. a volume sensor used to detect a volume of injectate 221 not delivered into tissue); pressure sensor; force sensor; sound sensor such as an ultrasound sensor; electromagnetic sensor such as an electromagnetic field sensor or an electrode; gas bubble detector such as an ultrasonic gas bubble detector; strain gauge; magnetic sensor; ultrasonic sensor; optical sensor such as a light sensor; chemical sensor; visual sensor such as a camera; temperature sensor such as a thermocouple, thermistor, resistance temperature detector or optical temperature sensor; impedance sensor such as a tissue impedance sensor; and combinations of one or more of these. Each sensor can be configured to produce a signal that directly correlates to or is otherwise related to a patient parameter or a system 10 parameter. One or more console settings 201 can be manually adjusted (e.g. by a clinician or other operator of system 10) and/or automatically (e.g. by an algorithm of system 10) based on the sensor signal.

In some embodiments, a functional element of functional element 109, 119, and/or 139 of catheter 100 comprises one, two, or more sensors configured to produce one or more signals related to tissue impedance. In these embodiments, algorithm 251 of console can be configured to analyze the one or more signals to assess ablation performed by catheter 100 (e.g. to assess the amount of tissue ablated), and/or to assess tissue expansion performed by catheter 100 (e.g. to assess the amount of submucosal tissue expansion achieved).

In some embodiments, one or more of functional elements 109, 119, 139, 209, 229 and/or 309 comprise a pressure sensor that produces a signal related to one or more of: pressure within functional assembly 130; the level of apposition of functional assembly 130 with the intestine; level of aspiration within a segment of the intestine; the diameter of the intestine proximate functional assembly 130; muscular contraction of the intestine; pressure within a reservoir 220; pressure within connecting assembly 300; pressure within a lumen of shaft 110; and combinations of one or more of these. One or more console settings 201 can be adjusted (e.g. manually or automatically) based on the pressure sensor signal. In some embodiments, a pressure sensor produces a signal related to the pressure within functional assembly 130, console 200 delivers and/or extracts fluids to and/or from functional assembly 130 via one or more conduits 111, and console 200 adjusts the pressure and/or volume of functional assembly 130, such as to maintain pressure in, volume of, and/or flow rate within functional assembly 130 below a threshold.

In some embodiments, one or more of functional elements 109, 119, 139, 209, 229 and/or 309 comprise a temperature sensor that produces a signal related to one or more of: temperature of fluid in console 200 (e.g. in one or more reservoirs 220); temperature of elongate shaft 110; temperature of fluid within elongate shaft 110; temperature of functional assembly 130; temperature of fluid within functional assembly 130; temperature of an ablative fluid; temperature of a neutralizing fluid; temperature of tissue proximate the functional assembly; temperature of target tissue; temperature of non-target tissue; and combinations of one or more of these. One or more console settings 201 can be adjusted (e.g. manually or automatically) based on the temperature sensor signal.

In some embodiments, system 10 comprises a sensor (e.g. a functional element 109, 119, 139, 209, 229 and/or 309 comprising a sensor) configured to detect a parameter related to a level of treatment of tissue, such as a parameter selected from the group consisting of: color, density and/or saturation of tissue (e.g. a color change to tissue that occurs during ablation or to an injectate 221 present in the tissue during ablation or other treatment); temperature of local tissue and/or temperature of other body tissue; texture, length and/or diameter of villi or other mucosal feature (e.g. as detected via a camera-based sensor, such as when ablation causes a blunting and/or drooping of villi or other intestinal tissue); electrical resistance, impedance and/or capacitance of tissue (e.g. as altered by ablation of tissue); pressure and/or force of peristaltic contractions (e.g. as altered by ablation of tissue); compliance of tissue and/or the entire duodenum in radial and/or axial directions (e.g. as altered by ablation of tissue); chemical composition of film adhered to mucosal tissue (e.g. as altered by ablation); types, quantities and/or locations of bacterial colonies present (e.g. as altered by ablation); and combinations of one or more of these. As described herein, system 10 can be configured to determine (e.g. assess) the quality of the treatment based on signals produced by the sensors. In some embodiments, the treatment of the target tissue comprises at least two treatments (e.g. a treatment of a first segment of intestinal mucosal tissue and a treatment of a second segment of intestinal mucosal tissue), and a subsequent treatment is modified based on signals produced by the sensors during a prior treatment.

In some embodiments, system 10 comprises a sensor (e.g. a functional element 109, 119, 139, 209, 229 and/or 309 comprising a sensor) configured to detect a parameter related to a level of tissue expansion, such as a parameter selected from the group consisting of: color, density and/or saturation related to injected dye or particles which alter tissue appearance (e.g. as determined via a camera-based sensor); temperature of tissue (e.g. that can be altered briefly due to delivery of injectate 221 and/or inflammation response due to injectate 221 delivery); texture, length and/or diameter of villi or mucosal features (e.g. as determined via a camera-based sensor) such as spacing between villi or other intestinal tissue features that can change (e.g. increased spacing, disappearance or reduction of plicae, blebs of injectate 221 present) due to submucosal tissue expansion; electrical resistance, impedance and/or capacitance of tissue (e.g. as altered by delivery of injectate 221); pressure and/or force of peristaltic contractions (e.g. as altered by delivery of injectate 221); compliance of tissue and/or the entire duodenum in radial and/or axial directions (e.g. as altered by injectate 221, such as to make tissue more compliant until the muscularis layer is contacted); chemical composition of film adhered to mucosa (e.g. as altered by injectate 221, such as when injectate 221 creates a biologic response that is detectable); types, quantities and/or locations of bacterial colonies present; and combinations of one or more of these. As described herein, the quality of the tissue expansion can be determined by system 10 based on signals produced by the sensors (e.g. an analysis of the sensor signals performed by algorithm 251). In some embodiments, the tissue expansion comprises at least two tissue expansions, and a subsequent tissue expansion is modified based on signals produced by the sensors during a prior tissue expansion.

In some embodiments, system 10 comprises a sensor (e.g. a functional element 109, 119, 139, 209, 229 and/or 309 comprising a sensor) configured to assess engagement of port 137 with tissue (e.g. to determine if adequate engagement is present during a tissue expansion or tissue ablation step in which vacuum is applied to port 137 to engage port 137 with tissue). In some embodiments, a sensor is positioned to detect injectate in a conduit 111 of catheter 100 in which the vacuum is applied. In these embodiments, detection of sufficient injectate (e.g. a minimum amount of injectate) can correlate to inadequate engagement with tissue. The detector can comprise an optical sensor, and/or a window which is visualizable by an operator (e.g. to see injectate that is recovered), such as when the injectate comprises visible material.

In some embodiments, one or more functional elements 109, 119, 139, 209, 229 and/or 309 comprises one or more temperature sensors that produces a signal related to a first temperature representing the temperature of ablative fluid delivered to functional assembly 130 and a second temperature related to the temperature of fluid extracted from functional assembly 130. In these embodiments, system 10 can be configured to assess (e.g. via algorithm 251) the effect (e.g. quantity) of tissue treated (e.g. depth of tissue ablated), such as by analyzing the first temperature and the second temperature (e.g. a comparison of the two). In some embodiments, the first and/or second temperature is measured by one or more sensors of connecting assembly 300 (e.g. two or more functional elements 309 comprising thermistors or other temperature sensors) and/or one or more sensors of catheter 100 (e.g. two or more functional elements 109, 119 and/or 139 comprising thermistors or other temperature sensors).

In some embodiments, one or more of functional elements 109, 119, 139, 209, 229 and/or 309 comprise a sensor configured to provide a signal related to lumen diameter information. In these embodiments, the sensor can comprise a sensor selected from the group consisting of: pressure sensor; optical sensor; sound sensor; ultrasound sensor; strain gauge; electromagnetic sensor; an imaging device such as a camera; and combinations of one or more of these. One or more console settings 201 can be adjusted (e.g. manually or automatically) based on the lumen diameter information.

In some embodiments, one or more of functional elements 109, 119, 139, 209, 229 and/or 309 comprise a sensor including an imaging device configured to provide a signal related to image information. The imaging device can comprise a device selected from the group consisting of: visible light camera; infrared camera; endoscope camera; MRI; Ct Scanner; X-ray camera; PET Scanner; ultrasound imaging device; and combinations of one or more of these. In these embodiments, controller 250 or another assembly of system 10 can comprise signal processor 252 and/or algorithm 251, each of which can be configured to analyze the image information provided by the imaging device. One or more console settings 201 can be adjusted (e.g. manually or automatically) based on the image information. Based on the image information, system 10 can be configured to modify a console setting 201 to cause an event selected from the group consisting of: stopping delivery of fluid and/or energy to functional assembly 130; delivering additional fluid into functional assembly 130 and/or into tissue; delivering neutralizing fluid into functional assembly 130 and/or into tissue; adjusting the pressure of functional assembly 130; adjusting the volume of functional assembly 130; and combinations of one or more of these.

In some embodiments, functional assembly 130 comprises one or more biasing members, such as biasing member 145 shown. Biasing member 145 can be constructed and arranged to apply a force to functional assembly 130, such as to place functional assembly 130 in tension along the axis of shaft 110 proximate functional assembly 130 (e.g. when functional assembly 130 is in an unexpanded state). Biasing member 145 can be constructed and arranged to bend as functional assembly 130 expands. Biasing member 145 can comprise an element selected from the group consisting of: spring; coil spring; leaf spring; flexible filament; flexible sheet; nickel titanium alloy component; and combinations of one or more of these. In some embodiments, functional assembly 130 comprises balloon 136, and biasing member 145 is configured to avoid contacting balloon 136 when functional assembly is in its unexpanded state.

In some embodiments, shaft 110 passes through all or a portion of functional assembly 130. In other embodiments, functional assembly 130 is positioned on a distal end of shaft 110 or on an external wall of shaft 110.

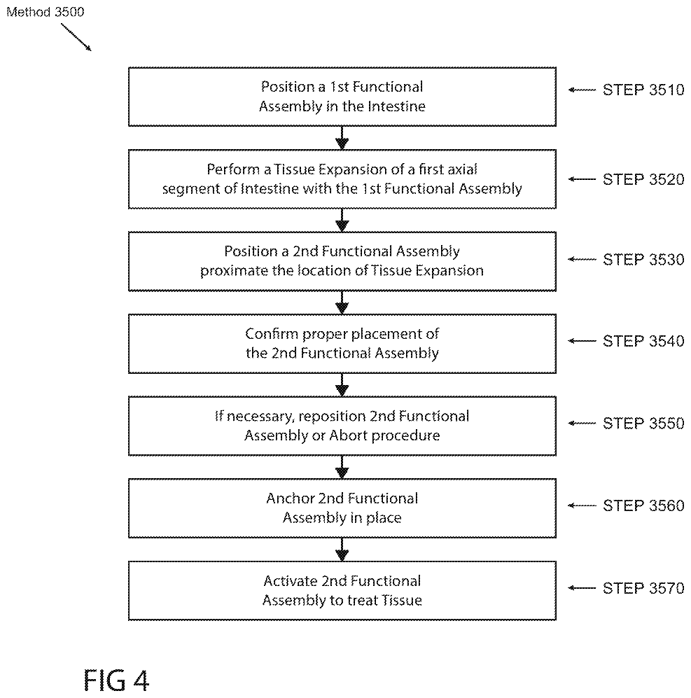

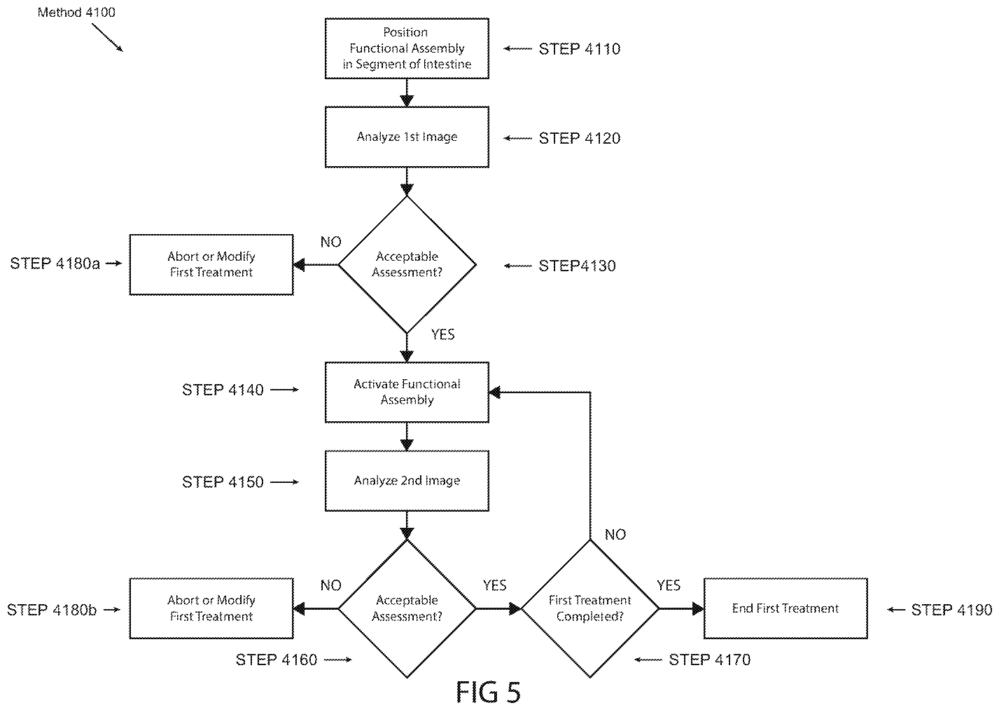

In some embodiments, functional assembly 130 comprises a shape constructed and arranged to prevent or otherwise reduce migration of functional assembly 130. In some embodiments, functional assembly 130 is constructed and arranged to perform a first procedure (e.g. a tissue expansion procedure), anchor in tissue (e.g. anchoring performed prior to the first procedure, during the first procedure and/or after the first procedure), and perform a second procedure (e.g. a tissue ablation procedure), such as is described herein in reference to FIG. 4.

In some embodiments, functional assembly 130 and/or other components of catheter 100, connecting assembly 300 and/or console 200 are configured to enhance mixing of one or more fluids within functional assembly 130 (e.g. one or more functional element 139 comprising a fluid mixing element). In some embodiments, one or more functional elements 139 within functional assembly 130 comprise a baffle configured to improve fluid mixing and/or occupy a volume (e.g. a baffle positioned within functional assembly 130). In some embodiments, one or more functional elements 139 comprise an expandable and/or compressible baffle. These baffles can be configured to “take up” volume within functional assembly 130, such as to decrease the amount of fluid (e.g. ablative fluid) delivered into functional assembly 130 during a tissue ablation and/or tissue expansion procedure. The baffles can be configured to reduce rise times or fall times of temperatures associated with functional assembly 130 (e.g. reduce rise times or fall times to or from ablative temperatures, respectively, during a tissue ablation procedure). The baffles can be configured to take up volume in between two or more ports 137, such as to minimize the overall diameter of a catheter 100 configured as a tissue expansion device. As described herein, the treatment can be modified (e.g. improved) by modifying the volume of fluid within functional assembly 130 and/or modifying the mixing of fluid within functional assembly 130 (e.g. via modifications as determined by algorithm 251 or otherwise by system 10). As described herein, the treatment of the target tissue can be modified according to the mixing of the contents (e.g. the fluid and/or other material) within functional assembly 130.

In some embodiments, system 10 is configured to provide enough mixing of fluid (e.g. ablative fluid and/or neutralizing fluid) within functional assembly 130 to achieve precise uniformity in depth of tissue ablation. In some embodiments, an ablative fluid with enhanced mixing comprises steam.

In some embodiments, a first conduit 111 can comprise an inflow tube configured to at least deliver fluid to functional assembly 130. A second conduit 111 can surround the first conduit 111, and an opening on the proximal end of the second (outer) conduit 111 can be closed off (e.g. a proximal end of second conduit 111 positioned near the proximal end of functional assembly 130). The distal end of the second conduit 111 can extend past the midpoint of functional assembly 130 but terminate proximal to the distal end of functional assembly 130, forming a collar around the inner first conduit 111 that channels the flow from the first conduit 111 to the distal portion of functional assembly 130, and improving mixing within all of the internal volume of functional assembly 130.

In some embodiments, catheter 100 comprises one or more insulating elements configured to avoid transfer of energy from shaft 110 to tissue, such as an insulating element comprising a full or partial layer of shaft 110 that comprises thermally insulating material and/or an insulating element comprising one or more conduits 111 which contain circulating fluid configured to dissipate heat from shaft 110.

Shaft 110 of catheter 100 can comprise one or more coatings, coating 118 shown, along all or a portion of its outer and/or inner surfaces. In some embodiments, coating 118 is positioned on at least a portion of the outer surface of shaft 110, and the coating 118 is configured to prevent or otherwise reduce inadvertent translation of catheter 100 through the intestine (e.g. a roughened surface or other anti-migration coating configured to reduce undesired translation and/or rotation of catheter 100). Alternatively or additionally (e.g. on a different portion), coating 118 can comprise a lubricous coating. In some embodiments, coating 118 is positioned on one or more lumens of shaft 110, such as a lubricous coating configured to assist in the translation of one or more filaments within the lumen. In some embodiments, coating 118 comprises a coating positioned on at least a portion of shaft 110 and selected from the group consisting of: a hydrophilic coating (e.g. to improve lubricity); a coating comprising bumps (e.g. atraumatic projections configured to roughen a surface to reduce friction); a coating comprising a surface exposed to grit blasting (e.g. to roughen a surface to reduce friction); an insulative coating: parylene; PTFE; PEEK; a coating comprising a colorant (e.g. to improve or otherwise improve visibility of shaft 110 in-vivo); and combinations of one or more of these. In some embodiments, coating 118 comprises a coating positioned on at least a portion of functional assembly 130 (e.g. on at least a portion of a balloon 136) and selected from the group consisting of: a lubricous coating; a surface roughening coating; a silicone coating; an insulative coating; and combinations of one or more of these.

In some embodiments, one or more functional elements 109, 119, 139, 209, 229 and/or 309 comprise a filter (e.g. a hydrophobic filter) positioned in a fluid pathway of system 10. The filter can be positioned between a sensor and the fluid pathway. In these embodiments, the associated functional element 109, 119, 139, 209, 229 and/or 309 can further comprise a valve, such as a valve configured to vent the fluid pathway proximate the filter.

In some embodiments, system 10 can be configured to deliver injectate 221 to tissue to cause tissue expansion via a body fluid (e.g. via osmotic pressure). For example, injectate 221 can comprise a salt solution delivered by one or more fluid delivery elements 139c that cause water or other fluid to migrate from submucosal capillaries into the submucosa.

In some embodiments, one or more of functional elements 109, 119, 139, 209, 229 and/or 309 comprise a sensor configured to detect gas-bubbles (e.g. an ultrasonic bubble detector), such as to detect a gas bubble present in one or more of conduits 111, 211, 212 and/or 311 and/or a gas bubble present in functional assembly 130. In some embodiments, one or more de-gassing procedures are performed on one or more components of system 10, and the one or more gas-bubble detector based functional elements 109, 119, 139, 209, 229 and/or 309 are used to confirm that the de-gassing procedure is adequately completed and/or to indicate a de-gassing procedure should be performed.

In some embodiments, multiple conduits 111 are in fluid communication with functional assembly 130 (e.g. to simultaneously or sequentially inflate and/or deflate functional assembly 130) and/or port 137 (e.g. to simultaneously or sequentially provide a vacuum to port 137). In these embodiments, simultaneous and/or redundant delivery or extraction of fluids (e.g. application of a vacuum) can be initiated based on the signal provided by one or more sensors of system 10. For example, if a sensor detects a first conduit 111 is fully or partially occluded, the second conduit 111 can be used to additionally or alternatively deliver and/or extract fluids.

In some embodiments, system 10 is configured to maintain the pressure of functional assembly 130 relative to a threshold (e.g. pressure is maintained below a pressure threshold, above a pressure threshold, and/or within a threshold comprising a range of pressures), such as during treatment and/or diagnosis of target tissue of the intestine (e.g. during a tissue expansion and/or tissue ablation procedure). Functional assembly 130 can comprise a balloon 136 comprising a compliant balloon; a non-compliant balloon; a pressure-thresholded balloon; and/or a balloon comprising compliant and non-compliant portions, as described herein. In some embodiments, functional assembly 130 comprises a balloon 136 that is a compliant balloon (e.g. a balloon with at least a portion made of a compliant material), such that balloon 136 can be inflated (e.g. via a pressure-based and/or volume based fluid delivery) to sufficiently make circumferential contact with a range of luminal diameters (e.g. diameters between 15 mm and 35 mm). In these embodiments, system 10 can be configured to perform target tissue ablation without the use of aspiration, such as when system 10 causes sufficient contact of balloon 136 with tissue by controlling the volumetric and/or pressure-based expansion of balloon 136. In some embodiments, the pressure within balloon 136 is maintained (e.g. during ablation) at a pressure between 0.7 psi and 2.0 psi. In some embodiments, balloon 136 comprises a non-compliant balloon (e.g. a balloon made of non-compliant material), and aspiration is similarly avoided by controlling the volume and/or pressure of fluid within the non-compliant balloon.

Pressure can be maintained within functional assembly 130 at a particular pressure or within a particular range of pressures by monitoring one or more sensors of system 10, such as sensor 139b and/or a sensor-based functional element 119, 109, 209 and/or 229. A lower pressure threshold can comprise a pressure of 0.3 psi, 0.5 psi or 0.7 psi. A lower pressure threshold can be selected to ensure sufficient contact of functional assembly 130 with tissue. An upper pressure threshold can comprise a pressure of 1.0 psi, 1.2 psi, 2.5 psi or 4.0 psi. An upper pressure threshold can be selected to avoid damage to tissue, such as damage to an outer layer of intestinal tissue (e.g. a serosal layer of the intestine). Alternatively or additionally, an upper pressure threshold can be selected to avoid undesirably “pushing” injectate 221 out of a tissue expanded region. Pressure can be monitored such that console 200 can modulate or otherwise control one or more inflow and/or outflow rates of fluid delivered to and/or extracted from functional assembly 130. Pressure can be monitored to maintain flow rates to or from functional assembly 130 to a minimum rate of at least 250 ml/min, 500 ml/min, 700 ml/min or 750 ml/min. In some embodiments, pressure is determined by a sensor positioned outside of balloon 136, such as when pressure is maintained in functional assembly 130 within a narrow range of pressures, such as at a pressure of between 1.05 psi and 0.55 psi. In these embodiments, a luminal sizing step can be avoided. In some embodiments, system 10 comprises one or more catheters 100 and/or one or more functional assemblies 130, such as to provide an array of functional assemblies 130 with different lengths and/or diameters. In these embodiments, the upper and/or lower pressure thresholds can be independent of functional assembly 130 size.

In some embodiments, conduits 111 comprise an inflow tube and an outflow tube fluidly connected to functional assembly 130. Fluid can be delivered to functional assembly 130 by console 200 via one or more conduits 111 at various flow rates, such as flow rates up to 500 ml/min, 1000 ml/min, 1500 ml/min, 2000 ml/min and/or 2500 ml/min. Fluid can be extracted from functional assembly 130 by console 200 via one or more conduits 111 at various flow rates, such as flow rates up to 500 ml/min, 750 ml/min, or 1000 ml/min.

In some embodiments, treatment element 139a can comprise fluid at a sufficiently high temperature to ablate tissue (such as liquid above 60° C. or steam). Delivery of superheated fluid through a conduit 111 can be performed, such as when functional element 119 comprises an orifice configured to cause the superheated fluid to boil upon entering functional assembly 130, providing steam at 100° C. Delivery of cooled fluids through a conduit 111 can be performed. In some embodiments, a fluid (cooled or otherwise) is introduced through a conduit 111 and through a functional element 119 comprising a valve, such that expansion of the fluid into functional assembly 130 results in a cooling effect.

In some embodiments, system 10 and catheter 100 are constructed and arranged to fill functional assembly 130 with neutralizing (e.g. chilled) fluid, and then thermally prime (e.g. heat) a first conduit 111 with ablative (e.g. hot) fluid, when the first conduit is positioned in a retracted state (e.g. preventing or otherwise reducing heating of functional assembly 130). After a full or partial fill of functional assembly 130 with neutralizing fluid, the first conduit 111 is advanced (i.e. first conduit 111 is constructed and arranged as a translatable conduit) and ablative fluid is introduced into functional assembly 130. This delivery of non-ablative fluid allows functional assembly 130 to be in a fully or partially expanded state prior to fluid at an ablative temperature residing in functional assembly 130, avoiding undesired “partial ablative contact” of functional assembly 130 with tissue. Another advantage of this configuration is that functional assembly 130 can be checked for leaks with non-ablative fluid prior to one or more subsequent steps (e.g. each ablation step).

In some embodiments, functional assembly 130 is constructed and arranged to both expand tissue (e.g. expand submucosal tissue) and treat target tissue (e.g. treat duodenal mucosal tissue), such as is described herein in reference to multi-function catheter 40 of FIG. 2. For example, functional assembly 130 can comprise fluid delivery element 139c which can be positioned to deliver fluid into tissue that has been drawn into (e.g. upon application of a vacuum) port 137, to expand one or more layers of tissue (e.g. one or more layers of submucosal tissue). Functional assembly 130 can further comprise treatment element 139a which can comprise ablative fluid which can be introduced into functional assembly 130 and/or an energy delivery element configured to deliver energy to tissue (e.g. RF energy, light energy, sound energy, chemical energy, thermal energy and/or electromagnetic energy), each configured to perform a therapeutic treatment on target tissue.

In some embodiments, system 10 and catheter 100 are configured to both expand tissue (e.g. expand submucosal tissue of the intestine) and treat target tissue (e.g. treat mucosal tissue of the intestine proximate the expanded submucosal tissue). Catheter 100 can comprise a single catheter 100 comprising one or more functional elements 139 configured to collectively expand tissue and treat target tissue, or a first catheter 100a configured to expand tissue and a second catheter 100b configured to treat target tissue. In these embodiments, injectate 221 can comprise a material configured to enhance or otherwise modify a target treatment step. For example, injectate 221 can comprise a conductive fluid (e.g. an electrically conductive fluid), such as saline configured to modify a subsequent target tissue treatment by treatment element 139a in which RF or other electrical energy is delivered to target tissue (e.g. when treatment element 139a comprises an array of electrodes). Similarly, injectate 221 can comprise a chromophore or other light absorbing material and/or a light scattering material configured to modify a subsequent target tissue treatment by treatment element 139a in which light energy is delivered to target tissue (e.g. when treatment element 139a comprises a lens, one or more conduits 111 comprise an optical fiber, and controller 250 comprises an energy delivery unit EDU 260 comprising a laser).

In some embodiments, fluid delivery element 139c comprises a needle and/or a catheter with two separate lumens (e.g. two lumens each fluidly connected to a different conduit 111), such that two different materials can be injected into tissue without the two fluids mixing prior to entering the tissue. Alternatively, fluid delivery element 139c can comprise two different needles and/or catheters directed toward a similar area. Injectate 221 can comprise a first material and a second material which form a hydrogel when mixed (e.g. the two materials crosslink to form an absorbable hydrogel). Alternatively or additionally, injectate 221 can comprise water soluble PEG reactive end groups and an amino acid with reactive end groups.

In some embodiments, injectate 221 comprises a material selected from the group consisting of: autologous fat; collagen; bovine collagen; human cadaveric dermis; hyaluronic acid; calcium hydroxylapatite; polylactic acid; semi-permanent PMMA; dermal filler; gelatin; and combinations of one or more of these. In some embodiments, injectate 221 comprises a material whose viscosity changes (e.g. increases) after delivery into tissue, such as a fluid whose viscosity increases as it is heated to body temperature.

In some embodiments, injectate 221 comprises a material including hollow materials and a carrier material, such as when system 10 is constructed and arranged to deliver injectate 221 to create a therapeutic restriction. In these embodiments, injectate 221 can comprise a material as described in US Patent Application US20080107744 or US Patent Application US20110091564. In some embodiments, injectate 221 comprises inorganic fibers and a carrier material. The inorganic fibers can be constructed and arranged to prevent or otherwise reduce their migration within tissue. The carrier material can be constructed and arranged to allow the inorganic fibers to be injectable (e.g. to pass through fluid delivery element 139c). In these embodiments, injectate 221 can comprise a material as described in US Patent Application US20140255458.

In some embodiments, system 10, console 200 and/or catheter 100 is constructed and arranged to reduce risk during injection of material into the wall of the duodenum. In some embodiments, a pre-determined volume of polymer or other material is injected using catheter 100 or a standard endoscopic needle device. A volume of at least 1 ml or 2.5 ml of a first material (e.g. a relatively inert material such as sterile saline), is injected into the wall first, creating a first expanded tissue volume, a “bleb” of expanded tissue and the saline. Subsequently, a second material, such as a pharmaceutical agent, a durable material (e.g. to create a therapeutic restriction as described herein), or other active material is injected into the first expanded tissue volume to further expand the tissue.

In some embodiments, system 10 includes one or more tools, tool 500 shown. Tool 500 can comprise a vacuum applying tool such as an endoscopic cap. Catheter 100 or a standard endoscopic needle device can inject a material into the wall of the duodenum while the endoscopic cap applies suction to the intestinal mucosa. A needle or other fluid delivery element of catheter 100 (e.g. fluid delivery element 139c) or a needle of a standard endoscopic needle device is delivered into intestinal tissue while the mucosa of the intestine is lifted by tool 500.

In some embodiments, injectate 221 comprises a material that can be detected by system 10 and/or an operator of system 10, such as a visualizable material, magnetic material, and/or other detectable material. In some embodiments, injectate 221 comprises one or more materials (e.g. a biocompatible polymer or copolymer such as ethylene vinyl alcohol), and it can further include a detectable material selected from the group consisting of: a radiopaque material; barium sulfate; tantalum; ultrasonically reflecting material; magnetic material; a visible dye; and combinations of one or more of these. In these embodiments, system 10 can comprise a fluid extraction assembly comprising one or more ports (e.g. ports 137 or other openings of catheter 100) that are constructed and arranged to withdraw fluids from within the intestine, such as via one or more conduits 111 and one or more pumping assemblies 225. One or more functional elements 109, 119, 139, 229 and/or 309 can comprise a sensor configured to produce a signal related to the quantity of injectate 221 recovered via the one or more ports 137, such as a sensor configured to detect a volume, mass, flow rate and/or other parameter of injectate 221. Signal processor 252 can be configured to assess tissue expansion based on an analysis of the recovered injectate 221.

In some embodiments, injectate 221 comprises one or more materials such as ethylene vinyl alcohol (EVOH) which is provided in a liquid solvent such as dimethyl sulfoxide (DMSO). In these embodiments, a visualizable material such as a radiopaque material (e.g. tantalum) can be further included. In these embodiments, catheter 100 can be configured to deliver this injectate 221 into tissue (e.g. via one or more fluid delivery elements 139c), after which the one or more materials, and the visualizable material if included, precipitate from the solution to form a spongy implant, which can remain in proximity to the injection site for a prolonged period of time.

In some embodiments, algorithm 251 is configured to determine an expanded size for functional assembly 130, such as when system 10 comprises multiple catheters 100 with different expanded diameters for functional assembly 130 and/or when the expanded diameter of functional assembly 130 can be varied by system 10 (e.g. by varying pressure and/or volume of fluid within functional assembly 130). In these embodiments, algorithm 251 can comprise a bias, such as a bias which tends toward lower diameters (e.g. rounds down to the next smaller size of a functional assembly 130 available after calculating a target value). In some embodiments, algorithm 251 is configured to select one catheter 100 for use in a patient, by selecting one of a kit of multiple catheters 100 comprising one or more different parameters (e.g. one or more functional assembly 130 parameters). In these embodiments, algorithm 251 can also include a bias, such as a bias toward choosing a smaller sized functional assembly 130, such as a functional assembly 130 with a smaller length and/or a smaller expanded diameter than the length and/or diameter, respectively, of the segment of the intestine of the patient to be treated (e.g. the segment of the intestine including the target tissue).

In some embodiments, algorithm 251 of console 200 comprises an image analysis algorithm configured to analyze one or more patient and/or system 10 images. For example, a tissue location can be analyzed prior to, during and/or after a desufflation (e.g. aspiration) step, such as to confirm adequate apposition of a functional assembly 130 with tissue of an axial segment of tubular tissue (e.g. an axial segment of the intestine). Algorithm 251 can comprise one or more image analysis algorithms configured to assess various conditions including but not limited to: apposition of functional assembly 130 with tissue (e.g. intestinal wall tissue); effectiveness of a desufflation procedure; effectiveness of an insufflation procedure; sufficiency of a tissue expansion procedure; sufficiency of a tissue ablation procedure; and combinations of one or more of these.

In some embodiments, one or more reservoirs 220 and/or one or more pumping assemblies 225 are constructed and arranged to provide a cryogenic gas or other cryogenic fluid to functional assembly 130, such as to perform a cryogenic ablation of target tissue and/or to cool target tissue that has been heated above body temperature. Cryogenic gas can be delivered through smaller diameter conduits 111 than would be required to sufficiently accommodate a liquid ablative or neutralizing fluid, which correlates to a reduced diameter of shaft 110. Balloon 136 can comprise a compliant balloon (e.g. a highly compliant balloon). Balloon 136 can be fluidly connected to multiple fluid transport conduits 111, singly or collectively providing inflow (i.e. delivery) and/or outflow (i.e. extraction) of the cryogenic gas. System 10 can be configured to control the pressure within balloon 136, such as at a pressure level sufficient, but not much greater than, that which would be required to simply inflate balloon 136. A highly compliant balloon 136 can be configured to reduce or avoid the need for a luminal sizing step to be performed. Temperature seen by the target tissue is driven by the temperature of the fluid in balloon 136. During treatment (i.e. cryogenic ablation) the pressure in balloon 136 can be maintained at a pressure at or below 20 inHg, such as below 18 inHg, 15 inHg or 10 inHg.

In some embodiments, system 10 comprises a first catheter 100 with a functional assembly 130 with a first diameter, and a second catheter 100 with a functional assembly 130 with a second diameter (e.g. a smaller expanded diameter than the first diameter). In these embodiments, system 10 can be constructed and arranged such that an operator (e.g. a clinician) inserts the first catheter 100 into the intestine of a patient and performs a first function, such as a function selected from the group consisting of: size (e.g. determine the diameter) of one or more axial locations of intestine; perform or at least attempt to perform a tissue expansion procedure in one or more axial segments of intestine; perform or at least attempt to perform a tissue treatment (e.g. tissue ablation) at one or more axial segments of intestine; and combinations of one or more of these. In some embodiments, during and/or after performance of the first function, a decision can be made to switch to the second catheter 100 with a different functional assembly 130, such as when it is determined the functional assembly 130 of the first catheter 100 is too large. In these embodiments, the first catheter 100 and the second catheter 100 can each be configured to perform both a tissue expansion procedure and an ablation procedure. In some embodiments, the functional assembly 130 of the first catheter 100 is constructed and arranged to expand to a pre-determined diameter, such as a diameter of between 19 mm and 32 mm, or between 23 mm and 28 mm, such as a diameter of approximately 24 mm, 26 mm or 28 mm. In some embodiments, functional assembly 130 is configured to perform a tissue treatment in a duodenum of a human patient, and functional assembly 130 (e.g. balloon 136) is configured to expand to a pre-determined diameter, as described hereabove, such as to avoid damaging the duodenal wall while providing sufficient contact with the luminal surface to perform a tissue treatment procedure (e.g. a tissue ablation procedure requiring the transfer of energy from functional assembly 130 to a majority of the surface of the segment of the duodenum in which functional assembly 130 is positioned). For example, functional assembly 130 (e.g. balloon 136) can be constructed of materials (e.g. non-compliant materials) to cause expansion to be limited to a predetermined diameter, such as a diameter as described hereabove. Alternatively or additionally, system 10 (e.g. console 200) can be configured to deliver fluid into functional assembly 130 (e.g. balloon 136) in a controlled manner, such as to cause expansion to a pressure and/or fluid volume driven diameter, such as a predetermined diameter as described hereabove. In some embodiments, algorithm 251 is configured to select the first catheter 100 and/or the second catheter 100 for use (e.g. use in the patient). Alternatively, the functional assembly 130 of the first catheter 100 can comprise an expanded diameter smaller than the expanded diameter of the functional assembly 130 of the second catheter 100, wherein the second catheter 100 is introduced into the patient if it is determined that the expanded diameter of the functional assembly 130 of the first catheter 100 is too small.

In some embodiments, pumping assembly 225 comprises at least two pumping assemblies 225 configured to propel fluid out of (i.e. extract fluid from) functional assembly 130 and/or another component of catheter 100, such as two pumping assemblies 225 which operate simultaneously during the performance of a functional assembly 130 drawdown procedure (e.g. an emergency radial contraction of functional assembly 130 that is initiated during an undesired situation, such as an emergency drawdown procedure initiated when a leak is detected). In some embodiments, two pumping assemblies 225 are configured to deliver fluid to functional assembly 130 (e.g. to balloon 136 and/or one or more fluid delivery elements 139c) or other component of catheter 100. In these embodiments, simultaneous fluid delivery can also be performed when a leak is detected, such as to simultaneously deliver a neutralizing fluid to tissue being undesirably exposed to ablative fluid. For example, if a leak is detected (e.g. by system 10 or otherwise), pumping assembly 225 can be configured to provide cold fluid to balloon 136 (e.g. automatically and/or immediately provide the cold fluid). Alternatively or additionally, a second pumping assembly 225 can be configured to begin fluid delivery and/or fluid extraction when the failure of a first pumping assembly 225 is detected. Two or more pumping assemblies 225 can be fluidly attached to one or more fluid transport conduits 211. As an example, a first pumping assembly 225 can be configured to extract ablative fluid from functional assembly 130 via a first conduit 111, and a second pumping assembly 225 can be configured to simultaneously deliver a neutralizing fluid to functional assembly 130 via a second conduit 111.

In some embodiments, console 200 is constructed and arranged to maintain a minimum volume (e.g. a minimum level of fluid) of one or more reservoirs 220. In some embodiments, console 200 is constructed and arranged to disable a pumping assembly 225 if an undesired condition is detected, such as by a signal recorded by a functional element 209, 229a, 229b, and/or 309 that comprises a sensor configured to monitor one or more system parameters (e.g. temperature, pressure, flow rate, and the like). As described herein, the console can be configured to disable one or more of pumping assemblies 225 when an undesired condition of the pumping assemblies and/or reservoirs 220 are detected by the sensor.

In some embodiments, console 200 is constructed and arranged to modify (e.g. limit) a treatment time or to modify (e.g. limit) another treatment parameter. In these embodiments, the treatment parameter can be limited by software, such as software of algorithm 251 and/or controller 250. Alternatively, the treatment parameter can be limited by hardware (e.g. a hardware-based algorithm 251), such as hardware of controller 250 such as a temperature controlled functional element which turns off a pumping assembly 225 and/or otherwise prevents or reverses energy being delivered by a functional assembly 130 of catheter 100.

In some embodiments, system 10 is constructed and arranged (e.g. via algorithm 251) to adjust one or more treatment parameters, such as an adjustment based on the expanded size of a functional assembly 130, such as when system 10 comprises multiple catheters 100, each comprising a different expanded size of its functional assembly 130. In these embodiments, system 10 can be constructed and arranged to adjust one or more treatment parameters selected from the group consisting of: temperature of ablative fluid; volume of ablative fluid; pressure of ablative fluid; amount of energy delivered such as peak amount of energy delivered and/or cumulative amount of energy delivered; duration of treatment; amount of fluid delivered into tissue (e.g. during a tissue expansion procedure or a tissue ablation procedure); and combinations of one or more of these. In some embodiments, algorithm 251 is configured to control the duration of the delivery of energy (e.g. the time in which ablative fluid resides within functional assembly 130 while functional assembly 130 is in contact with tissue), such as to control the depth of tissue ablated. In some embodiments, algorithm 251 controls the duration of the delivery of energy with a minimum duration of 500 msec and/or a maximum duration of 12 seconds.

In some embodiments, console 200 is constructed and arranged to provide a first fluid at an ablative temperature, and a second fluid at a neutralizing temperature. For example, a first fluid can be provided by a first reservoir 220 such that the first fluid enters functional assembly 130 at a sufficiently high temperature to ablate tissue, such as at a temperature above 44° C. or above 60° C. A second fluid can be provided by a second reservoir 220 such that the second fluid enters functional assembly 130 at a neutralizing temperature below body temperature, such as a temperature between room temperature and body temperature, or a temperature below room temperature. Alternatively, an ablative fluid can comprise a fluid of sufficiently low temperature to ablate tissue (e.g. below 5° C.), and an associated neutralizing fluid can comprise a warmer fluid (e.g. a fluid at body temperature) configured to reduce the tissue damaging effects of the ablative fluid, as described herein. In some embodiments, a neutralizing fluid is provided to functional assembly 130 prior to and/or after delivery of ablative fluid to functional assembly 130, as described in detail herein.

An ablative fluid and a neutralizing fluid can be transported to functional assembly 130 via the same or different conduits 111. Fluid can be extracted from functional assembly 130 via the same or different conduits used to deliver the first fluid and/or the second fluid. In some embodiments, conduits 111 used to deliver and/or extract an ablative fluid or a neutralizing fluid are configured to be translated (e.g. advanced and/or retracted), such that their distal end position within or otherwise relative to functional assembly 130 can be varied. In some embodiments, one or more conduits 111 and/or functional assembly 130 can be thermally primed prior to treating target tissue. In some embodiments, ablative fluid and/or neutralizing fluid is provided to functional assembly 130 in a recirculating manner. Alternatively, ablative fluid and/or neutralizing fluid can be provided to functional assembly 130 as a bolus (non-circulating volume of fluid). In some embodiments, functional element 119 comprises one or more valves constructed and arranged to control the flow of fluid through one or more conduits 111. In recirculating fluid embodiments, a conduit 111 supplying fluid can be manually or automatically changed to a fluid extraction conduit, such as when a separate conduit 111 is configured to normally extract fluid from functional assembly 130 becomes occluded, when a conduit 111 or functional assembly 130 begins to leak, or otherwise when it is desired to radially compact functional assembly 130 at an accelerated rate.

In some embodiments, at least a first conduit 111a provides ablative fluid to functional assembly 130 while at least a separate conduit 111b simultaneously withdraws ablative fluid from functional assembly 130, such as to recirculate ablative fluid within functional assembly 130. In these embodiments, functional assembly 130 can be radially expanded (e.g. initially or after a radial compacting step), by filling functional assembly 130 (e.g. with ablative fluid, neutralizing fluid and/or other fluid) by using both first conduit 111a and second conduit 111b.

In some embodiments, treatment element 139a comprises an energy delivery element including multiple layers of electrical conductors (e.g. conductors and/or semiconductors) configured to generate heat when electricity passes through one or more of the conductors. In these embodiments, functional element 139 can be electrically connected to one or more conduits 111 comprising one or more electrical wires. Functional assembly 130 can comprise a compliant or non-compliant balloon onto which functional element 139 is positioned. Treatment element 139a can comprise electrical conductors created by depositing one or more coatings on one or more substrates. When electricity is passed through the coating, heat is generated. The heat can be effectively transferred across the whole surface of functional element 139 mainly through conduction, but also via radiation and convection and into target tissue.

In some embodiments, balloon 136 comprises at least a porous portion or a portion otherwise constructed and arranged to allow material contained within balloon 136 to pass through at least a portion of balloon 136. In these embodiments, injectate 221 can comprise a material configured to pass through at least a portion of balloon 136, such as a conductive gel material configured to modify energy delivery, such as when treatment element 139a comprises one or more electrodes configured to delivery RF energy to target tissue. In other embodiments, agent 420 comprises one or more agents configured to be delivered into balloon 136 and to pass through at least a portion of balloon 136 and into the intestine.

In some embodiments, system 10 is constructed and arranged to deliver fluid into functional assembly 130 at a flow rate of at least 500 ml/min, at least 1000 ml/min, at least 2000 ml/min, or at least 2500 ml/min. In some embodiments, system 10 is constructed and arranged to extract fluid from functional assembly 130 at a flow rate of at least 500 ml/min, at least 750 ml/min, or at least 1000 ml/min. In some embodiments, system 10 is constructed and arranged to remove and extract fluids at approximately the same flow rate. In some embodiments, fluid in console 200 is provided to catheter 100 at a temperature of at least 60° C., 70° C. or 80° C. In some embodiments, system 10 is configured to treat at least three axial segments of intestinal tissue, such as at least three axial segments of tissue treated with a heat ablation and at least one cooling step (e.g. a cooling step performed prior to and/or after the heat ablation step).

In some embodiments, tool 500 comprises an insufflation and/or desufflation tool, such as a catheter comprising a port (e.g. an opening at a distal end or along a distal portion of the catheter) for delivering and/or extracting fluids from the intestine. Tool 500 can be insertable through the working channel of an introduction device 50 (e.g. through an endoscope). Delivery of insufflation fluids can be performed to move tissue away from functional assembly 130 and/or move tissue away from one or more functional elements 139 or other parts of catheter 100. In some embodiments, insufflation is performed to stop or limit a transfer of energy to tissue (e.g. in an emergency or insufflation-controlled ablation step).

In some embodiments, tool 500, catheter 100, introduction device 50 and/or another component of system 10 comprises a pressure-neutralizing assembly constructed and arranged to modify the pressure within a luminal segment of the intestine (e.g. a luminal segment proximate functional assembly 130). In these embodiments, tool 500 and/or catheter 100 can comprise one or more openings or other elements configured as vents, such as to vent the luminal segment to room pressure (e.g. clinical procedure room pressure) or otherwise maintain the pressure in a segment of the intestine below a threshold. In some embodiments, introduction device 50 comprises an endoscope comprising a biopsy port configured to vent the luminal segment to room pressure. The pressure-neutralizing assembly can be configured to extract gas from the intestinal segment, and/or to maintain the pressure within the intestinal segment below a threshold. In some embodiments, venting is activated automatically, such as when a pressure (e.g. as measured by a sensor of the present inventive concepts) reaches a threshold (e.g. as determined by algorithm 251).

In some embodiments, algorithm 251 comprises a pressure algorithm configured to modify a system parameter based on a measured pressure, such as a modification made based on the pressure within a luminal segment of the intestine in which functional assembly 130 is positioned or otherwise proximate (e.g. as measured or otherwise determined by analysis of a signal provided by a sensor of catheter 100, body introduction device 50 or another sensor of system 10 as described herein). In these embodiments, system 10 can be configured to modify the volume of fluid within functional assembly 130 and/or modify the pressure of functional assembly 130 based on the luminal segment pressure.

In some embodiments, functional assembly 130 is positioned in an axial segment of intestine, expanded to a diameter less than the average diameter of the axial segment, and activated (e.g. to deliver energy to tissue and/or fluids to tissue) during a contraction of the intestine. In these embodiments, the contraction of the intestine can be one or more of: a (natural) peristaltic contraction; a contraction caused by stimulation (e.g. electrical or chemical stimulation by catheter 100 and/or tool 500); a contraction caused during a desufflation procedure; and combinations of one or more of these. Contraction of the intestine can comprise a desufflation procedure performed by a device selected from the group consisting of: catheter 100; an endoscope or other body introduction device 50; a second catheter 100 inserted into the intestine; and combinations of one or more of these.

In some embodiments, tool 500 comprises a diagnostic tool, such as a diagnostic tool comprising a sensor. Tool 500 can be configured to perform a diagnostic test of the patient and/or a diagnostic test of all or a portion of system 10. Tool 500 can comprise a body-insertable tool. Tool 500 can be constructed and arranged to gather data (e.g. via an included sensor) related to a patient physiologic parameter selected from the group consisting of: blood pressure; heart rate; pulse distention; glucose level; blood glucose level; blood gas level; hormone level; GLP-1 level; GIP Level; EEG; LFP; respiration rate; breath distention; perspiration rate; temperature; gastric emptying rate; peristaltic frequency; peristaltic amplitude; and combinations of one or more of these.

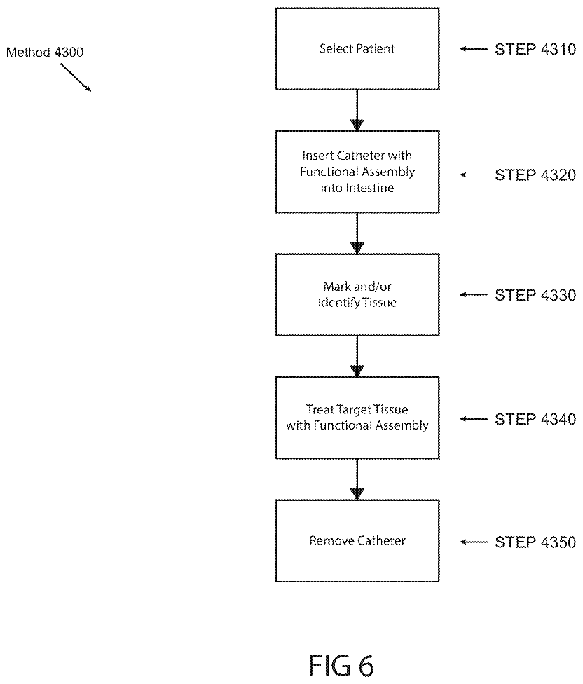

Alternatively or additionally, tool 500 can comprise a tissue marking tool, such as a tissue marking tool configured to be deployed through introduction device 50 (e.g. an endoscope). In some embodiments, system 10 comprises one or more markers, marker 490 shown, which can comprise a dye and/or other visualizable media configured to mark tissue (e.g. using a needle-based tool 500), and/or a visualizable temporary implant used to mark tissue, such as a small, temporary clip configured to be attached to tissue by tool 500 and removed at the end of the procedure (e.g. by tool 500) or otherwise passed by the natural digestive process of the patient shortly after procedure completion. Marker 490 can be deposited or deployed in reference to (e.g. to allow an operator to identify) non-target tissue (e.g. a marker positioned proximate the ampulla of Vater to be visualized by an operator to avoid damage to the ampulla of Vater), and/or to identify target tissue (e.g. tissue to be ablated). In some embodiments, tissue marker 490 is deposited or deployed in reference to tissue selected from the group consisting of: gastrointestinal adventitia; duodenal adventitia; the tunica serosa; the tunica muscularis; the outermost partial layer of the submucosa; ampulla of Vater; pancreas; bile duct; pylorus; and combinations of one or more of these. In some embodiments, tissue marking is performed as described herein in reference to FIG. 6.

Functional assembly 130 can be configured to perform a medical procedure (e.g. a tissue expansion procedure and/or a tissue ablation or other tissue treatment procedure) on multiple axial segments of intestinal tissue. Two or more of the multiple axial segments can be treated sequentially and/or simultaneously. In some embodiments, system 10 is configured to perform treatment of multiple axial segments without the need for a significant “recovery period” (e.g. without the need for significant system-required wait periods). For example, system 10 can be configured to initiate the treatment of a second axial segment of intestinal tissue within 30 minutes, 20 minutes, and/or 15 minutes of the completion of the treatment of a first axial segment of intestinal tissue. The two or more of the multiple axial segments can be relatively proximate each other, such as to share common boundaries or avoid significant gaps in untreated tissue. The multiple axial segments can comprise partial or full circumferential segments of intestinal tissue. The multiple axial segments can cumulatively comprise at least 2 cm in length or at least 4 cm in length, such as when between 1 and 8 treatments are performed (e.g. when between 2 and 6 treatments are performed and functional assembly 130 is repositioned between 1 and 5 times). The multiple axial segments can cumulatively comprise a length of at least 6 cm, such as when between 2 and 9 treatments are performed (e.g. functional assembly 130 is repositioned between 1 and 8 times). In these embodiments, system 10 can be configured to treat insulin resistance and/or its associated diseases, such as diabetes (e.g. Type 2 diabetes), NAFLD, NASH, and/or PCOS.

In some embodiments, system 10 is configured to initially expand functional assembly 130, with a fluid at a non-ablative temperature (e.g. a fluid configured to cool tissue without ablating it), after which a fluid at an ablative temperature can be introduced into functional assembly 130 (e.g. a fluid at sufficiently high temperature to ablate tissue).

In some embodiments, catheter 100 and/or another device of system 10 comprises an anchoring element, such as when port 137 is configured as an anchoring element that engages tissue when a vacuum is applied to port 137 (e.g. via one or more conduits 111). Alternatively or additionally, inflation of balloon 136 can be used to anchor functional assembly 130 at a particular intestinal location. One or more functional elements 139 can be configured as an anchor element, such as a high friction coating or surface treatment, or an extendable barb.

In some embodiments, system 10 is constructed and arranged to allow an operator to position the functional assembly within an axial segment of the intestine and perform a first procedure on intestinal tissue with functional assembly 130. System 10 is further constructed and arranged to anchor functional assembly 130 (prior to, during and/or after the first procedure). Subsequent to the performance of the first procedure and the anchoring of functional assembly 130, a second procedure is performed. The first procedure can comprise a tissue expansion procedure. The second procedure can comprise a tissue ablation procedure, such as a tissue ablation procedure which ablates mucosal tissue within or otherwise proximate previously expanded submucosal tissue. Repeating of the three steps (i.e. the first procedure, the anchoring of functional assembly 130, and the second procedure) can be performed at additional locations within the intestine.

As described herein, in some embodiments, catheter 100 or another device of system 10 such as catheter 30 of system 10 of FIG. 2, is constructed and arranged to perform a luminal sizing measurement (e.g. a measurement in which diameter and/or other cross sectional geometry is quantified), and produce luminal size information. In these embodiments, system 10 can include multiple catheters 100, one of which is selected and/or adjusted based on the luminal size information. Alternatively or additionally, system 10 can be configured to adjust one or more system parameters based on the luminal size information, such as a console setting 201 selected from the group consisting of: volume of fluid delivered into functional assembly 130; flow rate of fluid delivered into functional assembly 130; temperature of fluid delivered into functional assembly 130; pressure of functional assembly 130; and combinations of one or more of these.

In some embodiments, console 200 and system 10 are constructed and arranged to maintain functional assembly 130 of catheter 100 at or below a target level of a functional assembly 130 parameter, such as at or below a target diameter, pressure and/or volume for functional assembly 130. In some embodiments, functional assembly 130 is maintained below a target pressure of 0.9 psi (e.g. during a tissue expansion, tissue ablation and/or other tissue treatment step).

In some embodiments, catheter 100 and system 10 are constructed and arranged to compensate for muscle contraction of the intestine (e.g. peristalsis within the intestine). For example, algorithm 251 can be configured to actively regulate a functional assembly 130 parameter (e.g. diameter, pressure within and/or flowrate to and/or from), such as when algorithm 251 anticipates, recognizes and/or compensates for muscular contraction of the intestine. In some embodiments, expansion of functional assembly 130 can be timed to occur during the bottom (lower range) of a muscular contraction (e.g. peristalsis) cycle.

In some embodiments, catheter 100 and system 10 are constructed and arranged to perform a medical procedure in the intestine that is synchronized with one or more muscular contractions of the intestine, such as one or more peristaltic contractions used to contact intestinal wall tissue with an expanded or partially expanded functional assembly 130.

In some embodiments, system 10 is constructed and arranged to size a lumen of a first axial segment of the intestine (e.g. a segment including target tissue to be treated). System 10 can be further constructed and arranged to subsequently perform a tissue expansion of a portion of the first axial segment (e.g. a full or partial circumferential segment of the submucosa of the axial segment), by injecting fluid (e.g. a fixed volume of fluid) into tissue within or proximate the first axial segment. System 10 can be further constructed and arranged to subsequently perform a luminal sizing measurement of the first axial segment. System 10 can be further constructed and arranged to subsequently perform a target tissue treatment of the first axial segment (e.g. a treatment of a full or partial circumferential segment of the mucosal tissue of the first axial segment). The treatment performed by system 10 can comprise one or more treatment parameters (e.g. one or more ablation parameters) that are based on the luminal sizing measurement performed after tissue expansion, and as determined via algorithm 251.

In some embodiments, system 10 comprises one or more first catheters 100a, each with a first functional assembly 130a of a particular size and configured to treat target tissue. System 10 can further comprise one or more second catheters 100b, each with a functional assembly 130b and configured to perform a tissue expansion procedure. System 10 can be constructed and arranged to size a lumen of one or more axial segments of intestine (e.g. using a catheter 100 or other luminal sizing device as described herein) to determine the diameter at a relatively narrow (e.g. the smallest diameter) location within the one or more axial segments to be treated. System 10 can be further constructed and arranged to select a first catheter 100a based on the luminal sizing information (e.g. using algorithm 251). System 10 can be constructed and arranged to inflate or otherwise expand the functional assembly 130a of a catheter 100a (e.g. with an ablative fluid) to a diameter related to the smallest diameter location. System 10 can be constructed and arranged to inflate or otherwise expand the functional assembly 130b of a catheter 100b (e.g. with a gas) to a diameter corresponding to the expanded diameter of the selected catheter 100a (e.g. a diameter less than the proximate axial segment lumen size and/or to a diameter related to the smallest diameter location). System 10 can be further constructed and arranged to inflate the functional assembly 130b of a catheter 100b (e.g. with a gas) to a pressure sufficient to correlate to sufficient apposition with the axial segment luminal wall. System 10 can be constructed and arranged to apply a vacuum to one or more ports 137 of catheter 100b to engage neighboring tissue. System 10 can be constructed and arranged to inject fluid into tissue (e.g. submucosal tissue) until the pressure within the associated functional assembly 130b exceeds a threshold, at which time fluid (e.g. air) can be extracted from functional assembly 130b and fluid delivery by fluid delivery element 139c continues until the volume of functional assembly 130b reaches a pre-determined lower limit. System 10 can be further constructed and arranged to inject fluid into tissue (e.g. submucosal tissue) until the pressure within the associated functional assembly 130b exceeds a threshold, such as a threshold of 0.3 psi, 0.5 psi or 0.7 psi (e.g. but below a second threshold of 2.0 psi or 4.0 psi). System 10 can be constructed and arranged to subsequently disengage functional assembly 130b from the tissue (e.g. by removal of the vacuum from each port 137), and radially collapse balloon 136 (e.g. via extraction of fluid from balloon 136 via one or more conduits 111 of catheter 100b). System 10 can be constructed and arranged to similarly expand tissue at one or more other axial segments of the intestine. System 10 can be constructed and arranged to treat target tissue of the one or more axial segments (with expanded tissue) using the particular first catheter 100a whose expanded diameter was chosen based on the minimum diameter of the one or more axial segments. In some embodiments, multiple tissue expansion procedures are performed by catheter 100b sequentially, after which a series of target tissue treatments (e.g. tissue ablations) are performed by catheter 100a sequentially. Alternatively, a pattern of alternating between one or more tissue expansions and one or more tissue treatments can be performed.

As described herein, system 10 can be constructed and arranged to ablate or otherwise treat tissue with an expanded functional assembly 130 that is smaller than the native lumen diameter of an axial segment of intestine. The amount of fluid injected to expand tissue (e.g. submucosal tissue) can be determined in a closed-loop manner to achieve a post-expansion lumen size with a specific diameter along one or more axial segments of the intestine (e.g. the duodenum). System 10 can comprise a single functional assembly 130 configured to treat (e.g. ablate) multiple axial segments of intestine, each with a pre-expanded tissue layer (e.g. submucosal tissue layer expanded to a diameter approximating or otherwise related to the diameter of the expanded functional assembly 130). System 10 can comprise a functional assembly 130 configured to treat (e.g. ablate) multiple axial segments of intestine that are selected prior to the performance of tissue layer expansion, such as to reduce overall procedure time and/or time between tissue expansion and tissue treatment. System 10 can be constructed and arranged such that the difference between the native intestinal lumen diameter and the post-tissue expansion lumen diameter is known, such as to confirm acceptability of the tissue expansion step(s) prior to an ablation step being performed. System 10 can be constructed and arranged to eliminate one or more sizing steps, as described herein.

In some embodiments, system 10 is constructed and arranged to perform a medical procedure comprising a tissue treatment procedure for treating a patient disease or disorder, and the amount of tissue treated is based on the severity of the patient's disease or disorder (e.g. amount of tissue treated is proportional to the severity). In some embodiments, the disease treated is diabetes, and the severity is determined by measuring one or more of: HbA1c level; fasting glucose level; and combinations of one or more of these. In some embodiments, algorithm 251 is configured to determine the amount of tissue to be treated based on the severity of the patient's disease or disorder.

In some embodiments, system 10 is constructed and arranged to (e.g. via algorithm 251) to introduce fluid into functional assembly 130 (e.g. into a balloon 136 of functional assembly 130) until sufficient apposition against an intestinal wall is achieved (e.g. as determined by a pressure measurement and/or image analysis provided by a sensor of the present inventive concepts). Subsequently, fluid is extracted from functional assembly 130 (e.g. until a second, lesser volume of fluid resides within functional assembly 130), after which the intestinal wall is contracted (e.g. via desufflation as described herein) such that the intestinal wall again contacts functional assembly 130.

In some embodiments, desufflation is accomplished by applying vacuum to one or more fluid ports (also referred to as aspiration ports, insufflation ports, and/or desufflation ports) of a component of system 10, such as a fluid port configured to deliver fluids and/or extract fluids from the GI tract of the patient. A system 10 fluid port can include, but is not limited to: one or more ports 137; one or more openings of shaft 110 proximal or distal to functional assembly 130 (e.g. a functional element 119 constructed and arranged as a fluid delivery and/or fluid extraction port); and/or a fluid port of an endoscope or other introduction device 50.

In some embodiments, functional assembly 130 is anchored to the intestine (e.g. by expanding functional assembly 130 and/or by having port 137 engage tissue). In these embodiments, a tissue expansion procedure can be performed, such as by advancing at least one fluid delivery element 139c (e.g. at least three fluid delivery elements 139c) into tissue and delivering injectate 221 (e.g. into submucosal tissue). Alternatively or additionally, a tissue ablation procedure can be performed.

In some embodiments, system 10 is configured to deliver injectate 221 into tissue, such as via one or more fluid delivery elements 139c, each of which can be positioned in a port 137. The delivery of injectate 221 into tissue can produce a therapeutic restriction, occlude one or more body conduits (e.g. blood vessels), deliver a (single) bolus of drug or other agent into blood or other tissue, create a drug or other agent “depot” in tissue, and combinations of one or more of these. Injectate 221 can be configured to expand after delivery into tissue. Injectate 221 can be configured to remain relatively “in place” within tissue proximate the injection site for at least one month, three months, six months, or one year. Injectate 221 can be delivered into tissue (e.g. via fluid delivery element 139c) in a location selected from the group consisting of: lower stomach; pylorus; proximal small intestine; distal small intestine; duodenum; jejunum; terminal ileum; bowel; and combinations of one or more of these. In some embodiments, injectate 221 comprises a hydrogel, such as to create a hydrogel prosthesis within one or more tissue layers of the intestine (e.g. one or more submucosal tissue layers).

Injectate 221 can be delivered into tissue to create a therapeutic restriction, as described herein, such as to create a space occupying obstruction as a treatment for obesity, type 2 diabetes; hypercholesterolemia, hypertension; non-alcoholic fatty liver disease; non-alcoholic steatohepatitis; and/or other metabolic disease. Injectate 221 can be delivered to one or more tissue locations to create a sense of satiety, reduce chime throughput and/or reduce obesity. Injectate 221 can be injected into the gastric varices, such as when injectate 221 comprises an occlusive agent such as an adhesive such as cyanoacrylate. System 10 can be constructed and arranged such that delivery of injectate 221 into one or more tissue locations alters nutrient absorption and/or hormonal signaling from the mucosa. System 10 can be constructed and arranged to deliver injectate 221 into colon tissue (e.g. to expand colon submucosal tissue), such as to treat fecal incontinence.

As described above, system 10 can be constructed and arranged to deliver injectate 221 into tissue to deliver a bolus of medication and/or to create a drug or other agent depot within tissue of the patient, such as within mucosal tissue and/or submucosal tissue of the intestine. In some embodiments, an injectate 221 positioned within tissue is activated based on one or more signals produced by a sensor, such as a bioactive glucose sensor that responds to the detection of an analyte and leads to (e.g. via one or more components of system 10) release or other activation of injectate 221. For example, injectate 221 can comprise an anti-diabetic agent, such as insulin, and a sensor (e.g. an implant 192 described herein that is configured as a sensor) can comprise a glucose sensor that detects a glucose change, such as the higher glucose levels that occur after a meal. Injectate 221 can comprise a drug or other agent selected from the group consisting of: a steroid; an anti-inflammatory agent; a chemotherapeutic; a proton pump inhibitor; a sclerosant agent; a differentiation factor such as trans-retinoic acid; an anti-hyperglycemic agent such as GLP-1 analogue or others; an anti-obesity agent; an anti-hypertensive agent; an anti-cholesterol agent such as a statin or others; and combinations of one or more of these. In some embodiments, injectate 221 comprises a steroid or other anti-inflammatory agent delivered to a therapeutic restriction of the present inventive concepts (e.g. delivered into an existing restriction or to create a restriction). In some embodiments, injectate 221 comprises one or more steroids and/or other anti-inflammatory agents delivered to the site of chronic inflammation, such as a site of ulcerative colitis or Crohn's disease. In some embodiments, injectate 221 comprises one or more steroids or other anti-inflammatory agents delivered at the site of celiac disease (e.g. the proximal small intestine) and/or otherwise delivered to treat celiac disease. In some embodiments, injectate 221 comprises one or more chemotherapeutic agents delivered to the site of a cancerous or pre-cancerous lesion.

Injectate 221 can be injected into tissue in a single procedure or multiple procedures. System 10 can be configured to determine an injectate 221 delivery parameter (e.g. determined by algorithm 251), such as by performing an analysis based on a patient demographic parameter and/or a patient physiologic parameter, such as age, weight, HbA1c level and cholesterol level. The injectate delivery parameter can comprise a parameter selected from the group consisting of: volume of injectate 221 delivered; length and/or area of a tissue layer receiving injectate 221; type of material included in injectate 221; viscosity of injectate 221; titration result of injectate 221; and combinations of one or more of these.

In some embodiments, system 10 is constructed and arranged to both deliver a durable injectate 221 (e.g. injectate 221 remains in place for at least 1 month), as well as treat target tissue (e.g. a treatment comprising ablating duodenal and/or other intestinal mucosa). The two procedures can be performed on the same day or on different days.

In some embodiments, injectate 221 comprises a radiographic material, such as tantalum and/or barium sulfate, such as to be used in combination with X-ray or fluoroscopy to assess tissue expansion (e.g. submucosal tissue expansion), as described herein. Alternatively or additionally, injectate 221 can comprise a material that is visualizable under other imaging modalities (e.g. an imaging modality provided by imaging device 55), such as magnetic material; ferrous material; ultrasonically reflective material; and combinations of one or more of these.

In some embodiments, sensor 139b comprises a sensor configured to provide an impedance measurement, such as an impedance measurement used by algorithm 251 to enable closed-loop or otherwise adjust delivery of RF energy from treatment element 139a. In some embodiments, injectate 221 comprises a conductive substance, such as a conductive substance configured to enhance an impedance measurement recorded by sensor 139b. In these embodiments, injectate 221 can comprise one or more substances that are both conductive and visualizable (e.g. visualizable by imaging device 55 as described herein), such as tantalum.

In some embodiments, injectate 221 comprises a pharmaceutical drug or other agent (e.g. injectate 221 comprises agent 420) configured to provide a therapeutic benefit when delivered by one or more fluid delivery elements 139c into intestinal or other tissue. In these embodiments, injectate 221 can be injected into mucosal tissue and/or tissue proximate mucosal tissue (e.g. submucosal tissue). A major function of the mucosa is to bind or absorb certain molecules, and it can prevent or otherwise reduce the passage of all other molecules. Thus, insertion of injectate 221 directly into the submucosa can bypass the mucosal barrier, enabling the delivery of therapeutic large molecules that otherwise would be passed through the body completely or largely unabsorbed. This procedure also provides more precise dosage control, since the amount of absorption through the mucosa can be variable. Injectate 221 (e.g. injectate 221 comprising agent 420) can comprise any therapeutic biologic or biochemical entity. The entity of injectate 221 can have therapeutic effect by itself or it can be externally triggered, such as when injectate 221 comprises trigger materials, such as magnetic nanoparticles triggered by magnetic fields, gold nanoparticles triggered by light, optical or other fields, particles activated by light such as ultraviolet light or infrared light, and/or particles activated by heating or chilling. In some embodiments, tool 500 is configured to provide the triggering event, such as by generating a magnetic field, delivering light, and/or by delivering or extracting heat.

Local administration of drugs with high systemic toxicity and/or propensity for resistance by catheter 100 is advantageous, as much higher local concentrations of the drug and/or much lower systemic bioavailability can be achieved. Avoidance of skin-penetrating injections can be beneficial (e.g. avoiding associated pain, cosmetic issues and likely trauma to injection site). Catheter 100 can be used to deliver depot formulations of drugs or other agents to intestinal tissue (e.g. the intestinal submucosa) for the treatment of various GI or systemic illnesses. Submucosal delivery via catheter 100 can avoid the limitations associated with the mucosal barrier as described herein, and the limited bioavailability that is created. Submucosal delivery via catheter 100 can also allow the delivered drug to avoid chemical reactions or other adverse effects that result from interaction with various microbiological and pH environments in the patient's gut. In some embodiments, injectate 221 comprises an anti-reflux medication and/or an anti-acid medication, such as when injectate 221 is delivered into the mucosa or submucosa of the esophagus, intestine and/or stomach.

Systemic pharmaceutical therapy including immunomodulators to treat inflammatory bowel disease has issues with toxicity associated with immune suppression. This systemic therapy can also have limited efficacy once an individual develops antibodies against the monoclonal antibody therapies. In both cases, high systemic concentrations of the drugs limit the ability to achieve sufficiently effective doses in the GI tract itself, where the therapy needs to be most effective. A catheter 100 comprising one or more fluid delivery elements 139c can be used as a tool to perform site-specific delivery of drugs and other agents to treat GI illnesses, such as celiac disease and inflammatory bowel disease.

In some embodiments, system 10 comprises an implantable device, such as implant 192 shown. Implant 192 can comprise a medical device, such as a drug delivery depot or other drug delivery device. Implant 192 can comprise a sensor or sensing device. In some embodiments, system 10 is configured to deliver implant 192 via a functional element 139, such as fluid delivery element 139c (e.g. when fluid delivery element 139c comprises a needle comprising a lumen through which a sensor-based implant 192 can be deployed into tissue such as mucosal tissue, submucosal tissue, other intestinal tissue and/or other tissue of the patient). In some embodiments, system 10 is constructed and arranged to deliver one or more implants 192 into tissue that is not proximate to a significant number of pain-sensing nerves. In some embodiments, implant 192 can comprise a sensor configured to measure a physiologic parameter selected from the group consisting of: blood pressure; heart rate; pulse distention; glucose level; blood glucose level; blood gas level; hormone level; GLP-1 level; GIP Level; EEG; LFP; respiration rate; breath distention; perspiration rate; temperature; gastric emptying rate; peristaltic frequency; peristaltic amplitude; and combinations of one or more of these.

In some embodiments, implant 192 comprises a sensor, such as a sensor configured to be implanted in the submucosal tissue of the intestine. In some embodiments, catheter 100 is configured to deliver implant 192 into tissue via a fluid delivery element 139c and/or another functional element of catheter 100. Implant 192 can comprise a sensor configured to produce a signal related to a physiologic parameter related to the concentration of a material selected from the group consisting of: fat, sugar (e.g. glucose or fructose); protein; one or more amino acids; and combinations of one or more of these. In some embodiments, implant 192 comprises a wireless communication element, such as an RF or infrared element configured to transmit information (e.g. to a receiving component of system 10). System 10 can be configured to analyze the received information, such as an analysis performed by algorithm 251 used to manage obesity, insulin resistance and/or Type 2 diabetes.

In some embodiments, system 10 is constructed and arranged to expand tissue by delivering injectate 221 into tissue (e.g. submucosal tissue of the intestine) with fluid delivery element 139c. System 10 can be constructed and arranged to deliver injectate 221 at a constant or varied rate, in open loop or closed loop delivery configurations. In some embodiments, system 10 is configured to deliver fluid at an elevated flow rate and/or at an elevated pressure, such as with a flow rate and/or pressure which decreases over time. System 10 can be constructed and arranged to monitor one or more pressures achieved during delivery of injectate 221 into tissue. System 10 can be configured to measure a pressure using a pressure sensor-based functional element 109, 119, 139, 209, 229 and/or 309. Alternatively or additionally, system 10 can comprise a sensor positioned in tissue proximate the tissue to be expanded. In some embodiments, catheter 100 comprises multiple fluid delivery elements 139c, such as an array of three fluid delivery elements 139c equally spaced about functional assembly 130. In these embodiments, injectate 221 can be delivered into tissue by the multiple fluid delivery elements 139c simultaneously or sequentially. Pressure measured by system 10 can correlate to the quality of tissue expansion, or other tissue expansion parameter. In some embodiments, system 10 regulates delivery of injectate 221 (e.g. by regulation of one or more pumps 225 delivering injectate 221), and/or detects an undesired state in the delivery of injectate 221, based on pressure measured by system 10. System 10 can be configured to confirm that during delivery of injectate 221, a proper pressure increase occurs in the expanded tissue, within functional assembly 130 and/or at another system 10 location. The pressure at a first location can be measured directly (e.g. via a pressure sensor-based functional element located proximate the first location, or indirectly such as via a pressure sensor-based functional element located at a second location whose pressure can be correlated to the pressure at the first location, as described herein for measurement of pressure, temperature and/or any system 10 parameter). System 10 can prevent a pressure threshold from being surpassed at one or more locations, such as to prevent an undesired event such as an amount and/or location of expansion of tissue that can have a deleterious effect, such as expansion of serosal tissue of the intestine. In some embodiments, pressure information is processed (e.g. via algorithm 251), such that cumulative pressure information (e.g. time at pressure, pressure change rates, and the like) can be compared to one or more thresholds. In these embodiments, pressure information and/or processed pressure information (herein “pressure information”) can be used to confirm size or geometric shape of expanded tissue, such as to confirm full circumferentially of a tissue expansion. In some embodiments, system 10 correlates one or more pressure readings below a threshold to an adverse event selected from the group consisting of: fluid delivery element 139c not delivering fluid into the appropriate tissue (e.g. fluid delivery element 139c has not properly penetrated tissue); failure of a functional element such as failure of a functional element comprising a valve; leak in a conduit such as a leak in a conduit 111, 211, 212 and/or 311; and combinations of one or more of these.

In some embodiments, functional assembly 130 is expanded with fluid at a first pressure (e.g. a pressure of approximately 0.5 psi, 0.7, psi or 0.9 psi), and fluid is delivered into tissue by one or more fluid delivery elements 139c (e.g. three fluid delivery elements 139c). During fluid delivery, system 10 can monitor pressure (e.g. a sensor of the present inventive concepts monitors pressure within functional assembly 130 and/or within a conduit in fluid communication with functional assembly 130), and if the pressure exceeds a second pressure (e.g. a pressure of at least 0.7 psi, 0.9 psi 1.1 psi, or other pressure greater than the first pressure), system 10 can reduce the pressure within the functional assembly 130 (e.g. reduce the pressure to the first pressure).

In some embodiments, system 10, console 200 and/or catheter 100 are constructed and arranged to perform partial circumferential tissue expansion of one or more axial segments of the GI tract (e.g. less than 360° expansion of submucosal tissue of one or more axial segments of the intestine). In some embodiments, injectate 221 comprises a relatively viscous material and catheter 100 delivers injectate 221 to create focal (i.e. partial circumferential) or multi-focal expansions of tissue (e.g. multiple partial circumferential expansions of submucosal tissue). In some embodiments, a therapeutic restriction or other tissue expansion of the present inventive concepts can comprise two or more focal restrictions created around the circumference of an axial segment of tubular tissue that block more than 50% or more than 75% of the luminal diameter. In some embodiments, a full or near-full circumferential expansion of tissue is created by first expanding (e.g. inflating) a functional assembly 130 and creating one or more focal expansions, subsequently compacting (e.g. deflating) the functional assembly 130, re-expanding (e.g. re-inflating) the functional assembly 130 and creating additional focal expansions between the previously expanded areas to create a substantially circumferential expansion. Prior to re-expanding, functional assembly 130 can be repositioned (e.g. rotated). The compacting and re-expanding can be configured to allow multiple fluid delivery elements 139c to self-reposition during contact with the peaks of the focal expansions (e.g. reposition into valleys in between the focal expansions). Alternatively, the functional assembly 130 (e.g. shaft 110 of the catheter 100) can be rotated and/or otherwise repositioned (e.g. automatically and/or manually) after the initial focal expansions.

In some embodiments, functional assembly 130 comprises an expanded diameter of a magnitude (e.g. a small enough diameter) configured to accommodate a range of luminal diameters of the small intestine. Desufflation of the duodenum (e.g. using body introduction device 50 and desufflation techniques known to those of skill in the art) can be performed to collapse the inner wall of the intestine onto a fully expanded functional assembly 130. Functional assembly 130 can comprise one or more ports (e.g. ports 137 and/or a functional element 119 constructed and arranged as a fluid delivery/extraction port) configured to desufflate and/or aspirate (e.g. extract fluid) to collapse the inner wall of the intestine onto functional assembly 130. In some embodiments, system 10 comprises a separate desufflation tool (e.g. aspiration tool), such as tool 500 constructed and arranged to extract fluid (e.g. a liquid and/or a gas) from a segment of intestine, such as a segment comprising functional assembly 130. In these embodiments, tool 500 can comprise one or more holes, slots, slits or other openings (e.g. positioned in a distal portion of tool 500) that are configured to aspirate fluids from the intestine, such as to collapse the inner wall of the intestine onto a fully expanded functional assembly 130. As described herein, desufflation and/or aspiration can be used to impact or otherwise modify the target tissue treatment and/or tissue expansion.

In some embodiments, system 10 is configured to work in combination with a patient care practice, such as a patient diet that is maintained prior to and/or after performance of a medical device or diagnostic procedure performed using system 10. For example, a patient diet or other patient practice can be included prior to and/or after a tissue treatment procedure performed by system 10 to slow down healing (e.g. mucosal healing) and/or provide another enhancement to the therapy achieved. In some embodiments, mucosal healing is slowed down by a functional element 139, tool 500 and/or other component of system 10. In some embodiments, regrowth of treated mucosal tissue is enhanced by a pre-procedural and/or post-procedural patient diet. The diet can include: a liquid diet for at least one day; a low-sugar diet and/or a low-fat diet for at least one week; a standardized diabetic diet for at least 1 week; and/or nutritional counseling for at least 1 week.

In some embodiments, injectate 221 comprises an injectate configured to cause inflammation of tissue. In these embodiments, one or more fluid delivery elements 139c can be configured to deliver the injectate 221 to tissue to cause an inflammatory response in the tissue. The inflammatory response can result in a tissue layer that functions as a protective layer during a subsequent tissue treatment procedure (e.g. tissue ablation procedure) performed by a functional assembly 130 of catheter 100.

In some embodiments, system 10 includes a tool 500 comprising a mucus removal assembly constructed and arranged to remove mucus from one or more intestinal wall locations (e.g. a full or partial circumferential segment of intestine), such as to remove mucus prior to a tissue treatment performed by functional assembly 130. Alternatively or additionally, functional assembly 130, one or more functional elements 139 and/or one or more other components of catheter 100 can be constructed and arranged to similarly remove mucus. In some embodiments, mucus is removed mechanically. Alternatively or additionally, mucus is removed by delivery (e.g. via one or more fluid delivery elements 139c) of agent 420 to a tissue surface (e.g. when agent 420 comprises a mucolytic agent).

In some embodiments, system 10 comprises one or more materials or devices configured to modify tissue healing, such as when catheter 100 is constructed and arranged to treat intestinal mucosa (e.g. duodenal mucosa). For example, injectate 221, or implant 192 can be delivered in and/or proximate target tissue, such as at a time prior to, during and/or after target tissue treatment. In these embodiments, for example, injectate 221, agent 420 and/or implant 192 that is delivered (e.g. by fluid delivery element 139c or another component of catheter 100) can be configured to delay healing of treated tissue in the intestine, such as to provide enhanced therapeutic benefit to the patient and/or prolong the benefit (e.g. enhance or prolong HbA1c reduction). In some embodiments, injectate 221, agent 420 and/or implant 192 comprises a material selected from the group consisting of: a chemotherapeutic agent; a cytotoxic agent; 5 Fluorouracil; Mitomycin-c; Tretinoin topical (Retin-A, Retin-A Micro, Renova); Bleomycin; Doxorubicin (Adriamycin); Tamoxifen; Tacrolimus; Verapamil (Isoptin, Calan, Verelan PM); Interferon alfa-2b; Interferon beta 1a (Avonex, Rebif); Interferon alfa-n3 (Alferon N); Triamcinolone (Aristospan, Kenalog-10); Imiquimod (Aldara, Zyclara); and combinations of one or more of these.

In some embodiments, system 10 includes pressure neutralizing assembly, assembly 72 shown, which can be constructed and arranged to monitor and/or adjust (e.g. automatically or semi-automatically) the pressure within a segment of the intestine, such as to allow one or more therapeutic or diagnostic procedures to be performed by functional assembly 130 at a particular pressure or within a particular range of pressures. Pressure neutralizing assembly 72 can be configured to deliver or extract fluids from a segment of the intestine, such as to perform an insufflation procedure, a desufflation procedure, or to otherwise modify the pressure within the segment of the intestine proximate functional assembly 130.

In some embodiments, system 10 is constructed and arranged to produce an image (e.g. an image produced by an imaging device and/or other sensor of the present inventive concepts). Algorithm 251 can be configured to analyze one or more images of tissue that are visualized through one or more portions of functional assembly 130, such as to determine the level of tissue expansion and/or a level of tissue ablation, such as to assess completion adequacy of one or more steps of a medical procedure.

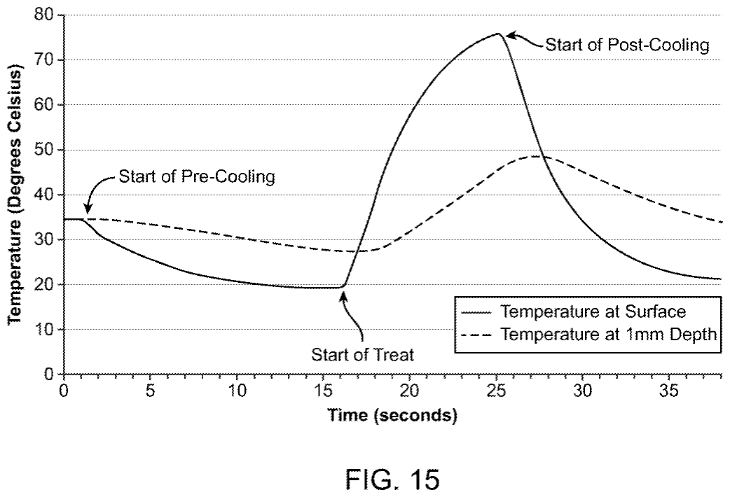

System 10 can comprise one or more test devices, test assembly 295, for performing a calibration and/or other system diagnostic function on one or more components of system 10. In some embodiments, test assembly 295 is configured to adjust one or more ablation parameters of system 10, such as is described herein in reference to FIG. 15. In some embodiments, test assembly 295 is configured to adjust one or more tissue expansion parameters of system 10.

In some embodiments, shaft 110 comprises an axial twist along at least a portion of its length, such that conduits 111 comprise a comparable path length when shaft 110 is positioned within a tortuous path (e.g. the curvilinear path found in the GI tract). The axial twist of shaft 110 can be created or otherwise manufactured during an extrusion process of shaft 110. In some embodiments, the axial twist is oriented in the counterclockwise direction (e.g. to provide advantageous insertion in the GI tract of a human patient). In some embodiments, the axial twist comprises a constant pitch along the length of shaft 110. In some embodiments, the axial twist comprises a variable pitch along the length of shaft 110. In some embodiments, shaft 110 comprises the axial twist along its distal portion (e.g. only its distal portion). In some embodiments, each 16-19 inch portion of shaft 110 comprises a pitch comprising one full rotation (e.g. a 360° rotation), such as a full counterclockwise rotation over each 16-19 inch portion of shaft 110. In some embodiments, shaft 110 comprises 3.5 to 4.2 full rotations (e.g. counterclockwise rotations) along the length of shaft 110.

In some embodiments, catheter 100 (e.g. handle 102) includes a spring compensation mechanism (not shown) configured to advance one, two, or more fluid delivery elements 139c. The spring compensation mechanism can be configured to compensate for a length difference between two or more components of catheter 100, for example, when a fluid delivery advancement knob or other control (e.g. control 104 of catheter 100 of FIG. 2 and/or control 24 of device 20 of FIG. 2) comprises a travel length that is greater than the advancement distance of fluid delivery elements 139c. In some embodiments, catheter 100 comprises a spring compensation mechanism as described in applicant's co-pending U.S. patent application Ser. No. 16/742,645, entitled “Intestinal Catheter Device and System”, filed Jan. 14, 2020.

In some embodiments, conduits 111 are constructed and arranged to prevent or otherwise reduce the occurrence of infection. One, two, or more conduits 111 can comprise single-use conduits (e.g. configured to be sterilized a single time). Alternatively or additionally, one, two, or more conduits 111 can comprise re-sterilizable conduits (e.g. conduits configured to be sterilized, used in a first patient, sterilized again, and used in a second patient).

In some embodiments, conduits 111 comprise one, two, or more relatively non-compliant materials configured to promote accurate (e.g. intended) fluid transmission from console 200, through shaft 110, and/or to functional assembly 130. For example, conduits 111 can comprise a construction to avoid significant expansion during delivery of injectate 221 during a tissue expansion procedure.

In some embodiments, conduits 111 comprise one, two, or more sensors (e.g. functional element 119 comprising one or more sensors) configured to detect liquid (e.g. injectate 221), gas (e.g. air), fluid color, flow rate, and/or an obstruction within conduit 111.

In some embodiments, at least one pumping assembly 225 comprises one, two, or more syringe pumps configured to manipulate (e.g. advance and/or retract) a plunger of a syringe operably attached to the syringe pump. Each syringe can comprise one or more fluids, such as injectate 221. At least two syringes can comprise dissimilar fluids (e.g. dissimilar injectates 221). At least two syringes can comprise similar fluids (e.g. similar injectates 221). A pumping assembly 225 can comprise multiple syringe pumps, such as one syringe pump (e.g. for delivery of a single syringe of fluid) for each fluid delivery element 139c. For example, catheter 100 can comprise three fluid delivery elements 139c and pumping assembly 225 can comprise three syringe pumps (e.g. for delivering fluid from three different syringes) corresponding to each fluid delivery element 139c. As described herein, each fluid delivery element 139c can be operably attached to a unique (e.g. separate) syringe pump (e.g. multiple syringe pumps comprising similar or dissimilar injectates 221). In other embodiments, two or more fluid delivery elements 139c are operably attached to the same syringe pump.

In some embodiments, fluid delivery elements 139c are operably attached (e.g. at least fluidly attached) to a unique (e.g. separate) conduit 111, such as to ensure an equal delivery of fluid to each fluid delivery element 139c. Each syringe pump of a pumping assembly 225 can comprise a distal and/or proximal sensor (e.g. sensors configured to detect travel at distal and proximal ends, respectively, of the stroke distance of the syringe pump) configured to ensure a full (e.g. complete) injection stroke during the delivery of fluid into tissue via fluid delivery element 139c. For example, the syringe pump can comprise a proximal sensor comprising a “homing sensor” configured to recognize a starting position of the syringe pump, and the syringe pump can comprise a distal sensor comprising a “finish sensor” configured to recognize a completion position of the syringe pump following a full injection stroke. System 10 can be configured to automatically refill the attached syringe following a delivery of fluid to tissue via the fluid delivery element 139c, such that the syringe is automatically prepared (e.g. without requiring operator action) for a subsequent delivery of fluid. Each syringe pump can be configured to operate at a pressure less than or equal to 110 psi (e.g. a threshold of 100 psi). Each syringe pump can be configured to operate at a speed that results in delivery of 10 mL of fluid over a duration of less than 60 seconds, such as 10 mL over a duration of less than 45 seconds, such as 10 mL over a duration of less than 30 seconds, such as 10 mL over a duration of approximately 10 seconds. In some embodiments, each syringe pump is configured to operate at a maximum speed, as long as the pressure of injectate (e.g. the pressure at the outlet of the syringe) is maintained below a threshold (e.g. a threshold of no more than 110 psi). If the pressure goes above the threshold, the syringe pump can be configured to operate at a slower speed such that the pressure remains below the threshold.

In some embodiments, a syringe pump of a pumping assembly 225 comprises a single mechanism configured to actuate three syringes simultaneously and/or at the same speed. For example, the flow path with the highest flow resistance will regulate (e.g. dictate) the speed of actuation for all three syringes, such as when the syringe pump is configured to drive to a target pressure (e.g. a pressure of no more than 110 psi) of any of the three syringes.

In some embodiments, each syringe pump of a pumping assembly 225 includes a linear encoder configured to detect and/or quantify the volume of fluid delivered to fluid delivery elements 139c.

In some embodiments, system 10 is configured to monitor one or more parameters of each of the syringe pumps of a pumping assembly 225 during the delivery of fluid (e.g. delivery of injectate 221 into tissue). System 10 can be configured to alert (e.g. provide an alarm) if an issue is detected during the delivery of fluid. In some embodiments, system 10 is configured to alert when a high pressure is present, such as when a syringe pump can generate a pressure that is at least 10% greater than the threshold used (e.g. at least 10% greater than a threshold of 100 psi). In some embodiments, system 10 is configured to alert when a high flow resistance is present, such as when a component of the syringe pump (e.g. a carriage of the syringe pump) does not translate or otherwise does not sufficiently move for at least 3 seconds. In some embodiments, system 10 is configured to alert when a low pressure is present (e.g. a pressure that is lower than expected under normal operating conditions of system 10), such as when air and/or a leak is detected within the associated flow path. In some embodiments, system 10 begins monitoring for this low pressure after a time delay, such as a time of at least 1 second, or at least 2 seconds after a flow of fluid (e.g. the flow of injectate 221) is initiated (e.g. and continues until that flow of fluid is completed). In some embodiments, a set of three conduits (e.g. a conduit with a higher flow resistance than the other two conduits), receives fluid at a higher pressure (e.g. a pressure of at least 90 psi, such as a pressure of approximately 110 psi) than the other two conduits (e.g. when the other two conduits receive fluid at a pressure less than 90 psi, such as a pressure of approximately 80 psi). As described herein, system 10 can be configured to shut down or otherwise reduce performance when a syringe pump parameter is not within a normal range.

In some embodiments, system 10 is configured to monitor an increase in pressure within each of the syringe pumps of a pumping assembly 225 during the delivery of fluid (e.g. injectate 221 into tissue). System 10 can be configured to alert (e.g. provide an alarm) if the pressure within a syringe pump increases at a rate below a threshold (e.g. the pressure increases slower than should be encountered during normal operation).

In some embodiments, one, two, or more components of system 10 are insulated or otherwise covered, such as to protect a user from contacting a surface of system 10 that increases in temperature during use.

In some embodiments, functional element 209 comprises two pressure sensors, such as a first pressure sensor constructed and arranged to measure the pressure of fluid leaving console 200 (e.g. delivered to catheter 100), and a second pressure sensor constructed and arranged to measure the pressure of fluid entering (e.g. via a return path) console 200.

In some embodiments, console 200 is configured (e.g. via algorithm 251) to check that a minimum level of fluid is present in a reservoir 220 prior to beginning the delivery of a fluid to catheter 100 (e.g. prior to performing a treatment step, a neutralizing step, and/or a tissue expansion step). For example, functional element 229a can comprise a fluid level sensor or other sensor configured to measure the amount of fluid in a reservoir 220. In some embodiments, console 200 prevents the initiation of a fluid delivery step if a reservoir 200 isn't in a full state.

In some embodiments, controller 250 comprises a software component and a firmware component. In some embodiments, algorithm 251 can be executed in either or both of the software and firmware of controller 250. In some embodiments, controller 250 provides a graphical user interface, GUI 207, for example a graphical user interface configured to be displayed on user interface 205. GUI 207 can comprise one or more controls (e.g. user selectable controls) configured to execute one or more operations of system 10. For example, during a treatment procedure, a user of system 10 can advance through a series of steps of the procedure by selecting one or more controls of GUI 207 to cause various actions of system 10 (e.g. one or more steps of method T10 described herein). In some embodiments, one or more controls of GUI 207 can be hidden, disabled, or otherwise unavailable to the user during one or more steps of a treatment procedure, such as to prevent one or more steps from being performed out of order. In some embodiments, GUI 207 provides visual instructions (e.g. diagrams and/or video instructions) to the user, such as when a physical action is required before, during, and or after a step of a treatment procedure. For example, GUI 207 can instruct the user to advance and/or retract control 104 of handle 102, described herein. In some embodiments, GUI 207 is configured to prevent an undesired “skipping” of procedural steps, such as when GUI 207 requires successful performance of a first step (e.g. initiation of the first step and/or operator or system 10 confirmation of completion of the first step) prior to providing access to a second step (e.g. prior to making the second step available for initiation).

In some embodiments, GUI 207 displays a progress bar or other visual indicator of the progress or other status of a step of a treatment procedure. In some embodiments, the progress bar can comprise a mapping that provides a visual indication of the status of the step being performed. For example, in some embodiments, the mapping can comprise a linear mapping, such as a mapping to time, wherein a step takes a fixed amount time and the progress bar indicator moves with a constant speed as the step progresses. Alternatively or additionally, the mapping can comprise a non-linear mapping, such as a mapping correlating to a temperature, wherein the temperature change is non-linear, but the progress bar indicator is configured to move with a relatively constant speed as the step progresses.

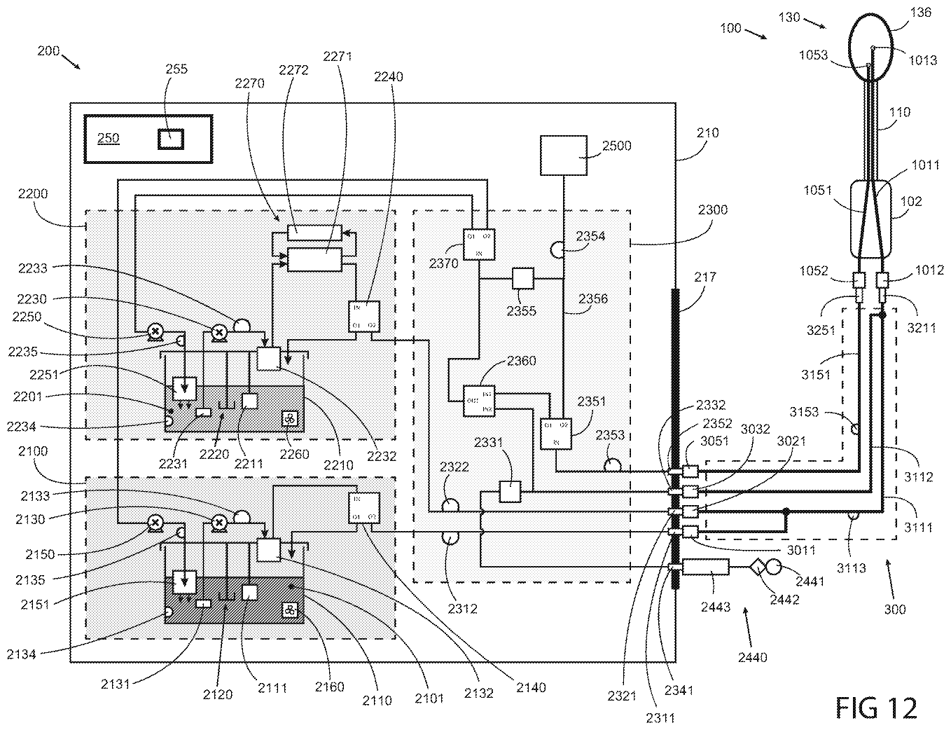

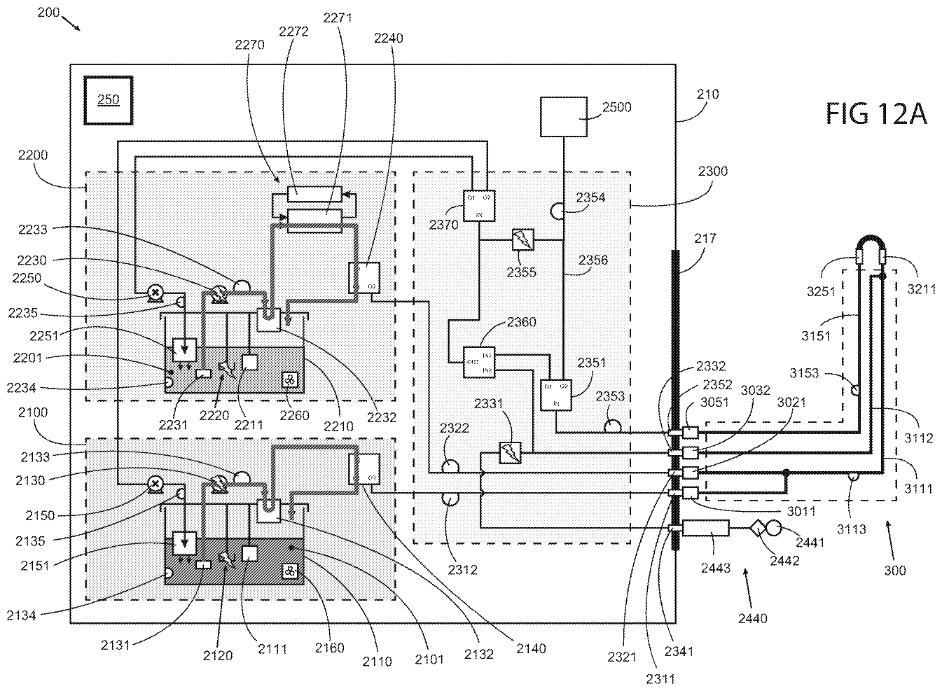

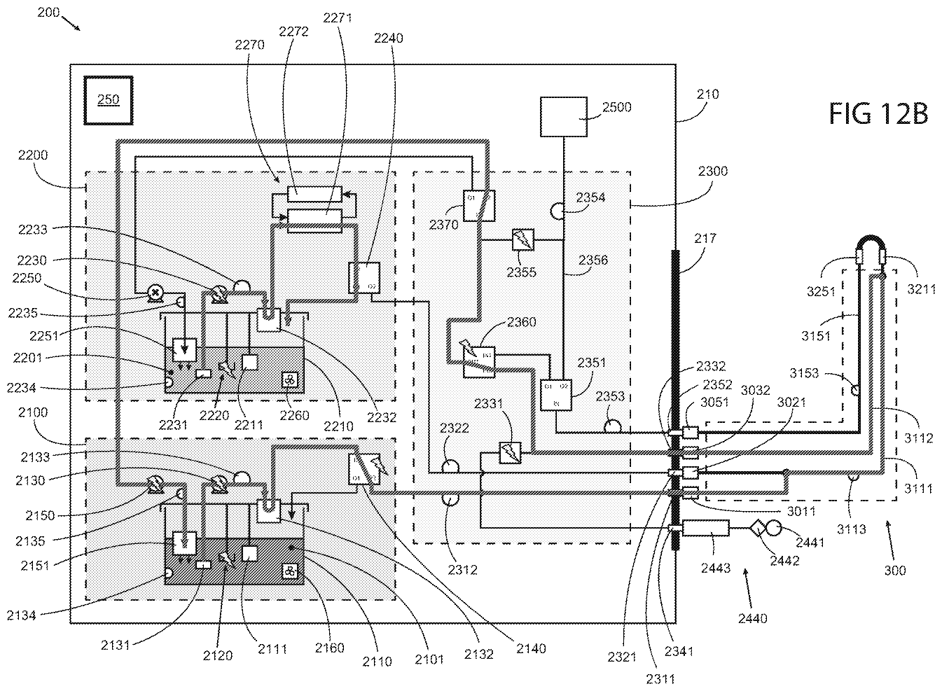

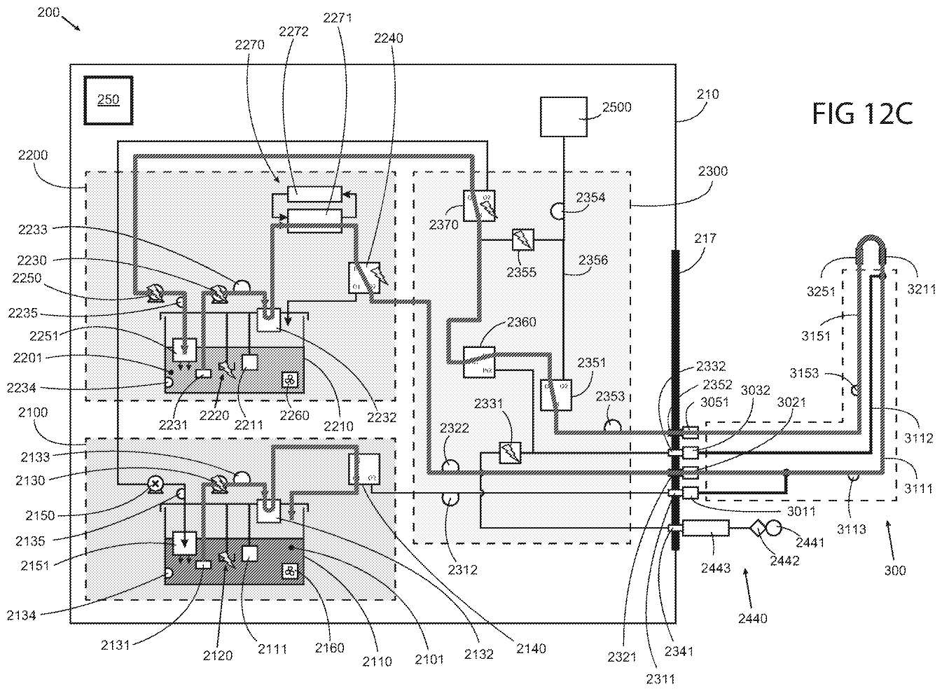

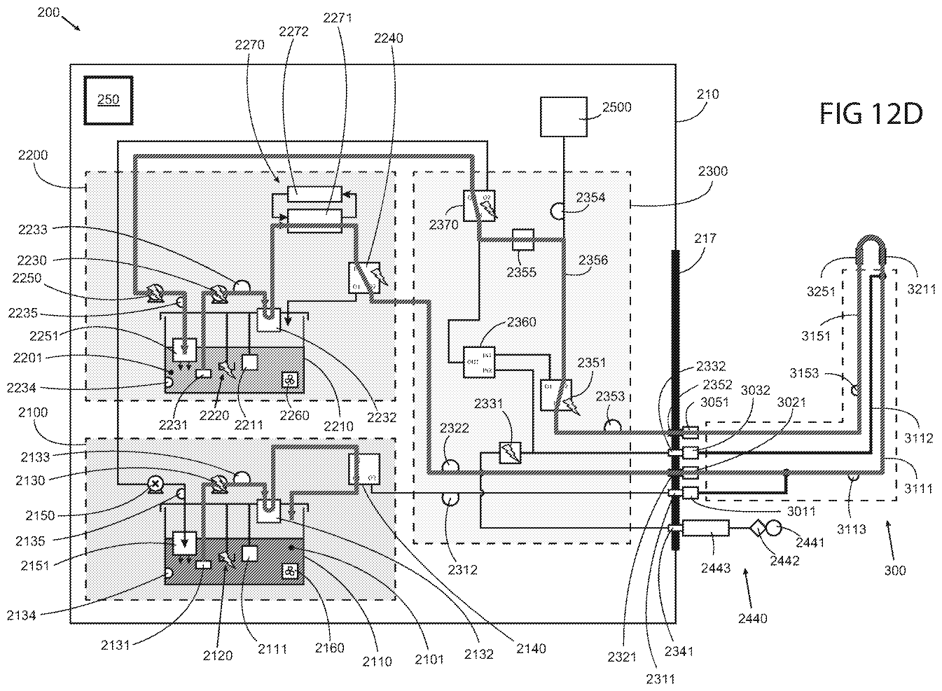

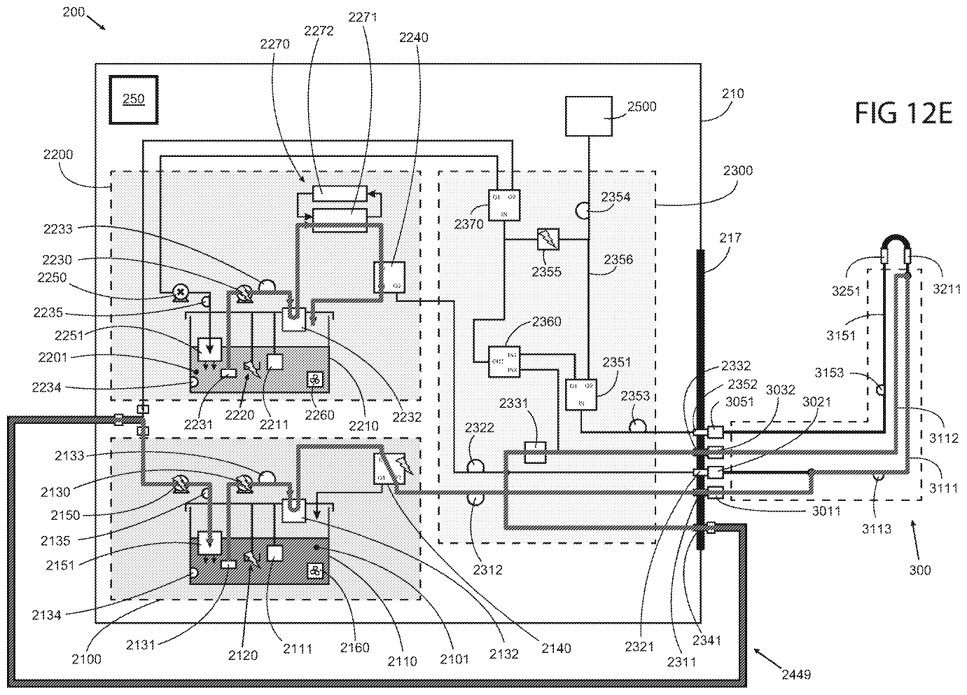

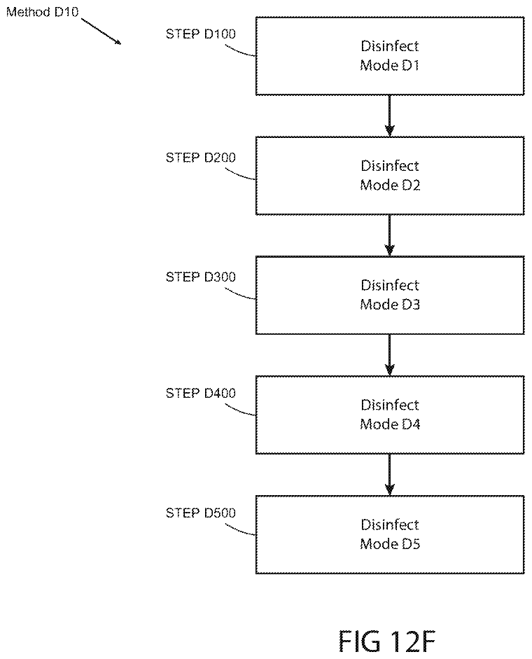

In some embodiments, GUI 207 displays the status of one or more components of system 10 during a step of a treatment procedure. For example, GUI 207 can display which fluid pathways are active, and the temperature and/or source of the fluid within the pathways during a treatment step, such as is described in FIGS. 12-14 herein. GUI 207 can display the temperature of a fluid (e.g. hot fluid 2101 and/or cold fluid 2201) within a reservoir (e.g. hot reservoir 2110 and/or cold reservoir 2210) and/or the level of the fluid within a reservoir, as described herein (e.g. as provided by a signal produced by a sensor-based functional element of system 10). In some embodiments, GUI 207 can display the relative position of a portion of catheter 100 (e.g. balloon 136) to an anatomical feature of the patient, for example relative to the ampula of Vater. In some embodiments GUI 207 can display the (current) total number of treatment steps (e.g. ablative treatment steps) performed during a procedure.

In some embodiments, GUI 207 and/or another component of user interface 205 (e.g. a speaker) is configured to indicate to the user when a step of a treatment procedure is completed, and/or when the user should begin a step of a treatment procedure, for example a step which requires a physical user action, such as advancing a control of catheter 100 or console 200. In some embodiments, user interface 205 produces a beep and/or other sound to indicate to the user that a control of catheter 100 should be advanced or otherwise activated, such as to advance one or more fluid delivery elements 139c to perform a tissue expansion procedure, as described herein.

In some embodiments, GUI 207 provides instructions to the user to perform a physical action (as described herein) if a step of a treatment procedure is canceled, such as to cause catheter 100 to be in a safe configuration following the canceled step (e.g. to retract one or more fluid delivery elements 139c, stop the flow of ablative fluid, and/or initiate the flow of a neutralizing fluid).

In some embodiments, GUI 207 provides one or more controls configured to execute two or more steps of a treatment procedure, such as when a step of a treatment procedure should be performed immediately following a proceeding step (e.g. a second step should always be performed after a first step). For example, GUI 207 can avoid including a control configured to activate a treatment step (e.g. an ablative treatment step), unless and until a tissue expansion step has been performed. In these embodiments, GUI 207 can comprise a control to perform a tissue expansion, and a control to perform a tissue expansion and an ablative treatment. For example, a user-selectable icon used to initiate an ablation step can be presented on GUI 207 only after a tissue expansion step is successfully completed.

In some embodiments, console 200 is configured to provide instructions to an operator of system 10 after an alarm condition occurs. For example, instructions can be provided via GUI 207 to recover from an alarm condition, such as to simply repeat a previous one or more steps, and/or to perform a specific recover procedure (e.g. replacement of catheter 100 and/or other system 10 component).

In some embodiments, algorithm 251 of console 200 is configured to log the progress of a procedure, for example to create and store in memory a log of actions, alerts, and/or other occurrences, and the timing of those occurrences.

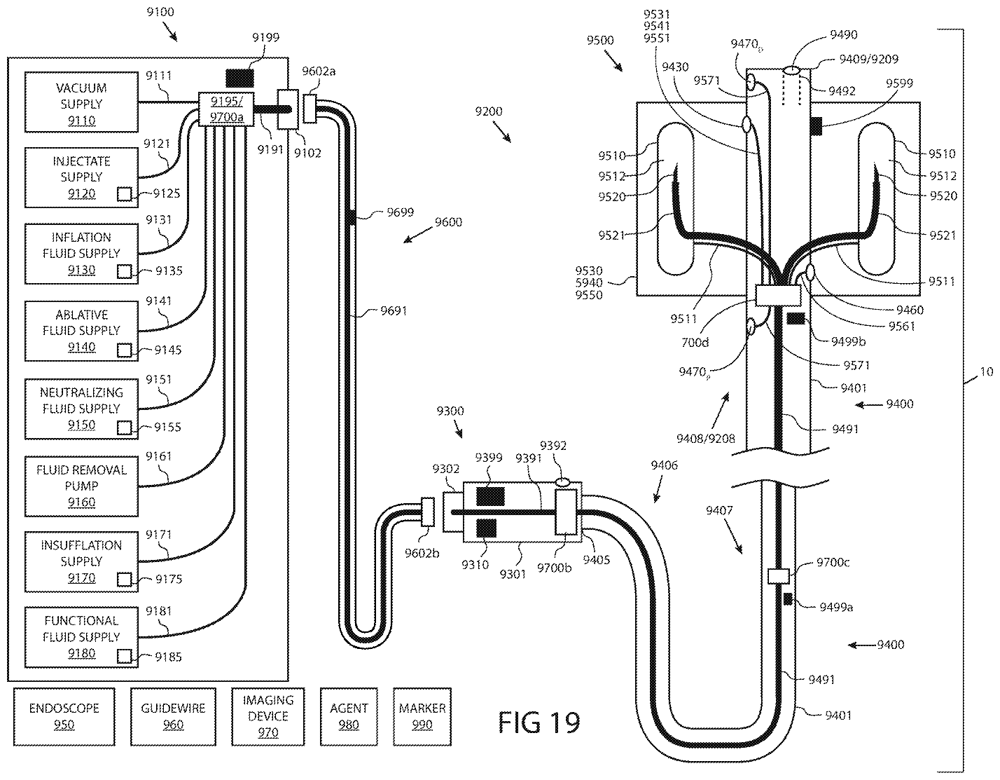

In some embodiments, system 10 of FIG. 1, and/or one or more of its components, is of similar construction and arrangement to system 10 of FIG. 19 described herein.

In some embodiments, system 10 is configured to ablate nerves of the small intestine, such as an ablation performed which also ablates the mucosal tissue of the intestine, or an ablation which avoids ablating (e.g. causing necrosis of) the mucosal tissue of the intestine. In these embodiments, one or more tissue expansion procedures, such as is described herein, can be performed prior to one or more (e.g. all) of the ablations of the nerves of the small intestine. In some embodiments, system 10, via functional assembly 130, is configured to deliver focused energy (e.g. focused light energy and/or focused sound energy such as high intensity focused ultrasound energy, HIFU) to the nerves of the small intestine (e.g. nerves in the submucosal layer), such as to ablate the nerves of the submucosal layer with or without also ablating tissue of the mucosal layer (e.g. without ablating all of the tissue of the mucosal layer proximate the one or more nerves being ablated). In some embodiments, nerves of the small intestine are ablated when fluid at an elevated temperature T_{H} (e.g. a temperature above patient body temperature, such as a temperature between 44° C. and 50° C.) is delivered to functional assembly 130 (e.g. to balloon 136) for a time period TP_{H} after which fluid at a temperature T_{C} (e.g. a temperature below temperature T_{H}, such as a temperature proximate but below temperature T_{H}, such as a temperature between 37° C. and 43° C.) is delivered to functional assembly 130 for a time period TP_{C}. Temperature T_{H}, temperature T_{C}, time period TP_{H}, and time period TP_{C} can be selected such that nerves of the intestine (e.g. in the submucosal tissue) are ablated (e.g. via heat received via thermal conduction) while tissue of the mucosal layer is not ablated (e.g. the mucosal tissue is not at an ablative temperature for a sufficient time period to cause necrosis).

In some embodiments, system 10 is configured to ablate nerves of the small intestine via injection of a neurolytic agent into the tissue, such as an injection of neurolytic agent into submucosal tissue that includes one or more nerves to be ablated, as described hereabove. For example, one or more fluid delivery elements 139c can be configured to deliver one or more agents selected from the group consisting of: a neurolytic agent; alcohol; ethanol; phenol; hypertonic saline; glycerol; an ammonium salt solution; an aminoglycoside; cholesterol; and combinations of these.

Referring now to FIG. 2, a schematic view of a system for performing a medical procedure on the small intestine of a patient is illustrated, consistent with the present inventive concepts. System 10 can comprise one or more components of similar construction and arrangement to similar components of system 10 of FIG. 1 described herein. System 10 comprises catheter 100 and console 200. Catheter 100 is constructed and arranged to treat target tissue, such as via the delivery of energy and/or an ablating agent to target tissue. Catheter 100 includes a port, connector 103 shown, which operably attaches to connector 203 of console 200. In some embodiments, system 10 further comprises tissue expansion catheter 20 which is constructed and arranged to expand one or more layers of tissue, such as one or more layers of target tissue and/or one or more layers of tissue proximate target tissue (e.g. one or more layers of safety-margin tissue as described herein). In some embodiments, system 10 further comprises lumen diameter sizing catheter 30 which is constructed and arranged to collect information correlated to the diameter of a portion of tubular tissue (e.g. one, two or more diameters of a GI lumen within and/or proximate target tissue). In some embodiments, system 10 comprises multi-function catheter 40, which is constructed and arranged to perform two or more functions selected from the group consisting of: tissue treatment (e.g. tissue ablation); tissue expansion; luminal diameter sizing; and combinations of one or more of these. In some embodiments, system 10 comprises multi-function catheter 40, and does not include one or more of: catheter 100, tissue expansion catheter 20 and/or sizing catheter 30.

System 10 can further comprise a body introduction device, such as a vascular introducer, laparoscopic port, and/or an endoscope, such as endoscope 50a shown. System 10 can further comprise one or more guidewires, such as guidewires 60a and 60b (singly or collectively guidewire 60). In some embodiments, one or more guidewires 60 comprise one, two, or more guidewires selected from the group consisting of: an 0.035″ guidewire; a Savary-Gilliard® 400 cm guidewire, a Dreamwire™ guidewire; a Jagwire™ guidewire; and/or a similar guidewire. In some embodiments, system 10 includes scope attached sheath, sheath 80 shown. Sheath 80 can comprise an elongate hollow tube which attaches (e.g. in a side-by-side manner) at one or more points along endoscope 50a. Sheath 80 can attach to endoscope 50a along a majority of its length. In some embodiments, sheath 80 comprises the Reach® overtube manufactured by U.S. Endoscopy, or similar.

Catheter 100, tissue expansion catheter 20, lumen diameter sizing catheter 30 and multi-function catheter 40 comprise handles 102, 22, 32 and 42, respectively. Handles 102, 22, 32 and 42 each comprise one or more controls, controls 104, 24, 34 and 44, respectively. Controls 104, 24, 34 and 44 can be configured to allow an operator to control one or more functions of the associated device, such as a function selected from the group consisting of: inflate or otherwise expand a functional assembly (e.g. functional assembly 130); deliver energy; modify energy delivery; deliver an insufflation fluid; insufflate a portion of the GI tract; desufflate a portion of the GI tract; deliver an injectate (e.g. into tissue and/or onto the surface of tissue); deliver a tissue expanding fluid (e.g. into tissue); steer the distal portion of a shaft; translate a control cable or control rod (hereinafter “control rod”); activate a sensor (e.g. record a signal); activate a transducer; and combinations of one or more of these. In some embodiments, handles 102, 22, 32 and/or 42 can comprise a user interface configured to control one or more components of system 10, such as controls 104, 24, 34 and/or 44, respectively, each of which can be constructed and arranged to control operation of one or more of: catheter 100, catheter 20, catheter 30, catheter 40 and/or console 200. In some embodiments, controls 104, 24, 34 and/or 44 can comprise one or more user input and/or user output components, such as a component selected from the group consisting of: screen; touchscreen; light; audible transducer such as a beeper or speaker; tactical transducer such as a vibratory motor assembly; a keyboard; a membrane keypad; a switch; a safety-switch, safety-switch 206 (e.g. a foot-activated switch); a mouse; a microphone; and combinations of one or more of these.

Handles 102, 22, 32 and 42 each are attached to the proximal end of shafts 110, 21, 31 and 41, respectively. Shafts 110, 21, 31 and 41 each typically comprise a relatively flexible shaft comprising one or more internal lumens or other passageways. In some embodiments, shafts 110, 21, 31, and/or 41 comprise a shaft with variable flexibility (e.g. a distal portion that is more flexible than a mid portion and/or a proximal portion). Shafts 110, 21, 31 and/or 41 can comprise a lumen, such as lumen 116 of shaft 110 shown, that is sized and configured to perform a function selected from the group consisting of: provide for the delivery or extraction of one or more fluids such as ablation fluids, cooling fluids, insufflation fluids, pneumatic fluids, hydraulic fluids and/or balloon expanding fluids; allow over the guidewire delivery of the associated device; surround an electrical wire providing electrical energy and/or signals; slidingly receive a control shaft or other control filament such as a control filament used to expand or contract a functional assembly (e.g. functional assembly 130) or otherwise modify the shape of a portion of the device; and combinations of one or more of these. Shafts 110, 21, 31 and/or 41 can comprise a braided or otherwise reinforced shaft or they can include one or more portions which are reinforced. Shafts 110, 21, 31 and/or 41 can comprise a multi-layer construction, such as a construction including a braid, a friction-reduced (e.g. PTFE) liner, a thermally insulating layer and/or an electrically insulating layer. Shafts 110, 21, 31 and/or 41 can include a bulbous distal end, such as bulbous tip 115 of shaft 110 shown, a circular or elliptical shaped enlarged end configured to improve traversing the innermost tissue of the duodenum or other luminal tissue of the GI tract (e.g. to smoothly advance within a lumen whose walls include villi and/or one or more folds). As described herein, shafts 110, 21, 31 and/or 41 can include a guidewire lumen, such as lumen 116 of shaft 110.

Positioned on the distal end or on a distal portion of shafts 110, 21, 31 and 41 is an expandable functional assembly, functional assemblies 130, 25, 35 and 45, respectively. Functional assemblies 130, 25, 35 and 45 are each constructed and arranged to be radially expanded and subsequently radially compacted (each shown in their radially expanded state in FIG. 2), one or more times during use. Each of functional assemblies 130, 25, 35 and 45 can include an expandable element selected from the group consisting of: an inflatable balloon; a radially expandable cage or stent; one or more radially deployable arms; an expandable helix; an unfurlable compacted coiled structure; an unfurlable sheet; an unfoldable compacted structure; and combinations of one or more of these. Functional assembly 130 can comprise a functional element, such as treatment element 135 shown, configured to treat target tissue. Treatment element 135 can be similar to one or more functional elements 139 described herein in reference to catheter 100 of FIG. 1.

In some embodiments, catheter 100, tissue expansion catheter 20, lumen diameter sizing catheter 30 and/or multi-function catheter 40, with their functional assemblies 130, 25, 35 and 45 (respectively) in their radially compacted state, are sized and configured to be inserted through a working channel of endoscope 50a and/or sheath 80, after endoscope 50a and/or sheath 80 have been inserted into a patient (e.g. through the mouth and advanced such that their distal end resides in the duodenum or other GI tract location). In some embodiments, catheter 100, tissue expansion catheter 20, sizing catheter 30 and/or multi-function catheter 40 are sized and configured to be inserted through the mouth and into a patient's GI tract alongside endoscope 50a. In some embodiments, catheter 100, tissue expansion catheter 20, lumen diameter sizing catheter 30 and/or multi-function catheter 40 are sized and configured to be inserted into a patient over one or more guidewires 60 (e.g. without the use of an endoscope). For insertion over a guidewire, the shafts 110, 21, 31 and/or 41 and the distal portions of the associated catheter 100, 20, 30 and/or 40 can comprise sufficient flexibility to traverse the pylorus and enter the duodenum, while having sufficient column and torsional strength to be advanced through the duodenum. In some embodiments, one or more portions of the shafts 110, 21, 31 and 41 have variable stiffness (e.g. stiffer in a proximal portion of the shaft) and/or include a lumen configured to accept a stiffening wire or other stiffening mandrel (e.g. a tapered mandrel), such as stiffening wire 67 shown. Alternatively or additionally, stiffening wire 67 can be inserted into endoscope 50a and/or sheath 80, such as to facilitate their advancement through the stomach and into the duodenum. In some embodiments, shaft 110 comprises at least a braided portion. In some embodiments, shaft 110 comprises a tapered portion (e.g. a taper such that the distal portion of shaft 110 is more flexible than its mid portion and/or proximal portion).

Console 200 can be constructed and arranged in a similar fashion to console 200 of FIG. 1 described herein. Console 200 can comprise an operator-accessible (e.g. clinician-accessible) user interface 205. User interface 205 can comprise one or more user output and/or user input components, such as a component selected from the group consisting of: screen; touchscreen; light; audible transducer such as a beeper or speaker; tactical transducer such as a vibratory motor assembly; a keyboard; a membrane keypad; a switch; safety-switch 206 (e.g. a foot-activated switch or other switch used to maintain safe operation, such as a switch configured as a dead man's switch); a mouse; a microphone; and combinations of one or more of these.

Console 200 can comprise a controller, such as controller 250 shown. Controller 250 can comprise one or more components or assemblies selected from the group consisting of: an electronics module; a power supply; memory (e.g. volatile or non-volatile memory circuitry); a microcontroller; a microprocessor; a signal analyzer; an analog to digital converter; a digital to analog converter; a sensor interface; transducer drive circuitry; software; and combinations of one or more of these. Controller 250 can comprise one or more algorithms, algorithm 251 shown, which can be constructed and arranged to automatically and/or manually control and/or monitor one or more devices, assemblies and/or components of system 10. Algorithm 251 of controller 250 can comprise an algorithm that is configured to determine one or more tissue expansion and/or tissue treatment parameters. In some embodiments, algorithm 251 processes one or more sensor signals (e.g. signals from functional elements 139, 29, 39 and/or 49 described herein) to modify one or more of: volume of tissue expansion fluid delivered; rate of tissue expansion fluid delivery; temperature of tissue expansion fluid delivery; amount of ablative fluid delivered; rate of ablative fluid delivery; energy delivered; power of energy delivered; voltage of energy delivered; current of energy delivered; temperature of ablative fluid or energy delivered; device and/or treatment element location within the GI tract; functional assembly pressure (e.g. balloon pressure); and combinations of one or more of these. Treatment element 135 can deliver energy to a surface of tissue, a delivery zone as described herein, which is a subset of the target tissue treated by that energy delivery (e.g. due to the conduction of heat or other energy to neighboring tissue). Algorithm 251 can comprise an algorithm configured to determine a delivery zone parameter such as a delivery zone parameter selected from the group consisting of: anatomical location of a delivery zone; size of delivery zone; percentage of delivery zone to receive energy; type of energy to be delivered to a delivery zone; amount of energy to be delivered to a delivery zone; and combinations of one or more of these. Information regarding the delivery zone parameter can be provided to an operator of system 10 (e.g. a clinician), such as via user interface 205. This information can be employed to set a delivery zone parameter, assist the operator in determining the completion status of the procedure (e.g. determining when the procedure is sufficiently complete) and/or to advise the operator to continue to complete a pre-specified area or volume of target tissue. The total area of treatment or number of delivery zones or number of treatments during a particular procedure (any of which can be employed in algorithm 251) can be defined by clinical and/or demographic data of the patient.

Console 200 can comprise one or more reservoirs or other sources of fluid, such as reservoir 220. Reservoir 220 can be configured to provide fluid at an ablative temperature (e.g. sufficiently hot or cold to ablate tissue), a treatment neutralizing (e.g. warming or cooling) fluid configured to reduce or otherwise limit ablative effects, an insufflation fluid, injectate 221 (e.g. similar to injectate 221 described herein in reference to FIG. 1), an agent (e.g. agent 420 described herein in reference to FIG. 1), and/or another fluid. Console 200 can comprise an energy delivery unit, such as EDU 260, configured to deliver energy to treatment element 135 and/or one or more other components of system 10, such as one or more components of catheters 100, 20, 30 and/or 40. Controller 250, reservoir 220 and/or EDU 260 can be of similar construction and arrangement as controller 250, reservoir 220 and/or EDU 260, respectively, of FIG. 1 described herein.

Console 200 can comprise one or more pressure sources, pumps, and/or other fluid drive assemblies, such as pumping assembly 225 shown. Pumping assembly 225 can be constructed and arranged to deliver positive pressure or vacuum pressure (e.g. any pressure below another pressure) to one or more fluid delivery elements or fluid pathways (e.g. lumens) of system 10. Pumping assembly 225 can be constructed and arranged to provide and/or extract fluid to radially expand and/or radially compact, respectively, one or more expandable assemblies, such as functional assemblies 130, 25, 35 and/or 45. Pumping assembly 225 can be configured to extract fluid from functional assembly 130 such as to radially compact functional assembly 130 prior to the removal of catheter 100 from within the patient. Pumping assembly 225 can comprise one or more pumps or other fluid delivery mechanisms, and/or other pressure or vacuum generators. In some embodiments, pumping assembly 225 is constructed and arranged to provide a recirculating ablative fluid (e.g. hot or cold) to catheter 100 and/or catheter 40. In these embodiments, pumping assembly 225 can be constructed and arranged to further provide a recirculating “neutralizing fluid” (e.g. a cooling or warming fluid, respectively, to counteract the ablative effects of the previously circulated ablative fluid) to a balloon of expandable assembly 35 or 45 respectively (e.g. to balloon 36 and/or 46, respectively). Pumping assembly 225 can be of similar construction and arrangement as pumping assembly 225 of FIG. 1 described herein. In some embodiments, pumping assembly 225 is constructed and arranged to deliver injectate 221 to a functional assembly 130, 25, 35 and/or 45, such as an injectate configured to expand tissue and/or to create a therapeutic restriction, as described herein, such as an injectate similar to injectate 221 described herein in reference to FIG. 1.

Console 200 includes connector 203, which is operably attached to one or more of: user interface 205 (e.g. safety-switch 206 or another component of user interface 205), controller 250, reservoir 220 and/or pumping assembly 225. Connector 203 is constructed and arranged to operably attach (e.g. fluidly, electrically, optically, acoustically, mechanically and/or otherwise operably attach) to one or more of connectors 103, 23, 33 and 43 of catheters 100, 20, 30 and 40, respectively. Console 200 can be constructed and arranged to deliver fluids, energy, and/or data via connector 203 to one or more of catheters 100, 20, 30 and 40. In some embodiments, an inflation fluid and/or a fluid at an ablative temperature is provided and/or recovered by console 200, such as a fluid at an ablative temperature delivered to functional assembly 130 of catheter 100 and/or functional assembly 45 of catheter 40. In some embodiments, insufflation, pneumatic and/or hydraulic fluids are delivered and/or recovered by console 200 via connector 203. In some embodiments, an injectate 221 is delivered by console 200, such as is described herein in reference to tissue expansion catheter 20 and multi-function catheter 40. In some embodiments, one or more control rods (not shown) are translated (e.g. advanced and/or retracted) within one or more lumens or other openings of catheter 100, 20, 30 and/or 40, such as to expand a cage, deploy a radially deployable arm, change the shape of an assembly, translate an assembly, rotate an assembly and/or otherwise control the position, shape and/or configuration of an assembly of system 10.

Console 200 can provide energy to, send information to, and/or record and/or receive a signal from one or more other elements of catheter 100, such as functional elements 139, 29, 39 and/or 49 described herein.

Catheter 100 can be constructed and arranged to treat target tissue of a patient. In some embodiments, catheter 100 is of similar construction and arrangement as catheter 100 of FIG. 1 described herein. Catheter 100 comprises handle 102 which attaches to a proximal end of shaft 110 and includes connector 103 for operable attachment to console 200. Positioned on the distal end or on a distal portion of shaft 110 is functional assembly 130. Functional assembly 130 can comprise an expandable element selected from the group consisting of: an inflatable balloon such as balloon 136 shown; a radially expandable cage or stent; one or more radially deployable arms; an expandable helix; an unfurlable compacted coiled structure; an unfurlable sheet; an unfoldable compacted structure; and combinations of one or more of these. Functional assembly 130 can comprise an energy delivery element or other tissue treatment element 135, such as an energy delivery element configured to deliver thermal, electrical, light, sound and/or ablative chemical energy to target tissue. In some embodiments, treatment element 135 comprises a mechanical abrader configured to treat tissue through abrasion. In some embodiments, functional assembly 130 comprises a balloon 136 which can be configured to receive one or more expansion and/or ablative fluids. Balloon 136 can comprise a compliant balloon, a non-compliant balloon, a pressure-thresholded balloon and/or otherwise be constructed and arranged as described in detail herein. Functional assembly 130 can be configured to both ablate (e.g. via a hot or cold ablative fluid) and neutralize the ablation (e.g. via a cooling or warming fluid, respectively), prior to and/or after the ablation, as described herein.

Via connector 103, console 200 can provide and/or extract one or more fluids to and/or from one or more lumens or other flow pathways of catheter 100, such as fluid provided by reservoir 220 and/or propelled by (i.e. delivered and/or extracted by) pumping assembly 225. Console 200, via EDU 260, can be configured to provide energy to one or more treatment elements 135 of catheter 100, such as energy contained in fluid at an ablative temperature (hot and/or cold), electrical energy (e.g. RF or microwave energy), light energy (e.g. laser light energy), or sound energy (e.g. subsonic or ultrasonic sound energy). In some embodiments, console 200 provides a fluid configured to treat target tissue with direct contact, such as an ablating agent (e.g. a sclerosant or other chemically ablative agent) and/or a fluid at an ablative temperature, either or both delivered directly to a target tissue surface.

In some embodiments, treatment element 135 comprises a fluid at an ablative temperature provided by console 200. In these embodiments, treatment element 135 can comprise a sufficiently hot fluid that is introduced into balloon 136 for a first time period to ablate target tissue, after which a cooling fluid is introduced into balloon 136, for a second time period, to extract heat from tissue (e.g. extract heat from target tissue and/or non-target tissue to reduce the ablation effect). Alternatively or additionally, the cooling fluid can be introduced into balloon 136 prior to the delivery of the hot fluid (e.g. for a third time period). In some embodiments, treatment element 135 comprises a sufficiently cold fluid (e.g. a fluid that is between 0° C. and −40° C.) that is introduced into balloon 136 for a first time period to ablate target tissue, after which a higher temperature fluid is introduced into balloon 136, for a second time period, to warm tissue (e.g. warm target tissue and/or non-target tissue to reduce the ablation effect). Alternatively or additionally, a warming fluid can be introduced into balloon 136 prior to the delivery of the cold fluid (e.g. for a third time period). Both the ablative and ablation-reducing fluids (neutralizing fluids) can be provided by console 200. As described herein, neutralizing fluids can be used (prior to, during, and/or post treatment) to prevent an over-treatment (e.g. to prevent undesired treatment of tissue, such as to prevent ablation of non-target tissue). These fluids can be provided in a recirculating manner as described in applicant's co-pending U.S. patent application Ser. No. 16/438,362, entitled “Heat Ablation Systems, Devices and Methods for the Treatment of Tissue”, filed Jun. 11, 2019. Alternatively or additionally, these fluids can be provided in a single bolus manner as described in applicant's co-pending U.S. patent application Ser. No. 14/917,243, entitled “Systems, Method and Devices for Treatment of Target Tissue”, filed Mar. 7, 2016. In some embodiments, thermal ablation is performed using system 10 as described herein.

As described herein, in some embodiments target tissue and/or tissue proximate the target tissue is cooled, heated and subsequently cooled again. In these embodiments, target tissue and/or tissue proximate the target tissue can be cooled during at least a portion of a first step, such as a first step including supplying a first fluid (e.g. a recirculating fluid) to functional assembly 130 for a first time period (e.g. a duration of at least 10 seconds or between 15-30 seconds), wherein the first fluid is supplied at a cooling temperature (e.g. continuously supplied by reservoir 220 at a temperature of 10° C. to 25° C. and/or a temperature approximating room temperature). In a subsequent second step, target tissue and/or tissue proximate the target tissue can be heated (e.g. ablated) during at least a portion of the second step, such as a second step including supplying a second fluid (e.g. a recirculating fluid) to functional assembly 130 for a second time period (e.g. a duration of at least 5 seconds or between 8-15 seconds), wherein the second fluid is supplied at a heat ablating temperature (e.g. continuously supplied by reservoir 220 at a temperature of 85° C.-95° C.). In a subsequent third step, target tissue and/or tissue proximate the target tissue can be cooled during at least a portion of the third step, such as a third step including supplying a third fluid (e.g. a recirculating fluid) to functional assembly 130 for a third time period (e.g. a duration of at least 10 seconds or between 15-30 seconds), wherein the third fluid is supplied at a cooling temperature (e.g. continuously supplied by reservoir 220 at a temperature of 10° C.-25° C. and/or a temperature of approximately room temperature). In some embodiments, other temperatures and/or durations for each heating or cooling cycle are used. In some embodiments, the second time period in which a hot fluid is supplied to functional assembly 130 comprises a time less than the first time period and/or the third time period. In some embodiments, the temperature of the fluid supplied to functional assembly 130 during the first time period and/or the third time period is at least 18° C. less and/or at least 60° C. less than the temperature of the fluid supplied to functional assembly 130 during the second time period. In some embodiments, the first temperature and the third temperature comprise a similar temperature. In some embodiments, a cooling fluid at approximately 10° C. is provided by console 200 (e.g. delivered to a proximal portion of catheter 100 or a proximal portion of connecting assembly 300, for subsequent delivery to functional assembly 130) for approximately 30 seconds, after which an ablative fluid at approximately 95° C. is provided by console 200 for approximately 12 seconds, after which a cooling fluid at approximately 10° C. is provided by console 200 for approximately 30 seconds. Alternatively, a warming fluid can be provided by console 200 (e.g. delivered to a proximal portion of catheter 100 or a proximal portion of connecting assembly 300, for subsequent delivery to functional assembly 130) prior to and/or after the delivery of a cryogenically ablative fluid (e.g. for the similar time periods as described herein in reference to heat ablation). In some embodiments, the volume, temperature and/or duration of fluid provided by console 200 (e.g. and eventually delivered to functional assembly 130) is automatically and/or dynamically adjusted, such as an adjustment performed based on a signal provided by one or more sensors as described herein. For example, a temperature and/or duration can be adjusted during a first ablation of an axial segment of intestine and/or during a subsequent second ablation of the same or different axial segment of intestine. In some embodiments, a pre-cooling and/or post-cooling step is used to avoid the need for a tissue expansion step (e.g. tissue expansion proximate tissue to be ablated in a heat ablation step). In other embodiments, a tissue expansion step is included.

In some embodiments, a first axial segment of tubular tissue is cooled (e.g. non-ablatively cooled), via functional assembly 130, for a first time period TP₁, and subsequently heat ablated for a second time period TP₂. A first reservoir 220_{A} includes the cooling fluid at a temperature T_{A}, (e.g. fluid continuously maintained or at least initially provided at temperature T_{A}) and a second reservoir 220_{B} includes the (heat) ablative fluid at a temperature T_{B} (e.g. fluid continuously maintained or at least initially provided at temperature T_{B}). In some embodiments, after the heat ablation during time period TP₂, an additional tissue cooling step is performed via functional assembly 130, for a third time period TP₃ (e.g. with cooling fluid at temperature T_{A}). Additionally axial segments of tubular tissue can subsequently be treated (e.g. additional axial segments treated via tissue cooling and subsequent heat ablation, with or without a subsequent tissue cooling step). T_{A} can comprise a temperature at or below 25° C., such as a temperature at or below room temperature, 20° C. and/or 15° C., and T_{B} can comprise a temperature at or above 65° C., such as a temperature at or above 75° C., 85° C. and/or 95° C. TP₁ can comprise a time duration of between 3 seconds and 60 seconds (e.g. between 20 seconds and 40 seconds); TP₂ can comprise a time duration of between 1 seconds and 30 seconds (e.g. between 5 seconds and 15 seconds); and TP₃ can comprise a time duration of between 3 seconds and 60 seconds (e.g. between 20 seconds and 40 seconds). In these embodiments, T_{A}, T_{B}, TP₁, TP₂ and/or TP₃ can be varied (e.g. automatically by system 10), based on information recorded by a sensor of the present inventive concepts (e.g. a sensor measuring temperature, pressure, flow rate and/or other parameter at one or more locations of catheter 100, console 200 or other component of system 10). One or more of T_{A}, T_{B}, TP₁, TP₂ and/or TP₃ can be held relatively constant or unchanged, during one or more axial tissue segment ablations. However, one or more of T_{A}, T_{B}, TP₁, TP₂ and/or TP₃ can vary (e.g. be allowed to vary), such as when T_{A} increases during an extraction of cooling fluid from catheter 100 (e.g. the recovered fluid warms the cooling fluid in the first reservoir 220_{A}). These variations (e.g. as measured by one or more sensors of system 10) can result in an adjustment (e.g. an automatic adjustment) to another parameter (e.g. T_{A}, T_{B}, TP₁, TP₂ and/or TP₃), such as an adjustment made by algorithm 251 (e.g. an algorithm comprising a lookup table including reservoir temperatures and corresponding treatment durations) based on a signal produced by one or more functional elements 109, 119, 139, 209, 229 and/or 309 described herein in reference to FIG. 1, that have been configured as a sensor. In some embodiments, T_{A}, T_{B} TP₁, TP₂ and/or TP₃ are varied based on the value of T_{A} and/or T_{B}. For example, if the temperature T_{A} of the cooling fluid were to increase during a multi-ablation procedure, the time period TP₂ and/or temperature T_{B} could be compensatingly adjusted (e.g. decreased). In some embodiments, time period TP₂ is decreased by up to 2 seconds (e.g. from an initial time period of 11 to 13 seconds, in one or more decrements), as the temperature T_{A} increases by up to 16° C. (e.g. from a starting temperature of approximately 9° C.), such as during a clinical procedure comprising ablation of two or more axial segments (e.g. ablation of between two and six axial segments). While the previous embodiments have been described in reference to a cooling of tissue followed by a heat ablation of tissue (which may also include a subsequent tissue cooling step), alternatively, system 10 can be configured to (non-ablatively) warm tissue, followed by cryogenic ablation of tissue (which can also include a subsequent tissue warming step).

In some embodiments, treatment element 135 comprises one or more energy or other tissue treatment elements positioned in, on and/or within functional assembly 130. Treatment element 135 can comprise one or more energy delivery elements configured to deliver energy to target tissue, such as an energy delivery element selected from the group consisting of: a fixed or recirculating volume of fluid at a high enough temperature to ablate tissue; a fixed or recirculating volume of fluid at a low enough temperature to ablate tissue; one or more thermal energy delivery elements such as one or more elements configured to deliver heat energy or cryogenic energy; an array of electrodes such as an array of electrodes configured to deliver radiofrequency (RF) energy; one or more electromagnetic energy delivery elements such as one or more elements configured to deliver microwave energy; one or more optical elements configured to deliver light energy such as laser light energy; one or more sound energy delivery elements such as one or more elements configured to deliver subsonic and/or ultrasonic sound energy; one or more chemical or other agent delivery elements; and combinations of one or more of these. In some embodiments, catheter 100 is constructed and arranged to deliver RF energy, such as is described in applicant's co-pending U.S. patent application Ser. No. 16/711,236, entitled “Electrical Energy Ablation Systems, Devices and Methods for the Treatment of Tissue”, filed Dec. 11, 2019; and/or to deliver ablative fluid directly to tissue, such as is described in applicant's co-pending U.S. patent application Ser. No. 14/609,334, entitled “Ablation Systems, Devices and Methods for the Treatment of Tissue”, filed Jan. 29, 2015.

In some embodiments, catheter 100 is further constructed and arranged to provide geometric information (e.g. diameter information) of a luminal structure such as the duodenum. In these embodiments, catheter 100 and functional assembly 130 can be of similar construction and arrangement as functional assembly 35 and lumen diameter sizing catheter 30 described herein.

In some embodiments, system 10 comprises one or more devices for expanding target tissue or tissue proximate target tissue, such as tissue expansion catheter 20. In some embodiments, target tissue to be treated comprises mucosal tissue and the tissue to be expanded comprises submucosal tissue proximate the mucosal tissue to be treated. In some embodiments, tissue expansion catheter 20 is of similar construction and arrangement as catheter 100 described herein in reference to FIG. 1. In some embodiments, tissue expansion catheter 20 is of similar construction and arrangement as a tissue expansion device described in applicant's co-pending U.S. patent application Ser. No. 16/900,563, entitled “Injectate Delivery Devices, Systems and Methods”, filed Jun. 12, 2020. Tissue expansion catheter 20 can be configured to expand a full or partial circumferential segment of luminal wall tissue, such as to expand one or more layers of submucosal tissue in one or more axial segments of the duodenum or other portion of the GI tract. Tissue expansion catheter 20 can be configured to expand multiple segments of GI tract tissue, such as multiple relatively contiguous segments of submucosal tissue expanded as described in detail herein.

Tissue expansion catheter 20 comprises handle 22 which attaches to a proximal end of shaft 21 and includes connector 23 for operable attachment to console 200. Positioned on the distal end of shaft 21 or on a distal portion of catheter 20 is functional assembly 25. Functional assembly 25 can comprise an expandable element selected from the group consisting of: an inflatable balloon such as balloon 26 shown; a radially expandable cage or stent; one or more radially deployable arms; an expandable helix; an unfurlable compacted coiled structure; an unfurlable sheet; an unfoldable compacted structure; and combinations of one or more of these. Balloon 26 can comprise a compliant balloon, a non-compliant balloon, a pressure-thresholded balloon and/or otherwise it can be constructed and arranged as described in detail herein. Balloon 26 can comprise a tissue-contacting length of between 15 mm and 26 mm, such as a tissue-contacting length of approximately 20 mm. Balloon 26 can comprise a wall thickness of between 0.00015″ and 0.020″, such as a wall thickness of between 0.0002″ and 0.0010″, such as a wall thickness of approximately 0.00075″. Functional assembly 25 can be configured to expand to a diameter between 18 mm and 30 mm, such as a diameter of approximately 24 mm. Functional assembly 25 can be configured to be expanded via control 24 and/or via user interface 205 of console 200 (e.g. inflated and deflated by delivery and extraction, respectively, of air, water and/or other fluids by console 200).

Functional assembly 25 can comprise one or more needles, fluid jets, and/or other fluid delivery elements, elements 28 shown. The one or more fluid delivery elements 28 can each comprise an element selected from the group consisting of: needle such as a straight needle or a curved needle; nozzle; fluid jet; iontophoretic fluid delivery element; and combinations of one or more of these. The one or more fluid delivery elements 28 are configured to deliver injectate 221 and/or another fluid to tissue when functional assembly 25 is expanded (e.g. at least partially expanded with inflation fluid provided by console 200), positioning the fluid delivery elements 28 proximate (e.g. in contact with or close to) tissue to be expanded, such as luminal wall tissue of the GI tract.

The one or more fluid delivery elements 28 can be configured to be advanced (e.g. advanced into tissue) and retracted via control 24 of catheter 20. The one or more fluid delivery elements 28 can be positioned in one or more ports 27 (e.g. one or more ports of similar construction and arrangement to port 137 of FIG. 1), as shown in FIG. 2. In some embodiments, a vacuum provided by console 200 causes tissue to tend toward and/or enter each port 27, such that each fluid delivery element 28 can inject fluid (e.g. injectate 221) into the engaged and/or captured tissue without having to extend significantly beyond the associated port 27 (e.g. all or at least a majority of fluid delivery element 28 can be configured to remain within port 27 during delivery of fluid into tissue captured within port 27). By limiting excursion of fluid delivery element 28 out of port 27, risk of fluid delivery element 28 and/or injectate 221 penetrating through the outer surface of the GI tract is prevented or at least significantly reduced. In some embodiments, fluid can be delivered into tissue by fluid delivery element 28 with or without advancement of fluid delivery element 28 into the captured tissue (e.g. tissue is drawn into a port 27 via an applied vacuum such that fluid delivery element 28 penetrates or otherwise engages the tissue for fluid delivery without advancement of the fluid delivery element 28). In some embodiments, fluid delivery elements 28, ports 27 and/or other portions of tissue expansion catheter 20 are of similar construction and arrangement as a tissue expansion device described in applicant's co-pending U.S. patent application Ser. No. 16/900,563, entitled “Injectate Delivery Devices, Systems and Methods”, filed Jun. 12, 2020.

In some embodiments, functional assembly 25 comprises three or more fluid delivery elements 28 arranged in a circumferential pattern, such as three fluid delivery elements 28 arranged along a circumference and separated by approximately 120°. The multiple fluid delivery elements 28 can be configured to be advanced individually (e.g. via multiple controls 24), or simultaneously (e.g. via a single control 24). In some embodiments, two fluid delivery elements 28 are separated by approximately 180°. In some embodiments, four fluid delivery elements 28 are separated by approximately 90°.

In some embodiments, system 10 includes injectate 221 which can be provided by console 200 to catheter 20, and injectate 221 can be delivered into tissue by the one or more fluid delivery elements 28. Injectate 221 can comprise one or more materials as described herein in reference to injectate 221 of FIG. 1.

In some embodiments, catheter 20 and/or console 200 are configured to reduce a volume of fluid (e.g. liquid or gas) within functional assembly 25 (e.g. within balloon 26) as injectate 221 is delivered into tissue (e.g. submucosal tissue), such as to prevent excessive force being applied by functional assembly 25 to tissue proximate the expanding tissue (i.e. due to the decreasing luminal diameter proximate the expanding tissue in contact with functional assembly 25). In some embodiments, system 10 is constructed and arranged to inflate or otherwise expand functional assembly 25 (e.g. balloon 26) to a first target pressure, such as a pressure of approximately 0.7 psi. Injectate 221 is delivered via one or more fluid delivery elements 28 into submucosal tissue (e.g. simultaneously or sequentially). Fluid contained within functional assembly 25 (e.g. within balloon 26) can be reduced or increased to maintain the pressure at a second target pressure, for example a pressure higher than the first target pressure such as a pressure between 0.8 psi and 0.9 psi. Fluid of up to 10 ml can be injected while maintaining the second target pressure in functional assembly 25 (e.g. by decreasing the amount of fluid in functional assembly 25 to cause 1 mm steps of diameter decrease of functional assembly 25).

In some embodiments, tissue expansion catheter 20 is further constructed and arranged to provide geometric information (e.g. diameter information) of a luminal structure such as the duodenum or other intestinal location. In these embodiments, catheter 20 and functional assembly 25 can be of similar construction and arrangement as lumen diameter sizing catheter 30 and functional assembly 35, respectively, described herein.

In some embodiments, system 10 comprises one or more separate devices for estimating or otherwise measuring (e.g. “sizing”) the diameter, average diameter, equivalent diameter, minimum diameter, cross sectional area and/or other geometric measure (herein “diameter”) of luminal tissue, such as lumen diameter sizing catheter 30. Sizing catheter 30 is constructed and arranged to be placed into one or more locations of the GI tract or other internal location of the patient and measure the diameter or other geometric parameter of tissue. In some embodiments, sizing catheter 30 is of similar construction and arrangement as catheter 100 described herein in reference to FIG. 1. Sizing catheter 30 can be configured to measure the diameter of multiple segments of intestinal or other GI tract tissue, such as to measure multiple diameters along the length of the duodenum.

Catheter 30 comprises handle 32 which attaches to a proximal end of shaft 31 and includes connector 33 for operable attachment to console 200. Positioned on the distal end of shaft 31 or on a distal portion of catheter 30 is functional assembly 35. Functional assembly 35 can comprise an expandable cage, balloon 36, or other expandable element as described herein, constructed and arranged to measure the inner surface diameter of tubular tissue (e.g. average diameter, equivalent diameter, minimum diameter, cross sectional area and/or other geometric measure of the inner surface of tubular tissue), such as a diameter of the duodenum, jejunum, ileum, and/or colon. Balloon 36 can comprise a compliant balloon, a non-compliant balloon, a pressure-thresholded balloon and/or otherwise be constructed and arranged as described in detail herein. Functional assembly 35 can be configured to be expanded via control 34 and/or via user interface 205 of console 200 (e.g. inflated and deflated by delivery and extraction, respectively, of fluids by console 200).

Fluids delivered by console 200 to functional assembly 35 (e.g. fluids supplied by reservoir 220) can be provided at one or more predetermined pressures, or pressure profiles. Diameter measurements can be accomplished by performing a visualization procedure (manual or automated) that assesses functional assembly 35 diameter. Alternatively or additionally, functional assembly 35 can be controllably filled with a fluid, and controller 250 can include an algorithm (e.g. algorithm 251 described herein in reference to FIG. 1) that correlates the fluid volume and/or fluid pressure to the diameter of tubular tissue in contact with functional assembly 35. In some embodiments, subsequent selection (e.g. device model or size selection) and/or expansion diameter (e.g. inflated diameter chosen for sufficient apposition) of functional assemblies 130, 25 and/or 45 of catheters 100, 20, and/or 40, respectively, can be determined using the information provided by sizing catheter 30 and/or console 200. In some embodiments, catheter 30 performs one or more sizing procedures as described herein.

In some embodiments, functional assembly 35 comprises a balloon, expandable cage and/or other expandable element that includes two or more electrodes configured to provide a tissue impedance measurement whose value can be correlated to a level of apposition of functional assembly 35, and whose expanded diameter (e.g. visually or otherwise measured) correlates to a diameter of tubular tissue in contact with the expandable element. Alternatively or additionally, functional assembly 130 of catheter 100, functional assembly 25 of catheter 20 and/or functional assembly 45 of catheter 40 can be used to measure a diameter of the inner surface of tubular tissue, such as has been described herein in reference to functional assembly 35 and catheter 30.

In some embodiments, system 10 comprises one or more devices, such as multi-function catheter 40 shown, that are constructed and arranged to perform two or more functions selected from the group consisting of: treat target tissue such as to deliver energy or otherwise ablate target tissue; expand tissue such as to expand one or more layers of submucosal tissue (e.g. proximate to and/or including target tissue); and determine or estimate a diameter (e.g. an average diameter, equivalent diameter, minimum diameter, cross sectional area and/or other geometric measure) of a lumen of tubular tissue; and combinations of one or more of these. Multi-function catheter 40 is constructed and arranged to be placed into one or more locations of the GI tract or other internal location of the patient and perform two or more of the functions listed above. In some embodiments, multi-function catheter 40 is of similar construction and arrangement as catheter 100 described herein in reference to FIG. 1. Multi-function catheter 40 can be configured to perform the multiple functions at multiple segments of GI tract, such as multiple relatively contiguous axial segments of the duodenum or other intestinal location as is described herein.

Catheter 40 comprises handle 42 which attaches to a proximal end of shaft 41 and includes connector 43 for operable attachment to console 200. Positioned on the distal end of shaft 41 or on a distal portion of catheter 40 is functional assembly 45. Functional assembly 45 can comprise an expandable cage, balloon 46, or other expandable element constructed and arranged to be positioned in apposition with and/or in close proximity to the inner wall of tubular tissue, such as tissue of the duodenum, jejunum and/or other intestinal location. Balloon 46 can comprise a compliant balloon, a non-compliant balloon, a pressure-thresholded balloon and/or otherwise be constructed and arranged as described in detail herein. Functional assembly 45 can be configured to be expanded via control 44 and/or via user interface 205 of console 200 (e.g. inflated and deflated by delivery and extraction, respectively, of fluids by console 200).

Functional assembly 45 can comprise treatment element 135′ shown, which can comprise a fluid at an ablative temperature delivered into functional assembly 45 by console 200 and/or an energy delivery element permanently positioned on, in and/or within functional assembly 45 (e.g. an energy delivery element configured to deliver thermal energy, electrical energy, light energy, sound energy and/or chemical energy as described herein). In some embodiments, treatment element 135′ comprises a mechanical abrader configured to treat tissue through abrasion. In some embodiments, treatment element 135′ is of similar construction and arrangement as functional element 139a of catheter 100 of FIG. 1. Functional assembly 45 can be configured to both ablate (e.g. via a hot or cold ablative fluid) and neutralize (e.g. via a cooling or warming fluid, respectively), prior to and/or after the ablation, as described herein.

Alternatively or additionally, functional assembly 45 can comprise one or more elements configured to expand tissue (e.g. to deliver fluid into tissue to be expanded), such as fluid delivery elements 48 shown. Fluid delivery elements 48 can each be positioned within one or more ports 47, as shown in FIG. 2. Fluid delivery elements 48 and ports 47 of catheter 40 can be constructed and arranged as described herein in reference to fluid delivery elements 28 and ports 27, respectively of catheter 20 of FIG. 2, and/or fluid delivery element 139c and ports 137, respectively, of catheter 100 of FIG. 1.

Catheters 100, 20, 30 and/or 40 can comprise one or more functional elements, such as functional elements 139, 29, 39 and/or 49, respectively, shown positioned in, on and/or within functional assemblies 130, 25, 35 and 45, respectively. Alternatively or additionally, one or more functional elements 139, 29, 39 and/or 49 can be located at a different location of the associated device, such as in, on and/or within the associated shaft and/or handle of the device. In some embodiments, one or more functional elements 139, 29, 39 and/or 49 comprise a sensor, such as a sensor selected from the group consisting of: physiologic sensor; blood glucose sensor; blood gas sensor; blood sensor; respiration sensor; EKG sensor; EEG sensor; neuronal activity sensor; blood pressure sensor; flow sensor such as a flow rate sensor; volume sensor; pressure sensor; force sensor; sound sensor such as an ultrasound sensor; electromagnetic sensor such as an electromagnetic field sensor or an electrode; gas bubble detector such as an ultrasonic gas bubble detector; strain gauge; magnetic sensor; ultrasonic sensor; optical sensor such as a light sensor; chemical sensor; visual sensor such as a camera; temperature sensor such as a thermocouple, thermistor, resistance temperature detector or optical temperature sensor; impedance sensor such as a tissue impedance sensor; and combinations of one or more of these. Alternatively or additionally, one or more functional elements 139, 29, 39 and/or 49 comprise a transducer, such as a transducer selected from the group consisting of: an energy converting transducer; a heating element; a cooling element such as a Peltier cooling element; a drug delivery element such as an iontophoretic drug delivery element; a magnetic transducer; a magnetic field generator; an ultrasound wave generator such as a piezo crystal; a light producing element such as a visible and/or infrared light emitting diode; a motor; a pressure transducer; a vibrational transducer; a solenoid; a fluid agitating element; and combinations of one or more of these. Functional elements 139, 29, 39 and/or 49 can be electrically connected to EDU 260 (e.g. to receive power, send signals and/or receive signals), such as via an electrical connection provided by connector 203. Functional elements 139, 29, 39 and/or 49 can send or receive signals from controller 250 of console 200, such as one or more sensor signals used to control ablation energy provided by console 200. Functional elements 139, 29, 39 and/or 49 can be activated and/or otherwise controlled via controls 104, 24, 34 and/or 44, respectively. Alternatively or additionally, user interface 205 of console 200 can be configured to allow operator control of functional elements 139, 29, 39 and/or 49.

In some embodiments, console 200 comprises one or more functional elements 209, comprising a sensor or transducer as described herein. Functional element 209 can comprise one or more pressure sensors, such as one or more pressure sensors configured to provide a signal used to regulate fluid delivery provided to one or more of catheters 100, 20, 30 and/or 40. Functional element 209 can comprise one or more temperature sensors, such as one or more temperature sensors that provide a signal used to regulate temperature of one or more fluids of console 200. Functional element 209 can be positioned to measure a parameter (e.g. temperature or pressure) of fluid within reservoir 220, within pumping assembly 225 and/or within a fluid conduit of console 200.

In some embodiments, system 10 comprises one or more agents configured to be delivered to the patient, such as agent 420. Agent 420 can be delivered by one or more of catheters 100, 20, 30, 40 and/or 50, or by a separate device such as a syringe or other medication delivery device. In some embodiments, injectate 221 comprises agent 420, such as when agent 420 is delivered by one or more fluid delivery elements 139c as described herein. In some embodiments, agent 420 comprises an anti-peristaltic agent, such as L-menthol (i.e. oil of peppermint). Alternatively or additionally, agent 420 can comprise glucagon, buscopan, hycosine, somatostatin, an opiod agent and/or any anti-peristaltic agent. Agent 420 can be delivered into the GI tract, such as via endoscope 50a, sheath 80 and/or catheters 100, 20, 30 and/or 40. Agent 420 can be delivered systemically, such as via an intravenous or intra-arterial access line, or injected directly into tissue. Agent 420 can comprise a drug or other agent as described herein in reference to agent 420 of FIG. 1.

As described above, user interface 205 can comprise safety-switch 206 such as a foot-activated switch. Safety-switch 206 can be configured to allow a clinician to activate or modify one or more processes of system 10 without having to use his or her hands (e.g. without having to use a digit of the hand). In some embodiments, system 10 is constructed and arranged to perform a function selected from the group consisting of: automatic contraction (e.g. deflation) of functional assembly 130 if safety-switch 206 is not activated (e.g. continuously or semi-continuously pushed, pressed or otherwise activated, such as by a foot or digit of an operator); automatic replacement of ablative fluid (e.g. hot fluid) with neutralizing fluid (e.g. cold fluid) if safety-switch 206 is not activated; initiate introduction of ablative fluid (e.g. hot fluid) into functional assembly 130 by activation of safety-switch 206 (e.g. after functional assembly 130 has been pre-expanded with cold fluid and an operator has confirmed proper position for treatment); allow hands-free activation (e.g. initiation) of a treatment step (e.g. a treatment step such as tissue expansion, application of vacuum, aspiration, and/or ablation) such that one or more operators can maintain their hands on one or more of endoscope 50a and/or catheters 100, 20, 30 and/or 40; allow hands-free activation (e.g. initiation) of a treatment step such that the required number of operators is reduced; and combinations of one or more of these.

Each of catheters 100, 20, 30 and/or 40 can be provided in one or more sizes, such as one or more lengths of the associated shaft 110, 21, 31 and/or 41, respectively, and/or one or more diameters (e.g. expanded diameter) of the associated functional assembly 130, 25, 35 and/or 45, respectively. Luminal sizing as described herein or other anatomical information can be used to select the appropriately sized device to treat the patient.

In some embodiments, system 10 of FIG. 2 comprises one or more sensors, such as one or more functional elements 109, 119, 139, 209, 229 and/or 309 described herein in reference to FIG. 1, that comprise a sensor. These one or more sensors can be configured to provide a signal, such as a signal used to adjust one or more console 200 settings (e.g. console settings 201) of the present inventive concepts.

In some embodiments, system 10 of FIG. 2 comprises one or more connecting assemblies for connecting one or more of catheters 100, 20, 30 and/or 40 to console 200, not shown but such as connecting assembly 300 described herein in reference to FIG. 1.

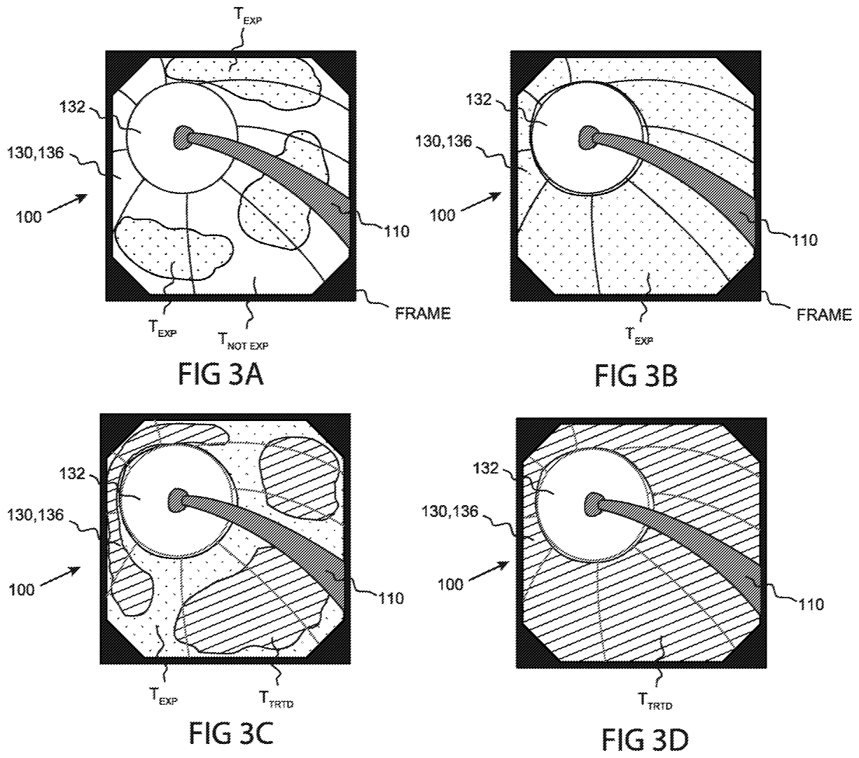

FIGS. 3-19 described herein illustrate various configurations of, and procedural methods for using, the systems and catheters of the present inventive concepts, such as system 10 and catheter 100 described herein in reference to FIG. 1, and system 10 and catheters 100, 20, 30 and/or 40 described herein in reference to FIG. 2. In FIGS. 3A-D, each system 10 and catheter 100 can comprise one or more components of similar construction and arrangement to system 10 and catheters 100, 20, 30 and/or 40 of FIG. 1 and/or FIG. 2, whether shown in the associated figure or not. In some of the figures, one or more conduits 111 have been removed for illustrative clarity, such as one or more fluid, translatable rod, signal and/or power transporting conduits attached to one or more functional elements, inflatable balloons or other expandable elements and/or other components of the system 10 and/or catheter 100 illustrated in the associated figure. Each of the functional assemblies 130 can be constructed and arranged to perform a first step of a medical procedure (e.g. a tissue ablation procedure, a tissue expansion procedure and/or a tissue diagnostic procedure) at a first axial segment of the intestine, and subsequently perform at least a second step of the medical procedure at a second axial segment of the intestine, at a location proximal or distal to the first axial segment of the intestine. In some embodiments, a sequence of three or more steps at three or more axial segments can be performed. In some embodiments, both a tissue expansion and a tissue ablation are performed at each selected axial segment of the intestine.

Each functional assembly 130 can comprise a balloon 136 or other expandable element, such as: a radially expandable cage or stent; one or more radially deployable arms; an expandable helix; an unfurlable compacted coiled structure; an unfurlable sheet; and/or an unfoldable compacted structure.

In some embodiments, system 10 of FIG. 2, and/or one or more of its components, is of similar construction and arrangement to system 10 of FIG. 19 described herein.

Referring now to FIGS. 3A-D, camera views of a series of steps for expanding tissue and treating target tissue at a single axial segment of intestine are illustrated, consistent with the present inventive concepts. A distal portion of catheter 100 is being viewed by a camera device (e.g. configured as a sensor of the present inventive concepts), such as an endoscopic camera such as a camera of endoscope 50a. Catheter 100 comprises shaft 110, functional assembly 130 (shown in its expanded state), and other components, such as one or more components of similar construction and arrangement to those described herein in reference to catheter 100 of FIG. 1 or FIG. 2, such as one or more conduits 111 which have been removed for illustrative clarity. Functional assembly 130 can comprise an expandable element, such as balloon 136 shown. Functional assembly 130 of catheter 100 has been introduced into the intestine, such as by being inserted via one or more of: through a working channel of an endoscope (e.g. the endoscope producing the camera view); over a guidewire; inserted alongside an endoscope (e.g. over a guidewire); through a sheath attached to or independent of an endoscope (e.g. over a guidewire); through a laparoscopic port (e.g. over a guidewire); and/or through any body introduction device.

Functional assembly 130 comprises a distal wall (e.g. distal wall 132 shown in the camera view), and a proximal wall (e.g. proximal wall 131 not shown), and a side-wall portion therebetween. Functional assembly 130 of catheter 100 has been inserted into an axial segment of the intestine, and it has been inflated such that a tissue-contacting portion of balloon 136 is in substantial contact with the inner wall of the axial segment of the intestine (i.e. all portions of functional assembly 130 shown in the camera view of FIGS. 3A-D except for distal wall 132). The image provided by the camera is looking through at least a portion of proximal wall 131 of functional assembly 130 (e.g. an endoscope or other camera device has been advanced to a location relatively proximate proximal wall 131 of functional assembly 130). At least the proximal wall of functional assembly 130 is constructed of materials to be transparent, or at least relatively transparent (hereinafter “transparent”) to the camera view, such as a clear material transparent with respect to the camera being used (e.g. transparent to visible light used by a visible light camera, transparent to infrared light used by an infrared camera, transparent to ultrasound waves as used by an ultrasound imager and/or transparent to radiation used by a radiation-based camera). At least one or more portions of the tissue-contacting surfaces (e.g. portions of the side-wall) of functional assembly 130 can also be transparent to the camera view, such as is shown in FIG. 3D, such as to view the tissue in contact with functional assembly 130 during a therapeutic or diagnostic procedure. In some embodiments, at least a portion of the distal wall 132 is transparent. For example, system 10 can be configured to detect a change of the tissue (e.g. a color change) and/or a change to a material delivered into the tissue, such as injectate 221 described herein, and to adjust one or more console settings 201 based on the color change or other image information (e.g. a console setting related to an injectate delivery parameter during tissue expansion and/or an energy delivery parameter during tissue ablation).

In some embodiments, functional assembly 130 is configured to both expand tissue and ablate tissue, such that a single functional assembly 130 can be positioned, anchored, and complete the tissue expansion steps described herein in reference to FIGS. 3A-B, and subsequently (while remaining anchored in the same axial location of intestine) complete the tissue ablation steps described herein in reference to FIGS. 3C-D. Functional assembly 130 can be anchored throughout the tissue expansion and tissue treatment steps by maintaining sufficient force against the lumen wall, such as by sufficient expansion of functional assembly 130 and/or by other anchoring means as described herein. In some embodiments, as described herein, a series of multiple (e.g. two) tissue expansion steps are performed sequentially, and then a single ablation step is performed. This process can be repeated where multiple ablations are performed, each preceded by multiple tissue expansion steps.

In other embodiments, a first functional assembly 130 is configured to expand tissue, and a second functional assembly 130 is configured to treat tissue. In these embodiments, the first functional assembly is positioned, anchored, and used to complete the tissue expansion steps described herein in reference to FIGS. 3A-B. Subsequently, the first functional assembly 130 is moved away from the axial segment, and a second functional assembly 130 (e.g. positioned on the same catheter 100 or a second catheter 100) is positioned in the same axial segment, anchored, and used to complete the tissue ablation steps described herein in reference to FIGS. 3C-D. Similarly, a single or multiple tissue expansion steps can be performed prior to each ablation step.

In FIG. 3A, a tissue expansion step has been initiated, such as a tissue expansion step in which one or more needles or other fluid delivery elements (e.g. three equally spaced fluid delivery elements 139c of FIG. 1) are delivering fluid into tissue (e.g. submucosal tissue of the intestine). As shown in FIG. 3A, certain areas of tissue have been expanded, areas of tissue T_{EXP}, while other areas in contact with the tissue-contacting portions (e.g. sidewalls) of functional assembly 130, T_{NOT EXP} are unexpanded (e.g. yet to be expanded). In some embodiments, the visual feedback of the camera view of FIG. 3A is provided to an operator (e.g. via a display of console 200 or endoscope 50a described herein in reference to FIG. 1), such that the operator can continue fluid delivery until all desired tissue is expanded. Alternatively or additionally, system 10 can be configured to assess completeness of tissue expansion, such as to continue or otherwise adjust fluid delivery (e.g. automatically), and/or notify the operator of a desire to continue fluid delivery, based on the visual feedback. In some embodiments, the fluid delivered (e.g. injectate 221 of FIG. 1) comprises visualizable material configured to be visualized, to enhance visualization of expanded tissue and/or to identify an adverse situation such as delivered fluid leaking into the intestinal lumen (e.g. versus a submucosal layer of tissue). System 10 can be configured to provide such visual feedback (e.g. via a camera of system 10) prior to, during, and/or after a tissue expansion procedure and/or a tissue ablation procedure.

In FIG. 3B, relatively all of the tissue desired to be expanded by functional assembly 130 has been expanded. In some embodiments, sufficient expansion of tissue is visually confirmed (e.g. an operator manually confirms that all, a majority, or any sufficient amount of the tissue in contact with functional assembly 130 and potentially beyond functional assembly 130 has been expanded). This confirmation can be performed prior to performing a subsequent step, such as an ablation step. In some embodiments, system 10 is configured to confirm sufficient tissue expansion has been completed (e.g. automatically or semi-automatically), such as via an image analysis algorithm, such as algorithm 251 of controller 250 described herein in reference to FIG. 1. In some embodiments, visualizable or other material of injectate 221 is detected by the camera device or one or more other sensors of system 10, and algorithm 251 correlates the amount and/or location of the detected material to a level of tissue expansion.

In FIG. 3C, a target tissue ablation step has been subsequently initiated, such as a target tissue ablation step in which energy is delivered to tissue (e.g. via ablative fluid being introduced into functional assembly 130 and/or by delivery of RF of other energy into tissue by functional assembly 130). In some embodiments, the target tissue treated comprises mucosal tissue of the duodenum or other intestinal mucosal tissue, such as to treat diabetes, hypercholesterolemia, and/or another patient disease or disorder. As shown in FIG. 3C, certain areas of tissue have been treated, areas of tissue T_{TRTD}, while other areas in contact with the tissue-contacting portions (e.g. sidewalls) of functional assembly 130 are simply expanded (T_{EXP} shown in FIG. 3C). In some embodiments, system 10 is configured to provide visual feedback of the camera view of FIG. 3C to an operator (e.g. via a display of console 200 or endoscope 50a described herein in reference to FIG. 1), such that the operator can continue tissue ablation until all desired tissue is treated. Alternatively or additionally, system 10 can be configured to assess completeness of tissue ablation and to adjust tissue ablation console settings (e.g. automatically), based on the visual feedback. In some embodiments, ablated tissue is identified by a color change that occurs during ablation. Alternatively or additionally, system 10 can be configured to further differentiate ablated tissue, such as by detecting a color or other change to injectate 221 delivered in the tissue expansion steps. System 10 can be configured to provide such visual feedback (e.g. via a camera of system 10) prior to, during, and/or after a tissue expansion procedure and/or a tissue ablation procedure.

In FIG. 3D, relatively all of the tissue desired to be ablated by functional assembly 130 has been treated. In some embodiments, sufficient treatment of tissue is visually confirmed (e.g. an operator manually confirms that all, a majority, or any sufficient amount of the tissue in contact with functional assembly 130 has been ablated or otherwise treated). This confirmation is performed prior to removing functional assembly 130 and/or repositioning functional assembly 130 at a different axial segment (e.g. when a medical procedure comprises ablating multiple axial segments, as described herein). In some embodiments, system 10 is configured to confirm sufficient tissue ablation or other treatment has been completed (e.g. automatically or semi-automatically), such as via an image analysis algorithm, such as algorithm 251 of controller 250 described herein in reference to FIG. 1. In some embodiments, visualizable changes or other changes to injectate 221 within tissue is detected by the camera device or one or more other sensors of system 10, and algorithm 251 correlates the amount and/or location of the changed injectate 221 to a level of tissue ablation.

Referring now to FIG. 4, a method of performing a medical procedure including performing a tissue expansion with a functional assembly, and subsequently treating target tissue with the same or a different functional assembly is illustrated, consistent with the present inventive concepts. Method 3500 of FIG. 4 can be performed using system 10 of the present inventive concepts, such as by using catheters 100, 20, 30, and/or 40 as described herein. Method 3500 will be described using system 10 and catheter 100 as described herein.

In Step 3510, the distal portion of a catheter 100, including a functional assembly 130, is inserted into the intestine of a patient, such as an insertion through a body introduction device 50, such as an endoscope or sheath, or an insertion alongside a body introduction device 50. In some embodiments, catheter 100 is inserted over a guidewire 60. Catheter 100 can be attached to console 200 of system 10. System 10 and/or catheter 100 can comprise one or more sensors configured to produce a signal, such as a signal used to set and/or change one or more console settings 201 of system 10.

In Step 3520, a first functional assembly 130 is positioned in the intestine (e.g. in the duodenum), and one or more layers of a first axial segment of intestinal tissue (e.g. submucosal tissue) is expanded. Tissue expansion can comprise expansion of a near full circumferential (e.g. between 320° and 360°) layer of tissue, and the expansion can be accomplished by one or more fluid delivery elements 139c delivering injectate 221 supplied by console 200 into one or more tissue locations (e.g. 3 tissue locations simultaneously or sequentially without repositioning functional assembly 130).

In Step 3530, a second functional assembly 130 is positioned in a location similar to (e.g. within) the first axial segment of expanded tissue. The first and second functional assemblies 130 can be included on a single catheter 100 (e.g. in two different locations on shaft 110), or on two different catheters 100.

In Step 3540, a confirmation of proper placement can be performed (e.g. a visual examination that all sufficient tissue in contact with the second functional assembly 130 has been expanded). If improper placement is identified, the second functional assembly 130 can be repositioned in Step 3550 and/or the procedure can be aborted.

In Step 3560, the second functional assembly 130 is anchored in place, such as by expanding second functional assembly 130, and/or deploying one or more anchoring mechanisms as described herein.

In Step 3570, the second functional assembly 130 is activated to treat (e.g. ablate tissue) proximate second functional assembly 130. The anchoring previously performed ensures that the treatment is performed in an area of expanded tissue (e.g. expanded submucosal tissue), such as to create a safety margin of tissue in all locations receiving energy or other treatment from second functional assembly 130 during the entire treatment. For example, the anchoring performed prevents unknown or otherwise unintended translation of the second functional assembly 130 prior to and/or during the treatment of Step 3570. Anchoring of functional assembly 130 can be performed a single time or multiple times, and the anchoring can be maintained during any or all of the Steps 3510 through 3570.

While the embodiment of FIG. 4 describes use of a first and second functional assembly 130, a single functional assembly 130 can be used as well. In a single functional assembly 130, the anchoring performed in Step 3560 can be performed during Step 3520 and/or during Step 3530, and maintained through Step 3570. The method of FIG. 4 can be included in any treatment and/or diagnostic procedure as described herein. In some embodiments, multiple tissue expansions are performed by repeating steps 3510 and 3520 one or more times, after which the method of FIG. 4 can be repeated at multiple axial segments of the intestine.

Referring now to FIG. 5, a method of performing a tissue treatment that includes activating a functional assembly based on an image is illustrated, consistent with the present inventive concepts. Method 4100 of FIG. 5 can be performed using system 10 of the present inventive concepts, such as by using catheters 100, 20, 30, and/or 40 as described herein. Method 4100 will be described using system 10 and catheter 100 as described herein.

In Step 4110, the distal portion of a catheter 100, including a functional assembly 130, is inserted into the intestine of a patient such that functional assembly 130 is positioned at a first axial segment of the intestine. Catheter 100 can be inserted through a body introduction device 50, such as an endoscope or sheath, or inserted alongside a body introduction device 50. In some embodiments, catheter 100 is inserted over a guidewire 60. Catheter 100 can be attached to console 200 of system 10. System 10 and/or catheter 100 can comprise one or more sensors configured to produce a signal, such as a signal used to set and/or change one or more console settings 201 of system 10.

In some embodiments, functional assembly 130 is at least partially expanded (e.g. via a fluid introduced into a balloon of functional assembly 130 or via a mechanical linkage configured to radially deploy a portion of functional assembly 130) within the first axial segment of the intestine in Step 4110.

In Step 4120, one or more images are captured, such as by camera 52 of introduction device 50 (e.g. a camera of an endoscope), by imaging device 55 and/or another imaging device of system 10. The one or more images can comprise an image including patient tissue, functional assembly 130 and/or another component of system 10. The one or more captured images can be analyzed, such as an analysis performed manually (e.g. by a clinician) and/or automatically (e.g. by one or more image processing algorithms of system 10, such as algorithm 251). In some embodiments, the image captured by camera 52 and/or imaging device 55 (e.g. and analyzed by algorithm 251) comprises an image selected from the group consisting of: a spectroscopy image, such as a Raman spectroscopy or other image configured to identify denatured proteins; a fluorescence image, such as a time-resolved fluorescence image; optical coherence tomography (OCT) image; ultrasound image; ultrasonic elastography image; colorimetry image; confocal endomicroscopy image; and combinations of one or more of these. In some embodiments, injectate 221 is present in tissue, and a color change or fluorescence is detected when injectate 221 is heated.

In Step 4130, the results of the image analysis performed in Step 4120 are compared to a level of acceptability. If an acceptable level is achieved, the method of FIG. 5 continues in Step 4140. If an acceptable level is not achieved, Step 4180a is performed in which the first treatment is aborted or modified. Unacceptable levels of acceptability can include images which identify: improper positioning of functional assembly 130 such as positioning of functional assembly 130 proximate non-target tissue (e.g. the ampulla of Vater); improper expansion of functional assembly 130; improper position of one or more fluid delivery elements 139c; presence of diseased tissue proximate functional assembly 130 or otherwise; presence of infected tissue proximate functional assembly 130 or otherwise; and combinations of one or more of these.

Aborting the first treatment in Step 4180a can comprise removing catheter 100 (and potentially one or more of devices of system 10) from the patient.

Modifying the first treatment in Step 4180a can comprise returning to step 4110 in which functional assembly 130 is repositioned in the intestine and/or another adjustment is made based on the unacceptable results of the image analysis of Step 4130.

In Step 4140, functional assembly 130 is activated (e.g. energy is delivered to tissue such as heat delivered from hot fluid, RF energy is delivered by one or more electrode-based functional elements 139, light energy is delivered by one or more optical component-based functional elements 139, and/or other energy is delivered as described herein).

In Step 4150, one or more images are captured, such as by camera 52 of introduction device 50 (e.g. a camera of an endoscope), by imaging device 55 and/or another imaging device of system 10. The one or more images can comprise an image including patient tissue, functional assembly 130 and/or another component of system 10. The one or more captured images are analyzed, such as an analysis performed manually (e.g. by a clinician) and/or automatically (e.g. by one or more image processing algorithms of system 10, such as algorithm 251).

In Step 4160, the results of the image analysis performed in Step 4150 are compared to a level of acceptability. If an acceptable level is achieved, the method of FIG. 5 continues in Step 4170. If an acceptable level is not achieved, Step 4180b is performed in which the first treatment is aborted or modified. Unacceptable levels of acceptability can include images which identify: inadequate expansion of tissue; inadequate treatment of target tissue; undesired treatment of target tissue; adverse effects upon non-target tissue; improper positioning of functional assembly 130 such as positioning of functional assembly 130 proximate non-target tissue (e.g. the ampulla of Vater); improper expansion of functional assembly 130; improper position of one or more fluid delivery elements 139c; presence of diseased tissue proximate functional assembly 130 or otherwise; presence of infected tissue proximate functional assembly 130 or otherwise; and combinations of one or more of these.

Aborting the first treatment in Step 4180b can comprise removing catheter 100 (and potentially one or more of devices of system 10) from the patient.

Modifying the first treatment in Step 4180b can comprise returning to Step 4110 in which functional assembly 130 is repositioned in the intestine and/or another adjustment is made based on the unacceptable results of the image analysis of Step 4160.

Alternatively, modifying the first treatment in Step 4180b can comprise returning to Step 4140 (as shown in FIG. 5), in which functional assembly 130 is reactivated, to deliver additional (similar or dissimilar) energy to tissue.

In Step 4170, an assessment of first treatment completeness is performed. The assessment can be performed manually (e.g. by a clinician) and/or automatically (e.g. by one or more algorithms of system 10, such as via one or more images produced as described herein). If it is determined that the first treatment is not complete, Step 4140 and subsequent steps are repeated, such as to deliver additional energy to tissue. If it is determined that the first treatment is complete, Step 4190 is performed in which the first treatment is ended. In some embodiments, Step 4190 comprises overall completion of a patient procedure, such as when catheter 100 and/or other components of system 10 are removed from the patient. In other embodiments, the steps of the method of FIG. 5 are repeated for a second treatment, such as by repeating Step 4110 at a second axial segment of intestinal tissue and continuing with the subsequent steps. In some embodiments, at least 2 or at least 3 axial segments of duodenal tissue are treated, such as is described herein to treat diabetes.

Referring now to FIG. 6, a method of marking tissue and performing a tissue treatment based on the tissue marking is illustrated, consistent with the present inventive concepts. Method 4300 of FIG. 6 can be performed using system 10 of the present inventive concepts, such as by using catheters 100, 20, 30, and/or 40 as described herein. Method 4300 will be described using system 10 and catheter 100 as described herein.

In Step 4310, a patient is selected for treatment by the systems, methods and devices of the present inventive concepts, such as a patient with insulin resistance and/or another metabolic condition that has been selected for ablation of duodenal mucosa.

In Step 4320, the distal portion of a catheter 100, including a functional assembly 130, is inserted into the intestine of a patient such that functional assembly 130 is positioned at a first axial segment of the intestine. Catheter 100 can be inserted through a body introduction device 50, such as an endoscope or sheath, or inserted alongside a body introduction device 50. In some embodiments, catheter 100 is inserted over a guidewire 60. Catheter 100 can be attached to console 200 of system 10. System 10 and/or catheter 100 can comprise one or more sensors configured to produce a signal, such as a signal used to set and/or change one or more console settings 201 of system 10.

In Step 4330, one or more portions of tissue are marked and/or identified. In some embodiments, Step 4330 is performed prior to Step 4320. In some embodiments, the tissue marking and/or identification of Step 4330 is performed with catheter 100 and/or another component of system 10, such as tool 500 as described herein. Tissue marking can comprise implantation of a temporary marker, and/or it can include marking of tissue with a tattoo or other tissue dyeing procedure, such as using marker 490, also described herein. In some embodiments, marked tissue comprises tissue selected from the group consisting of: target tissue; non-target tissue; tissue proximate non-target tissue (e.g. tissue proximate the ampulla of Vater or tissue proximate the pylorus); safety margin tissue; diseased tissue; healthy tissue; and combinations of one or more of these.

In Step 4340, target tissue is treated (e.g. target tissue comprising diseased tissue or otherwise adversely functioning tissue) with functional assembly 130 of catheter 100, such as a target tissue treatment comprising tissue ablation; tissue expansion; tissue expansion and ablation; and combinations of one or more of these. In some embodiments, multiple tissue expansion steps precede each ablation step. The treatment of target tissue is performed at a location selected based on the tissue identification and/or marking performed in Step 4330.

For example, in procedures treating mucosa of the duodenum, one or more markings can be made to prevent adversely affecting the ampulla of Vater (e.g. by preventing energy deliver to tissue within 1.5 cm, 1.0 cm or 0.5 cm of the ampulla of Vater) and/or to ensure treatment of tissue (e.g. mucosal tissue) within 5 cm, within 10 cm or within 15 cm of the ampulla of Vater.

In Step 4350, catheter 100 is removed. The markers may be removed, or they can be left in place (e.g. when a dye is used or when a deployed marker is constructed and arranged to pass through the GI system naturally).

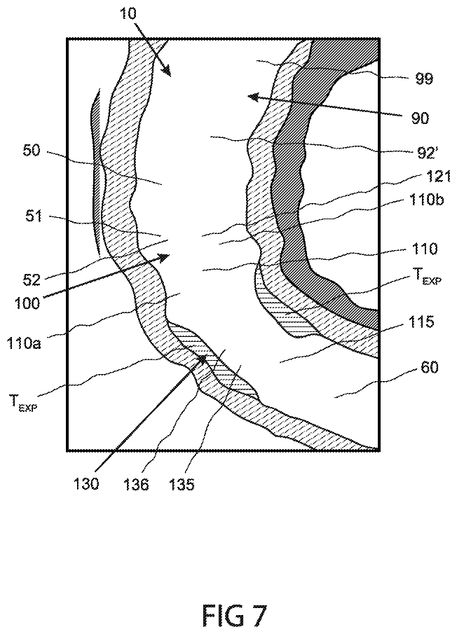

Referring now to FIG. 7, a sectional view of the distal portion of a system including an endoscope and a treatment device inserted into a duodenum of a patient is illustrated, consistent with the present inventive concepts. System 10 includes catheter 100, such as a catheter configured to both expand tissue (e.g. circumferentially expanded tissue T_{EXP} shown), as well as treat (e.g. ablate) target tissue. Catheter 100 and other components of system 10 can be of similar construction and arrangement to the similar components described herein in reference to FIGS. 1 and/or 2. Catheter 100 is shown positioned in a side-by-side arrangement with endoscope 50, which can include one or more working channels, lumen 51 shown, and a visible light and/or infrared camera, camera 52 also shown. Catheter 100 has been advanced over a guidewire 60 and through introducer 90 (e.g. to a location in the small intestine of the patient). Introducer 90 includes shaft 99 with expanded distal end 92′. Distal end 92′ can be sized to surround a bulbous distal end of catheter 100, such as tip 115 shown. In some embodiments, catheter 100 is advanced over guidewire 60 but not through introducer 90.

Catheter 100 includes a treatment assembly, functional assembly 130, which is shown in its expanded state, and positioned on a central shaft, shaft 110. Functional assembly 130 can include one or more fluid delivery elements, not shown but such as one or more (e.g. three) needles or other fluid delivery elements such as fluid delivery elements 139c described herein. The fluid delivery elements can be positioned in a circumferential arrangement (e.g. three needles positioned approximately 120° apart along functional assembly 130), each fluid delivery element fluidly attached to a fluid delivery tube, such as a conduit positioned within shafts 110a and 110b shown. The fluid delivery elements may each be positioned in a port, such as port 137, also not shown but described herein, such that a vacuum can be applied to tissue to cause the tissue to be drawn into the port 137, after which fluid can be injected into the tissue via the associated fluid delivery element. Functional assembly 130 can comprise a radially expandable assembly, such as balloon 136, into which an ablative-based treatment element 135 can be positioned (e.g. an electrode configured to deliver RF or other electromagnetic energy) and/or ablative fluid introduced (e.g. hot or cold ablative fluid introduced into balloon 136). Catheter 100 can comprise one or more visualizable markers, such as radiopaque or visible marker bands, circumferential marker 121 (3 shown). In some embodiments, a neutralizing fluid (e.g. a cooling or warming fluid) is introduced into balloon 136 prior to and/or after ablation of tissue, as described herein.

In the embodiment shown in FIG. 7, tissue surrounding and proximate functional assembly 130 has been expanded (circumferentially expanded tissue T_{EXP} shown), such that ablation or other treatment can be performed by functional assembly 130 on the mucosal layer of the axial segment of the small intestine (e.g. the duodenum) proximate functional assembly 130 (e.g. proximate balloon 136), such as is described herein. After the tissue treatment is performed, functional assembly 130 can be radially compacted (e.g. balloon 136 at least partially deflated), translated (e.g. advanced or retracted to a neighboring or distant axial segment), after which similar tissue expansion (e.g. submucosal tissue expansion) and tissue treatment (e.g. mucosal tissue ablation) can be performed, such as to treat a patient medical condition (e.g. a disease and/or disorder) as described herein. In some embodiments, multiple tissue expansions steps are performed prior to each ablation step.

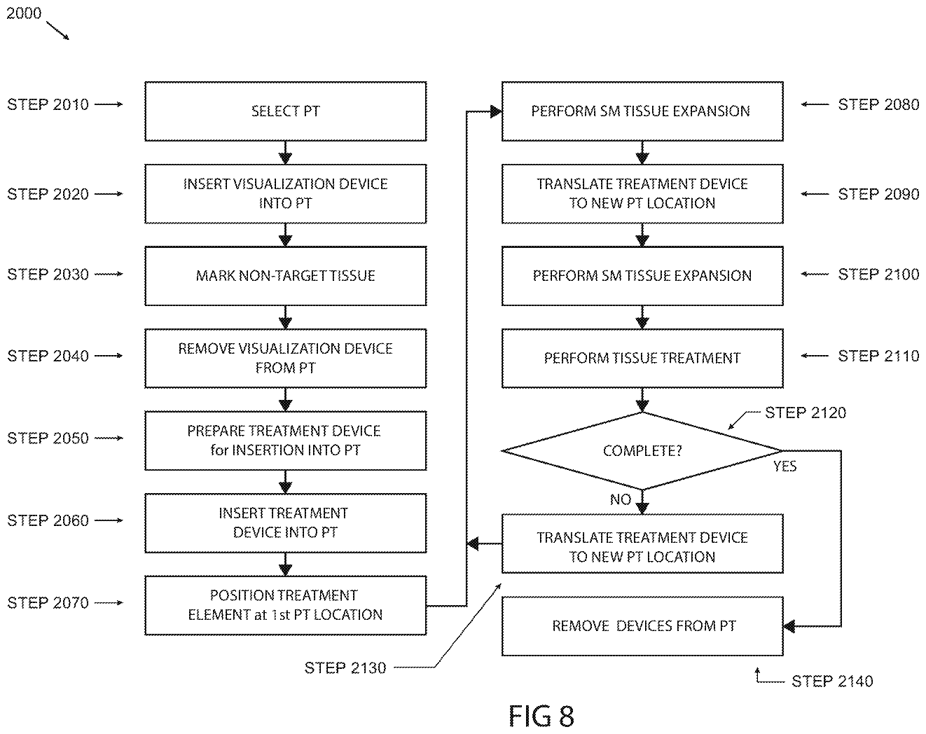

Referring now to FIG. 8, a flow chart of a method of treating a patient is illustrated, consistent with the present inventive concepts. Method 2000 of FIG. 8 can be performed using system 10 of the present inventive concepts, such as by using catheters 100, 20, 30, and/or 40 as described herein. Method 2000 will be described using system 10 and catheter 100 as described herein.

Throughout method 2000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), neutralizing fluid (e.g. cold or warm, respectively, neutralizing fluid), injectate 221, and/or other fluid.

In STEP 2010, a patient is selected for treatment. The patient can be selected to treat one or more of the medical conditions described herein. In some embodiments, the selected patient is inflicted with Type 2 diabetes and another medical condition, such as NAFLD/NASH and/or PCOS. One or more patient diagnostic tests can be performed such as to include or exclude a potential patient.

In STEP 2020, a visualization device is inserted into the patient by an operator of system 10 (e.g. a clinician of the patient). For example, the visualization device can comprise an endoscope (e.g. endoscope 50a described herein). Alternatively or additionally, the visualization device inserted in STEP 2020 can be a treatment device of the present inventive concepts, such as catheter 100 described herein, such as a treatment device that includes a camera or other visualization assembly (e.g. a functional element 119 comprising a visualization assembly). In these embodiments, the inserted catheter 100 has already been prepared for insertion via performance of STEP 2050 described herein.

In some embodiments, guidewire 60 is inserted into the patient (e.g. via a working channel of an endoscope and/or a guidewire lumen of a treatment device). Guidewire 60 can be used to introduce catheter 100 (in STEP 2020 or otherwise).

In some embodiments, the visualization device comprises an endoscope with a scope cap, such as cap 53 described herein in reference to FIG. 1. Scope cap 53 can prevent tissue (e.g. duodenal or other luminal wall tissue) from collapsing in front of a camera of the endoscope, such tissue collapse undesirably limiting the view provided by the visualization device.

The visualization device and other devices inserted in the various steps below, can be inserted into the patient via the mouth, such as to enter the small intestine by passing through the stomach. Alternatively, the device can be inserted via a surgical incision through the skin, and/or via minimally invasive access tools (e.g. one or more laparoscopic ports).

In STEP 2030, an optional step of marking non-target tissue is performed. Using the visualization device inserted in STEP 2020, the operator can identify the ampulla of Vater, such as to mark the ampulla of Vater to allow rapid, simplified visualization of the ampulla of Vater in later steps (e.g. to avoid adversely affecting the ampulla of Vater and its neighboring tissue). In some embodiments, the ampulla of Vater is visualized using a side-viewing visualization device (e.g. an endoscope with side-viewing capability). In some embodiments, the ampulla of Vater is marked through implantation of a marker, such as marker 490 described herein, such as a temporarily implantable marker, such as a hemostasis clip. Marker 490 can comprise a radiopaque marker (e.g. to be visualized by a fluoroscope), an ultrasonically visible marker (e.g. to be visualized by an ultrasound imaging device), and/or a magnetic marker. Marker 490 can comprise biocompatible ink.

In some embodiments, one or more patient screening procedures are performed in STEP 2030, such as to confirm that the target tissue to be treated, and/or tissue proximate the target tissue, is free of disease or other undesired conditions. If an undesired condition is identified, the procedure can be aborted (e.g. via step 2140 described herein).

In STEP 2040, the visualization device inserted in STEP 2020 can be removed from the patient, such as when the visualization device comprises endoscope 50a or other body introduction device 50. Removal of this type of visualization device can be performed leaving a guidewire (e.g. guidewire 60) in place. Alternatively, the visualization device inserted in STEP 2020 comprises a treatment device of the present inventive concepts, and the treatment device, such as a catheter 100 remains in the patient.

In STEP 2050, a treatment device, such as catheter 100, is prepared for insertion into the patient. In some embodiments, catheter 100 comprises the visualization device of STEP 2020, and STEP 2050 is performed prior to STEP 2020.

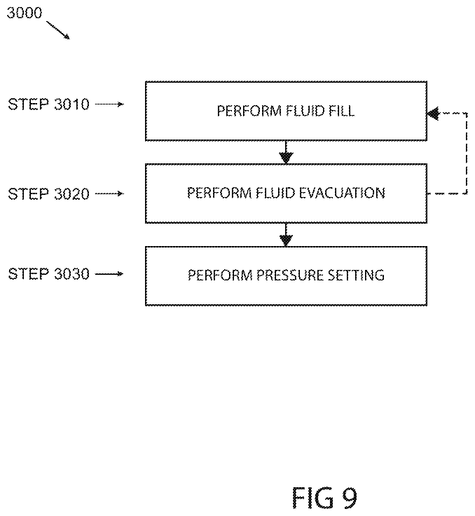

Catheter 100 is attached to console 200, such as via connecting assembly 300, and one or more procedures are performed such as to remove air from one or more lumens, balloons, and/or other spaces within catheter 100. In some embodiments, catheter 100 is prepared using method 3000 described herein in reference to FIG. 9.

In some embodiments, functional assembly 130 comprises balloon 136, and after the final procedure of STEP 2050 is performed, balloon 136 is filled with a small, but non-zero volume of fluid, at a pressure less than full vacuum, such that functional assembly 130 is in a preferred “translation state”, as described herein in reference to FIG. 9. This translation state provides numerous advantages for safe and effective translation of catheter 100 in the duodenum and other segments of the GI tract of the patient, also as described herein in reference to FIG. 9.

In STEP 2060, catheter 100 is inserted into the patient (e.g. if not already inserted in STEP 2020). In some embodiments, catheter 100 is inserted with the corresponding functional assembly 130 in the translation state described herein in STEP 2050 and herein in reference to method 3000 of FIG. 9. In some embodiments, catheter 100 is inserted over a guidewire, such as guidewire 60 which can be already in place as described herein.

In some embodiments, such as when duodenal mucosal tissue is to be treated (i.e. the target tissue comprises duodenal mucosa), functional assembly 130 of catheter 100 is positioned proximate the duodenal bulb or segment D1 of the duodenum.

In some embodiments, catheter 100 is inserted after a body introduction device, such as endoscope 50a, has been recently removed (e.g. in STEP 2040).

In some embodiments, after catheter 100 is introduced into the patient in STEP 2060, endoscope 50a is introduced (e.g. reintroduced) into the patient as well. Subsequent translations of catheter 100 can be performed with simultaneous translation of endoscope 50a.

In STEP 2070, a treatment assembly configured to perform a submucosal tissue expansion, such as functional assembly 130 of catheter 100, is positioned at a first location in the patient's small intestine, such as a location in the duodenum distal to the pylorus and proximal to the Ligament of Treitz. Alternatively or additionally, other GI locations can be selected for tissue expansion (e.g. submucosal tissue expansion). During positioning, catheter 100 (e.g. functional assembly 130) can be in a translation state as described herein.

In STEP 2080, a submucosal tissue expansion is performed, such as via functional assembly 130 of catheter 100, the expansion performed at the location established in STEP 2070.

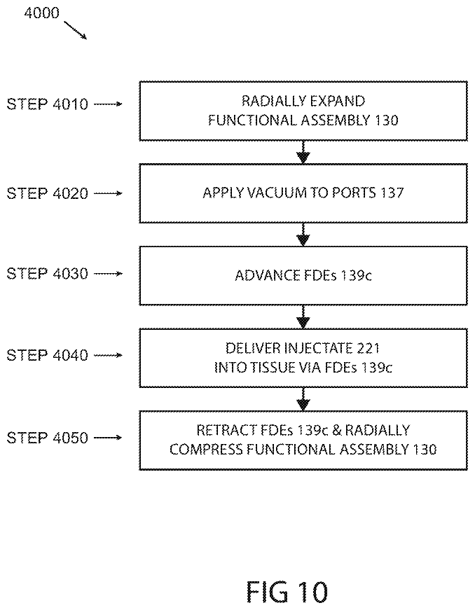

The tissue expansion performed in STEP 2080 can be performed using method 4000 described herein in reference to FIG. 10.

In STEP 2090, the treatment catheter is translated, such as a translation of catheter 100. In some embodiments, catheter 100 is translated such as to cause a corresponding translation of functional assembly 130 that is approximately one-half of the length of functional assembly 130 (e.g. approximately 1 cm when functional assembly 130 comprises a length of approximately 2 cm). In some embodiments, functional assembly 130 is translated distally (e.g. more distal in the duodenum, further away from the ampulla of Vater toward but not passing the ligament of Treitz). Alternatively, functional assembly 130 is translated proximally. During translation, catheter 100 (e.g. functional assembly 130) can be in a translation state as described herein.

In STEP 2100, another submucosal tissue expansion is performed, such as via functional assembly 130 of catheter 100. The tissue expansion is performed at the location established in STEP 2090. Catheter 100 (e.g. functional assembly 130) can be in a translation state as described herein.

The tissue expansion performed in STEP 2100 can be performed using method 4000 described herein in reference to FIG. 10.

The tissue expansion performed in STEP 2100 can be performed at a duodenal or other GI location that is proximate, yet distal to the location of tissue expansion performed in step 2080. Alternatively, the tissue expansion performed in STEP 2100 can be proximal to the location of STEP 2080.

In STEP 2110, a tissue treatment procedure is performed, such as via functional assembly 130 of catheter 100. The tissue treatment procedure can be performed in the same location of the tissue expansion performed in STEP 2100 (e.g. without translation of functional assembly 130).

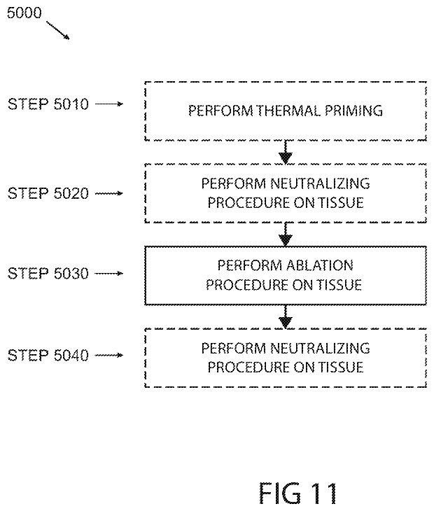

The tissue treatment performed in STEP 2110 can be performed using method 5000 described herein in reference to FIG. 11. In some embodiments, prior to performing method 5000, catheter 100 and functional assembly 130 are established in the translation state described herein.

The tissue treatment performed in STEP 2110 can include a neutralizing procedure and an ablation procedure, such as is described herein in reference to FIGS. 1 and/or 2. In some embodiments, a neutralizing procedure (e.g. a cooling or warming procedure) is performed prior to and/or after an ablation procedure (e.g. a heat or cryogenic ablation procedure, respectively) at a single axial location of the GI tract (e.g. and repeated for multiple axial locations). In some embodiments, a neutralizing procedure (e.g. a cooling or warming procedure) is performed only after (i.e. not prior to) an ablation procedure (e.g. a heat or cryogenic ablation procedure, respectively) at a single axial location of the GI tract (e.g. and repeated for multiple axial locations). In other embodiments, a neutralizing procedure (e.g. a cooling or warming procedure) is performed both prior to and after an ablation procedure (e.g. a heat or cryogenic ablation procedure, respectively) at a single axial location of the GI tract (e.g. and repeated for multiple axial locations).

In STEP 2120, a decision is made related to performing additional tissue treatments. If additional tissue treatments are desired, STEP 2130 is performed. If the procedure is complete, STEP 2140 is performed. In some embodiments, at least 2, 3, 4, 5, or 6 tissue treatments are performed. In some embodiments, at least 6 cm of cumulative axial length of duodenum is treated, such as to achieve a desired therapeutic benefit as described herein. The at least 6 cm of cumulative axial length can be treated via a single treatment step (e.g. a single ablation using functional assembly 130), or via multiple treatment steps (e.g. at least 3 ablations, at least 4 ablations, and/or at least 5 ablations using functional assembly 130). In these embodiments, functional assembly 130 can comprise a treatment length of at least 10 mm, such as a treatment length of no more than 100 mm.

In STEP 2130, catheter 100, including functional assembly 130, is translated to a new location within the GI tract, such as a location approximately 1 cm distal to the current location. Alternatively, functional assembly 130 can be translated proximally (e.g. 1 cm proximally). Subsequently, STEP 2080 is repeated.

In STEP 2140, the treatment device, and any other device (e.g. endoscope 50a and/or guidewire 60) is removed from the patient, and the procedure is complete.

In some embodiments, the tissue expansion procedures (STEPS 2080 and 2100) and the tissue treatment procedures (STEP 2110) are performed with the same catheter, such as catheter 100 and/or 40 described herein. In other embodiments, the tissue expansion procedures (STEPS 2080 and 2100) are performed with a first catheter, such as catheter 20 described herein, and the tissue treatment procedures (STEP 2110) are performed with a second, different catheter, such as catheter 100 of FIG. 2.

Referring now to FIG. 9, a flow chart of a method of preparing a treatment device is illustrated, consistent with the present inventive concepts. Method 3000 of FIG. 9 can be performed using system 10 of the present inventive concepts, such as by using catheters 100, 20, 30, and/or 40 as described herein. Method 3000 is described using system 10 and catheter 100 as described herein. Method 3000 is performed on a catheter that has been attached to console 200, such as via connecting assembly 300 as described herein in reference to FIGS. 1 and 2.

Throughout method 3000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), neutralizing fluid (e.g. cold or warm, respectively, neutralizing fluid), and/or other fluid.

In STEP 3010, a fluid fill procedure is performed, such as to fully or partially fill functional assembly 130 (e.g. balloon 136) with fluid. The fluid fill procedure can be performed: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as fluid is delivered into functional assembly 130 (e.g. via one or more lumens or other conduits of catheter 100), fluid is simultaneously evacuated (e.g. slowly removed) from functional assembly 130 (e.g. via one or more lumens or other conduits of catheter 100), such that functional assembly 130 achieves a desired level of expansion (e.g. functional assembly does not significantly expand) during the process. The fluid delivered to functional assembly 130 in STEP 3010 can be relatively cold fluid, such as fluid that is less than body temperature (e.g. room temperature) and/or less than room temperature. For example, the fluid provided by a reservoir 220 of console 200 can contain a fluid that is also a neutralizing fluid configured to perform a pre-cool and/or post-cool of an ablation treatment, such as is described herein in reference to method 5000 of FIG. 11.

The delivery and removal of various fluids to and/or from catheter 100 can be performed by one or more pumping assemblies 225 of console 200.

In STEP 3020, a fluid evacuation procedure is performed, such as to fully or partially evacuate functional assembly 130 (e.g. balloon 136) of fluid. The fluid evacuation procedure can be performed: for a pre-determined period of time (e.g. for less than 15 seconds, for less than 10 seconds, and/or for approximately 6 seconds); until a particular volume of fluid is removed from and/or remains within functional assembly 130; and/or until a pre-determined pressure is achieved within functional assembly 130. The evacuation can be performed at a particular pressure or range of pressures, and/or at a particular flow rate or range of flow rates. In some embodiments, fluid is evacuated from functional assembly 130 (e.g. via one or more lumens or other conduits of catheter 100) until a particular volume remains within functional assembly 130 (e.g. within balloon 136). Removal of fluids can be performed by one or more pumping assemblies 225 of console 200. In some embodiments, the pressure within functional assembly 130 is near full vacuum at the end of STEP 3020.

In some embodiments, STEPS 3010 and 3020 are repeated one or more times, prior to performing STEP 3030, such as when catheter 100 is initially prepared for insertion into the patient and STEPS 3010 and 3020 are performed at least two times each.

In STEP 3030, a pressure setting procedure is performed, which establishes functional assembly 130 in a preferred “translation state” (e.g. a state in which translation of functional assembly 130 within the GI tract is safe, effective, and relatively easy). In STEP 3030, the pressure within functional assembly 130 (e.g. within balloon 136) is brought to a particular level. Alternatively or additionally, a particular volume (e.g. a minimal volume) of fluid is caused to remain within functional assembly 130.

In some embodiments, prior to performing STEP 3030, the pressure within functional assembly 130 is at or near full vacuum (e.g. as caused in STEP 3020). In STEP 3030, fluid can be delivered (and/or evacuated) such as to cause the pressure within functional assembly 130 to reach a target level related to the desired translation state (e.g. a pressure in functional assembly 130 that is estimated based on a pressure reading performed by a functional element of system 10 present in catheter 100, connecting assembly 300, and/or console 200). In some embodiments, the target level is below room pressure, such as when a measure pressure (e.g. a pressure within console 200 or other system location) is at least 1 psi below room pressure (−1 psi), at least 2 psi below room pressure, or at approximately −2.7 psi. Establishing a slightly negative pressure causes functional assembly 130 to be partially compacted, but not to the extent that significant rigidity occurs. In some embodiments, the translation state target level for the pressure within functional assembly 130 is no more than 5 psi below room pressure, or no more than 4 psi below room pressure. In other embodiments, the target pressure level for functional assembly 130 is less than 1 psi (i.e. 1 psi above room pressure), or less than 0.5 psi, and/or the translation state is established via a maximum volume contained within functional assembly 130, such as a volume less than 5%, or less than 10% of the “full volume” of balloon 136 (e.g. the volume to rigidly inflate a relatively non-compliant balloon 136, or the volume to inflate a compliant balloon without significantly stretching the balloon), the maximum pressure and/or volume establishing a limited (e.g. small) expansion of functional assembly 130. In some embodiments, the volume of fluid in balloon 136 during the transition state is less than 3 ml, 2 ml, or 1 ml.

Advantages of the translation state established for catheter 100 in STEP 3030 are that functional assembly 130 (e.g. including balloon 136 and ports 137) is established with a relatively low profile (e.g. a relatively minimal diameter surrounds shaft 110), and its components in a relatively flexible condition (e.g. not fully compacted via a complete vacuum, such that the components of functional assembly 130 are able to move with relatively low force applied). In these low profile, non-rigid states, ease of translation of functional assembly 130 is maximized or at least improved.

In some embodiments, establishing of the translation state of a treatment device (e.g. catheter 100) via method 3000 is performed between each tissue treatment (e.g. ablation) step and a subsequent submucosal tissue expansion step. For example, method 3000 can be performed after completion of STEP 2110 and prior to a (repeated) STEP 2080, each of method 2000 of FIG. 8 described herein.

Referring now to FIG. 10, a flow chart of a method of expanding tissue with a treatment device is illustrated, consistent with the present inventive concepts. Method 4000 of FIG. 10 can be performed using system 10 of the present inventive concepts, such as by using catheters 100, 20, 30, and/or 40 as described herein. Method 4000 is described using system 10 and catheter 100 as described herein. Method 4000 is described using a catheter 100 that has been attached to console 200, such as via connecting assembly 300 as described herein in reference to FIGS. 1 and 2. Delivery and removal of fluids of method 4000 can be performed by one or more pumping assemblies 225 of console 200.

Throughout method 4000, system 10 (e.g. via user interface 205 of console 200) can provide (e.g. to an operator) information related to injectate 221 in one or more reservoirs 220, such as volume, pressure, and/or temperature information. Based on the provided information, the procedure may be aborted, modified, or proceeded as intended (e.g. manually by the operator and/or automatically by system 10).

In STEP 4010, functional assembly 130 of catheter 100 is radially expanded, such as by the delivery of fluid into balloon 136. For example, fluid can be delivered from one or more reservoirs 220 by a pumping assembly 225. The fluid delivered in STEP 4010 can be performed: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as fluid is delivered into functional assembly 130 (e.g. via one or more lumens or other conduits of catheter 100), fluid is simultaneously evacuated (e.g. slowly removed) from functional assembly 130 (e.g. via one or more lumens or other conduits of catheter 100), such that functional assembly 130 achieves a desired level of expansion (e.g. functional assembly does not significantly expand) during the process. The fluid delivered to functional assembly 130 in STEP 4010 can be relatively cold fluid, such as fluid that is less than body temperature and/or less than room temperature (e.g. fluid provided by a reservoir 220 of console 200) that contains a neutralizing fluid configured to perform a pre-cool and/or post-cool of an ablation treatment that includes and/or generates heat (e.g. a hot fluid ablation, an RF ablation, a light ablation, and/or an ultrasound ablation). Delivery and removal of fluids can be performed by one or more pumping assemblies 225 of console 200.

In some embodiments, a fixed volume of fluid is delivered to functional assembly 130, such as a volume of at least 4 ml, at least 6 ml, or approximately 8 ml. In some embodiments, fluid is delivered until functional assembly 130 is in relatively close apposition to the wall of the GI tract within which functional assembly 130 is positioned (e.g. automatically by system 10 or manually by an operator).

In STEP 4020, vacuum is applied to one or more (e.g. all) ports 137 of functional assembly 130, such that tissue proximate each port 137 is drawn into a cavity of port 137. In some embodiments, the pressure applied to port 137 is monitored (e.g. via a location within console 200, connecting assembly 300, and/or catheter 100). The monitoring of the pressure can be used to confirm that the pressure maintains a minimum vacuum (e.g. at least 2 psi, at least 4 psi, or at least 6 psi below room pressure). Alternatively or additionally, the pressure can be monitored to confirm that the vacuum level is relatively stable, such as a stability correlating to a pressure that does not vary more than 0.3 psi, 0.2 psi, and/or 0.1 psi within a time window of at least 2 seconds, at least 3 seconds, and/or at least 5 seconds. If the minimum vacuum level, or stability level is not maintained, system 10 can be configured to enter an alert state (e.g. a state in which the operator is notified and/or further treatment steps are prevented until resolution is achieved).

In STEP 4030, one or more fluid delivery elements 139c are advanced (e.g. multiple fluid delivery elements 139c that are simultaneously or sequentially advanced) into the tissue captured within each corresponding port 137. In some embodiments, multiple fluid delivery elements 139c are advanced by a single control (e.g. a control 104 on handle 102 of catheter 100, as described herein). In some embodiments, two or more fluid delivery elements 139c are advanced by separate, individual controls (e.g. two or more controls 104).

In STEP 4040, injectate 221 is delivered into the submucosal tissue by one or more needles or other fluid delivery elements 139c (e.g. into the tissue captured within each port 137). Injectate 221 is provided via one or more reservoirs 220 and delivered by one more pumping assembly 225, such as is described herein in reference to FIGS. 1 and/or 2. In some embodiments, a fixed volume of fluid is introduced through each fluid delivery element 139c, such as at least 3 ml, at least 5 ml, at least 7 ml, or approximately 10 ml injected into tissue via at least 2, at least 3, or at least 4 fluid delivery elements 139c. In some embodiments, the volume of fluid delivered into the submucosal tissue comprises a volume less than the volume of fluid that was provided by console 200, such as a loss of fluid following the priming of catheter 100 or a loss of fluid at a puncture site of fluid delivery element 139c. For example, at least 50% of the fluid provided by console 200 can be delivered into the submucosal tissue.

In some embodiments, pressure within the fluid pathway containing injectate 221 (e.g. within each associated reservoir 220 such as a syringe or other reservoir) is monitored during the delivery of injectate 221 to tissue. In some embodiments, injectate 221 is delivered at a flow rate that prevents the pressure within the fluid pathway from exceeding a maximum level, such as a level of no more than 150 psi, or no more than 110 psi at a fluid pathway location proximate console 200. In some embodiments, multiple fluid delivery elements 139c (e.g. needles) are each fluidly attached to individual, separate reservoirs 220, via separate fluid pathways, and if the associated fluid pathway pressure for a single fluid delivery element 139c exceeds the maximum level, the flow rate of injectate 221 delivery is reduced (e.g. reduced for all fluid delivery elements 139c). Pressure measurements above the maximum could relate to an occlusion or other restriction in the fluid pathway between console 200 and fluid delivery elements 139c and exceeding the pressure can result in system 10 entering an alert state. Configuration of system 10 to prevent exceeding the maximum pressure provides a safety measure (avoiding excessive pressure of injectate 221 delivery into the patient). In some embodiments, the pressure within each flow pathway containing injectate 221 is confirmed to be above a minimum pressure (e.g. such as a pressure of at least 20 psi). Pressure below the minimum (e.g. below the threshold for at least for a minimum time period such as at least one second or at least 2 seconds) can indicate air in the fluid pathway, or a leak, and system 10 can be configured to enter an alert state if the minimum threshold is exceeded.

In some embodiments, system 10 (via console 200) is configured to maintain a constant volume within functional assembly 130 (e.g. within balloon 136) throughout the injection of injectate 221 into tissue. For example, the volume within balloon 136 can be at a level less than the volume of balloon 136 when it is fully expanded. In some embodiments, the volume is no more than 90% of the full volume of balloon 136, such as no more than 80% of the full volume, or no more than 70% of the full volume (e.g. balloon 136 is filled with 8 ml when the full volume is 12 ml). In some embodiments, system 10 is configured to enter an alert state if the volume within functional assembly 130 is below a minimum and/or above a maximum.

In some embodiments, system 10 (via console 200) is configured to regulate the pressure (e.g. ensure the pressure is above a minimum and/or below a maximum) within functional assembly 130 (e.g. within balloon 136) during injection of injectate 221 into tissue. In some embodiments, system 10 is configured to enter an alert state if the pressure within functional assembly 130 is below a minimum and/or above a maximum.

In STEP 4050, all fluid delivery elements 139c are retracted, and functional assembly 130 is radially compressed. Retraction of fluid delivery elements 139c can be performed in a similar, typically opposite direction, to the method used to deploy them in STEP 4030 (e.g. via one or more controls 104 of handle 102 of catheter 100). Functional assembly 130 can be radially compressed via evacuation of the fluid within functional assembly 130, via one or more pumping assemblies 225 as described herein. In some embodiments, functional assembly 130 is radially compressed by evacuating a fixed volume of fluid (e.g. from balloon 136), such as the same or at least a similar volume to that introduced into functional assembly 130 in STEP 4010 (e.g. a volume of at least 4 ml, at least 6 ml, or approximately 8 ml).

Referring now to FIG. 11, a flow chart of a method of ablating or otherwise treating tissue with a treatment device is illustrated, consistent with the present inventive concepts. Method 5000 of FIG. 11 can be performed using system 10 of the present inventive concepts, such as by using catheters 100, 20, 30, and/or 40 as described herein. Method 5000 is described using system 10 and catheter 100 as described herein. Method 5000 is described using a catheter 100 that has been attached to console 200, such as via connecting assembly 300 as described herein in reference to FIGS. 1 and 2. Delivery and removal of fluids of method 5000 can be performed by one or more pumping assemblies 225 of console 200.

Throughout method 5000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), and/or neutralizing fluid (e.g. cold or warm, respectively, neutralizing fluid).

In the various steps of method 5000, a reservoir 220 can be filled with an ablative fluid at an elevated temperature, such as a temperature of at least 90° C., at least 93° C., or approximately 96° C. Alternatively or additionally this elevated temperature ablative fluid can be maintained at a temperature of no more than 99° C., such as no more than 98° C., or no more than 97° C. Another reservoir 220 can be filled with a neutralizing fluid that is maintained at a temperature less than body temperature, such as a temperature of approximately room temperature. Alternatively or additionally, a reservoir 220 can be filled with a chilled fluid. The chilled fluid can be maintained at a temperature of no more than 30° C., or no more than 25° C. Alternatively or additionally, this chilled fluid can be maintained at a temperature below room temperature but above 5° C., such as above 7.5° C., or above 9° C.

In STEP 5010, an optional step of a thermal priming procedure is performed on one or more of the fluid pathways of console 200, connecting assembly 300 (if present), and catheter 100. In some embodiments, the fluid pathways of connecting assembly 300 are warmed, such as to a temperature of at least 60° C., 70° C., or 80° C., such as a temperature of approximately 86° C. In these embodiments, fluid pathways of catheter 100 can also be warmed, or not.

In STEP 5020, an optional step of performing a pre-ablation neutralizing procedure on tissue is performed (e.g. to tissue in close proximity to functional assembly 130 and/or tissue proximate and/or somewhat remote from this tissue). For example, a cooling fluid can be delivered to functional assembly 130, such as when the ablation of STEP 5030 includes and/or generates heat, such as when the ablation includes a hot fluid ablation, an electromagnetic energy ablation (e.g. an RF ablation), a light energy ablation (e.g. a laser ablation), and/or a sound energy ablation (e.g. a high intensity or other ultrasound ablation).

Upon activation by an operator via user interface 205, neutralizing fluid is introduced into functional assembly 130 (e.g. into balloon 136).

The neutralizing fluid can be delivered to functional assembly 130: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as neutralizing fluid is delivered into functional assembly 130 (e.g. via one or more lumens or other conduits of catheter 100), fluid is simultaneously evacuated from functional assembly 130 (e.g. via one or more lumens or other conduits of catheter 100), at flow rates such that functional assembly 130 remains expanded (e.g. remains in contact with surrounding mucosal tissue of the duodenum or other GI mucosal tissue), but fluid within functional assembly 130 is recirculated.

Once functional assembly 130 is filled with the neutralizing fluid, that particular volume of neutralizing fluid can remain in place (e.g. without removal or replacement) throughout the remaining portion of STEP 5020, and/or it can be recirculated, as described herein, for the remaining portion of STEP 5020.

Tissue proximate functional assembly 130 is cooled or otherwise neutralized as long as neutralizing fluid is maintained within functional assembly 130 (e.g. in a stagnant or recirculating manner), and functional assembly 130 is in relative contact with the tissue.

In some embodiments, neutralizing fluid is delivered to functional assembly 130 in a recirculating manner, for a pre-determined time period, such as a time period of at least 5 seconds, 10 seconds, or 15 seconds. In these embodiments, for an initial period (e.g. a period of approximately 2 seconds), fluid is not evacuated from functional assembly 130, allowing functional assembly 130 to radially expand to contact tissue. Subsequently (e.g. for at least the next 3 seconds, 8 seconds, or 12 seconds), functional assembly 130 is in contact with mucosal tissue and neutralizing fluid cools the contacted mucosal tissue as well as other tissue in relative proximity to the contacted mucosal tissue (e.g. neighboring mucosal tissue, as well as deeper tissues including the neighboring submucosal tissue, gastrointestinal adventitia, the tunica serosa, and tunica muscularis).

During this tissue neutralizing procedure, one or more fluid pathway temperatures can be monitored, as described herein, such as to change temperature in a closed-loop fashion, and/or to enter an alert state if a temperature threshold is exceeded.

During this tissue neutralizing procedure, the pressure within one or more fluid pathways can be monitored, such as to adjust the pressure in a closed-loop fashion, and/or to enter an alert state if a pressure threshold is exceeded. For example, pressure below a minimum can represent a break of balloon 136 and/or other leak in the fluid pathway. Pressure above a maximum can represent an occlusion or restriction (e.g. a kink in catheter 100) has occurred.

Temperature and/or pressure can be monitored by one or more temperature sensor and/or pressure sensor-based functional elements of console 200, connecting assembly 300, and/or catheter 100, as described in detail herein in reference to FIGS. 1 and/or 2.

While STEP 5020 has primarily been described using a cooling fluid, in alternative embodiments, a warming fluid can be delivered to functional assembly 130 (e.g. to neutralize a cryogenic ablation) or an agent configured to neutralize a chemical ablation can be delivered directly to the mucosal tissue surface (e.g. a non-target tissue surface).

In STEP 5030, an ablation or other tissue treatment procedure is performed on target tissue (e.g. to tissue in close proximity to functional assembly 130 and/or tissue proximate this tissue). For example, an elevated temperature ablative fluid can be delivered to functional assembly 130, such as when the neutralizing fluid of STEP 5020 comprised fluid at a temperature below body temperature.

Ablative fluid is introduced into functional assembly 130 (e.g. into balloon 136), via manual activation by an operator or automatically by system 10 (e.g. an automatic initiation when STEP 5020 is completed).

The ablative fluid can be delivered to functional assembly 130: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as neutralizing fluid is delivered into functional assembly 130 (e.g. via one or more lumens or other conduits of catheter 100), fluid is simultaneously evacuated from functional assembly 130 (e.g. via one or more lumens or other conduits of catheter 100), at flow rates such that functional assembly 130 remains expanded (e.g. remains in contact with surrounding mucosal tissue of the duodenum or other GI mucosal tissue), but fluid within functional assembly 130 is recirculated.

Once functional assembly 130 is filled with the ablative fluid (e.g. for a time period of no more than 5 seconds, no more than 4 seconds, and/or no more than 3 seconds), that particular volume of ablative fluid can remain in place (e.g. without removal or replacement) throughout the remaining portion of STEP 5030, and/or it can be recirculated, as described herein, for the remaining portion of STEP 5030.

Tissue proximate functional assembly 130 is ablated or otherwise treated as long as ablative fluid is maintained within functional assembly 130 (e.g. in a stagnant or recirculating manner), and functional assembly 130 is in relative contact with the tissue.

In some embodiments, ablative fluid is delivered to functional assembly 130 in a recirculating manner, for a pre-determined time period, such as a time period of at least 5 seconds, 7 seconds, or 10 seconds.

In some embodiments, ablative fluid is introduced into functional assembly 130 immediately after completion of STEP 5020, without evacuation of the neutralizing fluid introduced in STEP 5020.

In some embodiments, ablative fluid is introduced into a tissue-contacting functional assembly 130 in a recirculating manner, for a calculated time period, the “ablation time”, that is based on the temperature of cold neutralizing fluid delivered to functional assembly 130 in STEP 5020. For example, the colder the temperature of the neutralizing fluid, the longer the ablation time, and vice versa. Alternatively or additionally, the ablation time can be based on the time that the neutralizing fluid cools (e.g. extracts heat from) the tissue, the “neutralizing time”. In some embodiments, the ablation time is also based on the temperature of the ablative fluid (e.g. the hotter the fluid the shorter the ablation time, and vice versa). In some embodiments, the ablation time is based on the information provided in Table A below, such as when the neutralizing time is at least 5 seconds, at least 10 seconds, or approximately 15 seconds:

TABLE A

| Cold Reservoir Temp (° C.) | Ablation Time (seconds) |
|---|---|
| 9.0-10.9 | 10.0 |
| 11.0-12.9 | 9.8 |
| 13.0-14.9 | 9.6 |
| 15.0-16.9 | 9.4 |
| 17.0-18.9 | 9.2 |
| 19.0-20.9 | 9.0 |
| 21.0-22.9 | 8.8 |
| 23.0-25.0 | 8.6 |

During this tissue ablation procedure, one or more fluid pathway temperatures can be monitored, as described herein, such as to change temperature in a closed-loop fashion, and/or to enter an alert state if a temperature threshold is exceeded.

During this tissue ablation procedure, the pressure within one or more fluid pathways can be monitored, such as to adjust the pressure in a closed-loop fashion, and/or to enter an alert state if a pressure threshold is exceeded. For example, pressure below a first minimum can represent a break of balloon 136 and/or other leak in the fluid pathway. Pressure below a second minimum (similar or dissimilar to the first), can represent that functional assembly 130 is not in adequate contact with the mucosal tissue. Pressure above a maximum can represent an occlusion or restriction (e.g. a kink in catheter 100) has occurred.

Temperature and/or pressure can be monitored by one or more temperature sensor and/or pressure sensor-based functional elements of console 200, connecting assembly 300, and/or catheter 100, as described in detail herein in reference to FIGS. 1 and/or 2.

While STEP 5020 has primarily been described using a cooling fluid, in alternative embodiments, a warming fluid can be delivered to functional assembly 130 (e.g. to neutralize a cryogenic ablation) or an agent configured to neutralize a chemical ablation can be delivered directly to the mucosal tissue surface (e.g. a non-target tissue surface).

As described in reference to a heat ablation, one or more ablation parameters of STEP 5030 can be based on one or more neutralizing parameters of STEP 5020, and vice versa. For example, a cryogenic ablation time can be based on a warming neutralizing temperature and/or neutralizing time. A chemical ablation concentration (e.g. pH), can be based on the concentration of a neutralizing procedure (e.g. a neutralizing procedure performed prior to and/or after the ablation step). An electromagnetic, light, and/or ultrasound ablation can be configured (e.g. adjustment of energy delivery and/or ablation time), based on a neutralizing procedure parameter.

In STEP 5040, an optional step of performing a post-ablation neutralizing procedure is performed (e.g. to tissue in close proximity to functional assembly 130 and/or tissue proximate and/or somewhat remote from this tissue).

The neutralizing procedure of STEP 5040 can be similar to the neutralizing step of STEP 5020. Similarly, the neutralizing step can be performed for a fixed period of time, such as a time of at least 5 seconds, at least 10 seconds, or at least 15 seconds. The neutralizing procedure of STEP 5040 can comprise one or more parameters that are determined by the parameters of the neutralizing procedure of step 5020 and/or the ablation procedure of STEP 5030. Alternatively or additionally, the neutralizing procedure of STEP 5040 can comprise one or more parameters that are used to determine one or more parameters of the procedures of STEPS 5020 and/or 5030.

In some embodiments, STEP 5040 is performed after the delivery of ablative fluid (e.g. STEP 5030) at each target tissue location. In other embodiments, STEP 5040 is performed after the delivery of ablative fluid (e.g. STEP 5030) at all target tissue locations.

After the completion of STEP 5040, or STEP 5030 (if neutralizing procedure of STEP 5040 is not performed), fluid can be withdrawn from functional assembly 130, such as for a fixed time period (e.g. no more than 10 seconds, no more than 8 seconds, and/or approximately 6 seconds), and/or until a particular volume of fluid is evacuated. After the fluid evacuation, functional assembly 130 can be transitioned to the translation state, as described herein.

In some embodiments, the functional assembly 130 of method 2000 of FIG. 8 is configured to deliver one or more different forms of energy to target tissue, such as when functional assembly 130 comprises one or more energy delivery elements configured to deliver an energy form selected from the group consisting of: electromagnetic energy; if energy; light energy; laser light energy; sound energy; ultrasound energy; chemical energy; and combinations thereof. The functional assembly 130 can comprise a balloon (e.g. balloon 136) and/or it can include an array of energy delivery elements (e.g. an array of balloons and/or an array of electrodes). The functional assembly 130 of method 2000 can comprise a treatment length of at least 10 mm long and/or no more than 100 mm long. The functional assembly 130 can comprise an expanded diameter of at least 20 mm and/or no more than 40 mm, or no more than 30 mm.

In some embodiments, the patient identified in STEP 2010 of method 2000 of FIG. 8 has been diagnosed with a medical condition selected from the group consisting of: Type 2 diabetes; Type 1 diabetes; “Double diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's disease; neuropathy; diabetic nephropathy; retinopathy; heart disease; diabetic heart disease; heart failure; diabetic heart failure; hirsutism; hyperandrogenism; fertility issues; menstrual dysfunction; cancer such as liver cancer, ovarian cancer, breast cancer, endometrial cancer, cholangiocarcinoma, adenocarcinoma, glandular tissue tumor(s), stomach cancer, large bowel cancer, and/or prostate cancer; diastolic dysfunction; hypertension; myocardial infarction; microvascular disease related to diabetes; sleep apnea; arthritis; rheumatoid arthritis; hypogonadism; insufficient total testosterone levels; insufficient free testosterone levels; and combinations of one or more of these. For example, system 10 and methods 2000, 3000, and/or 4000 of FIGS. 8, 9, and/or 10, respectively, can be used to treat patients with NAFLD/NASH and Type 2 diabetes, such as is described herein in reference to the NAFLD/NASH Study.

In some embodiments, method 2000 of FIG. 8 is performed on a patient with NAFLD/NASH and Type 2 diabetes, and one or more of the following results are achieved (e.g. at a time period of approximately 3 months); hepatic fat is lowered, such as by at least 10%, at least 20%, at least 30%, and/or approximately 36%; HbA1C is lowered, such as by at least 0.5%, at least 0.7%, and/or approximately 1%; both hepatic fat and HbA1C is lowered; triglycerides are lowered, such as by at least 10%, at least 20%, and/or approximately 28%; patient weight is lowered, such as by at least 2%, at least 3%, and/or at least 5% of weight present prior to performance of the procedure of the present inventive concepts (e.g. without lifestyle intervention); weight loss of approximately 3.1 kg is achieved (e.g. without lifestyle intervention); and combinations of these. In some embodiments, these and other clinical benefits described herein are achieved within 3 months of performance of the procedure, and the benefits are present at least 6 months after the performance of the procedure. In some embodiments, results achieved include both improved HbA1C (e.g. reduced at least 0.5%) and improved liver fat content (e.g. reduced at least 20%, such as determined via MRI-PDFF). In these embodiments, HbA1C can be reduced at least 0.7% and liver fat reduced at least 30%.

In some embodiments, the results achieved immediately herein are dependent on a minimum amount of mucosal tissue (e.g. duodenal mucosal tissue) being treated (e.g. ablated, denatured, removed, and/or otherwise treated). For example, a single or cumulative (multiple treatment) axial length of at least 3 cm, at least 6 cm, at least 8 cm, and/or at least 9 cm of duodenal mucosa is treated to achieve these clinical benefits. Alternatively, at least 30% of the post-papillary duodenal mucosa is treated.

The methods of the present inventive concepts can result in (e.g. cause) one or more of the following outcomes (e.g. outcomes related to the clinical benefits described herein): a reduction of surface area of mucosal tissue proximate the treated locations; an altering of hormonal signaling of the intestine proximate the treated location; replacement of the treated mucosal tissue with new tissue; a reduction in iron absorption; a reduction or increase in bile acid signaling; an altering of microbiome composition; a reduction in glucose, fat, and/or amino acid signaling and/or absorption; a reduction in GIP levels in the fasting state (e.g. by at least 5%, 10%, and/or 20%); a reduction in GIP levels in the post-prandial state (e.g. by at least 5%, 10%, and/or 20%); an increase in GLP-1 levels in the post-prandial state (e.g. by at least 5%, 10%, and/or 15%); an increase in GLP-1 levels in the post-prandial state (e.g. while not significantly altering GLP-1 levels in the fasting state); and combinations of one, two, three, or more of these.

Applicant has conducted a particular clinical study, the “NAFLD/NASH Study”, using system 10 of the present inventive concepts. In the NAFLD/NASH study, system 10 was used to treat patients afflicted with both NAFLD/NASH and Type 2 diabetes. The NAFLD/NASH study was performed using method 2000 described herein in reference to FIG. 8. The treatments of the present invention were performed in 2017 and 2018, in clinical sites in the UK, Italy, Belgium, and Brazil, and include 24 patients treated. The data described immediately herein represent data obtained during a follow-up procedure for each patient that occurred approximately 3 months after the patient's mucosal tissue treatment procedure.

In the NAFLD/NASH study, system 10 was used to treat mucosal tissue of each patient's duodenum. Previous studies by applicant in patients with Type 2 diabetes have demonstrated sustained improvements in blood glucose levels and insulin resistance measures through 1 year of follow-up. Results of the NAFLD/NASH study demonstrate improvements in hepatic fat fraction, glycemic profiles, and lipid profiles. Data from these patients show that the metabolic benefits extend to NAFLD/NASH, lowering liver fat by 36%. Nearly 95% of treated patients showed improvement in either glucose or liver fat, with 88% of patients showing improvement in both within 3 months of treatment. Additional benefits from the system 10 procedure were improvement in cardiovascular risk (lowering triglyceride/HDL ratio by 28%), and in weight, with 3.1 kg weight loss unaided by any lifestyle intervention. As with applicant's previous studies in Type 2 diabetes patients, the procedures performed using system 10 were well-tolerated and proved to be safe in this study of patients with both NAFLD/NASH and Type 2 diabetes.

Results of an applicant-sponsored human clinical study using the systems, devices, and methods of the present inventive concepts are presented immediately herein. In the study, 24 patients received 5 ablations via a functional assembly 130 with a treatment length of 20 mm. Procedure time was reduced from 67 minutes to 45 minutes. Successful ablations resulted in 97% of those intended. Results at 3 months for the treated patients were as follows: mean HbA1c level was reduced by 1% (e.g. an HbA1c relative reduction of 12%) with a responder rate of 19/23 (83%); mean c-Peptide level was reduced by 0.5 ng/ml (16% reduction) with a responder rate of 17/24 (70%); mean HOMA-IR level was reduced by 1.9 (31.6% reduction) with a responder rate of 17/21 (81%); mean ALT level was reduced by 10.4 units/liter (29% reduction) with a responder rate of 18/23 (78%); mean PDFF level was reduced by 7% (37.6% reduction) with a responder rate of 16/17 (94%); 10 yr-ASCVD had a responder rate of 11/21 (52%); mean TG/HDL ratio was reduced by 1.53 (33.5% reduction) with a responder rate of 20/23 (87%); mean ferritin level was reduced by 22 ng/ml (24% reduction) with a responder rate of 23/23 (100%); and mean weight level was reduced by 3.1 kg (3% reduction) with a responder rate of 19/24 (80%).

Referring now to FIG. 12, a schematic view of a console operably attached to a connecting assembly and a treatment device is illustrated, consistent with the present inventive concepts. Console 200 of FIG. 12 comprises hot module 2100, cold module 2200, and switching module 2300. Hot module 2100 comprises an assembly configured to provide heated ablative fluid, hot fluid 2101, to balloon 136 of catheter 100. In some embodiments, at least a portion of hot fluid 2101 that has been provided by hot module 2100 is returned to hot module 2100, to be subsequently provided by hot module 2100 as hot fluid 2101 (e.g. in a recirculating manner). In some embodiments, other fluids provided by console 200 are delivered to hot module 2100 for subsequent heating and delivery from hot module 2100 as hot fluid 2101. Cold module 2200 comprises an assembly configured to provide neutralizing fluid (e.g. cooled or room temperature fluid), cold fluid 2201, to balloon 136 of catheter 100. In some embodiments, at least a portion of cold fluid 2201 that has been provided by cold module 2200 is returned to cold module 2200, to be subsequently provided by cold module 2200 as cold fluid 2201 (e.g. in a recirculating manner). In some embodiments, other fluids provided by console 200 are delivered to cold module 2200 for subsequent cooling (if applicable) and delivery from cold module 2200 as cold fluid 2201. Hot fluid 2101 and/or cold fluid 2201 can each comprise water, saline, and/or other fluid. Switching module 2300 comprises an assembly including one or more valves (e.g. switching valves), as well as one or more sensors (e.g. pressure and/or temperature sensors). Switching module 2300 can be configured to monitor one or more parameters of and/or direct the flow of fluid (e.g. hot fluid 2101 and/or cold fluid 2201) between modules 2100, 2200, and catheter 100. Console 200 further comprises controller 250. Controller 250 can comprise a special purpose computer configured to receive signals from one or more sensors of console 200, and/or to control one or more fluid pumping components (e.g. pumps) and/or valves of console 200. Controller 250 can be of a similar construction and arrangement to controller 250 as described herein in reference to FIG. 1 and/or FIG. 2. Console 200 comprises various fluid pathways that fluidly connect the components of hot module 2100, cold module 2200, and switching module 2300.

Hot module 2100 can comprise a fluid-storage vessel, reservoir 2110, which can be configured to store a volume of hot fluid 2101. Reservoir 2110 can comprise a fluid level sensor, sensor 2111, configured to provide a signal to controller 250 relating to the level of hot fluid 2101 in reservoir 2110. Hot module 2100 can comprise heater 2120. Heater 2120 can be configured to heat hot fluid 2101 within reservoir 2110. Heater 2120 can comprise a heating element selected from the group consisting of: resistive, such as electrical; thermoelectric, such as an element positioned on the opposite end of a thermoelectric cooler; chemical, such as an element that utilizes an exothermic reaction; combustion-based, such as an element that burns fuel; microwave; and combinations of these. In some embodiments, heater 2120 comprises an inline heater, such as a heater configured to heat hot fluid 2101 as it is pumped through a portion of heater 2120. In the illustrated embodiment, heater 2120 comprises a heater configured to conductively heat hot fluid 2101 within reservoir 2110 by applying thermal energy to hot fluid 2101. Alternatively or additionally, heater 2120 can be constructed and arranged such that no component or element of heater 2120 contacts hot fluid 2101, such as when heater 2120 utilizes a microwave heating method. In this embodiment, the more expensive components or elements of heater 2120 are constructed and arranged for durability (e.g. reused as they are non-fluid-contacting) and the less expensive components or elements of heater 2120 are constructed and arranged for disposability.

Hot module 2100 can comprise a first fluid pump, pressurization source, and/or other fluid propulsion mechanism (“pump” or “fluid pump” herein), hot pump 2130. Hot pump 2130 can be configured to pump hot fluid 2101 from reservoir 2110. Hot module 2100 can comprise an intake manifold, intake 2131, which is connected to an intake portion of hot pump 2130. In some embodiments, intake 2131 includes a filter configured to prevent debris or other elements from reaching and/or accumulating in hot pump 2130 and/or catheter 100. Intake 2131 can be positioned within reservoir 2110, such that hot fluid 2101 can be drawn from reservoir 2110 by hot pump 2130. In some embodiments, intake 2131 is positioned proximate the center of reservoir 2110, such as to minimize temperature variations in hot fluid 2101 drawn via intake 2131, such as temperature variations which may be more prevalent proximate the walls of reservoir 2110. In some embodiments, hot module 2100 comprises regulator 2132 comprising an assembly fluidly attached to the output of hot pump 2130 and configured to regulate the pressure of hot fluid 2101. Regulator 2132 can be configured to regulate the pressure of hot fluid 2101 at the output of regulator 2132, such as to regulate the pressure to no more than 50 psi, such as no more than 60 psi. Hot module 2100 can further comprise one or more sensors, sensor 2133, fluidly connected to the output of hot pump 2130 (e.g. constructed and arranged between hot pump 2130 and regulator 2132, as shown), such as to monitor the fluid pressure and/or temperature at the output of hot pump 2130. Regulator 2132 can comprise an over-pressure valve, such as a valve configured to release hot fluid 2101 back into reservoir 2110 if the pressure of hot fluid 2101 exceeds a threshold. In some embodiments, the pressure threshold comprises a threshold of no more than 60 psi. In some embodiments, regulator 2132 comprises a mechanical pressure regulator configured to regulate the pressure of hot fluid 2101 at the output of regulator 2132 (e.g. when hot pump 2130 is configured to pump hot fluid 2101 at a pressure above the regulated pressure). In some embodiments, hot pump 2130 is configured to pump hot fluid 2101 at a pressure of at least 5 psi, such as at a pressure between 5 psi to 50 psi under normal operating conditions (e.g. conditions needed to perform the various system 10 procedures). Regulator 2132 can be configured to relieve (e.g. reduce) pressure within hot pump 2130, for example if hot pump 2130 malfunctions and/or if there is an obstruction in the fluid path, and the pressure within hot pump 2130 rises above that threshold.

Hot module 2100 can further comprise a switching valve, hot router 2140. Hot router 2140 can comprise an inlet 21401, a first outlet 214001, and a second outlet 214002. The inlet 21401 of hot router 2140 can be fluidly attached to the output of hot pump 2130 (e.g. via regulator 2132, as shown). Hot router 2140 can be configured in either of a recirculating arrangement or a sourcing arrangement. When in the recirculating arrangement, hot router 2140 is configured to direct hot fluid 2101 back to reservoir 2110, as the inlet 21401 is fluidly connected to the first outlet 214001, which is fluidly connected to reservoir 2110. When in the sourcing arrangement, hot router 2140 is configured to direct hot fluid 2101 to switching module 2300, as the inlet 21401 is fluidly connected to the second outlet 214002, and the fluid can be further directed as described herein. In some embodiments, hot router 2140 is in an unpowered or otherwise deactivated state when in the recirculating arrangement, and in an activated state (e.g. a powered state) when in the sourcing arrangement. Alternatively or additionally, hot router 2140 is in the sourcing arrangement when in a deactivated state, and/or hot router 2140 can comprise a valve configured to remain in a current state when power is withdrawn, and only change between circulating and sourcing arrangements under powered control.

In some embodiments, hot module 2100 further comprises a second fluid pump, hot return pump 2150. Hot return pump 2150 can be configured to actively draw fluid (e.g. returning hot fluid 2101) from switching module 2300, and can be configured to return hot fluid 2101 to reservoir 2110. Hot return pump 2150 can be configured to limit the pressure of hot fluid 2101 within balloon 136. In some embodiments, hot return pump 2150 limits the pressure of hot fluid 2101 within balloon 136 by actively drawing hot fluid 2101 from balloon 136, for example, when hot pump 2130 is pumping hot fluid 2101 into balloon 136, as described herein. In some embodiments, hot module 2100 comprises a buffer for the returning fluid, buffer 2151. Buffer 2151 can be configured to prevent returning fluid from concentrating within one or more localized regions of reservoir 2110, for example to prevent the returning fluid (e.g. at a lower temperature than hot fluid 2101) from concentrating at a region proximate intake 2131. Buffer 2151 can be configured to prevent the returning fluid from adversely altering (e.g. cooling) the temperature of fluid entering intake 2131 (e.g. adversely altering the temperature of hot fluid 2101). In some embodiments, buffer 2151 comprises a chamber positioned proximate (e.g. inside of) reservoir 2110, the chamber comprising at least one porous surface that can be configured to allow fluid within buffer 2151 to slowly mix with the fluid inside reservoir 2110 (e.g. to mix more slowly than returning fluid pumped directly into reservoir 2110). Alternatively or additionally, pump 2150 can be configured to pump returning fluid into reservoir 2110 at a location away from intake 2131, such as to decrease the probability of returning fluid flowing directly into intake 2131 (e.g. with or without buffer 2151). Additionally still, reservoir 2110 can comprise a fluid mixing element, mixer 2160, configured to continuously or semi-continuously stir hot fluid 2101 within reservoir 2110. The stirring of hot fluid 2101 within reservoir 2110 can help to maintain a homogeneous temperature of hot fluid 2101 within reservoir 2110 (e.g. regardless of temperature differences in fluid returned to reservoir 2110). In some embodiments, hot module 2100 further comprises one or more additional sensors, sensors 2134 and 2135, fluidly connected to reservoir 2110 and to the output of hot return pump 2150, respectively, such as to monitor the fluid pressure and/or temperature at those locations.

Cold module 2200 can comprise a fluid-storage vessel, reservoir 2210, which can be configured to store a volume of cold fluid 2201. Reservoir 2210 can comprise a fluid level sensor, sensor 2211, configured to provide a signal to controller 250 relating to the level of cold fluid 2201 in reservoir 2210.

Cold module 2200 can comprise a first fluid pump, cold pump 2230. Cold pump 2230 can be configured to pump cold fluid 2201 from reservoir 2210. Cold module 2200 can comprise an intake manifold, intake 2231, which is connected to an intake portion of cold pump 2230. Intake 2231 can be positioned within reservoir 2210 such that cold fluid 2201 can be drawn from reservoir 2210 by cold pump 2230. In some embodiments, intake 2231 is positioned proximate the center of reservoir 2210, such as to minimize temperature variations in cold fluid 2201 drawn via intake 2231, such as temperature variations which may be more prevalent proximate the walls of reservoir 2210. In some embodiments, cold module 2200 comprises regulator 2232 comprising an assembly fluidly attached to the output of cold pump 2230 and configured to regulate the pressure of cold fluid 2201. Regulator 2232 can be configured to regulate the pressure of cold fluid 2201 at the output of regulator 2232, such as to regulate the pressure to no more than 50 psi, such as no more than 60 psi. Cold module 2200 can further comprise one or more sensors, sensor 2233, fluidly connected to the output of cold pump 2230 (e.g. constructed and arranged between cold pump 2230 and regulator 2232 as shown), such as to monitor the fluid pressure and/or temperature at the output of cold pump 2230. Regulator 2232 can comprise an over-pressure valve, such as a valve configured to release cold fluid 2201 back into reservoir 2210 if the pressure of cold fluid 2201 exceeds a threshold. In some embodiments, the pressure threshold comprises a threshold of no more than 60 psi. In some embodiments, regulator 2232 comprises a mechanical pressure regulator configured to regulate the pressure of cold fluid 2201 at the output of regulator 2232 (e.g. when cold pump 2230 is configured to pump cold fluid 2201 at a pressure above the regulated pressure). In some embodiments, cold pump 2230 is configured to pump cold fluid 2201 at a pressure of between 5 psi to 50 psi under normal operating conditions. Regulator 2232 can be configured to relieve (e.g. reduce) pressure within cold pump 2230, for example if cold pump 2230 malfunctions and/or if there is an obstruction in the fluid path, and the pressure rises above that threshold.

Cold module 2200 can further comprise chiller assembly 2270, which is configured to remove thermal energy (e.g. chill) from cold fluid 2201 as cold fluid 2201 flows through a portion of chiller assembly 2270. Chiller assembly can comprise heat exchanger 2271 and chiller 2272. Heat exchanger 2271 can comprise at least one fluid lumen and can be configured to be placed inline with the fluid path of cold fluid 2201 within cold module 2200. Chiller 2272 can comprise a thermoelectric cooler, such as a Peltier cooler, operably attached to heat exchanger 2271 and configured to chill heat exchanger 2271. In some embodiments, chiller assembly 2270 includes a pump configured to circulate a coolant through heat exchanger 2271.

Cold module 2200 can further comprise a switching valve, cold router 2240. Cold router 2240 can comprise an inlet 22401, a first outlet 224001, and a second outlet 224002. The inlet 22401 of cold router 2240 can be fluidly attached to the output of cold pump 2230 (e.g. via regulator 2232 and chiller assembly 2270, as shown). Cold router 2240 can be configured in either of a recirculating arrangement or a sourcing arrangement. When in the recirculating arrangement, cold router 2240 is configured to direct cold fluid 2201 back to reservoir 2210, as the inlet 22041 is fluidly connected to the first outlet 224001, which is fluidly connected to reservoir 2210. When in the sourcing arrangement, cold router 2240 is configured to direct cold fluid 2201 to switching module 2300, as the inlet 22401 is fluidly connected to the second outlet 224002, and the fluid can be further directed as described herein. In some embodiments, cold router 2240 is in an unpowered or otherwise deactivated state when in the recirculating arrangement, and in an activated state (e.g. a powered state) when in the sourcing arrangement. Alternatively or additionally, cold router 2240 is in the sourcing arrangement when in a deactivated state, and/or cold router 2240 can comprise a valve configured to remain in a current state when power is withdrawn, and only change between circulating and sourcing arrangements under powered control.

In some embodiments, cold module 2200 further comprises a second fluid pump, cold return pump 2250. Cold return pump 2250 can be configured to actively draw fluid (e.g. returning cold fluid 2201) from switching module 2300, and to return cold fluid 2201 to reservoir 2210. Cold return pump 2250 can be configured to limit the pressure of cold fluid 2201 within balloon 136. In some embodiments, cold return pump 2250 limits the pressure of cold fluid 2201 within balloon 136 by actively drawing cold fluid 2201 from balloon 136, for example, when cold pump 2230 is pumping cold fluid 2201 into balloon 136, as described herein. In some embodiments, cold module 2200 comprises a buffer for the returning fluid, buffer 2251. Buffer 2251 can be configured to prevent returning fluid from concentrating within one or more localized regions of reservoir 2210, for example to prevent the returning fluid (e.g. at a higher temperature than cold fluid 2201) from concentrating at a region proximate intake 2231. Buffer 2251 can be configured to prevent this returning fluid from adversely altering (e.g. warming) the temperature of fluid entering intake 2231 (e.g. adversely altering the temperature of cold fluid 2201). In some embodiments, buffer 2251 comprises a chamber positioned proximate (e.g. inside of) reservoir 2210, the chamber comprising at least one porous surface that can be configured to allow fluid within buffer 2251 to slowly mix with the fluid inside reservoir 2210 (e.g. to mix more slowly than returning fluid pumped directly into reservoir 2210). Alternatively or additionally, pump 2250 can be configured to pump returning fluid into reservoir 2210 at a location away from intake 2231, such as to decrease the probability of returning fluid flowing directly into intake 2231 (e.g. with or without buffer 2251). Additionally still, reservoir 2210 can comprise a fluid mixing element, mixer 2260, configured to continuously or semi-continuously stir cold fluid 2201 within reservoir 2210. The stirring of cold fluid 2201 within reservoir 2210 can help to maintain a homogeneous temperature of cold fluid 2201 within reservoir 2210 (e.g. regardless of temperature differences in fluid returned to reservoir 2210). In some embodiments, cold module 2200 further comprises one or more additional sensors, sensors 2234 and 2235, fluidly connected to reservoir 2210 and to the output of cold return pump 2250, respectively, such as to monitor the fluid pressure and/or temperature at those locations.

In some embodiments, cold module 2200 comprises heater 2220. Heater 2220 can be configured to heat cold fluid 2201 within reservoir 2210. Heater 2220 can comprise a heating element selected from the group consisting of: resistive, such as electrical; thermoelectric, such as an element positioned on the opposite end of a thermoelectric cooler; chemical, such as an element that utilizes an exothermic reaction; combustion-based, such as an element that burns fuel;

microwave; and combinations of two or more of these. In some embodiments, heater 2220 comprises an inline heater, such as a heater configured to heat cold fluid 2201 as it is pumped through a portion of heater 2220. In the illustrated embodiment, heater 2220 comprises a heater configured to conductively heat cold fluid 2201 within reservoir 2210 by applying thermal energy to cold fluid 2201. In some embodiments, cold module 2200 is configured to continuously or semi-continuously recirculate cold fluid 2201 through chiller assembly 2270, and as such cold fluid 2201 may exceed a desired temperature threshold (e.g. become too cold). Heater 2220 can be configured to apply thermal energy to cold fluid 2201 should such a threshold be exceeded. Alternatively, cold module 2200 can be configured to selectively circulate cold fluid 2201 through chiller assembly 2270, such that cold fluid 2201 does not exceed the threshold of a desired temperature for cold fluid 2201. In some embodiments, cold module 2200 is configured to selectively circulate cold fluid 2201 through chiller assembly 2270 and/or to apply heat to cold fluid 2201, such as to maintain cold fluid 2201 below a viscosity threshold (e.g. to prevent the viscosity from increasing above the threshold due to a decrease in the temperature of cold fluid 2201). In some embodiments, cold fluid 2201 is maintained above a minimum temperature of 10° C., or 15° C., such as to be below a viscosity threshold.

Catheter 100 comprises handle 102, positioned at the proximal end of shaft 110. Attached to the distal portion of shaft 110, functional assembly 130 comprises balloon 136. Catheter 100 can comprise a first conduit, inflow conduit 1011, and a second conduit, outflow conduit 1051. The proximal ends of conduits 1011, 1051 each comprise a connector, inflow connector 1012 and outflow connector 1052, respectively. Connectors 1012, 1052 can be positioned proximate handle 102, such as when at least a portion of conduits 1011, 1051 extend beyond the proximal end of handle 102, as shown. Alternatively, connectors 1012, 1052 can be integral to handle 102. Conduits 1011, 1051 can each extend distally through handle 102, through shaft 110, to terminate at and/or within balloon 136. Conduit 1011 is fluidly connected to balloon 136 via a first port, inflow port 1013, positioned within balloon 136. Conduit 1051 is fluidly connected to balloon 136 via a second port, outflow port 1053, positioned within balloon 136.

Console 200 can comprise housing 210. Housing 210 can surround one or more components, assemblies, and/or modules of console 200. Housing 210 can comprise bulkhead 217, a portion of housing 210 through which one or more fluid connectors are positioned. As shown in FIG. 12, bulkhead 217 can comprise five connectors, each configured to fluidly connect a component of system 10 to switching module 2300 within console 200. In some embodiments, bulkhead 217 comprises more or less than five connectors. Bulkhead 217 can comprise a first connector, hot out connector 2311, and a second connector, cold out connector 2321. Switching module 2300 fluidly connects the second output of hot router 2140 to hot out connector 2311, and fluidly connects the second output of cold router 2240 to cold out connector 2321. Switching module 2300 can further comprise one or more sensors, sensor 2312, fluidly connected to hot out connector 2311, such as to monitor the pressure and/or temperature of hot fluid 2101 between hot router 2140 and hot out connector 2311 (e.g. the pressure and/or temperature of hot fluid 2101 at connector 2311 and leaving console 200). Switching module 2300 can further comprise an additional one or more sensors, sensor 2322, fluidly connected to cold out connector 2321, such as to monitor the pressure and/or temperature of cold fluid 2201 between cold router 2240 and cold out connector 2321 (e.g. the pressure and/or temperature of cold fluid 2201 at connector 2321 and leaving console 200). Hot out connector 2311 fluidly attaches to a first connector, hot connector 3011, of connecting assembly 300. Cold out connector 2321 fluidly attaches to a second connector, cold connector 3021, of connecting assembly 300. Hot connector 3011 and cold connector 3021 can each be attached to a fluid conduit within connecting assembly 300. In some embodiments, as shown in FIG. 12, the two fluid lumens of connector assembly 300 attached to connectors 3011, 3021 can merge (such as via a T-body or Y-body connector) within connecting assembly 300, such that for the majority of the length of connecting assembly 300, a single inflow conduit, conduit 3111, carries fluid (e.g. hot fluid 2101 and/or cold fluid 2201) distally, towards catheter 100. On its distal end, conduit 3111 fluidly attaches to a first distal connector of connecting assembly 300, feed connector 3211. Feed connector 3211 fluidly attaches to inflow connector 1012 of catheter 100. One or more fluids (e.g. hot fluid 2101 or cold fluid 2201) can be introduced into catheter 100 via conduit 3111, as described herein.

Switching module 2300 can comprise a first valve, evacuation valve 2331, configured to control a fluid connection between a source of negative pressure, vacuum assembly 2440, and a third connector, evacuation connector 2332, of bulkhead 217. Vacuum assembly 2440 can comprise a source of negative pressure, vacuum pump 2441, fluidly attached to drain canister 2442. Drain canister 2442 is fluidly (e.g. via a fluid conduit, such as a vacuum tube) attached to vacuum connector 2443, which is configured to operably attach to a fourth connector, vacuum connector 2341, of bulkhead 217. Vacuum connector 2341 can be fluidly attached to evacuation valve 2331. Evacuation connector 2332 is fluidly attached to evacuation valve 2331, such that when evacuation valve 2331 is in an open configuration, evacuation connector 2332 is fluidly attached to vacuum assembly 2440. Connecting assembly 300 can comprise a third connector, shunt connector 3032, configured to fluidly attach to evacuation connector 2332. Shunt connector 3032 is fluidly attached to a distal portion of conduit 3111, proximate the distal end of connection assembly 300 via a first return conduit, shunt conduit 3112. Conduit 3112 can be utilized to circulate fluid within connecting assembly 300 without fluid entering catheter 100, as described herein.

Switching module 2300 can further comprise a return switching valve, return bypass 2351. Return bypass 2351 can comprise an inlet 23511, a first outlet 235101, and a second outlet 235102. The inlet 23511 of return bypass 2351 can be fluidly attached to a fifth connector, return connector 2352, of bulkhead 217. Switching module 2300 can further comprise an additional one or more sensors, sensor 2353, fluidly connected to return connector 2352, such as to monitor the pressure and/or temperature of fluid returning to console 200 from catheter 100. Return connector 2352 fluidly attaches to a fourth connector, return connector 3051, of connecting assembly 300. Return connector 3051 fluidly attaches to a second distal connector of connecting assembly 300, return connector 3251, via a second return conduit, conduit 3151. Return connector 3251 fluidly attaches to outflow connector 1052 of catheter 100. One or more fluids (e.g. hot fluid 2101 and/or cold fluid 2201) can be removed from catheter 100 via conduit 3151, as described herein. In some embodiments, connector assembly 300 comprises one or more sensors, sensors 3113 and 3153, fluidly connected to conduit 3111 and to conduit 3151, respectively, such as to monitor the fluid pressure and/or temperature at those locations.

Console 200 can comprise an additional fluid source, low-pressure assembly 2500, configured to provide and/or withdraw fluid from catheter 100. Low-pressure assembly 2500 can be configured to provide and/or withdraw a fixed volume of fluid from catheter 100. In some embodiments, low-pressure assembly 2500 comprises a syringe pump. Low-pressure assembly 2500 can be fluidly attached to the second outlet 235102 of return bypass 2351, via a conduit, bypass conduit 2356. Switching module 2300 can further comprise an additional one or more sensors, sensor 2354, fluidly connected to the output of low-pressure assembly 2500, such as to monitor the pressure and/or temperature of fluid leaving and/or entering low-pressure assembly 2500.

Switching module 2300 can further comprise an additional return switching valve, return selector 2360. Return selector 2360 can comprise a first inlet 236011, a second inlet 236012, and an outlet 23600. The first inlet 236011 of return selector 2360 can be fluidly attached to the first outlet 235101 of return bypass 2351. The second inlet 236012 of return selector 2360 can be fluidly attached to evacuation connector 2332.

Switching module 2300 can further comprise an additional return switching valve, return router 2370. Return router 2370 can comprise a first inlet 23701, a first outlet 237001, and a second outlet 237002. The inlet 23701 of return router 2370 can be fluidly attached to the outlet 23600 of return selector 2360. The first outlet 237001 of return router 2370 can be fluidly attached to return pump 2250 of cold module 2200. The second outlet 237002 of return router 2370 can be fluidly attached to return pump 2150 of hot module 2100. Return router 2370 can be configured in either of a cold return arrangement or a hot return arrangement. When in the cold return arrangement, return router 2370 is configured to direct returning fluid back to cold reservoir 2210 (e.g. via cold return pump 2250), as the inlet 23701 is fluidly connected to the first outlet 237001. When in the hot return arrangement, return router 2370 is configured to direct returning fluid back to hot reservoir 2110 (e.g. via hot return pump 2150), as the inlet 23701 is fluidly connected to the second outlet 237002. In some embodiments, return router 2370 is in an unpowered or otherwise deactivated state when in the hot return arrangement, and in an activated state (e.g. a powered state) when in the cold return arrangement. Alternatively or additionally, return router 2370 is in the cold return arrangement when in a deactivated state, and/or return router 2370 can comprise a valve configured to remain in a current state when power is withdrawn, and only change between circulating and sourcing arrangements under powered control. Return selector 2360 can be configured in either of a balloon return arrangement or a shunt return arrangement. When in the balloon return arrangement, return selector 2360 is configured to fluidly connect the return router 2370 to return connector 2352 (e.g. via return bypass 2351), as the outlet 23600 is fluidly connected to the first inlet 236011. When in the shunt return arrangement, return selector 2360 is configured to fluidly connect the return router 2370 to evacuation connector 2332, as the outlet 23600 is fluidly connected to the second inlet 236012. In some embodiments, return selector 2360 is in an unpowered or otherwise deactivated state when in the balloon return arrangement, and in an activated state (e.g. a powered state) when in the shunt return arrangement. Alternatively or additionally, return selector 2360 is in the shunt return arrangement when in a deactivated state, and/or return selector 2360 can comprise a valve configured to remain in a current state when power is withdrawn, and only change between circulating and sourcing arrangements under powered control. Return bypass 2351 can be configured in either of a pass-through arrangement or a bypass arrangement. When in the pass-through arrangement, return bypass 2351 is configured to fluidly connect return connector 2352 to return selector 2360, as the inlet 23511 is fluidly connected to the first output 235101. When in the bypass arrangement, return bypass 2351 is configured to fluidly connect low-pressure assembly 2500 to return connector 2352, as the inlet 23511 is fluidly connected to the second output 235102. When in the bypass arrangement, low-pressure assembly 2500 is in direct fluid communication with balloon 136 via conduits 3151 and 1051, such that low-pressure assembly 2500 can provide and/or remove fluid directly from balloon 136 in a low-pressure and/or volumetric manner. In some embodiments, return bypass 2351 is in an unpowered or otherwise deactivated state when in the pass-through arrangement, and in an activated state (e.g. a powered state) when in the bypass arrangement. Alternatively or additionally, return bypass 2351 is in the bypass arrangement when in a deactivated state, and/or return bypass 2351 can comprise a valve configured to remain in a current state when power is withdrawn, and only change between circulating and sourcing arrangements under powered control.

In some embodiments, switching module 2300 comprises an additional valve, relief valve 2355. Relief valve 2355 can be configured to provide a fluid connection between the inlet 23701 of return router 2370 and low-pressure assembly 2500 (e.g. when relief valve 2355 is in an open arrangement). In some embodiments, relief valve 2355 comprises an overpressure valve, such as a valve configured to open or otherwise reduce pressure when a threshold pressure is reached and/or exceeded. Alternatively or additionally, relief valve 2355 can comprise an actuator which can be activated in response to a measured pressure reaching and/or exceeding a threshold (e.g. a pressure measured by sensor 2354). In some embodiments, relief valve 2355 is opened to equalize the pressure between second outlet 253102 of return bypass 2351 and inlet 23701 of return router 2370 and/or components (e.g. conduits) of console 200 fluidly attached to inlet 23701 of return router 2370. Relief valve 2355 can comprise a normally open valve, such that relief valve 2355 requires an active electrical signal (e.g. requires power to be applied) to remain in the closed configuration. In these embodiments, in the event of a power failure, relief valve 2355 would automatically open (if in the closed configuration at the time of the loss of power).

In some embodiments, controller 250 of console 200 comprises one or more algorithms, routines, and/or subroutines configured to monitor one or more parameters, functions, configurations, and/or arrangements (“parameters” herein) of system 10. For example, controller 250 can comprise a self-diagnostic routine, continuous system self-monitoring (CSSM) routine, CSSM 255, which can be configured to continuously and/or periodically monitor one, two, or more parameters of system 10. In some embodiments, CSSM 255 comprises a watchdog processor. CSSM 255 can be further configured to trigger an alert mode if a parameter of system 10 is determined to be out of specification. A console 200 alert mode can comprise a mode in which treatments are stopped, fluid is evacuated from catheter 100, treatment neutralizing fluid is delivered (e.g. to balloon 136), and/or one or more audible, visible, and/or tactile alerts are produced by console 200 to alert a clinician or other operator of system 10 of the alert condition. In some embodiments, controller 250 comprises a software component and a firmware component. In these embodiments, the software component of controller 250 can be configured to be executed on a Windows or other operating system, and can be embedded within or otherwise functionally connected to controller 250 and the firmware component of controller 250. In some embodiments, one or more functions of CSSM 255 and/or the control of one or more components of console 200 are executed in firmware. In some embodiments, if the connection between the software component of controller 250 and the firmware component of controller 250 is disconnected or otherwise lost (e.g. if the software shuts down or unexpectedly crashes), the firmware component can continue to run in order to ensure console 200 is in a safe condition (e.g. to ensure that if ablative fluid has been delivered to balloon 136, a neutralizing fluid is subsequently delivered).

In some embodiments, CSSM 255 is configured to cause console 200 and/or one or more other components of system 10 to enter an alert mode if one or more errors (e.g. undesired states) are detected. For example, CSSM 255 can enter an alert mode if one or more of the following is detected within a component of system 10: air; leak; obstruction; and/or occlusion. In some embodiments, when console 200 and/or system 10 is in an alert mode, hot return pump 2150 and/or cold return pump 2250 are turned on (e.g. to a maximum flow rate condition) such as to rapidly draw any fluid (e.g. hot fluid 2101 and/or cold fluid 2201) from catheter 100. Additionally or alternatively, evacuation valve 2331 can be switched to its open configuration, such that vacuum pump 2441 is fluidly connected to balloon 136. In some embodiments, evacuation valve 2331 comprises a normally open configuration, such that in the event of a power loss to console 200, evacuation valve 2331 switches from a powered, closed configuration, to the normal, open configuration. In some embodiments, vacuum assembly 2440 comprises a hospital central vacuum line. In the event of a console 200 power loss, vacuum provided by the hospital central vacuum line is applied to balloon 136. In some embodiments, console 200 comprises a pressure sensor fluidly attached between evacuation valve 2331 and vacuum connector 2341. In these embodiments, CSSM 255 can be configured to monitor the pressure sensor to ensure vacuum assembly 2440 is operably attached to, and providing a source of vacuum to, console 200.

In some embodiments, CSSM 255 monitors the pressure of hot fluid 2101 and/or cold fluid 2201 within console 200, such as via one or more sensors, such as sensor 2133 and/or 2233. CSSM 255 can be configured to cause console 200 to enter into an alert mode if the pressure of the fluid(s) within console 200 rises above a threshold. CSSM 255 can be configured detect a pressure level below the regulated pressure allowed by regulators 2132 and/or 2133, for example, a pressure threshold of at least 50 psi. In some embodiments, if the pressure monitored by CSSM 255 is above a threshold, hot router 2140 and/or cold router 2240 are automatically configured to enter their respective recirculating arrangements, such that high pressure fluid is not directed to balloon 136.

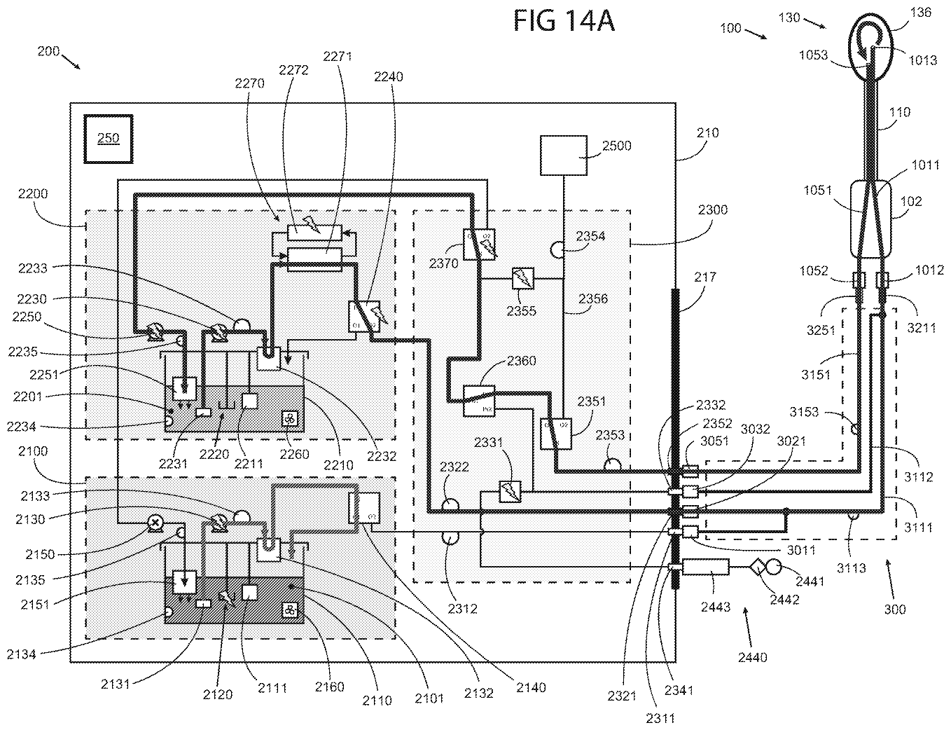

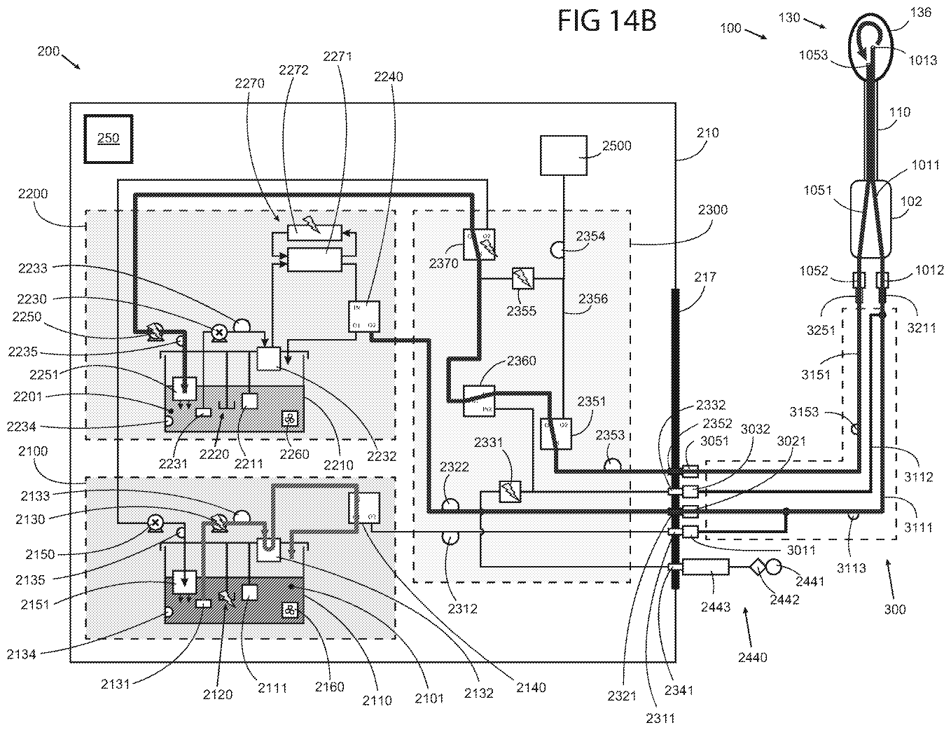

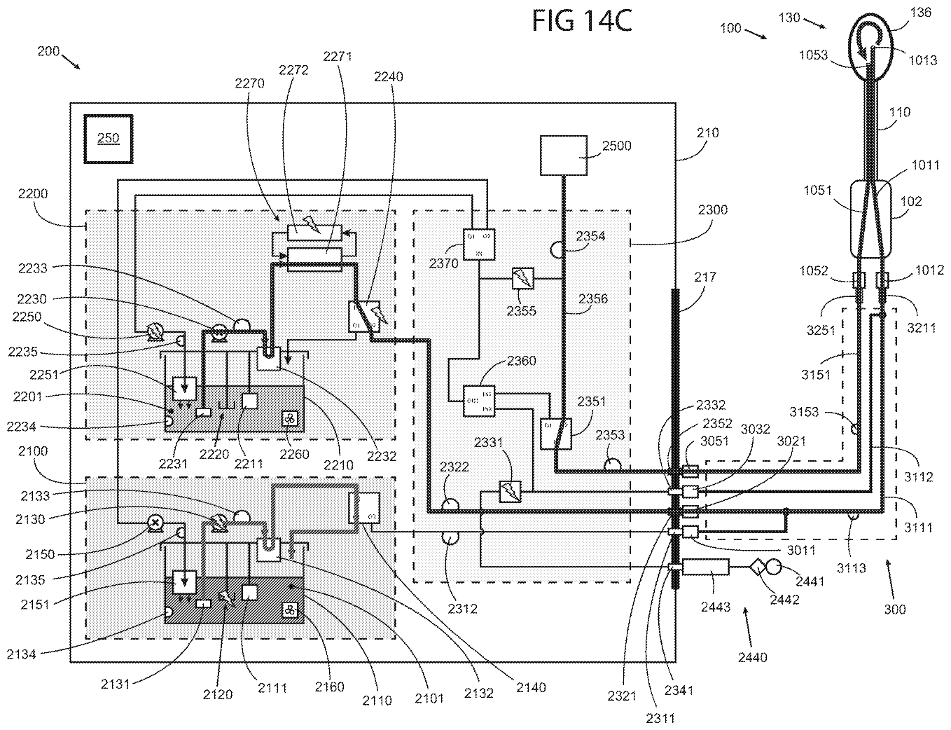

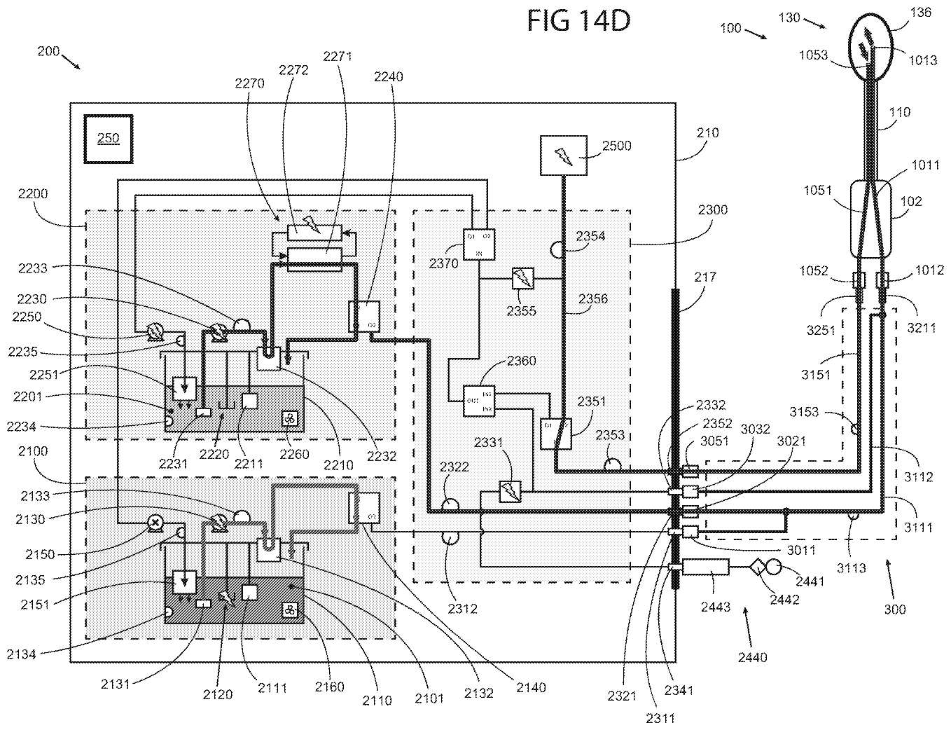

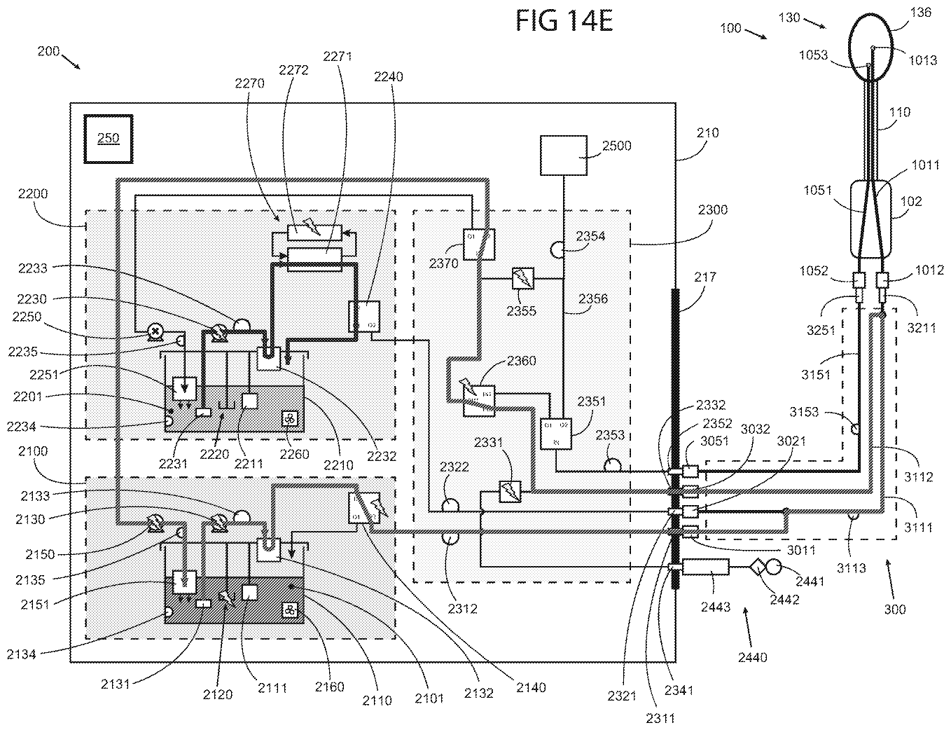

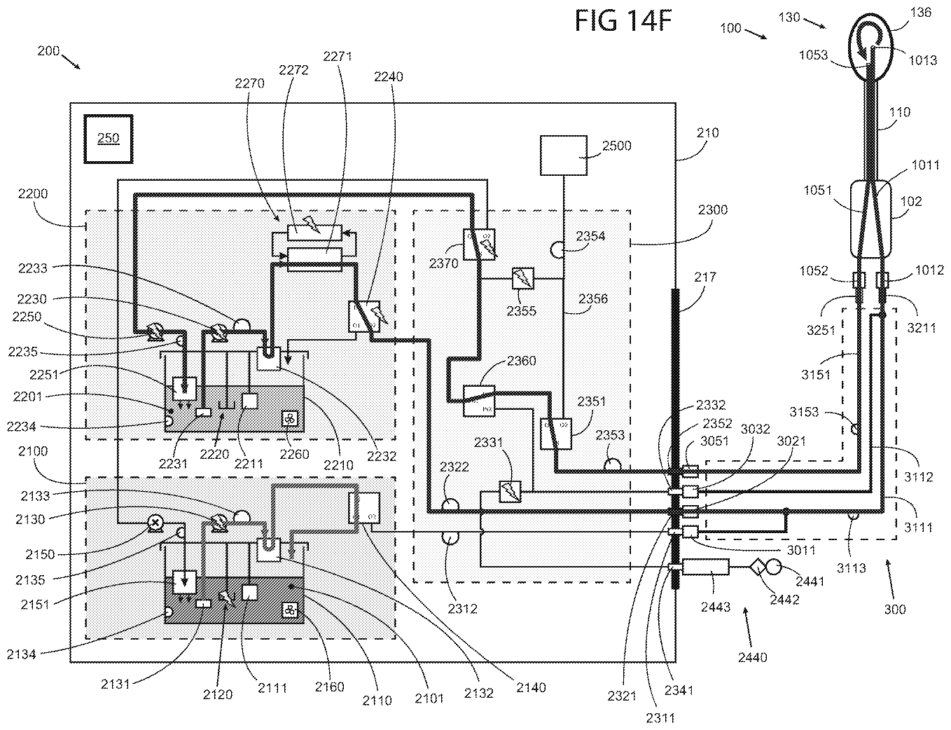

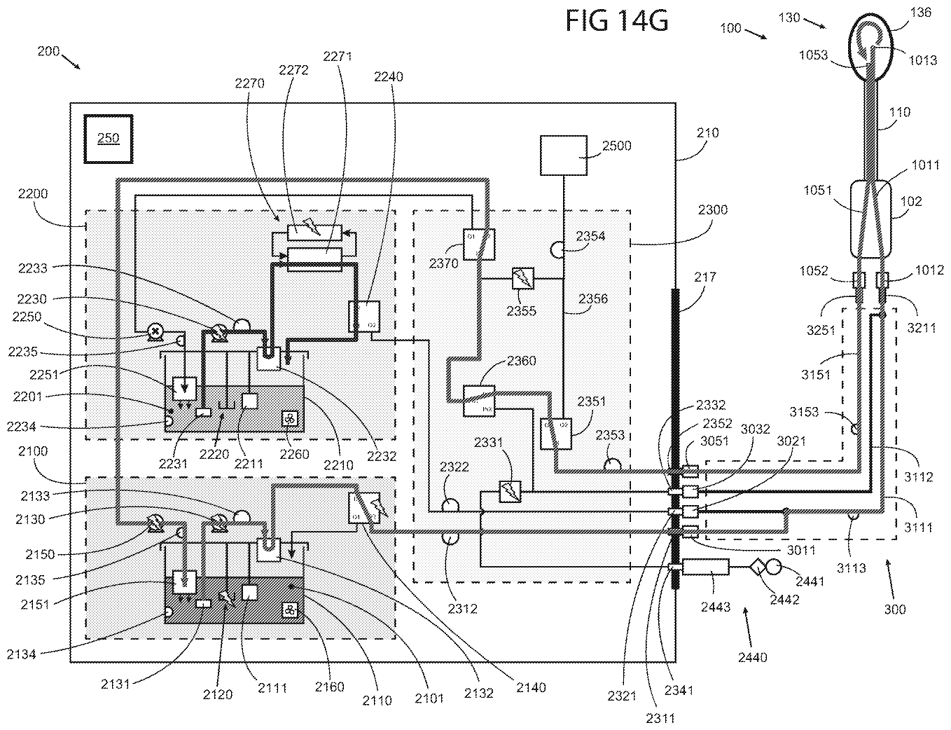

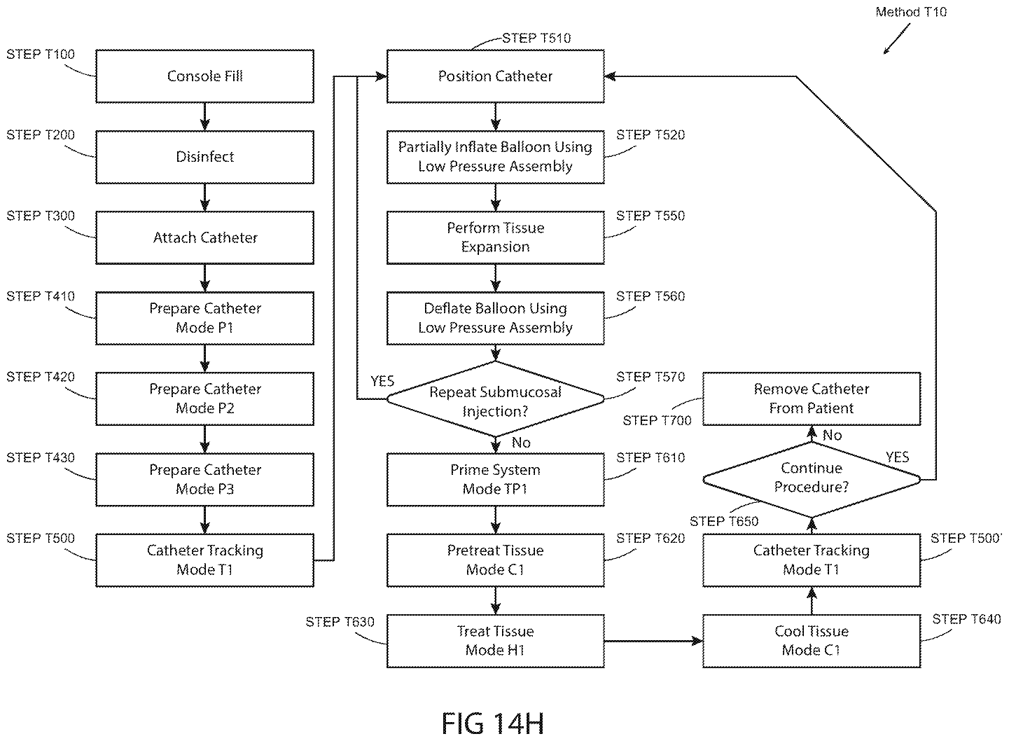

In some embodiments, CSSM 255 monitors the pressure at one or more locations within console 200 and/or connecting assembly 300 prior to and/or after one or more steps of a tissue treatment procedure of the present inventive concepts, such as described herein in reference to FIG. 14H. For example, prior to a treatment step wherein hot fluid 2101 is delivered into balloon 136, CSSM 255 can monitor the pressure in balloon 136 (e.g. via the pressure as measured by a sensor of console 200), such as to ensure no leak is present. In some embodiments, balloon 136 is pressurized with cold fluid 2201 prior to a treatment step during which hot fluid 2101 is delivered into balloon 136, such that any previously undetected leak can be detected using pressurized cold fluid 2201 (e.g. such that leaks are detected prior to balloon 136 being filled with hot fluid 2101). In some embodiments, CSSM 255 detects when a pressure level between 20 psi and 60 psi is present, such as a pressure level between 30 psi and 50 psi, prior to a treatment step in which hot fluid 2101 is delivered into balloon 136. In some embodiments, CSSM 255 is configured to determine the difference between a small leak (e.g. a slow leak), such as a loose connection or a pinhole leak, and a large leak (e.g. a burst balloon). For example, a pressure below 10 psi can indicated a large leak, and a pressure above 10 psi, but less than 30 psi can indicate a small leak.

In some embodiments, one or more sensors of console 200 and/or connecting assembly 300 comprise two or more sensors, such as two or more redundant sensors. For example, sensor 3113 of connecting assembly 300 can comprise two temperature sensors configured in a redundant arrangement. CSSM 255 can be configured to compare the readings from each of any redundant sensors, such as to confirm the functionality of the pair of sensors. In some embodiments, if the readings from any redundant sensors do not match, CSSM 255 is configured to cause console 200 to enter into an alert mode.

In some embodiments, CSSM 255 monitors the fluid temperature and pressure at sensor 2312 at the output of hot module 2100, sensor 2322 at the output of cold module 2200, and sensor 2353 fluidly connected to return connector 2352, comparing these measurements from each sensor to expected values based on the current state of console 200. In some embodiments, CSSM 255 detects clogs, leaks, failed pumps (e.g. hot pump 2130, hot return pump 2150, cold pump 2230, and/or cold return pump 2250), and or failed valves based on the temperature and/or pressure measurements from these sensors. For example, a pressure measurement that is higher than an expected value can indicate a clog in a fluid pathway of console 200 or catheter 100. A pressure measurement that is lower than an expected value can indicate a failed pump and/or a fluid pathway leak. In some embodiments, if a sufficient temperature change at a sensor (e.g. a temperature change at sensor 2353) is not detected within a time period during a particular mode, CSSM 255 identifies an error (e.g. and console 200 enters an alarm state). For example, during a step of a treatment procedure during which hot fluid 2101 is delivered to balloon 136, if the temperature of the returning fluid (e.g. as measured by sensor 2353) does not rise above a threshold within a time window from the start of that step, CSSM 255 identifies an error.

In some embodiments, CSSM 255 monitors one or more parameters of hot reservoir 2110 and/or cold reservoir 2210. CSSM 255 can be configured to trigger an alert mode if the parameters of either or both of reservoirs 2110,2210 are outside of an acceptable range. For example, CSSM 255 can be configured to monitor the fluid level within reservoirs 2110,2210. If the fluid level within either of reservoirs 2110,2210 is below a threshold (e.g. not enough fluid) or above a threshold (e.g. too much fluid), CSSM 255 can be configured to trigger an alert mode. For example, CSSM 255 can trigger an alert mode to lock out all functionality of console 200, such that a user can manually adjust the level of the reservoir. Additionally or alternatively, CSSM 255 can monitor the temperature of the fluid within reservoirs 2110,2210 and trigger an alert mode if the temperature of the fluid exceeds a threshold (e.g. the fluid is too hot or too cold). In some embodiments, CSSM 255 is configured to prevent a procedural step, such as treatment Step T630 described herein in reference to FIG. 14H. For example, CSSM 255 can prevent ablative hot fluid 2101 from being delivered to balloon 136 if the level and/or temperature of cold reservoir 2210 is insufficient to adequately neutralize the heat delivered to tissue during a treatment step. Alternatively or additionally, system 10 can be configured to ablate tissue using non-thermal ablative methods (e.g. chemical ablative methods), and in these embodiments, CSSM 255 can be configured to ensure a sufficient amount of a neutralizing agent (e.g. a base configured to neutralize an acid) is available prior to a treatment step during which an ablative agent is delivered to tissue.

One or more components of console 200, connecting assembly 300, and/or catheter 100 can be selected based on the thermal properties of the component. For example, various conduits of console 200 can comprise conduits with low thermal mass, such as thin walled conduits and/or conduits comprising a material with low specific heat. In some embodiments, one or more components of console 200 are selected to minimize the flow resistance within console 200. In some embodiments, one or more connectors and/or other components of console 200 comprise a plastic material. In some embodiments, one or more conduits and/or other components of console 200 and/or connecting assembly 300 are thermally insulated, such as to limit thermal cross talk between components. For example, conduits 3111, 3112, and 3151 of connecting assembly 300 can each comprise a thermally insulated conduit, such as to limit thermal cross talk between the conduits.

In some embodiments, CSSM 255 is configured to monitor the parameters and functionality of the components of console 200, and to limit the possibility of delivering an ablative fluid to balloon 136 without the ability to subsequently deliver a sufficient volume of neutralizing fluid. For example, CSSM 255 can disable delivery of hot fluid 2101 from hot module 2100 to balloon 136 in the event that any parameter of console 200 meets, exceeds, and/or is trending towards a threshold over time.

In some embodiments, CSSM 255 is configured to monitor use and performance of system 10 and provide fault protection, such as to enable a safe procedure independent of user intervention.

In some embodiments, system 10 includes a system clock which is used to record the duration of one or more system events, the time between two or more system events, and other temporal variables (e.g. variables related to tissue expansion and/or tissue ablation as described herein). In these embodiments, system 10 can be configured to disable delivery of energy to tissue (e.g. to target tissue) if a time limit has elapsed since the performance of a tissue expansion procedure (e.g. a time period has elapsed since the performance of the most recent tissue expansion procedure or since the performance of a tissue expansion procedure proximate the target tissue to receive the treatment energy). This time limit can comprise a maximum duration of 45 minutes, 30 minutes, 15 minutes, 10 minutes, and/or 5 minutes.

In some embodiments, system 10 comprises an energy delivery counter which keeps track of how many discrete energy delivery steps have been performed (e.g. how many segments of intestinal tissue have received ablation energy). In these embodiments, system 10 can be configured to disable energy delivery (e.g. target tissue ablation) after a maximum number of energy deliveries have been performed during the clinical procedure of the patient (e.g. limiting the clinical procedure to a maximum of 30 ablations, 20 ablations, and/or 15 ablations). In some embodiments, system 10 is configured to warn an operator of system 10 if the procedure is being ended (e.g. as indicated by an operator via a user interface of system 10), and a minimum number of energy deliveries (e.g. target tissue ablations) have not yet been performed, such as when a minimum of at least 2, 3, 4, and/or 5 energy deliveries have not yet been performed (e.g. the minimum number of ablations correlating to high likelihood of efficacy of the treatment).

In some embodiments, system 10 comprises a position sensing assembly (e.g. a magnetic, electrical, electromagnetic, radiographic, and/or other position sensing assembly) configured to monitor the position of at least functional assembly 130. The positioning assembly can be configured to track absolute position and/or relative position (e.g. movement between a first location and a second location) of functional assembly 130 or other system 10 component. In these embodiments, system 10 can be configured to prevent delivery of energy to tissue (e.g. prevent target tissue treatment) if functional assembly 130 has significantly moved (e.g. moved above a threshold distance, such as a distance of at least 2 mm, 5 mm, and/or 10 mm) since the performance of a tissue expansion procedure (e.g. functional assembly 130 has significantly moved from the location of a region of the intestine in which the last or at least a recent tissue expansion procedure was performed).

In some embodiments, system 10 is configured to prevent over ablation (e.g. ablation of non-target tissue), such as when algorithm 251 monitors signals from one or more sensors of system 10 (e.g. sensors of console 200, catheter 100, or other system component), and based on an analysis of the sensor signals can detect an undesired ablation state and modify energy delivery (e.g. stop delivery of ablation energy and/or deliver a neutralizing fluid). An undesired ablation state can comprise an undesired state of system 10, and/or an undesired state at the treatment location (e.g. as determined by a camera, temperature sensor, pressure sensor, and/or other sensor of system 10).

One, two, or more connectors described herein (e.g. connectors 2311, 2321, 2332, and/or 2352) can comprise valved connectors (e.g. connectors comprising a normally closed valve, configured to open when properly connected to a mating connector). In these embodiments, the one or more valved connectors are configured to prevent or otherwise reduce leaks.

Referring now to FIGS. 12A-E, schematic views of various operational states of console 200 are illustrated, consistent with the present inventive concepts. Console 200 can be configured to perform a disinfection procedure, whereby console 200 circulates fluid through one or more fluid lumens within console 200 and/or connecting assembly 300. The circulated fluid can comprise cold fluid 2201 and/or hot fluid 2101, which has been heated to a temperature above a threshold such that the fluid is hot enough to kill any living biological contaminants within the flushed lumens (e.g. bacteria, fungi, viruses, spores, and the like). In some embodiments, the fluid (e.g. cold fluid 2201 and/or hot fluid 2101) is heated to at least 70° C., such as at least 85° C. In some embodiments, a disinfection step (e.g. a step in which heated fluid is circulated through fluid lumens of console 200) comprises a duration of at least 30 sec, such as at least 1 minute, at least 3 minutes, or at least 5 minutes. In some embodiments, console 200 comprises a bactericidal or other disinfecting fluid that is used to disinfect one or more portions of console 200 or other system 10 fluid pathway. In the operational states (“modes” herein) described herein in reference to FIGS. 12A-XE (Modes D1-5), connector assembly 300 is operably (e.g. at least fluidly) attached to console 200. Connectors 3211 and 3251 are fluidly attached to each other, creating a fluid path between inflow conduit 3111 and return conduit 3151. In some embodiments, connectors 3211 and 3251 attach directly to each other, for example, when connector 3211 comprises a female connector and connector 3251 comprises a male connector. Alternatively or additionally, a temporary connecting conduit (not shown) can be attached between connectors 3211 and 3251.

Referring specifically to FIG. 12A, a first mode of a disinfection procedure, Mode D1, is illustrated. In Mode D1, hot module 2100 and cold module 2200 are each configured in a recirculating arrangement. Hot pump 2130 is activated (i.e. powered or otherwise activated to enter a pumping mode), drawing hot fluid 2101 from hot reservoir 2110. Hot pump 2130 drives fluid from hot reservoir 2110 via intake 2131, through regulator 2132, and through hot router 2140. Hot router 2140 is configured in the recirculating arrangement, such that the inlet 21401 of hot router 2140 is fluidly connected to the first outlet 214001 of hot router 2140, and hot fluid 2101 is directed back to hot reservoir 2110. Cold pump 2230 is also activated, drawing cold fluid 2201 from cold reservoir 2210. Cold pump 2230 drives fluid from cold reservoir 2210 via intake 2231, through overpressure valve 2232, through chiller assembly 2270, and through cold router 2240. Cold router 2240 is configured in the recirculating arrangement, such that the inlet 22401 of cold router 2240 is fluidly connected to the first outlet 224001 of cold router 2240, and cold fluid 2201 is directed back to cold reservoir 2210. In Mode D1, heaters 2120 and 2220 of hot module 2100 and cold module 2200, respectively, can each be activated (i.e. enter a heating mode), such as to heat hot fluid 2101 within reservoir 2110 and cold fluid 2201 within reservoir 2210. Chiller assembly 2270 can be disabled in Mode D1, such that heater 2220 is allowed to apply heat to cold fluid 2201.

Referring specifically to FIG. 12B, a second mode of a disinfection procedure, Mode D2, is illustrated. In Mode D2, hot module 2100 is in a sourcing arrangement, and cold module 2200 is in a recirculating arrangement. In Mode D2, cold module 2200 is configured similar to its configuration in Mode D1, as described herein in reference to FIG. 12A. In Mode D2, hot pump 2130 is activated, drawing hot fluid 2101 from hot reservoir 2110. Hot pump 2130 drives fluid from hot reservoir 2110 via intake 2131, through regulator 2132, and through hot router 2140. Hot router 2140 is activated, and configured in the sourcing arrangement, such that the inlet 21401 of hot router 2140 is fluidly connected to the second outlet 214002 of hot router 2140, and hot fluid 2101 is directed to switching module 2300. Hot fluid 2101 flows from hot router 2140, through switching module 2300, and into inflow conduit 3111 of connecting assembly 300. Hot fluid 2101 continues to the distal portion of inflow conduit 3111 and returns to switching module 2300 via shunt conduit 3112 to return selector 2360. Return router 2370 is deactivated and configured in a “hot return arrangement”, and return selector 2360 is activated and configured in a “shunt return arrangement”, these two arrangements causing hot return pump 2150 to be fluidly connected to shunt conduit 3112. Hot return pump 2150 is activated, drawing hot fluid 2101 from return router 2370, and returning hot fluid 2101 to hot reservoir 2110 (e.g. via buffer 2151). In Mode D2, hot return pump 2150 can be configured to operate at a higher flow rate than hot pump 2130, such that hot fluid 2101 is drawn back through shunt conduit 3112 at a lower pressure than hot fluid 2101 is driven through inflow conduit 3111, such that hot fluid 2101 does not flow into conduit 3151 of connecting assembly 300.

Referring specifically to FIG. 12C, a third mode of a disinfection procedure, Mode D3, is illustrated. In Mode D3, hot module 2100 is in a recirculating arrangement, and cold module 2200 is in a sourcing arrangement. In Mode D3, hot module 2100 is configured similar to its configuration in Mode D1, as described herein in reference to FIG. 12A. In Mode D3, cold pump 2230 is activated, drawing cold fluid 2201 (e.g. cold fluid 2201 which is heated to a temperature sufficient to disinfect) from cold reservoir 2210. Cold pump 2230 drives fluid from cold reservoir 2210 via intake 2231, through overpressure valve 2232, through chiller assembly 2270, and through cold router 2240. In Mode D3, chiller assembly 2270 can be disabled, such that the heated fluid passing through chiller assembly 2270 is not undesirably cooled. Cold router 2240 is activated and configured in the sourcing arrangement, such that the inlet 22401 of cold router 2240 is fluidly connected to the second outlet 224002 of cold router 2240, and cold fluid 2201 is directed to switching module 2300. Cold fluid 2201 flows from cold router 2240, through switching module 2300, and into inflow conduit 3111 of connecting assembly 300. Cold fluid 2201 continues through inflow conduit 3111, through connectors 3211 and 3251 (e.g. with connectors 3211 and 3251 fluidly connected to each other as described herein), and returns to switching module 2300 via return conduit 3151. Return router 2370 is activated and configured in a “cold return arrangement”, return selector 2360 is deactivated and configured in a “balloon return arrangement”, and return bypass 2351 is deactivated and configured in a “pass-through arrangement”, these three arrangements causing cold return pump 2250 to be fluidly connected to return conduit 3151. Cold return pump 2250 is activated, drawing cold fluid 2201 from return router 2370, and returning cold fluid 2201 to cold reservoir 2210 (e.g. via buffer 2251).

Referring specifically to FIG. 12D, a fourth mode of a disinfection procedure, Mode D4, is illustrated. In Mode D4, hot module 2100 and cold module 2200 are each configured similar to their configurations in Mode D3, as described herein in reference to FIG. 12C. In Mode D4, return router 2370 is activated and configured in the cold return arrangement, return bypass 2351 is activated and configured in the bypass arrangement, and relief valve 2355 is unpowered and configured in the open arrangement, such that cold return pump 2250 is fluidly connected to return conduit 3151 via at least a portion of bypass conduit 2356.

Referring specifically to FIG. 12E, a fifth mode of a disinfection procedure, Mode D5, is illustrated. In Mode D5, hot module 2100 and cold module 2200 are each configured similar to their configurations in Mode D2, such as is described herein in reference to FIG. 12B. In Mode D5, return selector 2360 is deactivated and configured in the balloon return arrangement, and evacuation valve 2331 is in the open configuration, such that evacuation connector 2332 is fluidly connected to vacuum connector 2341. During Mode D5, a bypass tubing assembly, bypass adapter 2449, can be attached to console 200 to fluidly connect vacuum connector 2341 to the conduit proximal to hot return pump 2150 (e.g. to a splice point between outlet 237002 of return selector 2370 and hot return pump 2150), such that hot return pump 2150 can draw fluid from shunt conduit 3112 via evacuation valve 2331.

Referring now to FIG. 12F, a flow chart of a method of performing a disinfecting procedure for a console and a connecting assembly, Method D10, is illustrated, consistent with the present inventive concepts. In Step D100, console 200 is configured as illustrated in FIG. 12A, as described herein. Hot module 2100 and cold module 2200 are each configured to heat the respective fluid (e.g. hot fluid 2101 and cold fluid 2201) within the respective reservoirs (e.g. hot reservoir 2110 and cold reservoir 2210). The fluids are heated to a temperature of at least 90° C., such as at least 95° C., for example a temperature between 94.5° C. and 95.5° C. Console 200 is configured as illustrated and described herein in reference to FIG. 12A. Once the temperature of hot fluid 2101 and cold fluid 2201 have reached the desired temperature, Step D100 can continue for a duration of at least 3 minutes, such as at least 5 minutes, as hot fluid 2101 and cold fluid 2201 are circulated as illustrated. In some embodiments, Step D100 continues until the temperature of the returning fluid (e.g. hot fluid 2101 and cold fluid 2201 returning to reservoirs 2110 and 2210, respectively) is above a threshold (e.g. as measured by sensors 2135 and 2235, respectively) for a period of time (e.g. a pre-determined period of time). After Step D100 is completed, the method continues to Step D200.

In Step D200, console 200 is configured as illustrated in FIG. 12B, as described herein. Cold module 2200 continues to recirculate the heated cold fluid 2201, such as to maintain the desired elevated temperature of cold fluid 2201. Hot module 2100 pumps hot fluid 2101 through switching module 2300, through inflow conduit 3111 of connecting assembly 300, and back to hot reservoir 2110 via shunt conduit 3112, return selector 2360, return router 2370, and hot return pump 2150. Mode D2 can continue for at least 3 minutes, such as at least 5 minutes. Mode D2 can continue for a per-determined period of time, such as a time period selected to ensure at least a 6-log reduction of biological contaminants within the fluid path. In some embodiments, Step D200 continues for a period of time (e.g. a pre-determined period of time) after hot fluid 2101 returning to reservoir 2110 has reached a temperature threshold (e.g. as determined via measurements from sensor 2135). For example, once the returning hot fluid 2101 has reached a threshold temperature (e.g. 90° C., indicative of the entire flow path being at least at that temperature), Step D200 can continue for an additional period of time (e.g. 5 minutes). After Step D200 has been completed, the method continues to Step D300.

In Step D300, console 200 is configured as illustrated in FIG. 12C, as described herein. Hot module 2100 is switched to a recirculating mode to recirculate hot fluid 2101, such as to maintain the desired temperature of hot fluid 2101. Cold module 2200 pumps heated cold fluid 2201 through switching module 2300, through inflow conduit 3111, connectors 3211 and 3251, return conduit 3151, return bypass 2351, return selector 2360, return router 2370, and cold return pump 2250. Mode D3 can continue for at least three minutes, such as at least five minutes. Mode D3 can continue for a period of time selected to ensure at least a 6-log reduction of biological contaminants within the fluid path. In some embodiments, Step D300 continues for a period of time after cold fluid 2201 returning to reservoir 2210 has reached a temperature threshold (e.g. as determined via measurements from sensor 2235). For example, once the returning cold fluid 2201 has reached a threshold temperature (e.g. 90° C., indicative of the entire flow path being at least at that temperature), Step D300 can continue for an additional period of time (e.g. 5 minutes). After Step D300 has been completed, the method continues to step D400.

In Step D400, console 200 is configured as illustrated in FIG. 12D, as described herein. Hot module 2100 continues to recirculate hot fluid 2101, such as to maintain the desired temperature of hot fluid 2101. Cold module 2200 continues to pump heated cold fluid 2201 through switching module 2300, inflow conduit 3111, connectors 3211 and 3251, and return conduit 3151. Return bypass 2351 is configured in a bypass arrangement, such that the heated cold fluid 2201 is directed through bypass conduit 2356, through relief valve 2355, through return router 2370, and cold return pump 2250. Mode D4 can continue for at least three minutes, such as at least five minutes. Mode D4 can continue for a period of time selected to ensure at least a 6-log reduction of biological contaminants within the fluid path. In some embodiments, Step D400 continues for a period of time after cold fluid 2201 returning to reservoir 2210 has reached a temperature threshold, such as determined via measurements from sensor 2235. For example, once the returning cold fluid 2201 has reached a threshold temperature (e.g. 90° C., indicative of the entire flow path being at least at that temperature), Step D400 can continue for an additional period of time (e.g. 5 minutes). After Step D400 has been completed, the method continues to Step D500.

In Step D500, console 200 is configured as illustrated in FIG. 12E, as described herein. Cold module 2200 recirculates cold fluid 2201, such as to maintain the desired temperature of cold fluid 2201. Hot module 2100 pumps hot fluid 2101 through switching module 2300, inflow conduit 3111, and shunt conduit 3112. Evacuation valve 2331 is configured in the open arrangement, such that hot fluid 2101 flows through evacuation valve 2331 to vacuum connector 2341, through bypass adapter 2449, to hot return pump 2150. Mode D5 can continue for a pre-determined period of time, such as a period of time selected to ensure at least a 6-log reduction of biological contaminants within the fluid path. In some embodiments, Step D500 continues for a period of time after hot fluid 2101 returning to reservoir 2110 has reached a temperature threshold, such as determined via measurements from sensor 2135. For example, once the returning hot fluid 2101 has reached a threshold temperature (e.g. 90° C., indicative of the entire flow path being at least at that temperature), Step D500 can continue for an additional period of time (e.g. 5 minutes). After Step D500 has been completed, the method ends, and console 200 and connecting assembly 300 are disinfected, and both are prepared for clinical use. The temperature and duration values and/or thresholds described herein for steps D100-D500 can be selected to ensure at least log-6 reduction of any biological contaminants within all fluid contacting paths and surfaces of console 200 and/or connecting assembly 300.

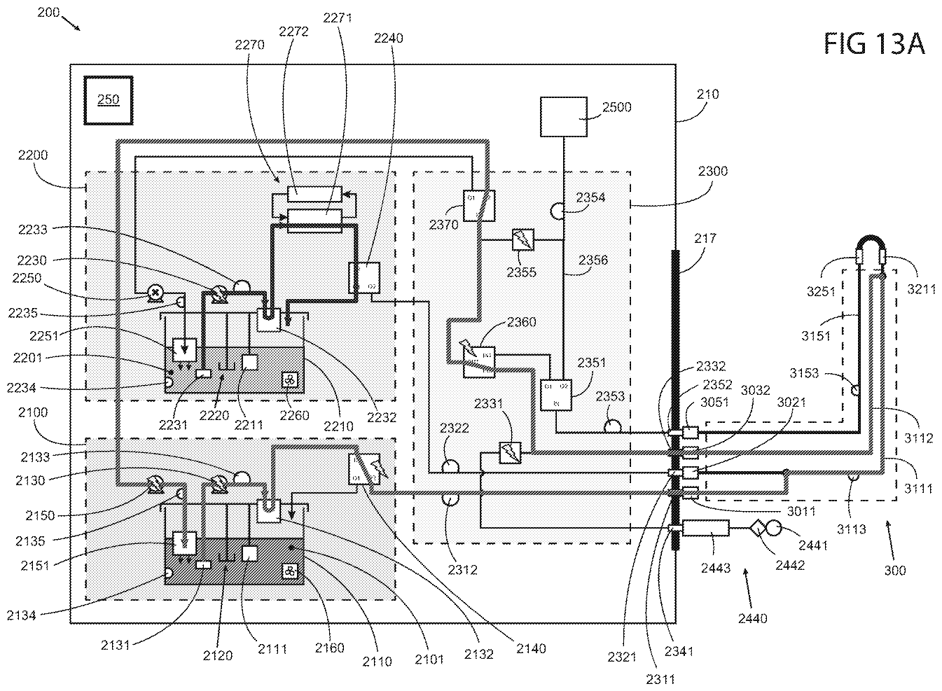

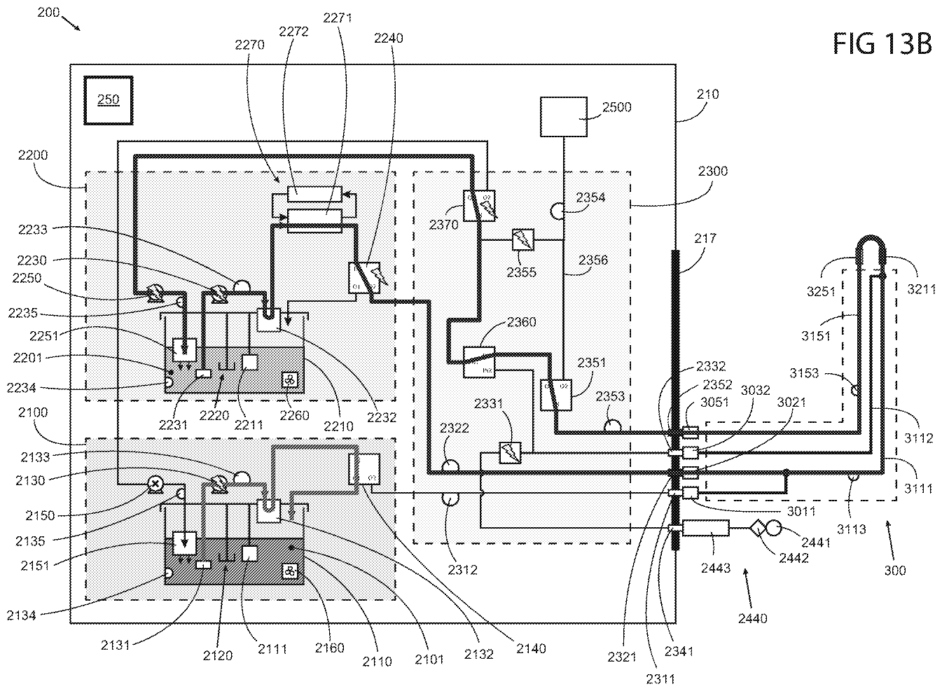

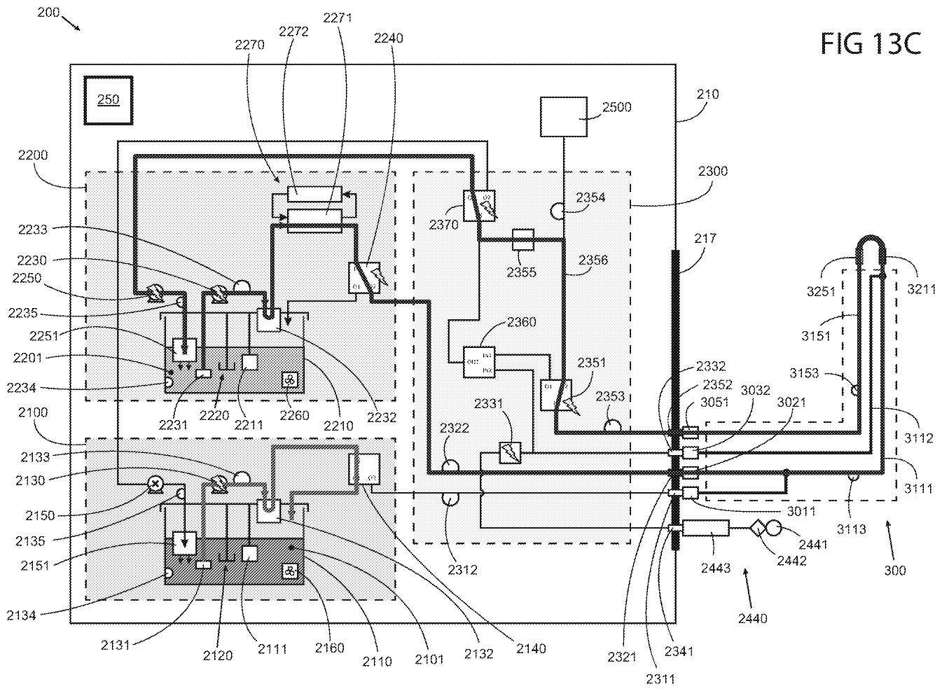

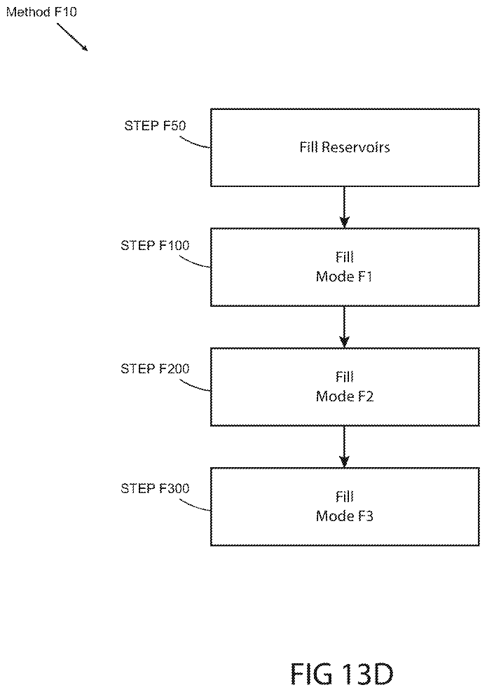

Referring now to FIGS. 13A-13C, schematic views of various operational states of console 200 are illustrated, consistent with the present inventive concepts. Console 200 can be configured to fill the various conduits (e.g. fill the lumens of conduits) and other components of console 200 and connecting assembly 300 with fluid (e.g. displacing air within said components) by circulating fluid from reservoirs 2110 and/or 2210 through said components. In the operational modes described herein in reference to FIGS. 13A-13C (Modes F1-F3), connector assembly 300 is operably (e.g. at least fluidly) attached to console 200. Connectors 3211 and 3251 are fluidly attached to each other, creating a fluid path between inflow conduit 3111 and return conduit 3151. In some embodiments, connectors 3211 and 3251 attach directly to each other, for example, when connector 3211 comprises a female connector and connector 3251 comprises a male connector. Alternatively or additionally, a temporary connecting conduit can be attached between connectors 3211 and 3251. Prior to operating in modes F1-F3, console 200 can be in an empty state (e.g. no fluid in console 200 and/or connecting assembly 300), or console 200 can be in an unknown state, during which there is fluid in the conduits and other components of console 200 and/or connecting assembly 300, however the presence of air within the system is unknown. Prior to operating in Modes F1-F3, hot reservoir 2110 and cold reservoir 2210 should be filled with fluid (e.g. filled to a maximum fill line indicated on the reservoirs, and/or filled from an empty state with a known amount of fluid).

Referring specifically to FIG. 13A, a first mode of a console fill procedure, Mode F1, is illustrated. In Mode F1, hot module 2100 is in a sourcing arrangement, and cold module 2200 is in a recirculating arrangement. In Mode F1, hot pump 2130 is activated, drawing hot fluid 2101 from hot reservoir 2110. Hot pump 2130 drives fluid from hot reservoir 2110 via intake 2131, through regulator 2132, and through hot router 2140. Hot router 2140 is activated, and configured in the sourcing arrangement, such that the inlet 21401 of hot router 2140 is fluidly connected to the second outlet 214002 of hot router 2140, and hot fluid 2101 is directed to switching module 2300. Hot fluid 2101 flows from hot router 2140, through switching module 2300, and into inflow conduit 3111 of connecting assembly 300. Hot fluid 2101 continues to the distal portion of inflow conduit 3111 and returns to switching module 2300 via shunt conduit 3112 to return selector 2360. Return router 2370 is deactivated and configured in the hot return arrangement, and return selector 2360 is activated and configured in the shunt return arrangement, these two arrangements causing hot return pump 2150 to be fluidly connected to shunt conduit 3112. Hot return pump 2150 is activated, drawing hot fluid 2101 from return router 2370, and returning hot fluid 2101 to hot reservoir 2110 (e.g. via buffer 2151). In Mode F1, hot return pump 2150 can be configured to operate at a higher flow rate than hot pump 2130, such that hot fluid 2101 is drawn back through shunt conduit 3112 at a lower pressure than hot fluid 2101 is driven through inflow conduit 3111, such that hot fluid 2101 does not flow into conduit 3151 of connecting assembly 300.

Referring specifically to FIG. 13B, a second mode of a console fill procedure, Mode F2, is illustrated. In Mode F2, hot module 2100 is in a recirculating arrangement, and cold module 2200 is in a sourcing arrangement. In Mode F2, cold pump 2230 is activated, drawing cold fluid 2201 from cold reservoir 2210. Cold pump 2230 drives fluid from cold reservoir 2210 via intake 2231, through overpressure valve 2232, through chiller assembly 2270, and through cold router 2240. Cold router 2240 is activated and configured in the sourcing arrangement, such that the inlet 22401 of cold router 2240 is fluidly connected to the second outlet 224002 of cold router 2240, and cold fluid 2201 is directed to switching module 2300. In some embodiments, the temperature of cold fluid 2201 is not regulated during Mode F2 (e.g. the temperature of cold fluid 2201 is not required to be below a temperature threshold). Cold fluid 2201 flows from cold router 2240, through switching module 2300, and into inflow conduit 3111 of connecting assembly 300. Cold fluid 2201 continues through inflow conduit 3111, through connectors 3211 and 3251 (e.g. with connectors 3211 and 3251 fluidly connected to each other as described herein), and returns to switching module 2300 via return conduit 3151. Return router 2370 is activated and configured in the cold return arrangement, return selector 2360 is deactivated and configured in the balloon return arrangement, and return bypass 2351 is deactivated and configured in the pass-through arrangement, these three arrangements causing cold return pump 2250 to be fluidly connected to return conduit 3151. Cold return pump 2250 is activated, drawing cold fluid 2201 from return router 2370, and returning cold fluid 2201 to cold reservoir 2210 (e.g. via buffer 2251).

Referring specifically to FIG. 13C, a third mode of a console fill procedure, Mode F3, is illustrated. In Mode F3, hot module 2100 and cold module 2200 are each configured similar to their configurations in Mode F2, as described herein in reference to FIG. 13B. In Mode F3, return router 2370 is activated and configured in the cold return arrangement, return bypass 2351 is activated and configured in the bypass arrangement, and relief valve 2355 is unpowered and configured in the open arrangement, such that cold return pump 2250 is fluidly connected to return conduit 3151 via at least a portion of bypass conduit 2356. In some embodiments, while cold fluid 2201 is circulated as illustrated, low pressure assembly 2500 is configured to draw cold fluid 2201 from conduit 2356, for example when low pressure assembly 2500 comprises a syringe pump assembly.

Referring now to FIG. 13D, a flow chart of a method of performing a procedure for filling the conduits and components of a console with fluid, Method F10, is illustrated, consistent with the present inventive concepts. In Step F50, hot reservoir 2110 and cold reservoir 2210 are each filled with fluid. Reservoirs 2110, 2210 can each be filled to a maximum fill line indicated on the reservoirs, and/or filled from an empty state with a known amount of fluid. In some embodiments, the method begins at Step F100, for example if reservoirs 2110, 2210 have previously been filled. The method of FIG. 13D is performed to remove all (or at least the majority) of any air from the conduits and other fluid routing components of console 200 and connecting assembly 300. Once and/or if reservoirs 2110, 2210 have been filled with fluid, the method continues to Step F100. In Steps F100-F300, the temperature of hot fluid 2101 and cold fluid 2201 are not required to be maintained at certain temperatures, and as such neither chiller assembly 2270 or either heater 2120 or 2220 are activated in steps F100-F300.

In Step F100, console 200 is configured as illustrated in FIG. 13A, as described herein. Cold module 2200 is configured in the recirculating arrangement, and hot module 2100 is configured in the sourcing arrangement. Hot module 2100 pumps hot fluid 2101 through switching module 2300, through inflow conduit 3111 of connecting assembly 300, and back to hot reservoir 2110 via shunt conduit 3112, return selector 2360, return router 2370, and hot return pump 2150. After Step F100, the method continues to Step F200.

In Step F200, console 200 is configured as illustrated in FIG. 13B, as described herein. Hot module 2100 is switched to the recirculating arrangement, and cold module 2200 is switched to the sourcing arrangement. Cold module 2200 pumps cold fluid 2201 through switching module 2300, through inflow conduit 3111, connectors 3211 and 3251, return conduit 3151, return bypass 2351, return selector 2360, return router 2370, and cold return pump 2250. After Step F200, the method continues to Step F300.

In Step F300, console 200 is configured as illustrated in FIG. 13C, as described herein. Cold module 2200 continues to pump cold fluid 2201 through switching module 2300, inflow conduit 3111, connectors 3211 and 3251, and return conduit 3151. Return bypass 2351 is configured in a bypass arrangement, such that the heated cold fluid 2201 is directed through bypass conduit 2356, through relief valve 2355, through return router 2370, and cold return pump 2250. In some embodiments, in Step F300, low pressure assembly 2500 can be filled with fluid, such as described herein in reference to FIG. 13C.

Referring now to FIGS. 14A-14G, schematic views of various procedural operational states of console 200 are illustrated, consistent with the present inventive concepts. Console 200 can be configured to pump hot fluid 2101 and cold fluid 2201 to and from catheter 100, such as to enable the usage of catheter 100 as described herein. In the procedural operational modes described herein in reference to FIGS. 14A-14G, catheter 100 is operably (e.g. at least fluidly) attached to console 200 via connector assembly 300, such as via connections described herein in reference to FIG. 12. For example, connector 3211 is attached to inflow connector 1012 of catheter 100, and connector 3251 is connected to outflow connector 1052, such that console 200 can deliver fluid to balloon 136 via conduit 1011, and can withdraw fluid from balloon 136 via conduit 1051. In some embodiments, console 200 is prepared for an attachment of catheter 100, such as when a console fill procedure is performed prior to the attachment of catheter 100, such as console fill procedure as described herein in reference to FIG. 14H.

Referring specifically to FIG. 14A, a first mode of a catheter preparation procedure, Mode P1, is illustrated. In Mode P1, hot module 2100 is configured in a recirculating arrangement. Hot pump 2130 is activated (i.e. powered or otherwise activated to enter a pumping mode), drawing hot fluid 2101 from hot reservoir 2110. Hot pump 2130 drives hot fluid 2101 from hot reservoir 2110 via intake 2131, through regulator 2132, and through hot router 2140. Hot router 2140 is configured in the recirculating arrangement, such that the inlet 21401 of hot router 2140 is fluidly connected to the first outlet 214001 of hot router 2140, and hot fluid 2101 is directed back to hot reservoir 2110. Heater 2120 can be activated such as to apply thermal energy to hot fluid 2101 as it is circulated as described, such that the temperature of hot fluid 2101 is increased towards a target temperature during Mode P1, such as a target ablation temperature described herein.

In Mode P1, cold module 2200 is configured in a sourcing arrangement. Cold pump 2230 is activated, drawing cold fluid 2201 from cold reservoir 2210. Cold pump 2230 drives fluid from cold reservoir 2210 via intake 2231, through overpressure valve 2232, through chiller assembly 2270, and through cold router 2240. Cold router 2240 is activated and configured in the sourcing arrangement, such that the inlet 22401 of cold router 2240 is fluidly connected to the second outlet 224002 of cold router 2240, and cold fluid 2201 is directed to switching module 2300. In some embodiments, the temperature of cold fluid 2201 is not regulated during Mode P1 (e.g. the temperature of cold fluid 2201 is not required to be below a temperature threshold). In some embodiments, chiller assembly 2270 is activated during Mode P1, such that as cold fluid 2201 is circulated, the temperature is continuously lowered, such as until a desired temperature is reached.

In Mode P1, cold fluid 2201 flows from cold router 2240, through switching module 2300, and into inflow conduit 3111 of connecting assembly 300. Cold fluid 2201 continues through inflow conduit 3111, through conduit 1011, and into balloon 136 via inflow port 1013. Cold return pump 2250 is fluidly attached to balloon 136 via return router 2370, return selector 2360, and return bypass 2351, each configured in the cold return, balloon return, and pass-through arrangements, respectively. Cold fluid 2201 is drawn from balloon 136 via outflow port 1053, which is fluidly attached to inlet 23511 of return bypass 2351 via conduits 1051 and 3151. In some embodiments, cold return pump 2250 is configured to operate at a higher speed than cold pump 2230, such that cold fluid 2201 is withdrawn from balloon 136 with a higher force than the pumping pressure of cold pump 2230, such that balloon 136 does not inflate with cold fluid 2201 while the fluid is circulated through the balloon. Cold return pump 2250 returns cold fluid 2201 to cold reservoir 2210 (e.g. via buffer 2251).

Referring specifically to FIG. 14B, a second mode of a catheter preparation procedure, Mode P2, is illustrated. In Mode P2, hot module 2100 and switching module 2300 are configured as described herein in reference to FIG. 14A. Cold pump 2230 is deactivated and cold router 2240 is configured in the recirculating arrangement, while cold return pump 2250 remains activated, such that cold return pump 2250 continues to draw cold fluid 2201 from the illustrated fluid path, causing a negative pressure to build (e.g. as cold fluid 2201 is drawn from the closed outlet 224002 of cold router 2240). This negative pressure can eliminate or at least reduce any air (e.g. air bubbles) within the illustrated fluid path, including balloon 136. In some embodiments, Mode P2 is configured such that the orientation of catheter 100 does not affect the efficacy of the removal of air from the illustrated fluid path (e.g. the negative pressure removes air from the path regardless of catheter orientation).

Referring specifically to FIG. 14C, a third mode of a catheter preparation procedure, Mode P3, is illustrated. In Mode P3, hot module 2100 is configured as described herein in reference to FIG. 14A. Return bypass 2351 is configured in the bypass arrangement, such that low pressure assembly 2500 is fluidly attached to balloon 136 via return router 2351 and conduits 3151 and 1051. Balloon 136 is fluidly attached to cold module 2200 via conduits 1011 and 3111, and switching module 2300. Cold router 2240 is configured in the sourcing arrangement, and cold pump 2230 is disabled, such that the negative pressure within the illustrated path is allowed to draw cold fluid 2201 from cold reservoir 2210 through cold pump 2230, such that the negative pressure is allowed to equalize. In some embodiments, cold router 2240 switches to the recirculating arrangement once the pressure has equalized, such that catheter 100 switches to a tracking mode, as described herein.

Referring specifically to FIG. 14D, a catheter tracking mode, Mode T1, is illustrated. Mode T1 can be used when catheter 100 is to be translated (i.e. advanced and/or retracted) within the patient's GI tract. In Mode T1, hot module 2100 and cold module 2200 are each configured in the recirculating arrangement. Return bypass 2351 is configured in the bypass arrangement, such that low pressure assembly 2500 is fluidly attached to balloon 136. Cold router 2240 is configured in the recirculating arrangement, such that outlet 224002 is a closed outlet (e.g. no fluid can flow to or from outlet 224002), creating a closed fluid path between outlet 224002 and low pressure assembly 2500, the closed path including balloon 136. In Mode T1, low pressure assembly 2500 can be configured to inject and/or withdraw fixed amounts of fluid from balloon 136 (e.g. when low pressure assembly 2500 comprises a syringe pump configured to provide and/or withdraw fluid).

Referring specifically to FIG. 14E, a thermal priming mode, Mode TP1, is illustrated. In Mode TP1, hot module 2100 is in a sourcing arrangement, and cold module 2200 is in a recirculating arrangement. In Mode TP1, hot pump 2130 is activated, drawing hot fluid 2101 from hot reservoir 2110. Hot pump 2130 drives fluid from hot reservoir 2110 via intake 2131, through regulator 2132, and through hot router 2140. Hot router 2140 is activated, and configured in the sourcing arrangement, such that the inlet 21401 of hot router 2140 is fluidly connected to the second outlet 214002 of hot router 2140, and hot fluid 2101 is directed to switching module 2300. Hot fluid 2101 flows from hot router 2140, through switching module 2300, and into inflow conduit 3111 of connecting assembly 300. Hot fluid 2101 continues to the distal portion of inflow conduit 3111 and returns to switching module 2300 via shunt conduit 3112 to return selector 2360. Return router 2370 is deactivated and configured in the hot return arrangement, and return selector 2360 is activated and configured in the shunt return arrangement, these two arrangements causing hot return pump 2150 to be fluidly connected to shunt conduit 3112. Hot return pump 2150 is activated, drawing hot fluid 2101 from return router 2370, and returning hot fluid 2101 to hot reservoir 2110 (e.g. via buffer 2151). In Mode TP1, hot return pump 2150 can be configured to operate at a higher flow rate than hot pump 2130, such that hot fluid 2101 is drawn back through shunt conduit 3112 at a lower pressure than hot fluid 2101 is driven through inflow conduit 3111, such that hot fluid 2101 does not flow into catheter 100 from connecting assembly 300.

Referring specifically to FIG. 14F, a cold treatment mode, Mode C1, is illustrated. In Mode C1, hot module 2100 is configured in a recirculating arrangement, as described herein. In Mode C1, cold module 2200 is configured in a sourcing arrangement. Cold pump 2230 is activated, drawing cold fluid 2201 from cold reservoir 2210. Cold pump 2230 drives fluid from cold reservoir 2210 via intake 2231, through overpressure valve 2232, through chiller assembly 2270, and through cold router 2240. Cold router 2240 is activated and configured in the sourcing arrangement, such that the inlet 22401 of cold router 2240 is fluidly connected to the second outlet 224002 of cold router 2240, and cold fluid 2201 is directed to switching module 2300.

In Mode C1, cold fluid 2201 flows from cold router 2240, through switching module 2300, and into inflow conduit 3111 of connecting assembly 300. Cold fluid 2201 continues through inflow conduit 3111, through conduit 1011, and into balloon 136 via inflow port 1013. Cold return pump 2250 is fluidly attached to balloon 136 via return router 2370, return selector 2360, and return bypass 2351, each configured in the cold return, balloon return, and pass-through arrangements, respectively. Cold fluid 2201 is drawn from balloon 136 via outflow port 1053, which is fluidly attached to inlet 23511 of return bypass 2351 via conduits 1051 and 3151. In Mode C1, cold pump 2230 is configured to operate at a higher speed than cold return pump 2250, such that cold fluid 2201 is pumped into balloon 136 at a higher pressure than the withdraw force applied by cold return pump 2250, such that balloon 136 inflates with cold fluid 2201. Cold return pump 2250 returns cold fluid 2201 to cold reservoir 2210 (e.g. via buffer 2251).

Referring specifically to FIG. 14G, a hot treatment mode, Mode H1, is illustrated. In Mode H1, cold module 2200 is configured in a recirculating arrangement, as described herein. In Mode H1, hot module 2100 is configured in a sourcing arrangement. Hot pump 2130 is activated drawing hot fluid 2101 from hot reservoir 2110. Hot pump 2130 drives fluid from hot reservoir 2110 via intake 2131, through regulator 2132, and through hot router 2140. Hot router 2140 is activated and configured in the sourcing arrangement, such that the inlet 21401 of hot router 2140 is fluidly connected to the second outlet 214002 of hot router 2140, and hot fluid 2101 is directed to switching module 2300.

In Mode H1, hot fluid 2101 flows from hot router 2140, through switching module 2300, and into inflow conduit 3111 of connecting assembly 300. Hot fluid 2101 continues through inflow conduit 3111, through conduit 1011, and into balloon 136 via inflow port 1013. Hot return pump 2150 is fluidly attached to balloon 136 via return router 2370, return selector 2360, and return bypass 2351, each configured in the hot return, balloon return, and pass-through arrangements, respectively. Hot fluid 2101 is drawn from balloon 136 via outflow port 1053, which is fluidly attached to inlet 23511 of return bypass 2351 via conduits 1051 and 3151. In Mode H1, hot pump 2130 is configured to operate at a higher speed than hot return pump 2150, such that hot fluid 2101 is pumped into balloon 136 at a higher pressure than the withdraw force applied by hot return pump 2150, such that balloon 136 inflates with hot fluid 2101. Hot return pump 2150 returns hot fluid 2101 to hot reservoir 2110 (e.g. via buffer 2151).

In some embodiments, the illustrated pathway comprises a closed pathway (e.g. fluid circulates from console 200 to balloon 136 and back) and the fluid does not make contact with the patient.

Referring now to FIG. 14H, a flow chart of a method of treating a patient is illustrated, consistent with the present inventive concepts. In Method T10, console 200 is prepared and catheter 100 is attached to console 200. Catheter 100 is inserted into the patient and positioned to treat a segment of patient tissue. A tissue expansion (e.g. a submucosal tissue expansion) can be performed, and the segment of tissue can be thermally treated. In Method T10, one, two, three or more segments of tissue can be treated, such as is described herein.

In Step T100, console 200 is filled with fluid, such as is described herein in reference to FIGS. 13A-13D. In Step T200, a disinfection procedure similar to that described herein in reference to FIGS. 12A-12F is performed. In some embodiments, Step T300 is performed a period of time after Step T200, for example at least 4 hours, at least 8 hours, or at least 12 hours after Step T200. For example, in some embodiments, console 200 is disinfected the day prior to a procedure. In some embodiments, Step T100 is repeated prior to Step T300, for example if Step T200 was performed the day before Step T300.

In Step T300, catheter 100 is attached to console 200 via connecting assembly 300, as described herein. In some embodiments, connectors 2311, 2321, 2332, 2341, and 2352 of console 200, and connectors 3011, 3021, 3032, and 3051 of connecting assembly 300 and vacuum connector 2443 can comprise unique, mating connectors, such that only proper connections can be made (e.g. an incorrect connector from connecting assembly 300 cannot physically attach to any connector but the proper corresponding connector of console 200). Additionally or alternatively, the connectors of console 200 and connecting assembly 300 can be color coded or otherwise visually marked to assist in proper connection. In some embodiments, connectors 3211 and 3251 comprise unique, mating connectors configured to mate with connectors 1012 and 1052, respectively, of catheter 100.

After catheter 100 is attached in Step T300, Method T10 continues to Step T410. In Step T410, console 200 is configured as illustrated in FIG. 14A. Hot module 2100 is configured in the recirculating arrangement, and cold module 2200 is configured in the sourcing arrangement. Cold module 2200 pumps cold fluid 2201 through switching module 2300, through inflow conduit 3111, through conduit 1011, and into balloon 136 via inflow port 1013. Cold return pump 2250 is fluidly attached to balloon 136 via return router 2370, return selector 2360, and return bypass 2351, each configured in the cold return, balloon return, and pass-through arrangements, respectively. Cold fluid 2201 is drawn from balloon 136 via outflow port 1053, which is fluidly attached to inlet 23511 of return bypass 2351 via conduits 1051 and 3151. In some embodiments, cold return pump 2250 is configured to operate at a higher speed than cold pump 2230, such that cold fluid 2201 is withdrawn from balloon 136 with a higher force than the pumping pressure of cold pump 2230, such that balloon 136 does not inflate with cold fluid 2201 while the fluid is circulated through the balloon.

After Step T410, Method T10 continues to Step T420, during which console 200 is configured as illustrated in FIG. 14B. In Step T420, cold pump 2230 is deactivated and cold router 2240 is switched to the recirculating arrangement. Cold return pump 2250 continues to draw fluid from balloon 136, drawing negative pressure through the fluid lumens and balloon of catheter 100. In some embodiments, Step T420 continues until the pressure within the lumens illustrated in FIG. 14B reaches a threshold, such as below a threshold of at least −5 psi, such as at least −10 psi. Alternatively or additionally, Step T420 can be performed for a predetermined duration, such as for approximately 6 seconds. After Step T420, Method T10 continues to Step T430.

In Step T430, console 200 is configured as illustrated in FIG. 14C. In Step T430, low pressure assembly 2500 is fluidly attached to balloon 136 as return bypass 2351 is switched to the bypass arrangement. Cold router 2240 is switched to the sourcing arrangement, and the negative pressure within the catheter and console lumens is allowed to equalize (e.g. towards 0 psi) by drawing cold fluid 2201 through cold pump 2230, despite cold pump 2230 being deactivated. In some embodiments, Step T430 continues until the negative pressure (e.g. as measured by sensor 2354) rises above a threshold, such as above a threshold of at least −2.7 psi-. After Step T430, Method T10 continues to Step T500.

In Step T500, console 200 is configured as illustrated in FIG. 14D. In Step T500, cold router 2240 is switched to the recirculating arrangement, and cold pump 2230 can be activated such as to recirculate cold fluid 2201 through chiller assembly 2270. Catheter 100 is in a tracking mode, during which balloon 136 is in a state configured to be inserted into and tracked through the anatomy (e.g. the GI track) of a patient. In Step T510, balloon 136 is positioned proximate target tissue, such as target tissue described herein. After balloon 136 is properly positioned proximate target tissue, Method T10 proceeds to Step T520.

In Step T520, balloon 136 is partially inflated by a volume of fluid delivered by low pressure assembly 2500. In some embodiments, low pressure assembly 2500 delivers between 5 ml and 15 ml, such as 8 ml or 10 ml of fluid to balloon 136. In some embodiments, low pressure assembly 2500 delivers a volume of fluid to balloon 136 that is sufficient to orient vacuum ports 137 outwardly (e.g. toward the luminal wall). After balloon 136 is partially inflated, Method T10 proceeds to Step T550.

In Step T550, a tissue expansion is performed. This tissue expansion (e.g. submucosal tissue expansion) can be performed by delivering injectate 221 into tissue, as described herein. In some embodiments, a vacuum can be applied to one, two, three or more of vacuum ports 137, such as to draw a portion of tissue into ports 137. Subsequently, one, two, three or more fluid delivery elements 139 can be advanced (e.g. advanced within respective vacuum ports 137) to engage tissue, such that a volume of fluid can be injected into the tissue. In some embodiments, during tissue expansion (e.g. in Step T520), system 10 does not monitor the pressure of the fluid within balloon 136. As described herein in reference to Step T550, a fixed amount of fluid can be delivered to balloon 136 by low pressure assembly 2500, such that the volume of fluid is less than a volume which would otherwise cause a significant increase in pressure within balloon 136 (e.g. the volume of fluid is less than the maximum volume of balloon 136). After tissue expansion has been performed, Method T10 proceeds to Step T560. In some embodiments, console 200 is configured to automatically prepare for a subsequent tissue expansion, such as by automatically refilling one, two, or more syringes as described herein.

In Step T560, balloon 136 is deflated using low pressure assembly 2500. After balloon 136 has been deflated, catheter 100 is configured in the tracking mode, and in Step T570, if another tissue expansion is to be performed, Method T10 returns to Step T510, and catheter 100 is repositioned for a subsequent tissue expansion. If a subsequent tissue expansion is not to be performed after Step T570, Method T10 proceeds to Step T610. In some embodiments, Method T10 only continues to Step T610 if the temperature of hot fluid 2101 within reservoir 2110 has reached a target temperature. For example, console 200 can be filled with room temperature fluid (or an otherwise unknown temperature fluid), and as such, console 200 requires an amount of time to raise the temperature of the fluid to an ablative temperature. In some embodiments, console 200 is configured to raise the temperature of hot fluid 2101 from an unknown temperature (e.g. a temperature greater than 1° C.) to an ablative temperature (e.g. a temperature greater than 80° C.) in less than 30 minutes, such as less than 20 minutes, such as less than 10 minutes (e.g. the time between Step T300 and Step T610). In some embodiments, Method T10 only continues to Step T610 if at least one tissue expansion has been performed (e.g. at least one tissue expansion has been performed since the last time Step T610 was performed). In some embodiments, Method T10 only continues to Step T610 if at least two tissue expansions have been performed, or if at least two tissue expansions have been performed since the most recent treatment (e.g. ablative treatment). In some embodiments, between Step T550 and Step 640, catheter 100 is not advanced and/or retracted (e.g. remains stationary).

In Step T610, console 200 is configured as illustrated in FIG. 14E. In Step T610, return selector 2360 is switched to the shunt return arrangement, and hot router 2140 is switched to the sourcing arrangement, such that hot fluid 2101 is circulated through console 200 and at least a portion of connecting assembly 300. Hot return pump 2150 can be configured to operate at a higher speed than hot pump 2130, such that hot fluid 2101 is drawn back to hot reservoir 2110 via shunt conduit 3112 with a higher force than the pressure from hot pump 2130, such that no hot fluid 2101 enters catheter 100. In some embodiments, Step T610 continues until hot fluid 2101 returning to hot reservoir 2110 reaches or exceeds a temperature threshold (e.g. as measured by sensor 3113), such as a temperature threshold of at least 86° C. In some embodiments, if the temperature threshold is not reached before a timeout initiated at the start of Step T610 is reached, an alert mode can be triggered. The alert mode can indicate a leak and/or other malfunction of at least a portion of console 200 and/or connecting assembly 300. For example, the timeout can comprise a time period of no more than 60 seconds, such as approximately 50 seconds. In some embodiments, Step T610 is performed such that at least a portion of console 200 and/or connecting assembly 300 is thermally primed (e.g. at least a portion of console 200 and/or connecting assembly 300 comprising a large thermal mass is heated to a sufficient temperature), such as to minimize the heat lost to those portions of console 200 and/or connecting assembly 300 during a thermal ablation (e.g. during Step T630). In some embodiments, during Step T610, CSSM 255 monitors the temperature of sensor 3153, such as to ensure there is no significant temperature increase within return conduit 3151. During Step T610, no fluid (e.g. hot fluid 2101 and/or cold fluid 2201) should be entering conduit 3151, and as such, a change in the temperature at sensor 3153 may indicate a leak and/or other issue with console 200, and CSSM 255 can be configured to trigger an alert mode. After Step T610, Method T10 proceeds to Step T620.

In Step T620, console 200 is configured as illustrated in FIG. 14F. In Step T620, hot router 2140 is switched to the recirculating arrangement, and cold router 2240 is switched to the sourcing arrangement such that cold fluid 2201 is pumped to catheter 100. Return selector 2360 is switched to the balloon return arrangement, such that cold fluid pumped from cold module 2200 is circulated through balloon 136 (e.g. via return bypass 2351 configured in the passthrough arrangement). Cold pump 2230 can be configured to operate at a higher speed than cold return pump 2250, such that balloon 136 fills with cold fluid 2201 with positive pressure, such that balloon 136 expands to contact surrounding tissue, the surrounding tissue being cooled by cold fluid 2201 recirculating in balloon 136. In some embodiments, Step T620 continues for a set period of time, such as at least 10 seconds, such as at least 15 seconds. Alternatively or additionally, Step T620 continues until cold fluid 2201 returning to cold reservoir 2210 reaches or exceeds a temperature threshold (e.g. as measured by sensor 2235), such as a temperature threshold of at most 25° C. In some embodiments, the temperature threshold is determined based on the temperature of cold fluid 2201 within cold reservoir 2210. For example, the threshold can comprise a temperature that is 5° C. greater than the temperature of cold fluid 2201 within reservoir 2210. In some embodiments, at the beginning of Step T620, return router 2370 is configured in the hot return arrangement for a period of time, before switching to the cold return arrangement. In these embodiments, as hot fluid 2101 existing within the lumens and components of console 200 and connecting assembly 300 is displaced by cold fluid 2201, the hot fluid 2101 is returned to hot reservoir 2110. After this hot fluid has been displaced, return router 2370 switches to the cold return arrangement, such that the circulating cold fluid 2201 is returned to cold reservoir 2210. In some embodiments, this delay comprises a duration of approximately 2 seconds. Additionally or alternatively, return router 2370 can be configured to switch to the cold return arrangement when the temperature of the fluid returning from balloon 136 reaches and/or exceeds a temperature threshold (e.g. as measured by sensor 2353). After Step T620, Method T10 proceeds to Step T630.

In Step T630, console 200 is configured as illustrated in FIG. 14G. In Step T630, cold router 2240 is switched to the recirculating arrangement, and hot router 2140 is switched to the sourcing arrangement such that hot fluid 2101 is pumped to catheter 100. Hot pump 2130 can be configured to operate at a higher speed than hot return pump 2150, such that balloon 136 fills with hot fluid 2101 with positive pressure, such that balloon 136 remains expanded and in contact with surrounding tissue, with at least a portion of the surrounding tissue receiving ablative thermal energy configured to ablate the tissue. In some embodiments, Step T630 continues for a set period of time, such as at least 8.6 seconds, such as at least 10 seconds. In some embodiments, the duration of Step T630 can be determined based on the temperature of either or both of hot fluid 2101 and cold fluid 2201. For example, the duration of Step T630 and the temperature of cold fluid 2201 can have an inverse relationship, where the colder the temperature of cold fluid 2201, the longer the duration of Step T630, such that the temperature of the surrounding tissue has sufficient time to rise to an ablative temperature. In some embodiments, at the beginning of Step T630, return router 2370 is configured in the cold return arrangement for a period of time, before switching to the hot return arrangement. In these embodiments, as cold fluid 2201 existing within the lumens and components of console 200, connecting assembly 300, and catheter 100 is displaced by hot fluid 2101, the cold fluid 2201 is returned to cold reservoir 2210. After this cold fluid has been displaced, return router 2370 switches to the hot return arrangement, such that the circulating hot fluid 2101 is returned to hot reservoir 2110. In some embodiments, this delay comprises a duration of approximately 8 seconds. Additionally or alternatively, return router 2370 can be configured to switch to the hot return arrangement when the temperature of the fluid returning from balloon 136 reaches and/or exceeds a temperature threshold (e.g. as measured by sensor 2353). After Step T630, Method T10 proceeds to Step T640. In some embodiments, prior to Steps T610, T620, and/or T630, the level and/or temperature of the fluid within hot reservoir 2110 and/or cold reservoir 2210 is measured to ensure there is a sufficient amount and/or adequate temperature of hot fluid 2101 and/or cold fluid 2201 to successfully perform each of Steps T610, T620, T630, and/or T640. In some embodiments, as described herein, Step T630 does not begin unless system 10 detects a sufficient amount and/or temperature of cold fluid 2201 within cold reservoir 2210 to adequately neutralize the thermal energy of hot fluid 2101 to be delivered in Step T630.

In Step T640, console 200 is configured as illustrated in FIG. 14F. In Step T640, hot router 2140 is switched to the recirculating arrangement, and cold router 2240 is switched to the sourcing arrangement such that cold fluid 2201 is pumped to catheter 100. Cold pump 2230 can be configured to operate at a higher speed than cold return pump 2250, such that balloon 136 fills with cold fluid 2201 with positive pressure, such that balloon 136 remains expanded and in contact with the surrounding tissue, the surrounding tissue being cooled by cold fluid 2201 recirculating in balloon 136. In some embodiments, Step T640 continues for a set period of time, such as at least 15 seconds, such as at least 20 seconds. Alternatively or additionally, Step T640 continues until cold fluid 2201 returning to cold reservoir 2210 reaches or exceeds a temperature threshold (e.g. as measured by sensor 2235), such as a temperature threshold of at most 25° C. In some embodiments, the temperature threshold is determined based on the temperature of cold fluid 2201 within cold reservoir 2210. For example, the threshold can comprise a temperature that is 5° C. greater than the temperature of cold fluid 2201 within reservoir 2210. In some embodiments, at the beginning of Step T640, return router 2370 is configured in the hot return arrangement for a period of time, before switching to the cold return arrangement. In these embodiments, as hot fluid 2101 existing within the lumens and components of console 200, connecting assembly 300, and catheter 100 is displaced by cold fluid 2201, the hot fluid 2101 is returned to hot reservoir 2110. After this hot fluid has been displaced, return router 2370 switches to the cold return arrangement, such that the circulating cold fluid 2201 is returned to cold reservoir 2210. In some embodiments, this delay comprises a duration of approximately 8.9 seconds. Additionally or alternatively, return router 2370 can be configured to switch to the cold return arrangement when the temperature of the fluid returning from balloon 136 reaches and/or exceeds a temperature threshold (e.g. as measured by sensor 2353). After Step T640, Method T10 proceeds to Step T500′.

In some embodiments, following any of steps T610, T620, T630, and/or T640, console 200 is configured to automatically prepare for one or more subsequent steps, and/or to repeat one or more steps. For example, following treatment Step T630, console 200 can automatically prepare for a subsequent step T610 and/or T630 by continuously reheating and/or maintaining hot fluid 2101 within hot reservoir 2110 at an ablative temperature, as described herein.

In some embodiments, system 10 is configured to insufflate and/or aspirate the gastrointestinal tract of the patient during one or more of steps T510, T520, T550, T560, T610, T620, T630, and/or T640. For example, system 10 can be configured to aspirate both proximal to and distal to balloon 136 during treatment step T630, such as to help ensure good apposition of balloon 136 to the tissue. Aspiration of the gastrointestinal tract can include Pulsatile aspiration comprising a frequency of greater than 1 pulse every 2 seconds, such as 1 pulse per second.

In Step T500′, console 200 is configured as illustrated in FIG. 14D. In Step T500′, cold router 2240 is switched to the recirculating arrangement, and return bypass 2351 is activated to switch to the bypass arrangement, such that low pressure assembly 2500 is fluidly attached to balloon 136, such that catheter 100 is in the tracking mode. In some embodiments, to start Step T500′, a drawdown procedure is performed, during which the fluid within balloon 136 is removed by cold module 2200, similar to as described herein in Steps T420 and T430. After Step T500′, in Step T650, if the procedure is to continue, Method T10 returns to Step T510. If the procedure is complete, Method T10 proceeds to Step T700. In some embodiments, if Method T10 returns to Step T510, system 10 is configured to prevent Step T610 from being performed within a time period of the completion of Step T640, such as a time period of at least 5 minutes. In some embodiments, system 10 prevents Step T610 from being performed if the temperature of hot fluid 2101 and/or cold fluid 2201 has not returned to an acceptable temperature (e.g. if the temperature of the fluid changes as fluid is circulated through balloon 136 and returned to the reservoir in any of Steps T610-T640). In some embodiments, one, two, three, four, five or more cycles of tissue expansion and ablation are performed as Method T10 cycles from Step T510 to Step T650. In some embodiments, in the first Step T510, catheter 100 is positioned such that balloon 136 is positioned proximate the most proximal desired ablation site, and catheter 100 is moved distally with each subsequent tissue expansion and/or ablation. For example, after a treatment step (e.g. an ablation and/or tissue expansion), the proximal end of balloon 136 can be positioned at the distal end of the previous treatment site (e.g. at the distal end of an immediately previous site of ablation). Performance of tissue treatments in this proximal to distal fashion can reduce the likelihood of intussusception of the intestine. In addition, the proximal to distal tissue treatment methodology allows visualization (e.g. via an endoscope) of a tissue treatment (e.g. an ablation) prior to the performance of a subsequent tissue treatment.

In some embodiments, Method T10 is configured to be canceled, such as canceled by a user to stop and/or reverse the process of one of the steps of Method T10. For example, Step T610 can be canceled by the user, and the flow of hot fluid 2101 through console 200 will be ceased, and hot module 2100 will switch to the recirculating mode. One or more steps of Method T10 can be configured such that the user cannot cancel the step, and/or if the step is canceled, the subsequent step will automatically begin. For example, if the user cancels Step T630 (e.g. during which ablative fluid is being delivered to balloon 136), Step T640 will automatically begin. Additionally or alternatively, Method T10 can be configured such that a neutralizing step, such as Step T640, cannot be canceled by the user.

Referring now to FIG. 15, a chart of tissue temperature during a treatment procedure is illustrated, consistent with the present inventive concepts. As described herein in reference to FIG. 14H, in Steps T620-T640, a tissue treatment can comprise cooling the tissue, then heating the tissue to an ablative temperature, and subsequently cooling the tissue (“cool-heat-cool ablation” herein). FIG. 15 illustrates the approximate temperature of the tissue surface proximate balloon 136 (e.g. the temperature of the fluid within balloon 136), as well as the temperature of tissue approximately 1 mm below the tissue surface during a treatment cycle. In some embodiments, the treatment comprises a non-desiccative treatment (e.g. the tissue proximate balloon 136 does not desiccate). In some embodiments, functional assembly 130 and other components of system 10 are configured to allow multiple energy deliveries to be performed (e.g. multiple portions of target tissue to be ablated sequentially), without the need to remove any material (e.g. char) from functional assembly 130, or otherwise reverse any adverse condition of catheter 100 that results from the performance of tissue expansion and/or treatment of target tissue. For example, system 10 can be configured such that all or at least multiple target tissue treatments of the clinical procedure (e.g. at least 3, 4, and/or 5 target tissue ablations) can be performed sequentially without the need to remove catheter 100 from the patient). In some embodiments, the treatment comprises a coagulatory treatment (e.g. a treatment in which the tissue proximate balloon 136 coagulates). As used herein, FIG. 15 illustrates the “ablation profile” provided by system 10 for a single cool-heat-cool ablation treatment.

In some embodiments, one or more of the temperature settings, temperature thresholds, and/or timing parameters (“settings” herein) of the treatment procedure described herein can be based on one, two, or more patient and/or system parameters. For example, one, two, or more of the settings of the treatment procedure can be based on a parameter selected from the group consisting of: room temperature; altitude; patient temperature; ambient temperature within console 200; the temperature of cold fluid 2201; the temperature of hot fluid 2101; time since the most recent treatment (e.g. ablative treatment); and combinations of two or more of these. In some embodiments, the settings of the treatment procedure are dynamically adjusted, such as when system 10 is configured to perform a closed loop treatment procedure. For example, as described herein, one, two, or more parameters of system 10 can be monitored during a treatment procedure (e.g. the temperature of fluid returning to console 200 from catheter 100), and one, two, or more settings of system 10 can be adjusted based on the monitored parameters. In some embodiments, the duration of an ablation step (e.g. Step T630 described herein) is adjusted based on the temperature of cold fluid 2201 (e.g. the colder fluid 2201 is, the longer an ablative step would be to heat tissue to an ablative temperature).

In some embodiments, console 200 is configured to control the flow rates of fluid to catheter 100 (e.g. the flow rate of hot fluid 2101 leaving hot module 2100 and/or the flow rate of cold fluid 2201 leaving cold module 2200). For example, pumps 2130 and/or 2230 can comprise gear pumps configured to pump fluid at a set flow rate (e.g. a rate determined based on the speed of the pump), so long as the resistance encountered by the pump is within a range of acceptable values. In some embodiments, the gear pumps go through a “burn in” process, such as in a manufacturing process of console 200. In some embodiment, hot pump 2130 is configured to pump at a flow rate of approximately 9.5 mL/s. In some embodiments, cold pump 2230 is configured to pump at a flow rate of approximately 9.2 mL/s. Alternatively or additionally, console 200 can be configured to monitor the pressure of fluid (e.g. pressure at hot pump 2130 or cold pump 2230) and adjust pumps 2130 and/or 2230 accordingly to achieve a desired pressure. One or more pumps of console 200 (e.g. pumps 2130 and/or 2230) can comprise gear pumps configured to pump fluid at a pressure of at least 30 psi, such as at least 40 psi, such as approximately 50 psi. In some embodiments, system 10 is configured such that the pressure of fluid within balloon 136 reaches at least 10 psi, such as at least 15 psi, such as at least 20 psi (e.g. during a treatment step when fluid is pumped at a high flow rate into balloon 136). In some embodiments, as described herein, one or more return pumps (e.g. pumps 2150 and/or 2250) are configured to draw fluid from balloon 136 while fluid is pumped into balloon 136 (e.g. via pumps 2130 and/or 2230). In these embodiments, the push and pull of fluid to and from balloon 136 enables a faster recirculating rate (e.g. the rate at which fluid recirculates through balloon 136) while limiting the pressure within balloon 136. A fast recirculating rate within balloon 136 helps ensure efficient and even thermal mixing and thermal delivery (e.g. transfer of thermal energy to the tissue to be ablated).

In some embodiments, multiple catheters 100 and/or connecting assemblies 300 are configured for use with console 200 (e.g. console 200 comprises reusable capital equipment, and catheters 100 and/or connecting assemblies 300 each comprise limited use disposable components), each of which can comprise varying flow resistances. In these embodiments, flow rate control may provide a more consistent ablation profile between various catheters 100 (e.g. more consistent as compared to pressure control). In some embodiments, one, two or more pumps of console 200 (e.g. pumps 2130, 2230, 2150 and/or 2250) are calibrated prior to a procedure, such as a treatment procedure described herein. For example, one, two or more pumps can be calibrated by measuring the flow rate of a fluid pumped through a fluid path with a known resistance, such as to determine the relationship between the RPM of the pump and the flow rate provided at that pump speed.

In some embodiments, one or more parameters of the treatment steps described herein are stored by controller 250 of console 200. For example, console 200 can store in memory of controller 250 a parameter selected from the group consisting of: the target temperature of hot fluid 2101; the duration of one or more steps, such as the duration of an ablative treatment step; the speed (e.g. flow rate) of one or more pumps during a treatment step; the duration of a cooling step; and combinations of these. In some embodiments, system 10 is configured to allow a user to perform a calibration procedure, such as to measure the performance of one or more treatment steps compared to a target performance level (e.g. an expected optimal performance), and to calibrate the parameters based on the comparison. In some embodiments, test assembly 295 (described herein in reference to FIG. 1) can comprise a calibration tool. In these embodiments, test assembly 295 can comprise a temperature sensor, a known thermal mass, and a known flow resistance element, and assembly 295 can be configured to fluidly attach to the inlet and outlet of console 200, such that a test ablative treatment step can be performed with the calibration fixture attached to console 200. In some embodiments, after the test ablative treatment step is performed, one or more parameters of the treatment step can be adjusted (e.g. the speed of hot pump 2130) to adjust (e.g. optimize or otherwise improve) the performance of one or more subsequent ablative treatment steps.

Referring now to FIGS. 16 and 16A-D, perspective, top, side, side sectional, and sectional views of an embodiment of a port for capturing tissue are illustrated, respectively, consistent with the present inventive concepts. FIG. 16D illustrates a sectional view along section A-A of FIG. 16C. Port 137 can be of similar construction and arrangement as port 137 as described herein in reference to FIG. 1 and/or port 27 as described herein in reference to FIG. 2. Port 137 comprises an elongate structure, port body 1371, with an opening 1372 positioned in the top surface of port 137 (e.g. the top surface being opposite a bottom surface that is oriented toward shaft 110 and attached to balloon 136 of functional assembly 130), where opening 1372 is sized and positioned to capture tissue in the performance of a tissue expansion procedure or other procedure in which fluid is to be delivered into tissue, as described herein. Port body 1371 can comprise a relatively flat bottom surface, and/or a bottom surface with a concave surface, such as to match the contour of and/or otherwise enhance attachment to balloon 136. Port body 1371 can comprise materials configured to provide a sufficient amount of rigidity to at least a portion of port body 1371 (e.g. the portion of port body 1371 surrounding opening 1372), such as when port body 1371 comprises one, two, or more materials that result in port body 1371 having a durometer of 63D or harder. Port body 1371 can be constructed of liquid crystal polymer.

Referring specifically to FIG. 16A, opening 1372 can comprise a width W1 that is less than or equal to 2.5 mm, or 2.0 mm, such as a width of approximately 1.58 mm. Opening 1372 can comprise a length L1 that is less than or equal to 5.0 mm, such as a length of approximately 3.55 mm. In some embodiments, opening 1372 comprises a cross sectional area of at least 3 mm², such as at least 4 mm², or at least 5 mm². Alternatively or additionally, opening 1372 can comprise a cross sectional area of no more than 30 mm², or no more than 15 mm². In some embodiments, opening 1372 is encompassed by one or more upward facing flat portions, surface 1374, comprising a portion of the wall of port 137 surrounding opening 1372 (e.g. flat portions created during a skiving or other procedure for creating opening 1372 in port 137). Surface 1374 can extend from the surface of port 137 at a controlled angle of between 90° and 175°, such as an angle between 90° and 150°, such as an angle between 132.5° and 137.5°, such as at an angle of approximately 135°. Alternatively, opening 1372 does not include surface 1374, such as when opening 1372 is created using a punch or other method leaving only vertical walls surrounding opening 1372. At the distal end of opening 1372 is a wall, surface 1375, which is positioned (e.g. relative to opening 1372) to prevent tissue that is captured within opening 1372 from traveling distally when a fluid delivery element 139c is advanced into tissue captured within opening 1372, such as to perform a tissue expansion procedure and/or otherwise deliver fluid into tissue, as described herein.

Referring specifically to FIG. 16B, opening 1372 can comprise a depth D1 of between 0.3 mm and 2.5 mm, such as 0.5 mm and 2.5 mm, such as a depth of approximately 1.4 mm.

Referring specifically to FIGS. 16C and 16D, port 137 can comprise lumens 1378 and 1379, such that lumen 1378 is positioned above lumen 1379 (e.g. lumens 1378 and 1379 are in a stacked arrangement). Lumens 1378 and 1379 can be constructed and arranged to terminate within or proximate opening 1372. Lumens 1378 and 1379 can each be operably attached to a conduit 111 of shaft 110. Lumen 1378 can comprise a relatively circular or other elliptical shaped cross sectional geometry, and lumen 1379 can comprise a crescent shaped cross sectional geometry, as shown. Lumen 1378 can be positioned above lumen 1379, such that the crescent shaped geometry of lumen 1379 relatively surrounds the cylindrical structure of lumen 1378. Opening 1372 includes vertical side walls 1373 (e.g. vertical walls created during a skiving, punch, molding, or other process for creating opening 1372 in port 137).

In some embodiments, as shown in FIGS. 16A-D, lumen 1378 is positioned between lumen 1379 and the top 137_{T} (shown in FIG. 16C) of port 137. This arrangement provides for improved penetration of tissue captured within opening 1372 by advancement of the associated fluid delivery element 139c (translating within lumen 1378) when vacuum is applied to lumen 1379. In some embodiments, lumen 1379 comprises a greater cross sectional area than lumen 1378. In some embodiments, lumen 1379 comprises a non-circular shape, such as a shape that partially surrounds lumen 1378 (as shown in FIG. 16D).

In some embodiments, the distance between the bottom of lumen 1378 and the bottom of opening 1372, distance D2 of FIG. 16C, comprises a distance greater than or equal to a minimum distance, such as to ensure that after intestinal tissue is captured within opening 1372 (e.g. as vacuum is applied via lumen 1379), advancement of fluid delivery element 139c causes the distal end of element 139c to reside within submucosal tissue (versus mucosal tissue) when fully advanced. In these embodiments, distance D2 can comprise a distance of at least 0.006″ and/or a distance greater than an average thickness of intestinal mucosa.

In some embodiments, the distance between the bottom of lumen 1378 and the bottom of opening 1372, distance D2 of FIG. 16C, comprises a distance less than or equal to a maximum distance, such as to ensure that after intestinal tissue is captured within opening 1372 (e.g. as vacuum is applied via lumen 1379), advancement of fluid delivery element 139c causes the distal end of element 139c to reside within submucosal tissue (versus deeper layers of intestinal tissue) when fully advanced. In these embodiments, distance D2 can comprise a distance of no more than 0.100″.

Referring now to FIGS. 17A-C, side sectional views of an embodiment of a port and an injectate delivery element advanced to different positions are illustrated, consistent with the present inventive concepts. Port 137 comprises opening 1372 and lumens 1378 and 1379. In some embodiments, a tube, such as a polyimide or other plastic tube, and/or a metal tube, sleeve 5040 shown, is positioned within the distal portion of lumen 1378. Sleeve 5040 includes a distal projection, distal stop 5041, and a proximal projection, proximal stop 5042. Distal stop 5041 and/or proximal stop 5042 can each comprise tubes (e.g. concentric tubes, such as concentric polyimide or other plastic tubes, and/or metal tubes) frictionally engaged within sleeve 5040, configured to reduce the inner diameter of sleeve 5040 at distal and proximal locations as shown. Sleeve 5040 can be constructed and arranged to slidingly receive a fluid delivery element 139c, such as needle 525 shown. Needle 525 is fluidly connected to conduit 521. For example, needle 525 is press fit into, bonded to, and/or welded to, conduit 521. Conduit 521 can be of similar construction and arrangement as conduit 111 as described herein in reference to FIG. 1. Needle 525 can comprise a lubricant, such as a silicone lubricant. In some embodiments, conduit 521 comprises an inner diameter that is less than the outer diameter of needle 525. Needle 525 can comprise a diameter that ranges from 16 gauge to 34 gauge, such as a needle with a 21 gauge diameter, a 23 gauge diameter, a 25 gauge diameter, or a 27 gauge diameter (e.g. with a 10° bevel angle). Needle 525 can comprise a beveled tip (as shown). The angle of the bevel of needle 525 can comprise an angle of between 5° and 45°, such as angle of approximately 10°. The length of the bevel of needle 525 can comprise a length that is less than the length of tissue captured in port 137. The rotational orientation of needle 525 about its longitudinal axis can be uncontrolled (e.g. the orientation is not controlled in the manufacturing process). Needle 525 can include a projection, needle ferrule 5045, such as a tube (e.g. a metal or plastic tube) frictionally engaged about a portion of needle 525. Ferrule 5045 can be slidingly received within sleeve 5040, between proximal stop 5042 and distal stop 5041. Sleeve 5040, proximal stop 5042, distal stop 5041, and ferrule 5045 can be sized such that ferrule 5045 (and therefore needle 525) slide freely proximally and distally between proximal stop 5042 and distal stop 5041, but proximal travel is limited when ferrule 5045 makes contact with proximal stop 5042 (e.g. when needle 525 is fully retracted as shown in FIG. 17A), and distal travel is limited when ferrule 5045 makes contact with distal stop 5041 (e.g. when needle 525 is fully advanced as shown in FIG. 17C). Proximal stop 5042, distal stop 5041, and ferrule 5045 are constructed and arranged to limit the distal most (e.g. retracted) and proximal most (e.g. advanced) position of needle 525.

In some embodiments, needle 525 is constructed and arranged to exit lumen 1378 between opening 1372 and lumen 1379, such that lumen 1379 can pull tissue from opening 1372 past needle 525 (e.g. past lumen 1378 from the top of). Additionally or alternatively, a vacuum applied via lumen 1379 can be configured to draw tissue proximally toward needle 525.

In FIG. 17A, needle 525 is fully retracted within sleeve 5040, such that ferrule 5045 is in contact with proximal stop 5042 and the tip of needle 525 is positioned within distal stop 5041 (i.e. does not extend into opening 1372). In this fully retracted position, vacuum can be applied to port 137, as described herein, causing tissue (e.g. tissue not shown for illustrative clarity, but at least mucosal and/or submucosal tissue of the intestine), to be drawn into opening 1372.

In FIG. 17B, needle 525 has been partially advanced within sleeve 5040, such that the tip of needle 525 slightly extends beyond the distal end of distal stop 5041, into opening 1372. In FIG. 17C, needle 525 has been fully advanced within sleeve 5040, such that the tip of needle 525 is extending into opening 1372 and ferrule 5045 is in contact with distal stop 5041. When needle 525 is fully advanced, the distal end of needle 525 can be relatively centered in opening 1372. When needle 525 is fully advanced, needle 525 does not exit port 137 (e.g. needle 525 remains within port body 1371). Needle 525 can be configured to traverse a travel length D1 of between 1 mm and 8 mm, such as a length of approximately 4 mm (e.g. needle 525 travels approximately 4 mm from the fully retracted position as shown in FIG. 17A to the fully advanced position as shown in FIG. 17C). In the fully advanced position, needle 525 can comprise an exposed length D2 of between 0.5 mm and 8 mm, such as a length between 2 mm and 2.5 mm (e.g. the tip of needle 525 extends into opening 1372 by between 2 mm and 2.5 mm). In the fully advanced position, when tissue has been captured in opening 1372, injectate 221 can be delivered via needle 525 into the tissue, as described herein, such as to expand the tissue to create a restriction (e.g. a therapeutic restriction), and/or prepare the tissue (e.g. create a safety margin of tissue) for a subsequent tissue ablation procedure.

In some embodiments, catheter 100 is configured to be used in a single procedure, wherein each needle 525 is advanced into tissue no more than 30 times per procedure, such as no more than 20 times per procedure.

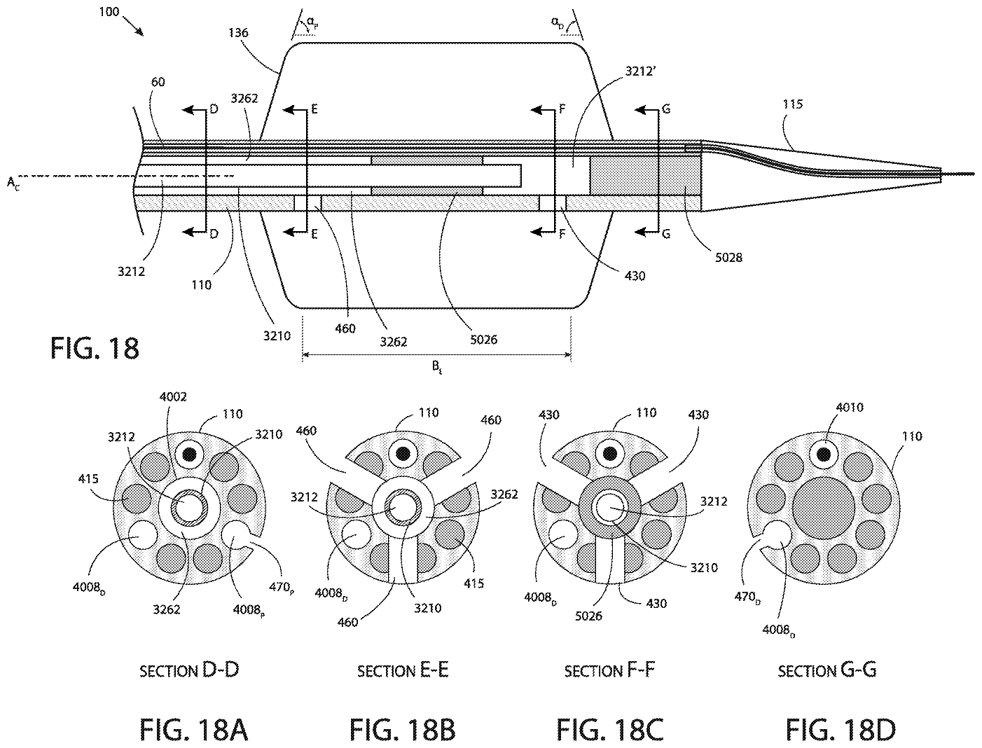

Referring now to FIG. 18, a side sectional view of an embodiment of the distal portion of catheter 100 is illustrated, consistent with the present inventive concepts. In FIGS. 18A-D, cross sectional views of the distal portion of catheter 100 is illustrated, where FIG. 18A is a sectional view at section D-D, FIG. 18B is a sectional view at section E-E, FIG. 18C is a sectional view at section F-F, and FIG. 18D is a sectional view at section G-G. Catheter 100 comprises a tapered distal tip, tip 115, which can comprise a material such as polyether block amide, and a cone-like (e.g. a hollow cone) construction, a double cone (e.g. an inner and an outer cone) construction, or a solid (e.g. not hollow) construction. Tip 115 can comprise a taper or other construction that causes tip 115 to have a variable stiffness, such as to improve translation of tip 115 in the GI tract (e.g. when catheter 100 is advanced over a guidewire 60).

In some embodiments, shaft 110 comprises a multi-lumen shaft, each lumen comprising a conduit, similar to conduits 111 described herein in reference to FIG. 1. Shaft 110 can comprise various conduits and/or lumens as shown in FIG. 18, such as: lumen 4010 configured to slidingly receive guidewire 60; lumen 4008_{D} configured to operably attach to console 200 to provide aspiration and/or insufflation distal to balloon 136; lumen 4008p configured to operably attach to console 200 to provide aspiration and/or insufflation proximal to balloon 136; central lumen 4002 configured to receive conduit 3210 which is configured to provide fluid to and/or extract fluid from balloon 136 via lumen 3212; lumen 3262 which surrounds conduit 3210 and is configured to provide fluid to and/or extract fluid from balloon 136; and one or more additional satellite lumens (e.g. lumens surrounding central lumen 4002 as shown).

In some embodiments, conduit 3210 comprises at least one insulative wall, such that a fluid within lumen 3212 is insulated from a fluid within lumen 3262.

At locations at and distal to Section D-D (“distal to Section D-D” herein), one or more lumens of shaft 110 are no longer used (e.g. to transport fluid or other material). In some embodiments, one or more of the distal segments (segments distal to section D-D) of the unused lumens are occluded, such as with a sealing element, element 415 (e.g. an adhesive, potting material, plug or filament), as shown in FIGS. 18A-D.

At location D-D, an opening, port 470p is positioned between lumen 4008p and the outer surface of shaft 110. Port 470p can be configured as a port for insufflation (e.g. providing fluids for insufflation and/or removing fluids for desufflation), at a luminal location (e.g. a GI luminal location) proximal to balloon 136. Distal to location D-D, lumen 4008p is also filled with a sealing element 415, as shown in FIGS. 18B-D, such as to direct (e.g. limit) the insufflation source of lumen 4008p to port 470p.

At location E-E, one or more openings, fluid removal ports 460, (three shown) are positioned between lumen 3262 and the outer surface of shaft 110, fluidly connecting lumen 3262 to the interior of balloon 136. Ports 460 are created (e.g. punched, skived, and/or drilled) from the outer surface of shaft 110, thru the wall of shaft 110, and thru at least one of the sealing elements 415 (two shown per port in FIG. 18B). Fluid removal ports 460 are fluidly connected to lumen 3262, such that fluid can be removed from balloon 136 via ports 460. Distal to location E-E, a sealing element 5026 is positioned within lumen 3262, between the outer surface of conduit 3210 and the inner surface of central lumen 4002, as shown in FIGS. 18 and 18C-D. Seal 5026 is configured to seal the distal end of lumen 3262 distal to ports 460, and proximal to ports 430, described herein.

In some embodiments, ports 460 comprise an opening that is larger than the cross sectional area of lumens 3212, 3262, such that the opening of ports 460 do not constrict the flow of fluid into lumens 3212, 3262. In some embodiments, ports 460 comprise 2, 3, 4 or more openings.

In some embodiments, ports 460 are equally spaced about the circumference of shaft 110. For example, three ports 460 can be separated by approximately 120 degrees to provide an equal distribution of ports 460 about shaft 110.

At location F-F, one or more openings, inflation ports 430, (3 shown) are positioned between the outer surface of shaft 110, and a space, chamber 3212′, created between seal 5026 and a distal seal 5028 shown in FIG. 18, configured to seal the distal end of lumen 4002. Chamber 3212′ is fluidly connected to lumen 3212 of conduit 3210. Ports 430 are created (e.g. punched, skived, and/or drilled) from the outer surface of shaft 110, thru the wall of the shaft, and thru at least one of the sealing elements 415 (two shown per port in FIG. 18C). Conduit 3210 terminates within and is fluidly attached to chamber 3212′, which is fluidly attached to the interior of balloon 136 via ports 430, such that fluid can be inserted into balloon 136 from console 200.

In some embodiments, ports 430 comprise an opening that is larger than the cross sectional area of lumens 3212, 3262, such that the openings of ports 430 do not constrict the flow of fluid. In some embodiments, ports 430 comprise 2, 3, 4 or more openings.

In some embodiments, ports 430 are equally spaced about the circumference of shaft 110. For example, three ports 430 can be separated by approximately 120 degrees to provide an equal distribution of ports 430 about shaft 110.

At location G-G, an opening, port 470_{D} is positioned between lumen 4008_{D} and the outer surface of shaft 110. Port 470_{D} can be configured as a port for insufflation (e.g. providing fluids for insufflation and/or removing fluids for desufflation), at a luminal location (e.g. a GI luminal location) distal to balloon 136. Distal to location G-G, lumen 4008_{D} is also filled with a sealing element 415, not shown, such as to direct (e.g. limit) the insufflation source of lumen 4008_{D} to port 470_{D}.

Balloon 136 can comprise a tissue contacting portion, BL. BL can comprise a length of between 15 mm and 25 mm, such as a tissue contacting length of approximately 20 mm.

Balloon 136 (e.g. a balloon when inflated) can comprise a tapered distal end (angle α_{D} as shown), and/or a tapered proximal end (angle α_{P} also as shown). Taper angles α_{D} and α_{P} can comprise similar or dissimilar angles. Balloon 136 can comprise a taper angle of at least 5° and/or no more than 120°, such as a taper angle of at least 30° and/or no more than 90°, such as a taper angle of at least 57° and/or no more than 63°, such as a taper angle of approximately 60°.

Referring now to FIG. 19, a schematic view of another embodiment of a system for performing a medical procedure in the intestine of a patient is illustrated, according to the present inventive concepts. In some embodiments, system 10 shown in FIG. 19 is of similar construction and arrangement as applicant's co-pending U.S. patent application Ser. No. 16/742,645, entitled “Intestinal Catheter Device and System”, filed Jan. 14, 2020.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth below not be construed as being order-specific unless such order specificity is expressly stated in the claim.

1. (canceled)

2. A system for performing a medical procedure in the intestine of a patient, the system comprising:
a catheter for insertion into the intestine, the catheter comprising:
a shaft including a distal portion; and
a functional assembly on the distal portion of the shaft, wherein the functional assembly is configured to receive a fluid; and
a console comprising:
a connector configured to operably attach the catheter to the console; and
at least one pumping assembly configured to deliver the fluid to the functional assembly, wherein the fluid comprising ablative fluid and neutralizing fluid;
wherein the system is configured to treat a first segment of the intestine via the console delivering the ablative fluid and the neutralizing fluid to the functional assembly while the functional assembly is positioned proximate the first segment of the intestine,
wherein the console delivers the ablative fluid to the functional assembly positioned in the first segment of the intestine based on one or more ablative fluid delivery parameters,
wherein the one or more ablative fluid delivery parameters are determined based on at least a first set of one or more system parameters, and
wherein the first set of one or more system parameters comprise at least one of:
ambient temperature within the console; and/or
temperature of the room in which the console is positioned.

3. The system according to claim 2, wherein the console is configured to deliver the neutralizing fluid to the functional assembly after having delivered the ablative fluid to the functional assembly.

4. The system according to claim 3, wherein the console is further configured to deliver the neutralizing fluid to the functional assembly prior to delivering the ablative fluid to the functional assembly.

5. The system according to claim 2, wherein the console is configured to deliver the neutralizing fluid to the functional assembly prior to delivering the ablative fluid to the functional assembly.

6. The system according to claim 2, wherein the ablative fluid comprises fluid at a temperature above body temperature of the patient.

7. The system according to claim 6, wherein the ablative fluid comprises fluid above 60° C.

8. The system according to claim 6, wherein the neutralizing fluid comprises fluid at a temperature below body temperature of the patient.

9. The system according to claim 8, wherein the neutralizing fluid comprises fluid at a temperature of approximately the temperature of the room in which the console is positioned.

10. The system according to claim 2, wherein the ablative fluid comprises cryogenic fluid.

11. The system according to claim 2, wherein the first set of one or more ablative fluid delivery parameters comprises at least the temperature of the ablative fluid provided by the console.

12. The system according to claim 2, wherein the first set of one or more ablative fluid delivery parameters comprises at least the flow rate that the ablative fluid is provided to the functional assembly by the console.

13. The system according to claim 2, wherein the first set of one or more ablative fluid delivery parameters comprises at least the duration in which the ablative fluid is positioned within the functional assembly during the treatment of the first segment of the intestine.

14. The system according to claim 2, wherein the first set of one or more ablative fluid delivery parameters comprises at least the duration in which the neutralizing fluid is positioned within the functional assembly during the treatment of the first segment of the intestine.

15. The system according to claim 2, wherein the first set of one or more ablative fluid delivery parameters comprises at least the pressure of fluid delivered to the functional assembly by the console.

16. The system according to claim 2, wherein the first set of one or more ablative fluid delivery parameters are dynamically adjusted during the treatment of the first segment of the intestine.

17. The system according to claim 2, wherein the first set of one or more ablative fluid delivery parameters are adjusted in a closed loop arrangement.

18. The system according to claim 2, wherein the first set of one or more system parameters further comprises at least one parameter selected from the group consisting of: altitude of the room in which the console is positioned; patient temperature; the temperature of the neutralizing fluid; the temperature of the ablative fluid; and combinations thereof.

19. The system according to claim 2, wherein the fluid delivered to the functional assembly is returned via the catheter shaft to the console, and wherein the first set of one or more system parameters further comprises the temperature of fluid returning from the catheter to the console.

20. The system according to claim 2, wherein the console delivers the neutralizing fluid to the functional assembly positioned in the first segment of the intestine based on one or more neutralizing fluid delivery parameters, and
wherein the one or more neutralizing fluid delivery parameters are determined based on the first set of one or more system parameters.

21. The system according to claim 2, wherein the system is further configured to treat a second segment of the intestine via the console delivering the ablative fluid and the neutralizing fluid to the functional assembly while the functional assembly is positioned proximate the second segment of the intestine, and wherein the console delivers the ablative fluid to the functional assembly positioned in the second segment of the intestine based a second set of one or more ablative fluid delivery parameters.

22. The system according to claim 21, wherein the second set of one or more system parameters comprise at least the duration of time since the treatment of the first segment of the intestine.

23. The system according to claim 21, wherein the fluid delivered to the functional assembly is returned via the catheter shaft to the console, and wherein the second set of one or more system parameters comprise at least the temperature of fluid returning from the catheter to the console.

24. The system according to claim 2, further comprising one or more sensors, wherein each sensor is configured to produce a signal, and wherein the system is configured to determine the first set of one or more system parameters based on the signal from each sensor.

25. The system according to claim 24, wherein the one or more sensors comprises at least a temperature sensor.

26. The system according to claim 25, wherein the temperature sensor comprises a resistance temperature detector or an optical temperature sensor.

27. The system according to claim 25, wherein the temperature sensor is configured to produce a signal related to one or more temperatures selected from the group consisting of: temperature of fluid in the console; temperature of the catheter shaft; temperature of fluid within the catheter shaft; temperature of the functional assembly; temperature of a fluid within the functional assembly of the catheter; temperature of the ablative fluid; temperature of the neutralizing fluid; temperature of tissue proximate the functional assembly; temperature of target tissue; temperature of non-target tissue; and combinations thereof.

28. The system according to claim 25, wherein the temperature sensor comprises a first temperature sensor and a second temperature sensor.

29. The system according to claim 28, wherein the first temperature sensor is configured to measure the temperature associated with a first parameter of the system and the second temperature sensor is configured to measure the temperature associated with a second parameter of the system.

30. The system according to claim 24, wherein the one or more sensors comprises at least a pressure sensor.

31. The system according to claim 2, wherein the at least one pumping assembly comprises at least one syringe pump.

32. The system according to claim 2, wherein the medical procedure performed by the system is configured to treat insulin resistance, diabetes, and/or a metabolic condition of the patient.

33. The system according to claim 2, wherein the functional assembly is configured to radially expand and/or contract.

34. The system according to claim 33, wherein the functional assembly comprises a balloon that receives the ablative fluid and the neutralizing fluid.

35. The system according to claim 33, wherein the functional assembly is configured to expand tissue of the first segment of the intestine prior to the delivery of the ablative fluid to the functional assembly.
